SEQUENCE LISTING (655 sequences)

SEQ ID NO: 1 - skipped

SEQ ID NO: 2 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSGIELRHFLNHLTRQRY

SEQ ID NO: 3 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGQRSYASLRHYYNWLTRQRY

SEQ ID NO: 4 - protein, 49 aa, synthetic construct
  SITE 2: note = Amino-isobutyric acid
  SITE 13: note = Amino-isobutyric acid
  SITE 20: note = Amino-isobutyric acid
  SITE 37: note = Amino-isobutyric acid
  SITE 41: note = Amino-isobutyric acid
  SITE 44: note = Amino-isobutyric acid
YXEGTFTSDYSIXLEKQAQXAFVEWLIKGGPSSGASXRHYXNRXTRQRY

SEQ ID NO: 5 - protein, 49 aa, synthetic construct
  SITE 2: note = Amino-isobutyric acid
  SITE 13: note = Amino-isobutyric acid
  SITE 20: note = Amino-isobutyric acid
  SITE 37: note = D-Leucine
  SITE 39: note = D-Histidine
  SITE 46: note = D-Arginine
  SITE 49: note = D-Tyrosine
YXEGTFTSDYSIXLEKQAQXAFVEWLIKGGPSSGASLRHYYNWLTRQRY

SEQ ID NO: 6 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGQRYYASLRHYYNWLTRQRY

SEQ ID NO: 7 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEEEAQXAFVQWLIKGGPSSGASLRHYYNWLTRQRY

SEQ ID NO: 8 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEEQAQXAFVQWLIKGGPSSGQSLRHYYNWLTRQRY

SEQ ID NO: 9 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSYVSIRHYYNWLTRQRY

SEQ ID NO: 10 - protein, 49 aa, synthetic construct
  SITE 2: note = Amino-isobutyric acid
  SITE 13: note = Amino-isobutyric acid
  SITE 20: note = Amino-isobutyric acid
  SITE 37: note = Amino-isobutyric acid
YXEGTFTSDYSIXLEKQAQXAFVEWLIKGGPSSPESXRHYYNWLTRQRY

SEQ ID NO: 11 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGPESPASLRHYYNWLTRQRY

SEQ ID NO: 12 - protein, 49 aa, synthetic construct; identical to SEQ ID NO: 11
  SITE 2: note = Amino-isobutyric acid
  SITE 13: note = Amino-isobutyric acid
  SITE 20: note = Amino-isobutyric acid

SEQ ID NO: 13 - protein, 49 aa, synthetic construct
  SITE 2: note = Amino-isobutyric acid
  SITE 13: note = Amino-isobutyric acid
  SITE 20: note = Amino-isobutyric acid
  SITE 37: note = Amino-isobutyric acid
  SITE 44: note = Amino-isobutyric acid
YXEGTFTSDYSIXLEKQAQXAFVEWLIKGGPSSGASXRHYYNRXTRQRY

SEQ ID NO: 14 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGPRYYASLRHYYNWLTRQRY

SEQ ID NO: 15 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGPSSYVSLRHYYNWLTRQRY

SEQ ID NO: 16 - protein, 49 aa, synthetic construct
  SITE 2: note = Amino-isobutyric acid
  SITE 13: note = Amino-isobutyric acid
  SITE 16: note = Amino-isobutyric acid
  SITE 20: note = Amino-isobutyric acid
YXEGTFTSDYSIXLEXEAQXKFVEWLIKGGPSSGASLRHYYNWLTRQRY

SEQ ID NO: 17 - protein, 49 aa, synthetic construct
  SITE 2: note = Amino-isobutyric acid
  SITE 13: note = Amino-isobutyric acid
  SITE 20: note = Amino-isobutyric acid
  SITE 44: note = Amino-isobutyric acid
YXEGTFTSDYSIXLEKQAQXAFVEWLIKGGPSSGASLRHYLNLXTRQRY

SEQ ID NO: 18 - protein, 49 aa, synthetic construct
  SITE 2: note = Amino-isobutyric acid
  SITE 13: note = Amino-isobutyric acid
  SITE 20: note = Amino-isobutyric acid
  SITE 41: note = Amino-isobutyric acid
  SITE 44: note = Amino-isobutyric acid
YXEGTFTSDYSIXLEKQAQXAFVEWLIKGGPSSGASLRHYXNRXTRQRY

SEQ ID NO: 19 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGPSYYVSLRHYYNWLTRQRY

SEQ ID NO: 20 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLAAAAQXAFVEWLIKGGPSSGASLRHYYNWLTRQRY

SEQ ID NO: 21 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 39: note = D-Histidine
  SITE 46: note = D-Arginine
  SITE 49: note = D-Tyrosine

SEQ ID NO: 22 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGQSSYASLRHYYNWLTRQRY

SEQ ID NO: 23 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGPEYYASLRHYYNWLTRQRY

SEQ ID NO: 24 - protein, 49 aa, synthetic construct
  SITE 2: note = Amino-isobutyric acid
  SITE 13: note = Amino-isobutyric acid
  SITE 16: note = Amino-isobutyric acid
  SITE 20: note = Amino-isobutyric acid
YXEGTFTSDYSIXLEXKAQXEFVEWLIKGGPSSGASLRHYYNWLTRQRY

SEQ ID NO: 25 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEAAKQXAFVEWLIKGGPSSGASLRHYYNWLTRQRY

SEQ ID NO: 26 - protein, 49 aa, synthetic construct
  SITE 2: note = Amino-isobutyric acid
  SITE 13: note = Amino-isobutyric acid
  SITE 20: note = Amino-isobutyric acid
  SITE 37: note = Amino-isobutyric acid
  SITE 46: note = Homo-L-arginine
  SITE 49: note = D-Tyrosine
YXEGTFTSDYSIXLEKQAQXAFVEWLIKGGPSSGASXRHYYNWLTXQRY

SEQ ID NO: 27 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSGVSIRHYYNWLTRQRY

SEQ ID NO: 28 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXEFVQWLIKGGPSSGASLRHYYNWLTRQRY

SEQ ID NO: 29 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGPESGASLRHYYNWLTRQRY

SEQ ID NO: 30 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEEQAQXAFVQWLIKGGPSSGASLRHYYNWLTRQRY

SEQ ID NO: 31 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEEQAQXAFVEWLIKGGPSSGQSLRHYYNWLTRQRY

SEQ ID NO: 32 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid

SEQ ID NO: 33 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSAVSLRHYYNWLTRQRY

SEQ ID NO: 34 - protein, 49 aa, synthetic construct; identical to SEQ ID NO: 30
  SITE 2: note = Amino-isobutyric acid
  SITE 13: note = Amino-isobutyric acid
  SITE 20: note = Amino-isobutyric acid

SEQ ID NO: 35 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGPSSGESLRHYYNWLTRQRY

SEQ ID NO: 36 - protein, 49 aa, synthetic construct; identical to SEQ ID NO: 28
  SITE 2: note = Amino-isobutyric acid
  SITE 13: note = Amino-isobutyric acid
  SITE 20: note = Amino-isobutyric acid

SEQ ID NO: 37 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid

SEQ ID NO: 38 - protein, 49 aa, synthetic construct
  SITE 2: note = Amino-isobutyric acid
  SITE 13: note = Amino-isobutyric acid
  SITE 20: note = Amino-isobutyric acid
  SITE 37: note = Amino-isobutyric acid
YXEGTFTSDYSIXLEKQAQXAFVEWLIKGGPSSGVSXRHYYNWLTRQRY

SEQ ID NO: 39 - protein, 49 aa, synthetic construct
  SITE 2: note = Amino-isobutyric acid
  SITE 13: note = Amino-isobutyric acid
  SITE 20: note = Amino-isobutyric acid
  SITE 37: note = Amino-isobutyric acid
  SITE 48: note = N-methyl-L-arginine
YXEGTFTSDYSIXLEKQAQXAFVEWLIKGGPSSGASXRHYYNWLTRQRY

SEQ ID NO: 40 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSYVSLRHYYNWLTRQRY

SEQ ID NO: 41 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSPQSLRHYYNWLTRQRY

SEQ ID NO: 42 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSEYVSIRHYYNWLTRQRY

SEQ ID NO: 43 - protein, 49 aa, synthetic construct; identical to SEQ ID NO: 28
  SITE 2: note = Amino-isobutyric acid
  SITE 13: note = Amino-isobutyric acid
  SITE 20: note = Amino-isobutyric acid

SEQ ID NO: 44 - protein, 49 aa, synthetic construct
  SITE 2: note = Amino-isobutyric acid
  SITE 13: note = Amino-isobutyric acid
  SITE 20: note = Amino-isobutyric acid
  SITE 37: note = Amino-isobutyric acid
YXEGTFTSDYSIXLEKQAQXAFVEWLIKGGPSSGQSXRHYYNWLTRQRY

SEQ ID NO: 45 - protein, 49 aa, synthetic construct; identical to SEQ ID NO: 30
  SITE 2: note = Amino-isobutyric acid
  SITE 13: note = Amino-isobutyric acid
  SITE 20: note = Amino-isobutyric acid

SEQ ID NO: 46 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKEAQXKFVEWLIKGGPSSGASLRHYYNWLTRQRY

SEQ ID NO: 47 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGPSSGVSLRHYYNWLTRQRY

SEQ ID NO: 48 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSEPQSIRHYYNWLTRQRY

SEQ ID NO: 49 - protein, 49 aa, synthetic construct
  SITE 2: note = Amino-isobutyric acid
  SITE 13: note = Amino-isobutyric acid
  SITE 20: note = Amino-isobutyric acid
  SITE 37: note = Amino-isobutyric acid
YXEGTFTSDYSIXLEKQAQXAFVEWLIKGGPSSGESXRHYYNWLTRQRY

SEQ ID NO: 50 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSYESLRHYYNWLTRQRY

SEQ ID NO: 51 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAEXAFVQWLIKGGPSSGASLRHYYNWLTRQRY

SEQ ID NO: 52 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid

SEQ ID NO: 53 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid

SEQ ID NO: 54 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAEXAFVQWLIKGGPSEPASLRHYYNWLTRQRY

SEQ ID NO: 55 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid

SEQ ID NO: 56 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXEFVEWLIKGGPSSPASLRHYYNWLTRQRY

SEQ ID NO: 57 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGPEEPASLRHYYNWLTRQRY

SEQ ID NO: 58 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid

SEQ ID NO: 59 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGPSYYASLRHYYNWLTRQRY

SEQ ID NO: 60 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid

SEQ ID NO: 61 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid

SEQ ID NO: 62 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPRYYASLRHYYNWLTRQRY

SEQ ID NO: 63 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid

SEQ ID NO: 64 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPESPASLRHYYNWLTRQRY

SEQ ID NO: 65 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGGSEYVSLRHYYNWLTRQRY

SEQ ID NO: 66 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEEEAQXAFVEWLIKGGPSSGASLRHYYNWLTRQRY

SEQ ID NO: 67 - protein, 49 aa, synthetic construct; identical to SEQ ID NO: 57
  SITE 2: note = Amino-isobutyric acid
  SITE 13: note = Amino-isobutyric acid
  SITE 20: note = Amino-isobutyric acid

SEQ ID NO: 68 - protein, 49 aa, synthetic construct
  SITE 2: note = Amino-isobutyric acid
  SITE 13: note = Amino-isobutyric acid
  SITE 20: note = Amino-isobutyric acid
  SITE 44: note = Amino-isobutyric acid
YXEGTFTSDYSIXLEKQAQXAFVEWLIKGGPSSGASLRHYYNRXTRQRY

SEQ ID NO: 69 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKEKQXAFVEWLIKGGPSSGASLRHYYNWLTRQRY

SEQ ID NO: 70 - protein, 49 aa, synthetic construct; identical to SEQ ID NO: 64
  SITE 2: note = Amino-isobutyric acid
  SITE 13: note = Amino-isobutyric acid
  SITE 20: note = Amino-isobutyric acid

SEQ ID NO: 71 - protein, 49 aa, synthetic construct
  SITE 2: note = Amino-isobutyric acid
  SITE 13: note = Amino-isobutyric acid
  SITE 20: note = Amino-isobutyric acid
  SITE 46: note = Homo-L-arginine
  SITE 49: note = D-Tyrosine
YXEGTFTSDYSIXLEKQAQXAFVEWLIKGGPSSGASLRHYYNWLTXQRY

SEQ ID NO: 72 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSGADLRHYLNWLTRQRY

SEQ ID NO: 73 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSPESLRHYYNWLTRQRY

SEQ ID NO: 74 - protein, 49 aa, synthetic construct; identical to SEQ ID NO: 39
  SITE 2: note = Amino-isobutyric acid
  SITE 13: note = Amino-isobutyric acid
  SITE 20: note = Amino-isobutyric acid
  SITE 37: note = Amino-isobutyric acid
  SITE 49: note = D-Tyrosine

SEQ ID NO: 75 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSYYVSLRHYYNWLTRQRY

SEQ ID NO: 76 - protein, 49 aa, synthetic construct
  SITE 2: note = Amino-isobutyric acid
  SITE 13: note = Amino-isobutyric acid
  SITE 20: note = Amino-isobutyric acid
  SITE 37: note = Amino-isobutyric acid
  SITE 41: note = Amino-isobutyric acid
YXEGTFTSDYSIXLEKQAQXAFVEWLIKGGPSSGASXRHYXNWLTRQRY

SEQ ID NO: 77 - protein, 49 aa, synthetic construct; identical to SEQ ID NO: 57
  SITE 2: note = Amino-isobutyric acid
  SITE 13: note = Amino-isobutyric acid
  SITE 20: note = Amino-isobutyric acid

SEQ ID NO: 78 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGPSEYVSLRHYYNWLTRQRY

SEQ ID NO: 79 - protein, 49 aa, synthetic construct
  SITE 2: note = Amino-isobutyric acid
  SITE 13: note = Amino-isobutyric acid
  SITE 20: note = Amino-isobutyric acid
  SITE 44: note = Amino-isobutyric acid
YXEGTFTSDYSIXLEKQAQXAFVEWLIKGGPSSGASLRHYYNLXTRQRY

SEQ ID NO: 80 - protein, 49 aa, synthetic construct; identical to SEQ ID NO: 75
  SITE 2: note = Amino-isobutyric acid
  SITE 13: note = Amino-isobutyric acid
  SITE 20: note = Amino-isobutyric acid

SEQ ID NO: 81 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXEFVEWLIKGGPESGASLRHYYNWLTRQRY

SEQ ID NO: 82 - protein, 49 aa, synthetic construct; identical to SEQ ID NO: 64
  SITE 2: note = Amino-isobutyric acid
  SITE 13: note = Amino-isobutyric acid
  SITE 20: note = Amino-isobutyric acid

SEQ ID NO: 83 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 48: note = N-methyl-L-arginine

SEQ ID NO: 84 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSGQSLRHYYNWLTRQRY

SEQ ID NO: 85 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 47: note = N-methyl-L-glutamine

SEQ ID NO: 86 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSGASLRHYLNWLTRQRY

SEQ ID NO: 87 - protein, 49 aa, synthetic construct
  SITE 2: note = Amino-isobutyric acid
  SITE 13: note = Amino-isobutyric acid
  SITE 20: note = Amino-isobutyric acid
YXDGTFTSDYSIXLEKQAQXAFVEWLIKGGPSSGASLRHYYNWLTRQRY

SEQ ID NO: 88 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXEFVEWLIKGGPSSGASLRHYYNWLTRQRY

SEQ ID NO: 89 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGPEEGASLRHYYNWLTRQRY

SEQ ID NO: 90 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEDQAQXAFVEWLIKGGPSSGASLRHYYNWLTRQRY

SEQ ID NO: 91 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSGATLRHYYNWLTRQRY

SEQ ID NO: 92 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGPSSGASLRHYYNWLTRQRY

SEQ ID NO: 93 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSEYESLRHYYNWLTRQRY

SEQ ID NO: 94 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSGISLRHYYNWLTRQRY

SEQ ID NO: 95 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSPASLRHYYNWLTRQRY

SEQ ID NO: 96 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSGVSLRHYYNWLTRQRY

SEQ ID NO: 97 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEEQAQXAFVEWLIKGGPSSGASLRHYYNWLTRQRY

SEQ ID NO: 98 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSAASLRHYYNWLTRQRY

SEQ ID NO: 99 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEGQAQXAFVEWLIKGGPSSGASLRHYYNWLTRQRY

SEQ ID NO: 100 - protein, 49 aa, synthetic construct; identical to SEQ ID NO: 97
  SITE 2: note = Amino-isobutyric acid
  SITE 13: note = Amino-isobutyric acid
  SITE 20: note = Amino-isobutyric acid

SEQ ID NO: 101 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGPSEPASLRHYYNWLTRQRY

SEQ ID NO: 102 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEAQAQXAFVEWLIKGGPSSGASLRHYYNWLTRQRY

SEQ ID NO: 103 - protein, 49 aa, synthetic construct; identical to SEQ ID NO: 39
  SITE 2: note = Amino-isobutyric acid
  SITE 13: note = Amino-isobutyric acid
  SITE 20: note = Amino-isobutyric acid
  SITE 37: note = L-tert-butylglycine

SEQ ID NO: 104 - protein, 49 aa, synthetic construct
  SITE 2: note = Amino-isobutyric acid
  SITE 20: note = Amino-isobutyric acid
YXEGTFTSDYSILLEKQAQXAFVEWLIKGGPSSGASLRHYYNWLTRQRY

SEQ ID NO: 105 - protein, 49 aa, synthetic construct; identical to SEQ ID NO: 97
  SITE 2: note = Amino-isobutyric acid
  SITE 13: note = Amino-isobutyric acid
  SITE 20: note = Amino-isobutyric acid

SEQ ID NO: 106 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSGASLRHYINWLTRQRY

SEQ ID NO: 107 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSGLSLRHYYNWLTRQRY

SEQ ID NO: 108 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid

SEQ ID NO: 109 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLLKGGPSSGASLRHYYNWLTRQRY

SEQ ID NO: 110 - protein, 49 aa, synthetic construct; identical to SEQ ID NO: 97
  SITE 2: note = Amino-isobutyric acid
  SITE 13: note = Amino-isobutyric acid
  SITE 20: note = Amino-isobutyric acid

SEQ ID NO: 111 - protein, 49 aa, synthetic construct; identical to SEQ ID NO: 101
  SITE 2: note = Amino-isobutyric acid
  SITE 13: note = Amino-isobutyric acid
  SITE 20: note = Amino-isobutyric acid

SEQ ID NO: 112 - protein, 49 aa, synthetic construct; identical to SEQ ID NO: 92
  SITE 2: note = Amino-isobutyric acid
  SITE 13: note = Amino-isobutyric acid
  SITE 20: note = Amino-isobutyric acid

SEQ ID NO: 113 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDLSIXLEKQAQXAFVQWLIKGGPSEGASLRHYYNWLTRQRY

SEQ ID NO: 114 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGPSEGVSLRHYYNWLTRQRY

SEQ ID NO: 115 - protein, 49 aa, synthetic construct
  SITE 2: note = Amino-isobutyric acid
  SITE 13: note = Amino-isobutyric acid
  SITE 20: note = Amino-isobutyric acid
  SITE 47: note = N-methyl-L-glutamine
YXEGTFTSDYSIXLEKQAQXAFVQWLIKGGPSEGASLRHYYNWLTRQRY

SEQ ID NO: 116 - protein, 49 aa, synthetic construct; identical to SEQ ID NO: 97
  SITE 2: note = Amino-isobutyric acid
  SITE 13: note = Amino-isobutyric acid
  SITE 20: note = Amino-isobutyric acid

SEQ ID NO: 117 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSGASVRHYYNWLTRQRY

SEQ ID NO: 118 - protein, 49 aa, synthetic construct; identical to SEQ ID NO: 39
  SITE 2: note = Amino-isobutyric acid
  SITE 13: note = Amino-isobutyric acid
  SITE 20: note = Amino-isobutyric acid
  SITE 37: note = Amino-isobutyric acid

SEQ ID NO: 119 - protein, 49 aa, synthetic construct
  SITE 2: note = Amino-isobutyric acid
  SITE 13: note = Amino-isobutyric acid
  SITE 20: note = Amino-isobutyric acid
  SITE 39: note = L-tert-butylglycine
YXEGTFTSDYSIXLEKQAQXAFVEWLIKGGPSSGASLRXYYNWLTRQRY

SEQ ID NO: 120 - protein, 49 aa, synthetic construct; identical to SEQ ID NO: 95
  SITE 2: note = Amino-isobutyric acid
  SITE 13: note = Amino-isobutyric acid
  SITE 20: note = Amino-isobutyric acid

SEQ ID NO: 121 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLESQAQXAFVEWLIKGGPSSGASLRHYYNWLTRQRY

SEQ ID NO: 122 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSEPESLRHYYNWLTRQRY

SEQ ID NO: 123 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGQSSGASLRHYYNWLTRQRY

SEQ ID NO: 124 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEQQAQXAFVEWLIKGGPSSGASLRHYYNWLTRQRY

SEQ ID NO: 125 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 48: note = N-methyl-L-arginine

SEQ ID NO: 126 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSYASLRHYYNWLTRQRY

SEQ ID NO: 127 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSGESLRHYYNWLTRQRY

SEQ ID NO: 128 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLDKQAQXAFVEWLIKGGPSSGASLRHYYNWLTRQRY

SEQ ID NO: 129 - protein, 49 aa, synthetic construct
  SITE 2: note = Amino-isobutyric acid
  SITE 13: note = Amino-isobutyric acid
  SITE 20: note = Amino-isobutyric acid
  SITE 38: note = Amino-isobutyric acid
YXEGTFTSDYSIXLEKQAQXAFVEWLIKGGPSSGASLXHYYNWLTRQRY

SEQ ID NO: 130 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFIEWLIKGGPSSGASLRHYYNWLTRQRY

SEQ ID NO: 131 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLERQAQXAFVEWLIKGGPSSGASLRHYYNWLTRQRY

SEQ ID NO: 132 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSGASIRHYYNWLTRQRY

SEQ ID NO: 133 - protein, 49 aa, synthetic construct
  SITE 2: note = Amino-isobutyric acid
  SITE 13: note = Amino-isobutyric acid
  SITE 20: note = Amino-isobutyric acid
  SITE 41: note = L-tert-butylglycine
YXEGTFTSDYSIXLEKQAQXAFVEWLIKGGPSSGASLRHYXNWLTRQRY

SEQ ID NO: 134 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSEGQSIRHYYNWLTRQRY

SEQ ID NO: 135 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEYLIKGGPSSGASLRHYYNWLTRQRY

SEQ ID NO: 136 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKAAQXAFVEWLIKGGPSSGASLRHYYNWLTRQRY

SEQ ID NO: 137 - protein, 49 aa, synthetic construct
  SITE 2: note = Amino-isobutyric acid
  SITE 13: note = Amino-isobutyric acid
  SITE 20: note = Amino-isobutyric acid
  SITE 44: note = L-tert-butylglycine
YXEGTFTSDYSIXLEKQAQXAFVEWLIKGGPSSGASLRHYYNWXTRQRY

SEQ ID NO: 138 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAEXAFVQWLIKGGPSEGASLRHYYNWLTRQRY

SEQ ID NO: 139 - protein, 49 aa, synthetic construct; identical to SEQ ID NO: 92
  SITE 2: note = Amino-isobutyric acid
  SITE 13: note = Amino-isobutyric acid
  SITE 20: note = Amino-isobutyric acid

SEQ ID NO: 140 - protein, 49 aa, synthetic construct; identical to SEQ ID NO: 95
  SITE 2: note = Amino-isobutyric acid
  SITE 13: note = Amino-isobutyric acid
  SITE 20: note = Amino-isobutyric acid

SEQ ID NO: 141 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXEFVQWLIKGGPSEGASLRHYYNWLTRQRY

SEQ ID NO: 142 - protein, 49 aa, synthetic construct; identical to SEQ ID NO: 101
  SITE 2: note = Amino-isobutyric acid
  SITE 13: note = Amino-isobutyric acid
  SITE 20: note = Amino-isobutyric acid

SEQ ID NO: 143 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid

SEQ ID NO: 144 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid

SEQ ID NO: 145 - protein, 49 aa, synthetic construct; identical to SEQ ID NO: 88
  SITE 2: note = Amino-isobutyric acid
  SITE 13: note = Amino-isobutyric acid
  SITE 20: note = Amino-isobutyric acid

SEQ ID NO: 146 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid

SEQ ID NO: 147 - protein, 49 aa, synthetic construct; identical to SEQ ID NO: 92
  SITE 2: note = Amino-isobutyric acid
  SITE 13: note = Amino-isobutyric acid
  SITE 20: note = Amino-isobutyric acid

SEQ ID NO: 148 - protein, 49 aa, synthetic construct; identical to SEQ ID NO: 101
  SITE 2: note = Amino-isobutyric acid
  SITE 13: note = Amino-isobutyric acid
  SITE 20: note = Amino-isobutyric acid

SEQ ID NO: 149 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid

SEQ ID NO: 150 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAEXAFVEWLIKGGPSSGASLRHYYNWLTRQRY

SEQ ID NO: 151 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid

SEQ ID NO: 152 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPESGASLRHYYNWLTRQRY

SEQ ID NO: 153 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid

SEQ ID NO: 154 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid

SEQ ID NO: 155 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid

SEQ ID NO: 156 - protein, 49 aa, synthetic construct; identical to SEQ ID NO: 138
  SITE 2: note = Amino-isobutyric acid
  SITE 13: note = Amino-isobutyric acid
  SITE 20: note = Amino-isobutyric acid

SEQ ID NO: 157 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid

SEQ ID NO: 158 - protein, 49 aa, synthetic construct; identical to SEQ ID NO: 95
  SITE 2: note = Amino-isobutyric acid
  SITE 13: note = Amino-isobutyric acid
  SITE 20: note = Amino-isobutyric acid

SEQ ID NO: 159 - protein, 49 aa, synthetic construct; identical to SEQ ID NO: 88
  SITE 2: note = Amino-isobutyric acid
  SITE 13: note = Amino-isobutyric acid
  SITE 20: note = Amino-isobutyric acid

SEQ ID NO: 160 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid

SEQ ID NO: 161 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPEEPASLRHYYNWLTRQRY

SEQ ID NO: 162 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid

SEQ ID NO: 163 - protein, 49 aa, synthetic construct; identical to SEQ ID NO: 152
  SITE 2: note = Amino-isobutyric acid
  SITE 13: note = Amino-isobutyric acid
  SITE 20: note = Amino-isobutyric acid

SEQ ID NO: 164 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid

SEQ ID NO: 165 - protein, 49 aa, synthetic construct; identical to SEQ ID NO: 101
  SITE 2: note = Amino-isobutyric acid
  SITE 13: note = Amino-isobutyric acid
  SITE 20: note = Amino-isobutyric acid

SEQ ID NO: 166 - protein, 49 aa, synthetic construct; identical to SEQ ID NO: 152
  SITE 2: note = Amino-isobutyric acid
  SITE 13: note = Amino-isobutyric acid
  SITE 20: note = Amino-isobutyric acid

SEQ ID NO: 167 - protein, 49 aa, synthetic construct; identical to SEQ ID NO: 92
  SITE 2: note = Amino-isobutyric acid
  SITE 13: note = Amino-isobutyric acid
  SITE 20: note = Amino-isobutyric acid

SEQ ID NO: 168 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPRSGASLRHYYNWLTRQRY

SEQ ID NO: 169 - protein, 49 aa, synthetic construct; identical to SEQ ID NO: 101
  SITE 2: note = Amino-isobutyric acid
  SITE 13: note = Amino-isobutyric acid
  SITE 20: note = Amino-isobutyric acid

SEQ ID NO: 170 - protein, 49 aa, synthetic construct; identical to SEQ ID NO: 92
  SITE 2: note = Amino-isobutyric acid
  SITE 13: note = Amino-isobutyric acid
  SITE 20: note = Amino-isobutyric acid

SEQ ID NO: 171 - protein, 49 aa, synthetic construct; identical to SEQ ID NO: 88
  SITE 2: note = Amino-isobutyric acid
  SITE 13: note = Amino-isobutyric acid
  SITE 20: note = Amino-isobutyric acid

SEQ ID NO: 172 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDLSIXLEKQAQXAFVEWLIKGGPSSGASLRHYYNWLTRQRY

SEQ ID NO: 173 - protein, 49 aa, synthetic construct; identical to SEQ ID NO: 101
  SITE 2: note = Amino-isobutyric acid
  SITE 13: note = Amino-isobutyric acid
  SITE 20: note = Amino-isobutyric acid

SEQ ID NO: 174 - protein, 49 aa, synthetic construct; identical to SEQ ID NO: 138
  SITE 2: note = Amino-isobutyric acid
  SITE 13: note = Amino-isobutyric acid
  SITE 20: note = Amino-isobutyric acid

SEQ ID NO: 175 - protein, 49 aa, synthetic construct; identical to SEQ ID NO: 95
  SITE 2: note = Amino-isobutyric acid
  SITE 13: note = Amino-isobutyric acid
  SITE 20: note = Amino-isobutyric acid

SEQ ID NO: 176 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid

SEQ ID NO: 177 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid

SEQ ID NO: 178 - protein, 49 aa, synthetic construct; identical to SEQ ID NO: 95
  SITE 2: note = Amino-isobutyric acid
  SITE 13: note = Amino-isobutyric acid
  SITE 20: note = Amino-isobutyric acid

SEQ ID NO: 179 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid

SEQ ID NO: 180 - protein, 49 aa, synthetic construct; identical to SEQ ID NO: 95
  SITE 2: note = Amino-isobutyric acid
  SITE 13: note = Amino-isobutyric acid
  SITE 20: note = Amino-isobutyric acid

SEQ ID NO: 181 - protein, 49 aa, synthetic construct; identical to SEQ ID NO: 97
  SITE 2: note = Amino-isobutyric acid
  SITE 13: note = Amino-isobutyric acid
  SITE 20: note = Amino-isobutyric acid

SEQ ID NO: 182 - protein, 49 aa, synthetic construct; identical to SEQ ID NO: 119
  SITE 2: note = Amino-isobutyric acid
  SITE 13: note = Amino-isobutyric acid
  SITE 20: note = Amino-isobutyric acid
  SITE 39: note = Amino-isobutyric acid

SEQ ID NO: 183 - protein, 49 aa, synthetic construct; identical to SEQ ID NO: 126
  SITE 2: note = Amino-isobutyric acid
  SITE 13: note = Amino-isobutyric acid
  SITE 20: note = Amino-isobutyric acid

SEQ ID NO: 184 - protein, 49 aa, synthetic construct; identical to SEQ ID NO: 133
  SITE 2: note = Amino-isobutyric acid
  SITE 13: note = Amino-isobutyric acid
  SITE 20: note = Amino-isobutyric acid
  SITE 41: note = Amino-isobutyric acid

SEQ ID NO: 185 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid

SEQ ID NO: 186 - protein, 49 aa, synthetic construct
  SITE 2: note = Amino-isobutyric acid
  SITE 13: note = Amino-isobutyric acid
  SITE 20: note = Amino-isobutyric acid
  SITE 47: note = Beta-homo-L-glutamine
YXEGTFTSDYSIXLEKQAQXAFVQWLIKGGPSEGASLRHYYNWLTRXRY

SEQ ID NO: 187 - protein, 49 aa, synthetic construct; identical to SEQ ID NO: 88
  SITE 2: note = Amino-isobutyric acid
  SITE 13: note = Amino-isobutyric acid
  SITE 20: note = Amino-isobutyric acid

SEQ ID NO: 188 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid

SEQ ID NO: 189 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVQWLIKGGPSEYASLRHYYNWLTRQRY

SEQ ID NO: 190 - protein, 49 aa, synthetic construct
  SITE 2: note = Amino-isobutyric acid
  SITE 13: note = Amino-isobutyric acid
  SITE 20: note = Amino-isobutyric acid
  SITE 44: note = D-Arginine
YXEGTFTSDYSIXLEKQAQXAFVEWLIKGGPSSGASLRHYYNWRTRQRY

SEQ ID NO: 191 - protein, 49 aa, synthetic construct
  SITE 2: note = Amino-isobutyric acid
  SITE 13: note = Amino-isobutyric acid
  SITE 20: note = Amino-isobutyric acid
  SITE 49: note = L-tert-butylglycine
YXEGTFTSDYSIXLEKQAQXAFVEWLIKGGPSSGASLRHYYNWLTRQRX

SEQ ID NO: 192 - protein, 49 aa, synthetic construct; identical to SEQ ID NO: 5
  SITE 2: note = Amino-isobutyric acid
  SITE 13: note = Amino-isobutyric acid
  SITE 16: note = Acetylated lysine
  SITE 20: note = Amino-isobutyric acid

SEQ ID NO: 193 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid

SEQ ID NO: 194 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid

SEQ ID NO: 195 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSGASLRHYYNLLTRQRY

SEQ ID NO: 196 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid

SEQ ID NO: 197 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSGALLRHYYNWLTRQRY

SEQ ID NO: 198 - protein, 49 aa, synthetic construct; identical to SEQ ID NO: 88
  SITE 2: note = Amino-isobutyric acid
  SITE 13: note = Amino-isobutyric acid
  SITE 20: note = Amino-isobutyric acid

SEQ ID NO: 199 - protein, 49 aa, synthetic construct
  SITE 2: note = Amino-isobutyric acid
  SITE 13: note = Amino-isobutyric acid
  SITE 16: note = Amino-isobutyric acid
  SITE 20: note = Amino-isobutyric acid
YXEGTFTSDYSIXLEXQAQXAFVEWLIKGGPSSGASLRHYYNWLTRQRY

SEQ ID NO: 200 - protein, 49 aa, synthetic construct; identical to SEQ ID NO: 138
  SITE 2: note = Amino-isobutyric acid
  SITE 13: note = Amino-isobutyric acid
  SITE 20: note = Amino-isobutyric acid

SEQ ID NO: 201 - protein, 49 aa, synthetic construct; identical to SEQ ID NO: 88
  SITE 2: note = Amino-isobutyric acid
  SITE 13: note = Amino-isobutyric acid
  SITE 20: note = Amino-isobutyric acid

SEQ ID NO: 202 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid

SEQ ID NO: 203 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSYYASLRHYYNWLTRQRY

SEQ ID NO: 204 - protein, 49 aa, synthetic construct
  SITE 2: note = Amino-isobutyric acid
  SITE 13: note = Amino-isobutyric acid
  SITE 20: note = Amino-isobutyric acid
  SITE 44: note = D-Aspartic acid
YXEGTFTSDYSIXLEKQAQXAFVEWLIKGGPSSGASLRHYYNWDTRQRY

SEQ ID NO: 205 - protein, 49 aa, synthetic construct; identical to SEQ ID NO: 88
  SITE 2: note = Amino-isobutyric acid
  SITE 13: note = Amino-isobutyric acid
  SITE 20: note = Amino-isobutyric acid

SEQ ID NO: 206 - protein, 49 aa, synthetic construct; identical to SEQ ID NO: 161
  SITE 2: note = Amino-isobutyric acid
  SITE 13: note = Amino-isobutyric acid
  SITE 20: note = Amino-isobutyric acid

SEQ ID NO: 207 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid

SEQ ID NO: 208 - protein, 49 aa, synthetic construct
  SITE 2: note = Amino-isobutyric acid
  SITE 13: note = Amino-isobutyric acid
  SITE 20: note = Amino-isobutyric acid
  SITE 47: note = Beta-homo-L-glutamine
YXEGTFTSDYSIXLEKQAQXAFVEWLIKGGPSSGASLRHYYNWLTRXRY

SEQ ID NO: 209 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLELQAQXAFVEWLIKGGPSSGASLRHYYNWLTRQRY

SEQ ID NO: 210 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSSGASLLHYYNWLTRQRY

SEQ ID NO: 211 - protein, 49 aa, synthetic construct
  SITE 2: note = Amino-isobutyric acid
  SITE 13: note = Amino-isobutyric acid
  SITE 20: note = D-Aspartic acid
YXEGTFTSDYSIXLEKQAQDAFVEWLIKGGPSSGASLRHYYNWLTRQRY

SEQ ID NO: 212 - protein, 49 aa, synthetic construct; identical to SEQ ID NO: 96
  SITE 2: note = Amino-isobutyric acid
  SITE 13: note = Amino-isobutyric acid
  SITE 20: note = Amino-isobutyric acid

SEQ ID NO: 213 - protein, 49 aa, synthetic construct
  SITE 2: note = Amino-isobutyric acid
  SITE 13: note = Amino-isobutyric acid
  SITE 20: note = Amino-isobutyric acid
  SITE 42: note = Amino-isobutyric acid
YXEGTFTSDYSIXLEKQAQXAFVEWLIKGGPSSGASLRHYYXWLTRQRY

SEQ ID NO: 214 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 49: note = D-Tyrosine

SEQ ID NO: 215 - protein, 49 aa, synthetic construct; identical to SEQ ID NO: 88
  SITE 2: note = Amino-isobutyric acid
  SITE 13: note = Amino-isobutyric acid
  SITE 20: note = Amino-isobutyric acid

SEQ ID NO: 216 - protein, 49 aa, synthetic construct
  SITE 2: note = Amino-isobutyric acid
  SITE 13: note = Amino-isobutyric acid
  SITE 20: note = D-Arginine
YXEGTFTSDYSIXLEKQAQRAFVEWLIKGGPSSGASLRHYYNWLTRQRY

SEQ ID NO: 217 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSEYVSLRHYYNWLTRQRY

SEQ ID NO: 218 - protein, 49 aa, synthetic construct
  SITE 2: note = Amino-isobutyric acid
  SITE 13: note = Amino-isobutyric acid
  SITE 20: note = Amino-isobutyric acid
  SITE 43: note = Amino-isobutyric acid
YXEGTFTSDYSIXLEKQAQXAFVEWLIKGGPSSGASLRHYYNXLTRQRY

SEQ ID NO: 219 - protein, 49 aa, synthetic construct; identical to SEQ ID NO: 138
  SITE 2: note = Amino-isobutyric acid
  SITE 13: note = Amino-isobutyric acid
  SITE 20: note = Amino-isobutyric acid

SEQ ID NO: 220 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 221 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid

SEQ ID NO: 222 - protein, 49 aa, synthetic construct
  SITE 2: note = Amino-isobutyric acid
  SITE 13: note = Amino-isobutyric acid
  SITE 20: note = Amino-isobutyric acid
  SITE 47: note = N-methyl-L-glutamine
YXEGTFTSDYSIXLEKQAQXAFVEWLIKGGPSEGASLRHYYNWLTRQRY

SEQ ID NO: 223 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 224 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 225 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid

SEQ ID NO: 226 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSEPASLRHYYNWLTRQRY

SEQ ID NO: 227 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSEGQSLRHYYNWLTRQRY

SEQ ID NO: 228 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 229 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid

SEQ ID NO: 230 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPEEGASLRHYYNWLTRQRY

SEQ ID NO: 231 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFIEWLIKGGPSEGASLRHYYNWLTRQRY

SEQ ID NO: 232 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSYGASLRHYYNWLTRQRY

SEQ ID NO: 233 - protein, 49 aa, synthetic construct; identical to SEQ ID NO: 226
  SITE 2: note = Amino-isobutyric acid
  SITE 13: note = Amino-isobutyric acid
  SITE 20: note = Amino-isobutyric acid

SEQ ID NO: 234 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDLSIXLEKQAQXAFVEWLIKGGPSEGASLRHYYNWLTRQRY

SEQ ID NO: 235 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid

SEQ ID NO: 236 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 48: note = N-methyl-L-arginine

SEQ ID NO: 237 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSEGESLRHYYNWLTRQRY

SEQ ID NO: 238 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid

SEQ ID NO: 239 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 240 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 241 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSEGVSLRHYYNWLTRQRY

SEQ ID NO: 242 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 243 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 244 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIKGGPSTGASLRHYYNWLTRQRY

SEQ ID NO: 245 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 246 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 247 - protein, 49 aa, synthetic construct; identical to SEQ ID NO: 231
  SITE 2: note = Amino-isobutyric acid
  SITE 13: note = Amino-isobutyric acid
  SITE 20: note = Amino-isobutyric acid

SEQ ID NO: 248 - protein, 49 aa, synthetic construct; identical to SEQ ID NO: 230
  SITE 2: note = Amino-isobutyric acid
  SITE 13: note = Amino-isobutyric acid
  SITE 20: note = Amino-isobutyric acid

SEQ ID NO: 249 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 250 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 251 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAEXAFVEWLIKGGPSEGASLRHYYNWLTRQRY

SEQ ID NO: 252 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 253 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid

SEQ ID NO: 254 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid

SEQ ID NO: 255 - protein, 49 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXEFVEWLIKGGPSEGASLRHYYNWLTRQRY

SEQ ID NO: 256 - protein, 49 aa, synthetic construct; identical to SEQ ID NO: 226
  SITE 2: note = Amino-isobutyric acid
  SITE 13: note = Amino-isobutyric acid
  SITE 20: note = Amino-isobutyric acid

SEQ ID NO: 257 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 258 - protein, 49 aa, synthetic construct; identical to SEQ ID NO: 231
  SITE 2: note = Amino-isobutyric acid
  SITE 13: note = Amino-isobutyric acid
  SITE 20: note = Amino-isobutyric acid

SEQ ID NO: 259 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid

SEQ ID NO: 260 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid

SEQ ID NO: 261 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid

SEQ ID NO: 262 - protein, 49 aa, synthetic construct; identical to SEQ ID NO: 251
  SITE 2: note = Amino-isobutyric acid
  SITE 13: note = Amino-isobutyric acid
  SITE 20: note = Amino-isobutyric acid

SEQ ID NO: 263 - protein, 49 aa, synthetic construct; identical to SEQ ID NO: 226
  SITE 2: note = Amino-isobutyric acid
  SITE 13: note = Amino-isobutyric acid
  SITE 20: note = Amino-isobutyric acid

SEQ ID NO: 264 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 265 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 266 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 267 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 268 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 269 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 270 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 271 - protein, 49 aa, synthetic construct; identical to SEQ ID NO: 255
  SITE 2: note = Amino-isobutyric acid
  SITE 13: note = Amino-isobutyric acid
  SITE 20: note = Amino-isobutyric acid

SEQ ID NO: 272 - protein, 49 aa, synthetic construct; identical to SEQ ID NO: 230
  SITE 2: note = Amino-isobutyric acid
  SITE 13: note = Amino-isobutyric acid
  SITE 20: note = Amino-isobutyric acid

SEQ ID NO: 273 - protein, 49 aa, synthetic construct; identical to SEQ ID NO: 255
  SITE 2: note = Amino-isobutyric acid
  SITE 13: note = Amino-isobutyric acid
  SITE 20: note = Amino-isobutyric acid

SEQ ID NO: 274 - protein, 49 aa, synthetic construct; identical to SEQ ID NO: 255
  SITE 2: note = Amino-isobutyric acid
  SITE 13: note = Amino-isobutyric acid
  SITE 20: note = Amino-isobutyric acid

SEQ ID NO: 275 - protein, 49 aa, synthetic construct; identical to SEQ ID NO: 255
  SITE 2: note = Amino-isobutyric acid
  SITE 13: note = Amino-isobutyric acid
  SITE 20: note = Amino-isobutyric acid

SEQ ID NO: 276 - protein, 49 aa, synthetic construct; identical to SEQ ID NO: 255
  SITE 2: note = Amino-isobutyric acid
  SITE 13: note = Amino-isobutyric acid
  SITE 20: note = Amino-isobutyric acid

SEQ ID NO: 277 - protein, 49 aa, synthetic construct
  SITE 2: note = Amino-isobutyric acid
  SITE 13: note = Amino-isobutyric acid
  SITE 20: note = Amino-isobutyric acid
  SITE 47: note = Beta-homo-L-glutamine
YXEGTFTSDYSIXLEKQAQXAFVEWLIKGGPSEGASLRHYYNWLTRXRY

SEQ ID NO: 278 - protein, 49 aa, synthetic construct; identical to SEQ ID NO: 251
  SITE 2: note = Amino-isobutyric acid
  SITE 13: note = Amino-isobutyric acid
  SITE 20: note = Amino-isobutyric acid

SEQ ID NO: 279 - protein, 49 aa, synthetic construct; identical to SEQ ID NO: 255
  SITE 2: note = Amino-isobutyric acid
  SITE 13: note = Amino-isobutyric acid
  SITE 20: note = Amino-isobutyric acid

SEQ ID NO: 280 - protein, 49 aa, synthetic construct; identical to SEQ ID NO: 251
  SITE 2: note = Amino-isobutyric acid
  SITE 13: note = Amino-isobutyric acid
  SITE 20: note = Amino-isobutyric acid

SEQ ID NO: 281 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid

SEQ ID NO: 282 - protein, 49 aa, synthetic construct; identical to SEQ ID NO: 255
  SITE 2: note = Amino-isobutyric acid
  SITE 13: note = Amino-isobutyric acid
  SITE 20: note = Amino-isobutyric acid

SEQ ID NO: 283 - protein, 49 aa, synthetic construct; identical to SEQ ID NO: 231
  SITE 2: note = Amino-isobutyric acid
  SITE 13: note = Amino-isobutyric acid
  SITE 20: note = Amino-isobutyric acid

SEQ ID NO: 284 - protein, 49 aa, synthetic construct; identical to SEQ ID NO: 255
  SITE 2: note = Amino-isobutyric acid
  SITE 13: note = Amino-isobutyric acid
  SITE 20: note = Amino-isobutyric acid

SEQ ID NO: 285 - protein, 49 aa, synthetic construct; identical to SEQ ID NO: 230
  SITE 2: note = Amino-isobutyric acid
  SITE 13: note = Amino-isobutyric acid
  SITE 20: note = Amino-isobutyric acid

SEQ ID NO: 286 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 287 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 288 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 289 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 290 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 291 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 292 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 293 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 294 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 295 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 296 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 297 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 298 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 299 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 300 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 301 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 302 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 303 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 304 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 305 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid

SEQ ID NO: 306 - protein, 28 aa, synthetic construct
  SITE 2: note = Amino-isobutyric acid
  SITE 13: note = Amino-isobutyric acid
  SITE 20: note = Amino-isobutyric acid
YXEGTFTSDYSIXLEKQAQXAFVEWLIK

SEQ ID NO: 307 - protein, 15 aa, synthetic construct
ASLRHYYNWLTRQRY

SEQ ID NO: 308 - protein, 36 aa, Homo sapiens
YPIKPEAPREDASPEELNRYYASLRHYLNLVTRQRY

SEQ ID NO: 309 - protein, 31 aa, Homo sapiens
HAEGTFTSDVSSYLEGQAAKEFIAWLVKGRG

SEQ ID NO: 310 - protein, 42 aa, Homo sapiens
YAEGTFISDYSIAMDKIHQQDFVNWLLAQKGKKNDWKHNITQ

SEQ ID NO: 311 - protein, 6 aa, synthetic construct
GGPSEG

SEQ ID NO: 312 - protein, 16 aa, synthetic construct
YASLRHYYNWLTRQRY

SEQ ID NO: 313 - protein, 6 aa, synthetic construct
GGPEEG

SEQ ID NO: 314 - protein, 6 aa, synthetic construct
GGPEEP

SEQ ID NO: 315 - protein, 6 aa, synthetic construct
GGPESG

SEQ ID NO: 316 - protein, 6 aa, synthetic construct
GGPESP

SEQ ID NO: 317 - protein, 6 aa, synthetic construct
GGPSEP

SEQ ID NO: 318 - protein, 6 aa, synthetic construct
GGPSEY

SEQ ID NO: 319 - protein, 6 aa, synthetic construct
GGPSSA

SEQ ID NO: 320 - protein, 6 aa, synthetic construct
GGPSSG

SEQ ID NO: 321 - protein, 6 aa, synthetic construct
GGPSSP

SEQ ID NO: 322 - protein, 6 aa, synthetic construct
GGPSSY

SEQ ID NO: 323 - protein, 6 aa, synthetic construct
GGPSTG

SEQ ID NO: 324 - protein, 6 aa, synthetic construct
GGPSYG

SEQ ID NO: 325 - protein, 6 aa, synthetic construct
GGQSSG

SEQ ID NO: 326 - protein, 4 aa, synthetic construct
YNWL

SEQ ID NO: 327 - protein, 4 aa, synthetic construct
RQRY

SEQ ID NO: 328 - protein, 49 aa, synthetic construct; identical to SEQ ID NO: 2
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 329 - protein, 49 aa, synthetic construct; identical to SEQ ID NO: 3
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 330 - protein, 49 aa, synthetic construct; identical to SEQ ID NO: 4
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 37: note = Amino-isobutyric acid
  SITE 41: note = Amino-isobutyric acid
  SITE 44: note = Amino-isobutyric acid
  SITE 49: note = Tyrosine amide

SEQ ID NO: 331 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 37: note = D-Leucine
  SITE 39: note = D-Histidine
  SITE 46: note = D-Arginine
  SITE 49: note = D-Tyrosine amide

SEQ ID NO: 332 - protein, 49 aa, synthetic construct; identical to SEQ ID NO: 6
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 333 - protein, 49 aa, synthetic construct; identical to SEQ ID NO: 7
  SITE 2: note = Amino-isobutyric acid
  SITE 13: note = Amino-isobutyric acid
  SITE 20: note = Amino-isobutyric acid
  SITE 28: note = (2S)-2-amino-6-[(2S)-2-amino-6-[(4S)-4-carboxy-4-[( 4S)-4-carboxy-4-[(4S)-4-carboxy-4-[(4S)-4-carboxy-4-[(4S)- 4-carboxy-4-(19-carboxynonadecanamido)butanamido]butanamid o]butanamido]butanamido]butanamido]hexanamido]hexanoyl attached to lysine via a side chain
  SITE 49: note = Tyrosine amide

SEQ ID NO: 334 - protein, 49 aa, synthetic construct; identical to SEQ ID NO: 8
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 335 - protein, 49 aa, synthetic construct; identical to SEQ ID NO: 9
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 336 - protein, 49 aa, synthetic construct; identical to SEQ ID NO: 10
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 37: note = Amino-isobutyric acid
  SITE 49: note = Tyrosine amide

SEQ ID NO: 337 - protein, 49 aa, synthetic construct; identical to SEQ ID NO: 11
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]butanoyl attached to lysine via a side chain
  SITE 49: note = Tyrosine amide

SEQ ID NO: 338 - protein, 49 aa, synthetic construct; identical to SEQ ID NO: 11
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]butanoy l attached to lysine via a side chain
  SITE 49: note = Tyrosine amide

SEQ ID NO: 339 - protein, 49 aa, synthetic construct; identical to SEQ ID NO: 13
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 37: note = Amino-isobutyric acid
  SITE 44: note = Amino-isobutyric acid
  SITE 49: note = Tyrosine amide

SEQ ID NO: 340 - protein, 49 aa, synthetic construct; identical to SEQ ID NO: 14
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 341 - protein, 49 aa, synthetic construct; identical to SEQ ID NO: 15
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 342 - protein, 49 aa, synthetic construct; identical to SEQ ID NO: 16
  SITE 2: note = Amino-isobutyric acid
  SITE 13: note = Amino-isobutyric acid
  SITE 16: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 343 - protein, 49 aa, synthetic construct; identical to SEQ ID NO: 17
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 44: note = Amino-isobutyric acid
  SITE 49: note = Tyrosine amide

SEQ ID NO: 344 - protein, 49 aa, synthetic construct; identical to SEQ ID NO: 18
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 41: note = Amino-isobutyric acid
  SITE 44: note = Amino-isobutyric acid
  SITE 49: note = Tyrosine amide

SEQ ID NO: 345 - protein, 49 aa, synthetic construct; identical to SEQ ID NO: 19
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 346 - protein, 49 aa, synthetic construct; identical to SEQ ID NO: 20
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 347 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 39: note = D-Histidine
  SITE 46: note = D-Arginine
  SITE 49: note = D-Tyrosine amide

SEQ ID NO: 348 - protein, 49 aa, synthetic construct; identical to SEQ ID NO: 22
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 349 - protein, 49 aa, synthetic construct; identical to SEQ ID NO: 23
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(19- carboxynonadecanamido)butanamido]butanamido]butanamido]but anamido]butanamido]hexanoyl attached to lysine via a side chain
  SITE 49: note = Tyrosine amide

SEQ ID NO: 350 - protein, 49 aa, synthetic construct; identical to SEQ ID NO: 24
  SITE 2: note = Amino-isobutyric acid
  SITE 13: note = Amino-isobutyric acid
  SITE 16: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 351 - protein, 49 aa, synthetic construct; identical to SEQ ID NO: 25
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 352 - protein, 49 aa, synthetic construct; identical to SEQ ID NO: 26
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 37: note = Amino-isobutyric acid
  SITE 46: note = Homo-L-arginine
  SITE 49: note = D-Tyrosine amide

SEQ ID NO: 353 - protein, 49 aa, synthetic construct; identical to SEQ ID NO: 27
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 354 - protein, 49 aa, synthetic construct; identical to SEQ ID NO: 28
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-[(4S)-4-carboxy-4-(1 9-carboxynonadecanamido)butanamido]butanamido]butanamido]b utanamido]butanamido]butanoyl attached to lysine via a side chain
  SITE 49: note = Tyrosine amide

SEQ ID NO: 355 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]butanoyl attached to lysine via a side chain
  SITE 49: note = Tyrosine amide

SEQ ID NO: 356 - protein, 49 aa, synthetic construct; identical to SEQ ID NO: 30
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 357 - protein, 49 aa, synthetic construct; identical to SEQ ID NO: 31
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 358 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-[(4S)-4-carboxy-4-(1 9-carboxynonadecanamido)butanamido]butanamido]butanamido]b utanamido]butanamido]ethoxy}ethoxy)acetamido]ethoxy}ethoxy )acetyl attached to lysine via a side chain
  SITE 49: note = Tyrosine amide

SEQ ID NO: 359 - protein, 49 aa, synthetic construct; identical to SEQ ID NO: 33
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 360 - protein, 49 aa, synthetic construct; identical to SEQ ID NO: 30
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(19- carboxynonadecanamido)butanamido]butanamido]butanamido]but anamido]butanamido]hexanoyl attached to lysine via a side chain
  SITE 49: note = Tyrosine amide

SEQ ID NO: 361 - protein, 49 aa, synthetic construct; identical to SEQ ID NO: 35
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 362 - protein, 49 aa, synthetic construct; identical to SEQ ID NO: 28
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-[(4S)-4-carboxy-4-(1 9-carboxynonadecanamido)butanamido]butanamido]butanamido]b utanamido]butanamido]ethoxy}ethoxy)acetamido]ethoxy}ethoxy )acetyl attached to lysine via a side chain
  SITE 49: note = Tyrosine amide

SEQ ID NO: 363 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 364 - protein, 49 aa, synthetic construct; identical to SEQ ID NO: 38
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 37: note = Amino-isobutyric acid
  SITE 49: note = Tyrosine amide

SEQ ID NO: 365 - protein, 49 aa, synthetic construct; identical to SEQ ID NO: 39
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 37: note = Amino-isobutyric acid
  SITE 48: note = N-methyl-L-arginine
  SITE 49: note = Tyrosine amide

SEQ ID NO: 366 - protein, 49 aa, synthetic construct; identical to SEQ ID NO: 40
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 367 - protein, 49 aa, synthetic construct; identical to SEQ ID NO: 41
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 368 - protein, 49 aa, synthetic construct; identical to SEQ ID NO: 42
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 369 - protein, 49 aa, synthetic construct; identical to SEQ ID NO: 28
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-[(4S)-4-carboxy-4-(19-carboxynonadecanamido) butanamido]butanamido]butanamido]butanamido]butanamido]but anamido]butanamido]hexanoyl attached to lysine via a side chain
  SITE 49: note = Tyrosine amide

SEQ ID NO: 370 - protein, 49 aa, synthetic construct; identical to SEQ ID NO: 44
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 37: note = Amino-isobutyric acid
  SITE 49: note = Tyrosine amide

SEQ ID NO: 371 - protein, 49 aa, synthetic construct; identical to SEQ ID NO: 30
  SITE 2: note = Amino-isobutyric acid
  SITE 13: note = Amino-isobutyric acid
  SITE 20: note = Amino-isobutyric acid
  SITE 28: note = (2S)-2-amino-6-[2-(2-{2-[(4S)-4-carboxy-4-[(4S)-4-c arboxy-4-[(4S)-4-carboxy-4-[(4S)-4-carboxy-4-[(4S)-4-carbo xy-4-(19-carboxynonadecanamido)butanamido]butanamido]butan amido]butanamido]butanamido]ethoxy}ethoxy)acetamido]hexano yl attached to lysine via a side chain
  SITE 49: note = Tyrosine amide

SEQ ID NO: 372 - protein, 49 aa, synthetic construct; identical to SEQ ID NO: 46
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 373 - protein, 49 aa, synthetic construct; identical to SEQ ID NO: 47
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 374 - protein, 49 aa, synthetic construct; identical to SEQ ID NO: 48
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 375 - protein, 49 aa, synthetic construct; identical to SEQ ID NO: 49
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 37: note = Amino-isobutyric acid
  SITE 49: note = Tyrosine amide

SEQ ID NO: 376 - protein, 49 aa, synthetic construct; identical to SEQ ID NO: 50
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 377 - protein, 49 aa, synthetic construct; identical to SEQ ID NO: 51
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(17-carboxyheptadecanamido)butanamido]butana mido]butanamido]butanamido]butanamido]butanamido]hexanoyl attached to lysine via a side chain
  SITE 49: note = Tyrosine amide

SEQ ID NO: 378 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid
  SITE 28: note = Lysine attached via its side chain to the C-terminus of SEQ ID NO: 634
  SITE 49: note = Tyrosine amide

SEQ ID NO: 379 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]butanoy l attached to lysine via a side chain
  SITE 49: note = Tyrosine amide

SEQ ID NO: 380 - protein, 49 aa, synthetic construct; identical to SEQ ID NO: 54
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-(17-carboxyheptadecanamido)butanamido] butanamido]butanamido]butanoyl attached to lysine via a side chain
  SITE 49: note = Tyrosine amide

SEQ ID NO: 381 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-[(4S)-4-carboxy-4-(1 7-carboxyheptadecanamido)butanamido]butanamido]butanamido] butanamido]butanamido]ethoxy}ethoxy)acetamido]ethoxy}ethox y)acetyl attached to lysine via a side chain
  SITE 49: note = Tyrosine amide

SEQ ID NO: 382 - protein, 49 aa, synthetic construct; identical to SEQ ID NO: 56
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]ethoxy} ethoxy)acetamido]ethoxy}ethoxy)acetyl attached to lysine via a side chain
  SITE 49: note = Tyrosine amide

SEQ ID NO: 383 - protein, 49 aa, synthetic construct; identical to SEQ ID NO: 57
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]ethoxy} ethoxy)acetamido]ethoxy}ethoxy)acetyl attached to lysine via a side chain
  SITE 49: note = Tyrosine amide

SEQ ID NO: 384 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]ethoxy} ethoxy)acetamido]ethoxy}ethoxy)acetyl attached to lysine via a side chain
  SITE 49: note = Tyrosine amide

SEQ ID NO: 385 - protein, 49 aa, synthetic construct; identical to SEQ ID NO: 59
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(19- carboxynonadecanamido)butanamido]butanamido]butanamido]but anamido]butanamido]hexanoyl attached to lysine via a side chain
  SITE 49: note = Tyrosine amide

SEQ ID NO: 386 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(17- carboxyheptadecanamido)butanamido]butanamido]butanamido]bu tanamido]butanamido]hexanoyl attached to lysine via a side chain
  SITE 49: note = Tyrosine amide

SEQ ID NO: 387 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(19- carboxynonadecanamido)butanamido]butanamido]butanamido]but anamido]butanamido]hexanoyl attached to lysine via a side chain
  SITE 49: note = Tyrosine amide

SEQ ID NO: 388 - protein, 49 aa, synthetic construct; identical to SEQ ID NO: 62
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 389 - protein, 49 aa, synthetic construct; identical to SEQ ID NO: 29
  SITE 2: note = Amino-isobutyric acid
  SITE 13: note = Amino-isobutyric acid
  SITE 20: note = Amino-isobutyric acid
  SITE 28: note = Lysine attached via its side chain to the C-terminus of SEQ ID NO: 635
  SITE 49: note = Tyrosine amide

SEQ ID NO: 390 - protein, 49 aa, synthetic construct; identical to SEQ ID NO: 64
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]ethoxy} ethoxy)acetamido]ethoxy}ethoxy)acetyl attached to lysine via a side chain
  SITE 49: note = Tyrosine amide

SEQ ID NO: 391 - protein, 49 aa, synthetic construct; identical to SEQ ID NO: 65
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-[(4S)-4-carboxy-4-(1 9-carboxynonadecanamido)butanamido]butanamido]butanamido]b utanamido]butanamido]butanoyl attached to lysine via a side chain
  SITE 49: note = Tyrosine amide

SEQ ID NO: 392 - protein, 49 aa, synthetic construct; identical to SEQ ID NO: 66
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 393 - protein, 49 aa, synthetic construct; identical to SEQ ID NO: 57
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(19- carboxynonadecanamido)butanamido]butanamido]butanamido]but anamido]butanamido]hexanoyl attached to lysine via a side chain
  SITE 49: note = Tyrosine amide

SEQ ID NO: 394 - protein, 49 aa, synthetic construct; identical to SEQ ID NO: 68
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 44: note = Amino-isobutyric acid
  SITE 49: note = Tyrosine amide

SEQ ID NO: 395 - protein, 49 aa, synthetic construct; identical to SEQ ID NO: 69
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 396 - protein, 49 aa, synthetic construct; identical to SEQ ID NO: 64
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(19- carboxynonadecanamido)butanamido]butanamido]butanamido]but anamido]butanamido]hexanoyl attached to lysine via a side chain
  SITE 49: note = Tyrosine amide

SEQ ID NO: 397 - protein, 49 aa, synthetic construct; identical to SEQ ID NO: 71
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 46: note = Homo-L-arginine
  SITE 49: note = D-Tyrosine amide

SEQ ID NO: 398 - protein, 49 aa, synthetic construct; identical to SEQ ID NO: 72
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 399 - protein, 49 aa, synthetic construct; identical to SEQ ID NO: 73
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 400 - protein, 49 aa, synthetic construct; identical to SEQ ID NO: 39
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 37: note = Amino-isobutyric acid
  SITE 49: note = D-Tyrosine amide

SEQ ID NO: 401 - protein, 49 aa, synthetic construct; identical to SEQ ID NO: 75
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(19- carboxynonadecanamido)butanamido]butanamido]butanamido]but anamido]butanamido]hexanoyl attached to lysine via a side chain
  SITE 49: note = Tyrosine amide

SEQ ID NO: 402 - protein, 49 aa, synthetic construct; identical to SEQ ID NO: 76
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 37: note = Amino-isobutyric acid
  SITE 41: note = Amino-isobutyric acid
  SITE 49: note = Tyrosine amide

SEQ ID NO: 403 - protein, 49 aa, synthetic construct; identical to SEQ ID NO: 57
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]ethoxy}e thoxy)acetamido]ethoxy}ethoxy)acetyl attached to lysine via a side chain
  SITE 49: note = Tyrosine amide

SEQ ID NO: 404 - protein, 49 aa, synthetic construct; identical to SEQ ID NO: 78
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 405 - protein, 49 aa, synthetic construct; identical to SEQ ID NO: 79
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 44: note = Amino-isobutyric acid
  SITE 49: note = Tyrosine amide

SEQ ID NO: 406 - protein, 49 aa, synthetic construct; identical to SEQ ID NO: 75
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 407 - protein, 49 aa, synthetic construct; identical to SEQ ID NO: 81
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-(17-carboxyheptadecanamido)butanamido] butanamido]butanamido]ethoxy}ethoxy)acetamido]ethoxy}ethox y)acetyl attached to lysine via a side chain
  SITE 49: note = Tyrosine amide

SEQ ID NO: 408 - protein, 49 aa, synthetic construct; identical to SEQ ID NO: 64
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]ethoxy}e thoxy)acetamido]ethoxy}ethoxy)acetyl attached to lysine via a side chain
  SITE 49: note = Tyrosine amide

SEQ ID NO: 409 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 48: note = N-methyl-L-arginine
  SITE 49: note = Tyrosine amide

SEQ ID NO: 410 - protein, 49 aa, synthetic construct; identical to SEQ ID NO: 84
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 411 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 47: note = N-methyl-L-glutamine
  SITE 49: note = Tyrosine amide

SEQ ID NO: 412 - protein, 49 aa, synthetic construct; identical to SEQ ID NO: 86
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 413 - protein, 49 aa, synthetic construct; identical to SEQ ID NO: 87
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 414 - protein, 49 aa, synthetic construct; identical to SEQ ID NO: 88
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-[(4S)-4-carboxy-4-(19-carboxynonadecanamido) butanamido]butanamido]butanamido]butanamido]butanamido]but anamido]butanamido]hexanoyl attached to lysine via a side chain
  SITE 49: note = Tyrosine amide

SEQ ID NO: 415 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 416 - protein, 49 aa, synthetic construct; identical to SEQ ID NO: 90
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 417 - protein, 49 aa, synthetic construct; identical to SEQ ID NO: 91
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 418 - protein, 49 aa, synthetic construct; identical to SEQ ID NO: 92
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-[(4S)-4-carboxy-4-(19-carboxynonadecanamido) butanamido]butanamido]butanamido]butanamido]butanamido]but anamido]butanamido]hexanoyl attached to lysine via a side chain
  SITE 49: note = Tyrosine amide

SEQ ID NO: 419 - protein, 49 aa, synthetic construct; identical to SEQ ID NO: 93
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 420 - protein, 49 aa, synthetic construct; identical to SEQ ID NO: 94
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 421 - protein, 49 aa, synthetic construct; identical to SEQ ID NO: 95
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 422 - protein, 49 aa, synthetic construct; identical to SEQ ID NO: 96
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 423 - protein, 49 aa, synthetic construct; identical to SEQ ID NO: 97
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-(19-carboxynonadecanam ido)butanamido]butanamido]butanamido]butanamido]hexanoyl attached to lysine via a side chain
  SITE 49: note = Tyrosine amide

SEQ ID NO: 424 - protein, 49 aa, synthetic construct; identical to SEQ ID NO: 98
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 425 - protein, 49 aa, synthetic construct; identical to SEQ ID NO: 99
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 426 - protein, 49 aa, synthetic construct; identical to SEQ ID NO: 97
  SITE 2: note = Amino-isobutyric acid
  SITE 13: note = Amino-isobutyric acid
  SITE 20: note = Amino-isobutyric acid
  SITE 28: note = (2S)-2-amino-6-[2-(2-{2-[2-(2-{2-[(4S)-4-carboxy-4- [(4S)-4-carboxy-4-[(4S)-4-carboxy-4-[(4S)-4-carboxy-4-(19- carboxynonadecanamido)butanamido]butanamido]butanamido]but anamido]ethoxy}ethoxy)acetamido]ethoxy}ethoxy)acetamido]he xanoyl attached to lysine via a side chain
  SITE 49: note = Tyrosine amide

SEQ ID NO: 427 - protein, 49 aa, synthetic construct; identical to SEQ ID NO: 101
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 428 - protein, 49 aa, synthetic construct; identical to SEQ ID NO: 102
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 429 - protein, 49 aa, synthetic construct; identical to SEQ ID NO: 39
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 37: note = L-tert-butylglycine
  SITE 49: note = Tyrosine amide

SEQ ID NO: 430 - protein, 49 aa, synthetic construct; identical to SEQ ID NO: 104
  SITE 2: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 431 - protein, 49 aa, synthetic construct; identical to SEQ ID NO: 97
  SITE 2: note = Amino-isobutyric acid
  SITE 13: note = Amino-isobutyric acid
  SITE 20: note = Amino-isobutyric acid
  SITE 28: note = (2S)-2-amino-6-[(2S)-2-amino-6-[(4S)-4-carboxy-4-[( 4S)-4-carboxy-4-[(4S)-4-carboxy-4-[(4S)-4-carboxy-4-[(4S)- 4-carboxy-4-(19-carboxynonadecanamido)butanamido]butanamid o]butanamido]butanamido]butanamido]hexanamido]hexanoyl attached to lysine via a side chain
  SITE 49: note = Tyrosine amide

SEQ ID NO: 432 - protein, 49 aa, synthetic construct; identical to SEQ ID NO: 106
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 433 - protein, 49 aa, synthetic construct; identical to SEQ ID NO: 107
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 434 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(19- carboxynonadecanamido)butanamido]butanamido]butanamido]but anamido]butanamido]hexanoyl attached to lysine via a side chain
  SITE 49: note = Tyrosine amide

SEQ ID NO: 435 - protein, 49 aa, synthetic construct; identical to SEQ ID NO: 109
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 436 - protein, 49 aa, synthetic construct; identical to SEQ ID NO: 97
  SITE 2: note = Amino-isobutyric acid
  SITE 13: note = Amino-isobutyric acid
  SITE 20: note = Amino-isobutyric acid
  SITE 28: note = (2S)-2-amino-6-[2-(2-{2-[(4S)-4-carboxy-4-[(4S)-4-c arboxy-4-[(4S)-4-carboxy-4-[(4S)-4-carboxy-4-[(4S)-4-carbo xy-4-(19-carboxynonadecanamido)butanamido]butanamido]butan amido]butanamido]butanamido]ethoxy}ethoxy)acetamido]hexano yl attached to lysine via a side chain
  SITE 49: note = Tyrosine amide

SEQ ID NO: 437 - protein, 49 aa, synthetic construct; identical to SEQ ID NO: 101
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-[(4S)-4-carboxy-4-(1 9-carboxynonadecanamido)butanamido]butanamido]butanamido]b utanamido]butanamido]ethoxy}ethoxy)acetamido]ethoxy}ethoxy )acetyl attached to lysine via a side chain
  SITE 49: note = Tyrosine amide

SEQ ID NO: 438 - protein, 49 aa, synthetic construct; identical to SEQ ID NO: 92
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-[(4S)-4-carboxy-4-(1 9-carboxynonadecanamido)butanamido]butanamido]butanamido]b utanamido]butanamido]ethoxy}ethoxy)acetamido]ethoxy}ethoxy )acetyl attached to lysine via a side chain
  SITE 49: note = Tyrosine amide

SEQ ID NO: 439 - protein, 49 aa, synthetic construct; identical to SEQ ID NO: 113
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 440 - protein, 49 aa, synthetic construct; identical to SEQ ID NO: 114
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 441 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 47: note = N-methyl-L-glutamine
  SITE 49: note = Tyrosine amide

SEQ ID NO: 442 - protein, 49 aa, synthetic construct; identical to SEQ ID NO: 97
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 443 - protein, 49 aa, synthetic construct; identical to SEQ ID NO: 117
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 444 - protein, 49 aa, synthetic construct; identical to SEQ ID NO: 39
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 37: note = Amino-isobutyric acid
  SITE 49: note = Tyrosine amide

SEQ ID NO: 445 - protein, 49 aa, synthetic construct; identical to SEQ ID NO: 119
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 39: note = L-tert-butylglycine
  SITE 49: note = Tyrosine amide

SEQ ID NO: 446 - protein, 49 aa, synthetic construct; identical to SEQ ID NO: 95
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-[(4S)-4-carboxy-4-(1 9-carboxynonadecanamido)butanamido]butanamido]butanamido]b utanamido]butanamido]ethoxy}ethoxy)acetamido]ethoxy}ethoxy )acetyl attached to lysine via a side chain
  SITE 49: note = Tyrosine amide

SEQ ID NO: 447 - protein, 49 aa, synthetic construct; identical to SEQ ID NO: 121
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 448 - protein, 49 aa, synthetic construct; identical to SEQ ID NO: 122
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 449 - protein, 49 aa, synthetic construct; identical to SEQ ID NO: 123
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 450 - protein, 49 aa, synthetic construct; identical to SEQ ID NO: 124
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 451 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 48: note = N-methyl-L-arginine
  SITE 49: note = Tyrosine amide

SEQ ID NO: 452 - protein, 49 aa, synthetic construct; identical to SEQ ID NO: 126
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 453 - protein, 49 aa, synthetic construct; identical to SEQ ID NO: 127
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 454 - protein, 49 aa, synthetic construct; identical to SEQ ID NO: 128
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 455 - protein, 49 aa, synthetic construct; identical to SEQ ID NO: 129
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 38: note = Amino-isobutyric acid
  SITE 49: note = Tyrosine amide

SEQ ID NO: 456 - protein, 49 aa, synthetic construct; identical to SEQ ID NO: 130
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 457 - protein, 49 aa, synthetic construct; identical to SEQ ID NO: 131
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 458 - protein, 49 aa, synthetic construct; identical to SEQ ID NO: 132
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 459 - protein, 49 aa, synthetic construct; identical to SEQ ID NO: 133
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 41: note = L-tert-butylglycine
  SITE 49: note = Tyrosine amide

SEQ ID NO: 460 - protein, 49 aa, synthetic construct; identical to SEQ ID NO: 134
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 461 - protein, 49 aa, synthetic construct; identical to SEQ ID NO: 135
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 462 - protein, 49 aa, synthetic construct; identical to SEQ ID NO: 136
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 463 - protein, 49 aa, synthetic construct; identical to SEQ ID NO: 137
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 44: note = L-tert-butylglycine
  SITE 49: note = Tyrosine amide

SEQ ID NO: 464 - protein, 49 aa, synthetic construct; identical to SEQ ID NO: 138
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(17- carboxyheptadecanamido)butanamido]butanamido]butanamido]bu tanamido]butanamido]hexanoyl attached to lysine via a side chain
  SITE 49: note = Tyrosine amide

SEQ ID NO: 465 - protein, 49 aa, synthetic construct; identical to SEQ ID NO: 92
  SITE 2: note = Amino-isobutyric acid
  SITE 13: note = Amino-isobutyric acid
  SITE 20: note = Amino-isobutyric acid
  SITE 28: note = 6-[(4S)-4-carboxy-4-[(4S)-4-carboxy-4-[(4S)-4-carbo xy-4-[(4S)-4-carboxy-4-[(4S)-4-carboxy-4-[(4S)-4-carboxy-4 -(17-carboxyheptadecanamido)butanamido]butanamido]butanami do]butanamido]butanamido]butanamido]hexanoyl attached to lysine via a side chain
  SITE 49: note = Tyrosine amide

SEQ ID NO: 466 - protein, 49 aa, synthetic construct; identical to SEQ ID NO: 95
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-[(4S)-4-carboxy-4-(1 7-carboxyheptadecanamido)butanamido]butanamido]butanamido] butanamido]butanamido]ethoxy}ethoxy)acetamido]ethoxy}ethox y)acetyl attached to lysine via a side chain
  SITE 49: note = Tyrosine amide

SEQ ID NO: 467 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]butanoy l attached to lysine via a side chain
  SITE 49: note = Tyrosine amide

SEQ ID NO: 468 - protein, 49 aa, synthetic construct; identical to SEQ ID NO: 101
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-[(4S)-4-carboxy-4-(1 7-carboxyheptadecanamido)butanamido]butanamido]butanamido] butanamido]butanamido]ethoxy}ethoxy)acetamido]ethoxy}ethox y)acetyl attached to lysine via a side chain
  SITE 49: note = Tyrosine amide

SEQ ID NO: 469 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(17- carboxyheptadecanamido)butanamido]butanamido]butanamido]bu tanamido]butanamido]hexanoyl attached to lysine via a side chain
  SITE 49: note = Tyrosine amide

SEQ ID NO: 470 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-(17-carboxyheptadecanamido)butanamido] butanamido]butanamido]ethoxy}ethoxy)acetamido]ethoxy}ethox y)acetyl attached to lysine via a side chain
  SITE 49: note = Tyrosine amide

SEQ ID NO: 471 - protein, 49 aa, synthetic construct; identical to SEQ ID NO: 88
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]ethoxy} ethoxy)acetamido]ethoxy}ethoxy)acetyl attached to lysine via a side chain
  SITE 49: note = Tyrosine amide

SEQ ID NO: 472 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]ethoxy} ethoxy)acetamido]ethoxy}ethoxy)acetyl attached to lysine via a side chain
  SITE 49: note = Tyrosine amide

SEQ ID NO: 473 - protein, 49 aa, synthetic construct; identical to SEQ ID NO: 92
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-[(4S)-4-carboxy-4-(1 7-carboxyheptadecanamido)butanamido]butanamido]butanamido] butanamido]butanamido]butanoyl attached to lysine via a side chain
  SITE 49: note = Tyrosine amide

SEQ ID NO: 474 - protein, 49 aa, synthetic construct; identical to SEQ ID NO: 101
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(17- carboxyheptadecanamido)butanamido]butanamido]butanamido]bu tanamido]butanamido]hexanoyl attached to lysine via a side chain
  SITE 49: note = Tyrosine amide

SEQ ID NO: 475 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(17-carboxyheptadecanamido)butanamido]butana mido]butanamido]butanamido]butanamido]butanamido]hexanoyl attached to lysine via a side chain
  SITE 49: note = Tyrosine amide

SEQ ID NO: 476 - protein, 49 aa, synthetic construct; identical to SEQ ID NO: 150
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]ethoxy} ethoxy)acetamido]ethoxy}ethoxy)acetyl attached to lysine via a side chain
  SITE 49: note = Tyrosine amide

SEQ ID NO: 477 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-[(4S)-4-carboxy-4-(1 7-carboxyheptadecanamido)butanamido]butanamido]butanamido] butanamido]butanamido]butanoyl attached to lysine via a side chain
  SITE 49: note = Tyrosine amide

SEQ ID NO: 478 - protein, 49 aa, synthetic construct; identical to SEQ ID NO: 152
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(17- carboxyheptadecanamido)butanamido]butanamido]butanamido]bu tanamido]butanamido]hexanoyl attached to lysine via a side chain
  SITE 49: note = Tyrosine amide

SEQ ID NO: 479 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(17-carboxyheptadecanamido)butanamido]butana mido]butanamido]butanamido]butanamido]butanamido]hexanoyl attached to lysine via a side chain
  SITE 49: note = tyrosine amide

SEQ ID NO: 480 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(17- carboxyheptadecanamido)butanamido]butanamido]butanamido]bu tanamido]butanamido]hexanoyl attached to lysine via a side chain
  SITE 49: note = Tyrosine amide

SEQ ID NO: 481 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid
  SITE 28: note = Lysine attached via its side chain to the C-terminus of SEQ ID NO: 635
  SITE 49: note = Tyrosine amide

SEQ ID NO: 482 - protein, 49 aa, synthetic construct; identical to SEQ ID NO: 138
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]ethoxy} ethoxy)acetamido]ethoxy}ethoxy)acetyl attached to lysine via a side chain
  SITE 49: note = Tyrosine amide

SEQ ID NO: 483 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(2S)-4-carboxy-2-[(2S)-4-carboxy- 2-[(2S)-4-carboxy-2-(17-carboxyheptadecanamido)butanamido] butanamido]butanamido]ethoxy}ethoxy)acetamido]ethoxy}ethox y)acetyl attached to lysine via a side chain
  SITE 49: note = Tyrosine amide

SEQ ID NO: 484 - protein, 49 aa, synthetic construct; identical to SEQ ID NO: 95
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]ethoxy} ethoxy)acetamido]ethoxy}ethoxy)acetyl attached to lysine via a side chain
  SITE 49: note = Tyrosine amide

SEQ ID NO: 485 - protein, 49 aa, synthetic construct; identical to SEQ ID NO: 88
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(17- carboxyheptadecanamido)butanamido]butanamido]butanamido]bu tanamido]butanamido]hexanoyl attached to lysine via a side chain
  SITE 49: note = Tyrosine amide

SEQ ID NO: 486 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-(17-carboxyheptadecanamido)butanamido] butanamido]butanamido]ethoxy}ethoxy)acetamido]ethoxy}ethox y)acetyl attached to lysine via a side chain
  SITE 49: note = Tyrosine amide

SEQ ID NO: 487 - protein, 49 aa, synthetic construct; identical to SEQ ID NO: 161
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]ethoxy} ethoxy)acetamido]ethoxy}ethoxy)acetyl attached to lysine via a side chain
  SITE 49: note = Tyrosine amide

SEQ ID NO: 488 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid
  SITE 28: note = 6-[(4S)-4-carboxy-4-[(4S)-4-carboxy-4-[(4S)-4-carbo xy-4-[(4S)-4-carboxy-4-[(4S)-4-carboxy-4-(17-carboxyheptad ecanamido)butanamido]butanamido]butanamido]butanamido]buta namido]hexanoyl attached to lysine via a side chain
  SITE 49: note = Tyrosine amide

SEQ ID NO: 489 - protein, 49 aa, synthetic construct; identical to SEQ ID NO: 152
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]ethoxy} ethoxy)acetamido]ethoxy}ethoxy)acetyl attached to lysine via a side chain
  SITE 49: note = Tyrosine amide

SEQ ID NO: 490 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid
  SITE 28: note = 6-[(4S)-4-carboxy-4-[(4S)-4-carboxy-4-[(4S)-4-carbo xy-4-[(4S)-4-carboxy-4-[(4S)-4-carboxy-4-[(4S)-4-carboxy-4 -(17-carboxyheptadecanamido)butanamido]butanamido]butanami do]butanamido]butanamido]butanamido]hexanoyl attached to lysine via a side chain
  SITE 49: note = Tyrosine amide

SEQ ID NO: 491 - protein, 49 aa, synthetic construct; identical to SEQ ID NO: 101
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]ethoxy}e thoxy)acetamido]ethoxy}ethoxy)acetyl attached to lysine via a side chain
  SITE 49: note = Tyrosine amide

SEQ ID NO: 492 - protein, 49 aa, synthetic construct; identical to SEQ ID NO: 152
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-(17-carboxyheptadecanamido)butanamido] butanamido]butanamido]ethoxy}ethoxy)acetamido]ethoxy}ethox y)acetyl attached to lysine via a side chain
  SITE 49: note = Tyrosine amide

SEQ ID NO: 493 - protein, 49 aa, synthetic construct; identical to SEQ ID NO: 92
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-[(4S)-4-carboxy-4-(1 7-carboxyheptadecanamido)butanamido]butanamido]butanamido] butanamido]butanamido]ethoxy}ethoxy)acetamido]ethoxy}ethox y)acetyl attached to lysine via a side chain
  SITE 49: note = Tyrosine amide

SEQ ID NO: 494 - protein, 49 aa, synthetic construct; identical to SEQ ID NO: 168
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 495 - protein, 49 aa, synthetic construct; identical to SEQ ID NO: 101
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(19- carboxynonadecanamido)butanamido]butanamido]butanamido]but anamido]butanamido]hexanoyl attached to lysine via a side chain
  SITE 49: note = Tyrosine amide

SEQ ID NO: 496 - protein, 49 aa, synthetic construct; identical to SEQ ID NO: 92
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(17-carboxyheptadecanamido)butanamido]butana mido]butanamido]butanamido]butanamido]butanamido]hexanoyl attached to lysine via a side chain
  SITE 49: note = Tyrosine amide

SEQ ID NO: 497 - protein, 49 aa, synthetic construct; identical to SEQ ID NO: 88
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-(17-carboxyheptadecanamido)butanamido] butanamido]butanamido]ethoxy}ethoxy)acetamido]ethoxy}ethox y)acetyl attached to lysine via a side chain
  SITE 49: note = Tyrosine amide

SEQ ID NO: 498 - protein, 49 aa, synthetic construct; identical to SEQ ID NO: 172
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 499 - protein, 49 aa, synthetic construct; identical to SEQ ID NO: 101
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]ethoxy} ethoxy)acetamido]ethoxy}ethoxy)acetyl attached to lysine via a side chain
  SITE 49: note = Tyrosine amide

SEQ ID NO: 500 - protein, 49 aa, synthetic construct; identical to SEQ ID NO: 138
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-(17-carboxyheptadecanamido)butanamido] butanamido]butanamido]butanoyl attached to lysine via a side chain
  SITE 49: note = Tyrosine amide

SEQ ID NO: 501 - protein, 49 aa, synthetic construct; identical to SEQ ID NO: 95
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]ethoxy}e thoxy)acetamido]ethoxy}ethoxy)acetyl attached to lysine via a side chain
  SITE 49: note = Tyrosine amide

SEQ ID NO: 502 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid
  SITE 28: note = Lysine attached via its side chain to the C-terminus of SEQ ID NO: 636
  SITE 49: note = Tyrosine amide

SEQ ID NO: 503 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid
  SITE 28: note = Lysine attached via its side chain to the C-terminus of SEQ ID NO: 634
  SITE 49: note = Tyrosine amide

SEQ ID NO: 504 - protein, 49 aa, synthetic construct; identical to SEQ ID NO: 95
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]butanoyl attached to lysine via a side chain
  SITE 49: note = Tyrosine amide

SEQ ID NO: 505 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid
  SITE 28: note = Lysine attached via its side chain to the C-terminus of SEQ ID NO: 637
  SITE 49: note = Tyrosine amide

SEQ ID NO: 506 - protein, 49 aa, synthetic construct; identical to SEQ ID NO: 95
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]butanoy l attached to lysine via a side chain
  SITE 49: note = Tyrosine amide

SEQ ID NO: 507 - protein, 49 aa, synthetic construct; identical to SEQ ID NO: 97
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(19- carboxynonadecanamido)butanamido]butanamido]butanamido]but anamido]butanamido]hexanoyl attached to lysine via a side chain
  SITE 49: note = Tyrosine amide

SEQ ID NO: 508 - protein, 49 aa, synthetic construct; identical to SEQ ID NO: 119
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 39: note = Amino-isobutyric acid
  SITE 49: note = Tyrosine amide

SEQ ID NO: 509 - protein, 49 aa, synthetic construct; identical to SEQ ID NO: 126
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(19- carboxynonadecanamido)butanamido]butanamido]butanamido]but anamido]butanamido]hexanoyl attached to lysine via a side chain
  SITE 49: note = Tyrosine amide

SEQ ID NO: 510 - protein, 49 aa, synthetic construct; identical to SEQ ID NO: 133
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 41: note = Amino-isobutyric acid
  SITE 49: note = Tyrosine amide

SEQ ID NO: 511 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-[(4S)-4-carboxy-4-(1 9-carboxynonadecanamido)butanamido]butanamido]butanamido]b utanamido]butanamido]butanoyl attached to lysine via a side chain
  SITE 49: note = Tyrosine amide

SEQ ID NO: 512 - protein, 49 aa, synthetic construct; identical to SEQ ID NO: 186
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 47: note = Beta-homo-L-glutamine
  SITE 49: note = Tyrosine amide

SEQ ID NO: 513 - protein, 49 aa, synthetic construct; identical to SEQ ID NO: 88
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(17-carboxyheptadecanamido)butanamido]butana mido]butanamido]butanamido]butanamido]butanamido]hexanoyl attached to lysine via a side chain
  SITE 49: note = Tyrosine amide

SEQ ID NO: 514 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(2S)-4-carboxy-2-[(2S)-4-carboxy- 2-[(2S)-4-carboxy-2-(17-carboxyheptadecanamido)butanamido] butanamido]butanamido]ethoxy}ethoxy)acetamido]ethoxy}ethox y)acetyl attached to lysine via a side chain
  SITE 49: note = Tyrosine amide

SEQ ID NO: 515 - protein, 49 aa, synthetic construct; identical to SEQ ID NO: 189
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(19- carboxynonadecanamido)butanamido]butanamido]butanamido]but anamido]butanamido]hexanoyl attached to lysine via a side chain
  SITE 49: note = Tyrosine amide

SEQ ID NO: 516 - protein, 49 aa, synthetic construct; identical to SEQ ID NO: 190
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 44: note = D-Arginine
  SITE 49: note = Tyrosine amide

SEQ ID NO: 517 - protein, 49 aa, synthetic construct; identical to SEQ ID NO: 191
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = L-tert-butylglycine amide

SEQ ID NO: 518 - protein, 49 aa, synthetic construct; identical to SEQ ID NO: 5
  SITE 2: note = Amino-isobutyric acid
  SITE 13: note = Amino-isobutyric acid
  SITE 16: note = Acetylated lysine
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 519 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]ethoxy}e thoxy)acetamido]ethoxy}ethoxy)acetyl attached to lysine via a side chain
  SITE 49: note = Tyrosine amide

SEQ ID NO: 520 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-(19-carboxynonadecanamido)butanamido]b utanamido]butanamido]butanoyl attached to lysine via a side chain
  SITE 49: note = Tyrosine amide

SEQ ID NO: 521 - protein, 49 aa, synthetic construct; identical to SEQ ID NO: 195
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 522 - protein, 49 aa, synthetic construct; identical to SEQ ID NO: 89
  SITE 2: note = Amino-isobutyric acid
  SITE 13: note = Amino-isobutyric acid
  SITE 20: note = Amino-isobutyric acid
  SITE 28: note = Lysine attached via its side chain to the C-terminus of SEQ ID NO: 643
  SITE 49: note = Tyrosine amide

SEQ ID NO: 523 - protein, 49 aa, synthetic construct; identical to SEQ ID NO: 197
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 524 - protein, 49 aa, synthetic construct; identical to SEQ ID NO: 88
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-[(4S)-4-carboxy-4-(1 9-carboxynonadecanamido)butanamido]butanamido]butanamido]b utanamido]butanamido]butanoyl attached to lysine via a side chain
  SITE 49: note = Tyrosine amide

SEQ ID NO: 525 - protein, 49 aa, synthetic construct; identical to SEQ ID NO: 199
  SITE 2: note = Amino-isobutyric acid
  SITE 13: note = Amino-isobutyric acid
  SITE 16: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 526 - protein, 49 aa, synthetic construct; identical to SEQ ID NO: 138
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(19- carboxynonadecanamido)butanamido]butanamido]butanamido]but anamido]butanamido]hexanoyl attached to lysine via a side chain
  SITE 49: note = Tyrosine amide

SEQ ID NO: 527 - protein, 49 aa, synthetic construct; identical to SEQ ID NO: 88
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-[(4S)-4-carboxy-4-(1 7-carboxyheptadecanamido)butanamido]butanamido]butanamido] butanamido]butanamido]ethoxy}ethoxy)acetamido]ethoxy}ethox y)acetyl attached to lysine via a side chain
  SITE 49: note = Tyrosine amide

SEQ ID NO: 528 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-(17-carboxyheptadecanamido)butanamido] butanamido]butanamido]butanoyl attached to lysine via a side chain
  SITE 49: note = Tyrosine amide

SEQ ID NO: 529 - protein, 49 aa, synthetic construct; identical to SEQ ID NO: 203
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(19- carboxynonadecanamido)butanamido]butanamido]butanamido]but anamido]butanamido]hexanoyl attached to lysine via a side chain
  SITE 49: note = Tyrosine amide

SEQ ID NO: 530 - protein, 49 aa, synthetic construct; identical to SEQ ID NO: 204
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 44: note = D-Aspartic acid
  SITE 49: note = Tyrosine amide

SEQ ID NO: 531 - protein, 49 aa, synthetic construct; identical to SEQ ID NO: 88
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]ethoxy}e thoxy)acetamido]ethoxy}ethoxy)acetyl attached to lysine via a side chain
  SITE 49: note = Tyrosine amide

SEQ ID NO: 532 - protein, 49 aa, synthetic construct; identical to SEQ ID NO: 161
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]ethoxy}e thoxy)acetamido]ethoxy}ethoxy)acetyl attached to lysine via a side chain
  SITE 49: note = Tyrosine amide

SEQ ID NO: 533 - protein, 49 aa, synthetic construct; identical to SEQ ID NO: 141
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-[(4S)-4-carboxy-4-(19-carboxynonadecanamido) butanamido]butanamido]butanamido]butanamido]butanamido]but anamido]butanamido]hexanoyl attached to lysine via a side chain
  SITE 49: note = Tyrosine amide

SEQ ID NO: 534 - protein, 49 aa, synthetic construct; identical to SEQ ID NO: 208
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 47: note = Beta-homo-L-glutamine
  SITE 49: note = Tyrosine amide

SEQ ID NO: 535 - protein, 49 aa, synthetic construct; identical to SEQ ID NO: 209
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 536 - protein, 49 aa, synthetic construct; identical to SEQ ID NO: 210
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 537 - protein, 49 aa, synthetic construct; identical to SEQ ID NO: 211
  SITE 2: note = Amino-isobutyric acid
  SITE 13: note = Amino-isobutyric acid
  SITE 20: note = D-Aspartic acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 538 - protein, 49 aa, synthetic construct; identical to SEQ ID NO: 96
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-[(4S)-4-carboxy-4-(1 9-carboxynonadecanamido)butanamido]butanamido]butanamido]b utanamido]butanamido]butanoyl attached to lysine via a side chain
  SITE 49: note = Tyrosine amide

SEQ ID NO: 539 - protein, 49 aa, synthetic construct; identical to SEQ ID NO: 213
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 42: note = Amino-isobutyric acid
  SITE 49: note = Tyrosine amide

SEQ ID NO: 540 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = D-Tyrosine amide

SEQ ID NO: 541 - protein, 49 aa, synthetic construct; identical to SEQ ID NO: 88
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 542 - protein, 49 aa, synthetic construct; identical to SEQ ID NO: 216
  SITE 2: note = Amino-isobutyric acid
  SITE 13: note = Amino-isobutyric acid
  SITE 20: note = D-Arginine
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 543 - protein, 49 aa, synthetic construct; identical to SEQ ID NO: 217
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 544 - protein, 49 aa, synthetic construct; identical to SEQ ID NO: 218
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 43: note = Amino-isobutyric acid
  SITE 49: note = Tyrosine amide

SEQ ID NO: 545 - protein, 49 aa, synthetic construct; identical to SEQ ID NO: 138
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]ethoxy}e thoxy)acetamido]ethoxy}ethoxy)acetyl attached to lysine via a side chain
  SITE 49: note = Tyrosine amide

SEQ ID NO: 546 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 547 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 548 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 47: note = N-methyl-L-glutamine
  SITE 49: note = Tyrosine amide

SEQ ID NO: 549 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-[(4S)-4-carboxy-4-(1 9-carboxynonadecanamido)butanamido]butanamido]butanamido]b utanamido]butanamido]butanoyl attached to lysine via a side chain
  SITE 49: note = Tyrosine amide

SEQ ID NO: 550 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-[(4S)-4-carboxy-4-(19-carboxynonadecanamido) butanamido]butanamido]butanamido]butanamido]butanamido]but anamido]butanamido]hexanoyl attached to lysine via a side chain
  SITE 49: note = Tyrosine amide

SEQ ID NO: 551 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-[(4S)-4-carboxy-4-(19-carboxynonadecanamido) butanamido]butanamido]butanamido]butanamido]butanamido]but anamido]butanamido]hexanoyl attached to lysine via a side chain
  SITE 49: note = Tyrosine amide

SEQ ID NO: 552 - protein, 49 aa, synthetic construct; identical to SEQ ID NO: 226
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]ethoxy}e thoxy)acetamido]ethoxy}ethoxy)acetyl attached to lysine via a side chain
  SITE 49: note = Tyrosine amide

SEQ ID NO: 553 - protein, 49 aa, synthetic construct; identical to SEQ ID NO: 227
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 554 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-[(4S)-4-carboxy-4-(1 9-carboxynonadecanamido)butanamido]butanamido]butanamido]b utanamido]butanamido]ethoxy}ethoxy)acetamido]ethoxy}ethoxy )acetyl attached to lysine via a side chain
  SITE 49: note = Tyrosine amide

SEQ ID NO: 555 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-[(4S)-4-carboxy-4-(1 9-carboxynonadecanamido)butanamido]butanamido]butanamido]b utanamido]butanamido]ethoxy}ethoxy)acetamido]ethoxy}ethoxy )acetyl attached to lysine via a side chain
  SITE 49: note = Tyrosine amide

SEQ ID NO: 556 - protein, 49 aa, synthetic construct; identical to SEQ ID NO: 230
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(19- carboxynonadecanamido)butanamido]butanamido]butanamido]but anamido]butanamido]hexanoyl attached to lysine via a side chain
  SITE 49: note = Tyrosine amide

SEQ ID NO: 557 - protein, 49 aa, synthetic construct; identical to SEQ ID NO: 231
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]ethoxy}e thoxy)acetamido]ethoxy}ethoxy)acetyl attached to lysine via a side chain
  SITE 49: note = Tyrosine amide

SEQ ID NO: 558 - protein, 49 aa, synthetic construct; identical to SEQ ID NO: 232
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 559 - protein, 49 aa, synthetic construct; identical to SEQ ID NO: 226
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 560 - protein, 49 aa, synthetic construct; identical to SEQ ID NO: 234
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 561 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-[(4S)-4-carboxy-4-(1 9-carboxynonadecanamido)butanamido]butanamido]butanamido]b utanamido]butanamido]butanoyl attached to lysine via a side chain
  SITE 49: note = Tyrosine amide

SEQ ID NO: 562 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 48: note = N-methyl-L-arginine
  SITE 49: note = Tyrosine amide

SEQ ID NO: 563 - protein, 49 aa, synthetic construct; identical to SEQ ID NO: 237
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 564 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]butanoyl attached to lysine via a side chain
  SITE 49: note = Tyrosine amide

SEQ ID NO: 565 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = Lysine attached via its side chain to the C-terminus of SEQ ID NO: 638
  SITE 49: note = Tyrosine amide

SEQ ID NO: 566 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = Lysine attached via its side chain to the C-terminus of SEQ ID NO: 639
  SITE 49: note = Tyrosine amide

SEQ ID NO: 567 - protein, 49 aa, synthetic construct; identical to SEQ ID NO: 241
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 568 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(19- carboxynonadecanamido)butanamido]butanamido]butanamido]but anamido]butanamido]hexanoyl attached to lysine via a side chain
  SITE 49: note = Tyrosine amide

SEQ ID NO: 569 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]butanoyl attached to lysine via a side chain
  SITE 49: note = Tyrosine amide

SEQ ID NO: 570 - protein, 49 aa, synthetic construct; identical to SEQ ID NO: 244
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 571 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = 6-[(4S)-4-carboxy-4-[(4S)-4-carboxy-4-[(4S)-4-carbo xy-4-[(4S)-4-carboxy-4-[(4S)-4-carboxy-4-[(4S)-4-carboxy-4 -(17-carboxyheptadecanamido)butanamido]butanamido]butanami do]butanamido]butanamido]butanamido]hexanoyl attached to lysine via a side chain
  SITE 49: note = Tyrosine amide

SEQ ID NO: 572 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-[(4S)-4-carboxy-4-(1 7-carboxyheptadecanamido)butanamido]butanamido]butanamido] butanamido]butanamido]ethoxy}ethoxy)acetamido]ethoxy}ethox y)acetyl attached to lysine via a side chain
  SITE 49: note = Tyrosine amide

SEQ ID NO: 573 - protein, 49 aa, synthetic construct; identical to SEQ ID NO: 231
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-(17-carboxyheptadecanamido)butanamido] butanamido]butanamido]butanoyl attached to lysine via a side chain
  SITE 49: note = Tyrosine amide

SEQ ID NO: 574 - protein, 49 aa, synthetic construct; identical to SEQ ID NO: 230
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-(17-carboxyheptadecanamido)butanamido] butanamido]butanamido]ethoxy}ethoxy)acetamido]ethoxy}ethox y)acetyl attached to lysine via a side chain
  SITE 49: note = Tyrosine amide

SEQ ID NO: 575 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = Lysine attached via its side chain to the C-terminus of SEQ ID NO: 643
  SITE 49: note = Tyrosine amide

SEQ ID NO: 576 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]butanoy l attached to lysine via a side chain
  SITE 49: note = Tyrosine amide

SEQ ID NO: 577 - protein, 49 aa, synthetic construct; identical to SEQ ID NO: 251
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]ethoxy} ethoxy)acetamido]ethoxy}ethoxy)acetyl attached to lysine via a side chain
  SITE 49: note = Tyrosine amide

SEQ ID NO: 578 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-[(4S)-4-carboxy-4-(1 7-carboxyheptadecanamido)butanamido]butanamido]butanamido] butanamido]butanamido]butanoyl attached to lysine via a side chain
  SITE 49: note = Tyrosine amide

SEQ ID NO: 579 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]butanoy l attached to lysine via a side chain
  SITE 49: note = Tyrosine amide

SEQ ID NO: 580 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]ethoxy} ethoxy)acetamido]ethoxy}ethoxy)acetyl attached to lysine via a side chain
  SITE 49: note = Tyrosine amide

SEQ ID NO: 581 - protein, 49 aa, synthetic construct; identical to SEQ ID NO: 255
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(17-carboxyheptadecanamido)butanamido]butana mido]butanamido]butanamido]butanamido]butanamido]hexanoyl attached to lysine via a side chain
  SITE 49: note = Tyrosine amide

SEQ ID NO: 582 - protein, 49 aa, synthetic construct; identical to SEQ ID NO: 226
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]ethoxy} ethoxy)acetamido]ethoxy}ethoxy)acetyl attached to lysine via a side chain
  SITE 49: note = Tyrosine amide

SEQ ID NO: 583 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = Lysine attached via its side chain to the C-terminus of SEQ ID NO: 636
  SITE 49: note = Tyrosine amide

SEQ ID NO: 584 - protein, 49 aa, synthetic construct; identical to SEQ ID NO: 231
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]ethoxy} ethoxy)acetamido]ethoxy}ethoxy)acetyl attached to lysine via a side chain
  SITE 49: note = Tyrosine amide

SEQ ID NO: 585 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(17-carboxyheptadecanamido)butanamido]butana mido]butanamido]butanamido]butanamido]butanamido]hexanoyl attached to lysine via a side chain
  SITE 49: note = Tyrosine amide

SEQ ID NO: 586 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 28: note = 6-[(4S)-4-carboxy-4-[(4S)-4-carboxy-4-[(4S)-4-carbo xy-4-[(4S)-4-carboxy-4-[(4S)-4-carboxy-4-(17-carboxyheptad ecanamido)butanamido]butanamido]butanamido]butanamido]buta namido]hexanoyl attached to lysine via a side chain
  SITE 49: note = Tyrosine amide

SEQ ID NO: 587 - protein, 49 aa, synthetic construct; identical to SEQ ID NO: 5
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-[(4S)-4-carboxy-4-(1 7-carboxyheptadecanamido)butanamido]butanamido]butanamido] butanamido]butanamido]ethoxy}ethoxy)acetamido]ethoxy}ethox y)acetyl attached to lysine via a side chain
  SITE 49: note = Tyrosine amide

SEQ ID NO: 588 - protein, 49 aa, synthetic construct; identical to SEQ ID NO: 251
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-(17-carboxyheptadecanamido)butanamido] butanamido]butanamido]butanoyl attached to lysine via a side chain
  SITE 49: note = Tyrosine amide

SEQ ID NO: 589 - protein, 49 aa, synthetic construct; identical to SEQ ID NO: 226
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(17- carboxyheptadecanamido)butanamido]butanamido]butanamido]bu tanamido]butanamido]hexanoyl attached to lysine via a side chain
  SITE 49: note = Tyrosine amide

SEQ ID NO: 590 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = Lysine attached via its side chain to the C-terminus of SEQ ID NO: 637
  SITE 49: note = Tyrosine amide

SEQ ID NO: 591 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(17-carboxyheptadecanamido)butanamido]butana mido]butanamido]butanamido]butanamido]butanamido]hexanoyl attached to lysine via a side chain
  SITE 49: note = Tyrosine amide

SEQ ID NO: 592 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-(17-carboxyheptadecanamido)butanamido] butanamido]butanamido]ethoxy}ethoxy)acetamido]ethoxy}ethox y)acetyl attached to lysine via a side chain
  SITE 49: note = Tyrosine amide

SEQ ID NO: 593 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(17- carboxyheptadecanamido)butanamido]butanamido]butanamido]bu tanamido]butanamido]hexanoyl attached to lysine via a side chain
  SITE 49: note = Tyrosine amide

SEQ ID NO: 594 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-(17-carboxyheptadecanamido)butanamido] butanamido]butanamido]butanoyl attached to lysine via a side chain
  SITE 49: note = Tyrosine amide

SEQ ID NO: 595 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]ethoxy}e thoxy)acetamido]ethoxy}ethoxy)acetyl attached to lysine via a side chain
  SITE 49: note = Tyrosine amide

SEQ ID NO: 596 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]ethoxy} ethoxy)acetamido]ethoxy}ethoxy)acetyl attached to lysine via a side chain
  SITE 49: note = Tyrosine amide

SEQ ID NO: 597 - protein, 49 aa, synthetic construct; identical to SEQ ID NO: 255
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-(19-carboxynonadecanamido)butanamido]b utanamido]butanamido]butanoyl attached to lysine via a side chain
  SITE 49: note = Tyrosine amide

SEQ ID NO: 598 - protein, 49 aa, synthetic construct; identical to SEQ ID NO: 230
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-(17-carboxyheptadecanamido)butanamido] butanamido]butanamido]butanoyl attached to lysine via a side chain
  SITE 49: note = Tyrosine amide

SEQ ID NO: 599 - protein, 49 aa, synthetic construct; identical to SEQ ID NO: 255
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-(19-carboxynonadecanamido)butanamido]b utanamido]butanamido]ethoxy}ethoxy)acetamido]ethoxy}ethoxy )acetyl attached to lysine via a side chain
  SITE 49: note = Tyrosine amide

SEQ ID NO: 600 - protein, 49 aa, synthetic construct; identical to SEQ ID NO: 255
  SITE 2: note = Amino-isobutyric acid
  SITE 13: note = Amino-isobutyric acid
  SITE 20: note = Amino-isobutyric acid
  SITE 28: note = 2-(2-{2-[(4S)-4-carboxy-4-[(4S)-4-carboxy-4-[(4S)-4 -carboxy-4-(17-carboxyheptadecanamido)butanamido]butanamid o]butanamido]ethoxy}ethoxy)acetyl attached to lysine via a side chain
  SITE 49: note = Tyrosine amide

SEQ ID NO: 601 - protein, 49 aa, synthetic construct; identical to SEQ ID NO: 255
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 602 - protein, 49 aa, synthetic construct; identical to SEQ ID NO: 255
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-(17-carboxyheptadecanamido)butanamido] butanamido]butanamido]butanoyl attached to lysine via a side chain
  SITE 49: note = Tyrosine amide

SEQ ID NO: 603 - protein, 49 aa, synthetic construct; identical to SEQ ID NO: 277
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 47: note = Beta-homo-L-glutamine
  SITE 49: note = Tyrosine amide

SEQ ID NO: 604 - protein, 49 aa, synthetic construct; identical to SEQ ID NO: 251
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]ethoxy}e thoxy)acetamido]ethoxy}ethoxy)acetyl attached to lysine via a side chain
  SITE 49: note = Tyrosine amide

SEQ ID NO: 605 - protein, 49 aa, synthetic construct; identical to SEQ ID NO: 255
  SITE 2: note = Amino-isobutyric acid
  SITE 13: note = Amino-isobutyric acid
  SITE 20: note = Amino-isobutyric acid
  SITE 28: note = 6-[(4S)-4-carboxy-4-[(4S)-4-carboxy-4-[(4S)-4-carbo xy-4-[(4S)-4-carboxy-4-[(4S)-4-carboxy-4-[(4S)-4-carboxy-4 -(17-carboxyheptadecanamido)butanamido]butanamido]butanami do]butanamido]butanamido]butanamido]hexanoyl attached to lysine via a side chain
  SITE 49: note = Tyrosine amide

SEQ ID NO: 606 - protein, 49 aa, synthetic construct; identical to SEQ ID NO: 251
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-(19-carboxynonadecanamido)butanamido]b utanamido]butanamido]butanoyl attached to lysine via a side chain
  SITE 49: note = Tyrosine amide

SEQ ID NO: 607 - protein, 49 aa, synthetic construct; identical to SEQ ID NO: 115
  SITE 2: note = Amino-isobutyric acid
  SITE 13: note = Amino-isobutyric acid
  SITE 20: note = Amino-isobutyric acid
  SITE 28: note = Lysine attached via its side chain to the C-terminus of SEQ ID NO: 640
  SITE 49: note = Tyrosine amide

SEQ ID NO: 608 - protein, 49 aa, synthetic construct; identical to SEQ ID NO: 255
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-(17-carboxyhepta decanamido)butanamido]ethoxy}ethoxy)acetamido]ethoxy}ethox y)acetyl attached to lysine via a side chain
  SITE 49: note = Tyrosine amide

SEQ ID NO: 609 - protein, 49 aa, synthetic construct; identical to SEQ ID NO: 231
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-(19-carboxynonadecanamido)butanamido]b utanamido]butanamido]butanoyl attached to lysine via a side chain
  SITE 49: note = Tyrosine amide

SEQ ID NO: 610 - protein, 49 aa, synthetic construct; identical to SEQ ID NO: 255
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-(17-carboxyheptadecanamido)butanamido] butanamido]butanamido]ethoxy}ethoxy)acetamido]ethoxy}ethox y)acetyl attached to lysine via a side chain
  SITE 49: note = Tyrosine amide

SEQ ID NO: 611 - protein, 49 aa, synthetic construct; identical to SEQ ID NO: 230
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 612 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(19-carboxynonadecanamido)butanamido]butanam ido]butanamido]butanamido]butanamido]butanamido]hexanoyl attached to lysine via a side chain
  SITE 49: note = Tyrosine amide

SEQ ID NO: 613 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]ethoxy}e thoxy)acetamido]ethoxy}ethoxy)acetyl attached to lysine via a side chain
  SITE 49: note = Tyrosine amide

SEQ ID NO: 614 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-[(4S)-4-carboxy-4-(19-carboxynonadecanamido) butanamido]butanamido]butanamido]butanamido]butanamido]but anamido]butanamido]hexanoyl attached to lysine via a side chain
  SITE 49: note = Tyrosine amide

SEQ ID NO: 615 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-(19-carboxynonadecan amido)butanamido]butanamido]butanamido]butanamido]butanoyl attached to lysine via a side chain
  SITE 49: note = Tyrosine amide

SEQ ID NO: 616 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(19- carboxynonadecanamido)butanamido]butanamido]butanamido]but anamido]butanamido]hexanoyl attached to lysine via a side chain
  SITE 49: note = Tyrosine amide

SEQ ID NO: 617 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = Lysine attached via its side chain to the C-terminus of SEQ ID NO: 640
  SITE 49: note = Tyrosine amide

SEQ ID NO: 618 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]butanoy l attached to lysine via a side chain
  SITE 49: note = Tyrosine amide

SEQ ID NO: 619 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-[(4S )-4-carboxy-4-(17-carboxyheptadecanamido)butanamido]butana mido]butanamido]butanamido]butanamido]butanamido]hexanoyl attached to lysine via a side chain
  SITE 49: note = Tyrosine amide

SEQ ID NO: 620 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-(17-carboxyheptadecanamido)butanamido] butanamido]butanamido]butanoyl attached to lysine via a side chain
  SITE 49: note = Tyrosine amide

SEQ ID NO: 621 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = (2S)-2-amino-6-[(4S)-4-carboxy-4-[(4S)-4-carboxy-4- [(4S)-4-carboxy-4-[(4S)-4-carboxy-4-[(4S)-4-carboxy-4-(17- carboxyheptadecanamido)butanamido]butanamido]butanamido]bu tanamido]butanamido]hexanoyl attached to lysine via a side chain
  SITE 49: note = Tyrosine amide

SEQ ID NO: 622 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-[(4S)-4-carboxy-4-(1 7-carboxyheptadecanamido)butanamido]butanamido]butanamido] butanamido]butanamido]butanoyl attached to lysine via a side chain
  SITE 49: note = Tyrosine amide

SEQ ID NO: 623 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-[(4S)-4-carboxy-4-(17-carboxyheptadeca namido)butanamido]butanamido]butanamido]butanamido]ethoxy} ethoxy)acetamido]ethoxy}ethoxy)acetyl attached to lysine via a side chain
  SITE 49: note = Tyrosine amide

SEQ ID NO: 624 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(2S)-4-carboxy-2-[(2S)-4-carboxy- 2-[(2S)-4-carboxy-2-(17-carboxyheptadecanamido)butanamido] butanamido]butanamido]ethoxy}ethoxy)acetamido]ethoxy}ethox y)acetyl attached to lysine via a side chain
  SITE 49: note = Tyrosine amide

SEQ ID NO: 625 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = 2-(2-{2-[2-(2-{2-[(4S)-4-carboxy-4-[(4S)-4-carboxy- 4-[(4S)-4-carboxy-4-(17-carboxyheptadecanamido)butanamido] butanamido]butanamido]ethoxy}ethoxy)acetamido]ethoxy}ethox y)acetyl attached to lysine via a side chain
  SITE 49: note = Tyrosine amide

SEQ ID NO: 626 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = 2-(2-{2-[(4S)-4-carboxy-4-[(4S)-4-carboxy-4-[(4S)-4 -carboxy-4-(17-carboxyheptadecanamido)butanamido]butanamid o]butanamido]ethoxy}ethoxy)acetyl attached to lysine via a side chain
  SITE 49: note = Tyrosine amide

SEQ ID NO: 627 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = 6-[(4S)-4-carboxy-4-[(4S)-4-carboxy-4-[(4S)-4-carbo xy-4-[(4S)-4-carboxy-4-[(4S)-4-carboxy-4-(19-carboxynonade canamido)butanamido]butanamido]butanamido]butanamido]butan amido]hexanoyl attached to lysine via a side chain
  SITE 49: note = Tyrosine amide

SEQ ID NO: 628 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = Lysine attached via its side chain to the C-terminus of SEQ ID NO: 637
  SITE 49: note = Tyrosine amide

SEQ ID NO: 629 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = 6-[(4S)-4-carboxy-4-[(4S)-4-carboxy-4-[(4S)-4-carbo xy-4-[(4S)-4-carboxy-4-[(4S)-4-carboxy-4-[(4S)-4-carboxy-4 -(17-carboxyheptadecanamido)butanamido]butanamido]butanami do]butanamido]butanamido]butanamido]hexanoyl attached to lysine via a side chain
  SITE 49: note = Tyrosine amide

SEQ ID NO: 630 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = 6-[(4S)-4-carboxy-4-[(4S)-4-carboxy-4-[(4S)-4-carbo xy-4-[(4S)-4-carboxy-4-[(4S)-4-carboxy-4-(17-carboxyheptad ecanamido)butanamido]butanamido]butanamido]butanamido]buta namido]hexanoyl attached to lysine via a side chain
  SITE 49: note = Tyrosine amide

SEQ ID NO: 631 - protein, 49 aa, synthetic construct; identical to SEQ ID NO: 222
  SITE 2: note = Amino-isobutyric acid
  SITE 13: note = Amino-isobutyric acid
  SITE 20: note = Amino-isobutyric acid
  SITE 28: note = (4S)-4-carboxy-4-[(4S)-4-carboxy-4-[(4S)-4-carboxy- 4-[(4S)-4-carboxy-4-[(4S)-4-carboxy-4-[(4S)-4-carboxy-4-(1 9-carboxynonadecanamido)butanamido]butanamido]butanamido]b utanamido]butanamido]butanoyl attached to lysine via a side chain
  SITE 49: note = Tyrosine amide

SEQ ID NO: 632 - protein, 35 aa, synthetic construct
  SITE 1: note = N-methyl-L-tyrosine
  SITE 2: note = Amino-isobutyric acid
  SITE 6: note = Alpha-methyl-L-phenylalanine
  SITE 10: note = Lysine attached via its side chain to the C-terminus of SEQ ID NO: 641
  SITE 13: note = Amino-isobutyric acid
  SITE 20: note = 2-amino-2-methylbutanoic acid
  SITE 30: note = Amino-isobutyric acid
  SITE 35: note = Tyrosine amide
YXEGTFTSDKSIXLEKQRQXEFVRHLLNKXTRQRY

SEQ ID NO: 633 - protein, 35 aa, synthetic construct; identical to SEQ ID NO: 632
  SITE 1: note = N-methyl-L-tyrosine
  SITE 2: note = Amino-isobutyric acid
  SITE 6: note = Alpha-methyl-L-phenylalanine
  SITE 10: note = Lysine attached via its side chain to the C-terminus of SEQ ID NO: 642
  SITE 13: note = Amino-isobutyric acid
  SITE 20: note = 2-amino-2-methylbutanoic acid
  SITE 30: note = Amino-isobutyric acid
  SITE 35: note = Tyrosine amide

SEQ ID NO: 634 - protein, 4 aa, synthetic construct
  SITE 1: note = 17-carboxy-heptadecanoyl glutamic acid
  SITE 4: note = 2-[2-(2-{2-[2-(2-aminoethoxy)ethoxy]acetamido}ethox y)ethoxy]acetic acid glutamic acid
EEEE

SEQ ID NO: 635 - protein, 5 aa, synthetic construct
  SITE 1: note = 17-carboxy-heptadecanoyl glutamic acid
  SITE 5: note = glutamic acid attached to the side chain of lysine
EEEEE

SEQ ID NO: 636 - protein, 5 aa, synthetic construct
  SITE 1: note = 17-carboxy-heptadecanoyl glutamic acid
EEEEE

SEQ ID NO: 637 - protein, 6 aa, synthetic construct
  SITE 1: note = 17-carboxy-heptadecanoyl glutamic acid
EEEEEE

SEQ ID NO: 638 - protein, 4 aa, synthetic construct
  SITE 1: note = 19-carboxy-nonadecanoyl glutamic acid
  SITE 4: note = 2-[2-(2-{2-[2-(2-aminoethoxy)ethoxy]acetamido}ethox y)ethoxy]acetic acid glutamic acid
EEEE

SEQ ID NO: 639 - protein, 5 aa, synthetic construct
  SITE 1: note = 19-carboxy-nonadecanoyl glutamic acid
  SITE 5: note = glutamic acid attached to the side chain of lysine
EEEEE

SEQ ID NO: 640 - protein, 4 aa, synthetic construct
  SITE 1: note = 17-carboxy-heptadecanoyl glutamic acid
EEEE

SEQ ID NO: 641 - protein, 4 aa, synthetic construct
  SITE 1: note = hexadecanoyl glycine
GGGG

SEQ ID NO: 642 - protein, 4 aa, synthetic construct
  SITE 1: note = 17-carboxy-heptadecanoyl glycine
GGGG

SEQ ID NO: 643 - protein, 6 aa, synthetic construct
  SITE 1: note = 17-carboxy-heptadecanoyl glutamic acid
  SITE 6: note = glutamic acid attached to the side chain of lysine
EEEEEE

SEQ ID NO: 644 - protein, 4 aa, synthetic construct
PSEG

SEQ ID NO: 645 - protein, 5 aa, synthetic construct
ASLRH

SEQ ID NO: 646 - protein, 28 aa, synthetic construct
  SITE 2: note = Amino-isobutyric acid
  VARIANT 3: note = E or D
  VARIANT 10: note = Y or L
  VARIANT 13: note = Amino-isobutyric acid or L
  VARIANT 15: note = E, D, or A
  VARIANT 16: note = K, E, D, Amino-isobutyric acid, G, Acetylated lysine, A, L, S, Q, or R
  VARIANT 17: note = Q, E, K, or A
  VARIANT 19: note = Q or E
  VARIANT 20: note = Amino-isobutyric acid, D-Aspartic acid, or D-Arginine
  VARIANT 21: note = A, E or K
  VARIANT 23: note = V or I
  VARIANT 24: note = E or Q
  VARIANT 25: note = W or Y
  VARIANT 27: note = I or L
YXXGTFTSDXSIXLXXXAXXXFXXXLXK

SEQ ID NO: 647 - protein, 49 aa, synthetic construct
  SITE 2: note = Amino-isobutyric acid
  VARIANT 3: note = E or D
  VARIANT 10: note = Y or L
  VARIANT 13: note = Amino-isobutyric acid or L
  VARIANT 15: note = E, D, or A
  VARIANT 16: note = K, E, D, Amino-isobutyric acid, G, Acetylated lysine, A, L, S, Q, or R
  VARIANT 17: note = Q, E, K, or A
  VARIANT 19: note = Q or E
  VARIANT 20: note = Amino-isobutyric acid, D-Aspartic acid, or D-Arginine
  VARIANT 21: note = A, E, or K
  VARIANT 23: note = V or I
  VARIANT 24: note = E or Q
  VARIANT 25: note = W or Y
  VARIANT 27: note = I or L
  VARIANT 31: note = P, G, or Q
  VARIANT 32: note = S, E, or R
  VARIANT 33: note = S, E, Y, or T
  VARIANT 34: note = G, P, Y, or A
  VARIANT 35: note = A, Q, I, V, E, or L
  VARIANT 36: note = S, E, T, D, or L
  VARIANT 37: note = L, amino-isobutyric acid, V, D-Leucine, I, or L-tert-butylglycine
  VARIANT 38: note = R, L, or amino-isobutyric acid
  VARIANT 39: note = H, amino-isobutyric acid, D-Histidine, or L-tert-butylglycine
  VARIANT 41: note = L, Y, amino-isobutyric acid, I, or L-tert-butylglycine
  VARIANT 42: note = N or amino-isobutyric acid
  VARIANT 43: note = W, H, L, R, or amino-isobutyric acid
  VARIANT 44: note = L, amino-isobutyric acid, D-Arginine, D-Aspartic acid, or L-tert-butylglycine
  VARIANT 46: note = R, homo-L-arginine, or D-Arginine
  VARIANT 47: note = Q, beta-homo-L-glutamine, or N-methyl-L-glutamine
  VARIANT 48: note = R or N-methyl-L-arginine
  VARIANT 49: note = Y, L-tert-butylglycine, cyclohexyl-glycine, D-Tyrosine, S-phenylglycine
YXXGTFTSDXSIXLXXXAXXXFXXXLXKGGXXXXXXXXXYXXXXTXXXX

SEQ ID NO: 648 - protein, 6 aa, synthetic construct
GGGSEY

SEQ ID NO: 649 - protein, 6 aa, synthetic construct
GGPEYY

SEQ ID NO: 650 - protein, 6 aa, Synthetic construct
GGPRSG

SEQ ID NO: 651 - protein, 6 aa, synthetic construct
GGPRYY

SEQ ID NO: 652 - protein, 6 aa, synthetic construct
GGPSYY

SEQ ID NO: 653 - protein, 6 aa, synthetic construct
GGQRSY

SEQ ID NO: 654 - protein, 6 aa, synthetic construct
GGQRYY

SEQ ID NO: 655 - protein, 6 aa, synthetic construct
GGQSSY

(19) United States
(12) Patent Application Publication
(10) Pub. No.: US 2024/0247080 A1
(43) Pub. Date: Jul. 25, 2024

(54) GLP1/GIP/NPY2 RECEPTOR TRIPLE AGONISTS

(71) Applicant: Boehringer Ingelheim International GmbH, Ingelheim am Rhein (DE)

(72) Inventors: Peter Wilhelm HAEBEL, Mittelbiberach (DE); Robert AUGUSTIN, Biberach (DE); Charlotte Stahl MADSEN, Måløv (DK); Anthony MURRAY, Charlottenlund (DK); Daniel Paul TEUFEL, Hergatz (DE)

(21) Appl. No.: 18/389,892

(22) Filed: Dec. 20, 2023

(30) Foreign Application Priority Data
Dec. 21, 2022 (EP) 22215510.3

(51) Int. Cl. C07K 19/00; A61P 3/04; C07K 7/06; C07K 7/08; C07K 14/605

(52) U.S. Cl. CPC C07K 19/00; A61P 3/04; C07K 7/06; C07K 7/08; C07K 14/605; A61K 38/00

(57) ABSTRACT

Hybrid polypeptides that agonize the GIP, GLP-1 and neuropeptide Y2 (NPY2) receptors and their medical use in the treatment of a variety of diseases, conditions or disorders, such as obesity, diabetes, and/or NASH are disclosed. The polypeptides have the general structure Z1-Z2-Z3, wherein Z1 is a hybrid polypeptide providing GIPR and GLP1R agonism, Z2 is a linker, and Z3 is a polypeptide providing hY2R agonism.

### FIELD OF THE INVENTION

The present invention relates to hybrid polypeptides that agonize the GIP, GLP-1 and neuropeptide Y2 (NPY2) receptors and to their medical use in the treatment of a variety of diseases, conditions or disorders, such as obesity, diabetes, and/or NASH.

### BACKGROUND INFORMATION

Overweight and obesity are defined as abnormal or excessive fat accumulation that presents a risk to health. A body mass index (BMI) over 27 kg/m² is considered as overweight, and a BMI over 30 kg/m² is considered as obese. The BMI is calculated based on body weight and height. Obesity has in the past 50 years reached pandemic levels. Worldwide obesity has nearly tripled since 1975. In 2016, more than 1.9 billion adults and more than 340 million children and adolescents were overweight or obese. Both overweight and obesity are a major risk factor for a number of chronic diseases, including type 2 diabetes, cardiovascular diseases and cancer which are the leading causes of morbidity and death in the United States. Obesity is thus a serious condition and associated with poorer mental health outcomes, reduced quality of life, and contributes to a decline in life expectancy (Abdelaal 2017). According to the WHO overweight, and obesity are no longer considered a problem limited to high income countries but are now dramatically on the rise in low- and middle-income countries. WHO's Global Health Observatory indicate that, in 2016, 39% of women or men aged 18 and over were overweight and 11% of men and 15% of women were obese.

Peptide YY (PYY) is a 36-amino acid peptide with the sequence YPIKPEAPREDASPEELNRYYASLRHYLNLVTRQRY (SEQ ID NO: 308), found in endocrine L cells in the mucosa of the gastrointestinal tract, especially in the ileum and colon. PYY belongs to the pancreatic polypeptide (PP) family together with neuropeptide Y (NPY) and pancreatic polypeptide (PP). This family of peptides act upon the NPY receptors designated NPY1R (Y1), NPY2R (Y2), NPY4R (Y4), and NPY5R (Y5). The receptor family belongs to a class of G protein-coupled receptors (GPCRs) that are expressed in the CNS, especially in regions of the hypothalamus. The receptors NPY1R-NPY5R exhibit both anorectic (NPY2R, NPY4R) and orexigenic effects (NPY5R, NPY1R). The two major forms of peptide YY are PYY₁₋₃₆ and PYY₃₋₃₆. PYY₁₋₃₆ is released postprandially from intestinal L cells in proportion to energy intake and in part truncated to PYY₃₋₃₆, which is the main circulating form of PYY and a relatively selective Y2 receptor agonist. PYY₁₋₃₆ and PYY₃₋₃₆ inhibit gastric acid secretion, gastrointestinal transit and food intake. Food intake is inhibited both via a stimulant effect on Y2 receptors on vagal afferent neurons and an interaction with Y2 receptors in the hypothalamus, which is consistent with the ability of PYY to gain access to the brain via circumventricular organs such as the area postrema and subfornical organ. Furthermore, it is known that PYY concentrations in the blood of people with obesity are lower than those of healthy individuals. Thus, NPY2R and/or NPY4R agonists may hold potential in the treatment of obesity.

Glucagon-like peptide-1 (GLP-1) and glucose-dependent insulinotropic polypeptide (GIP), belong to the family of incretins. GLP-1(7-37) is a 31-amino acid peptide, with the sequence HAEGTFTSDVSSYLEGQAAKEFIAWLVKGRG (SEQ ID NO: 309) and GIP is a 42-amino acid peptide, with the sequence YAEGTFISDYSIAMDKIHQQDFVNWLLAQKGKKNDWKHNITQ (SEQ ID NO: 310). GLP-1 and GIP are secreted from small intestinal L cells and K cells, respectively. GLP-1 acts via GLP-1 receptors and is known to have a sugar-dependent insulinotropic action (i.e. stimulate insulin release) and a feeding suppressive action. GIP acts via GIP receptors and is also known to have a sugar-dependent insulinotropic action, though its influence on feeding is less clear. GLP-1 receptor/GIP receptor coagonist peptide has been reported to show a stronger hypoglycemic action and body weight-lowering action than those of a GLP-1 receptor agonist alone. Therefore, research efforts have been made to develop GLP-1/GIP receptor co-agonists for the treatment of obesity and/or diabetes, based on the structure of natural glucagon, GIP, or GLP-1.

The most noteworthy effects of GLP-1 agonists are their ability to promote insulin secretion in a glucose-dependent manner by binding to GLP-1 receptors expressed on the pancreatic p cells. Almost as importantly, GLP-1 agonists have been shown to inhibit glucagon secretion at glucose levels above fasting levels. Critically, this does not affect the glucagon response to hypoglycemia, making GLP-1 agonists a safe anti-diabetic drug with very low incidence of hypoglycemia compared to insulin. In June 2021, Semaglutide, a GLP-1 agonist, was approved by the FDA for chronic weight management in adults with obesity or overweight with at least one weight-related condition (such as high blood pressure, type 2 diabetes, or high cholesterol). As an anti-obesity drug, GLP-1 agonists work by binding to GLP-1 receptors in hypothalamus thereby supressing appetite. Furthermore, GLP-1 agonists bind to GLP-1 receptors in the stomach, inhibiting gastric emptying, acid secretion, and motility, which collectively promote satiety. Consequently, diabetic subjects treated with GLP-1 receptor agonists often also experience a beneficial weight loss in addition to a control of their blood sugar levels.

However, despite long-standing efforts, the number of overweight and obese patients is still growing. First line therapy for overweight and obese patients comprises diet and exercise but often are not sufficiently efficacious. Second line treatment options are bariatric surgery and pharmacotherapy. Available pharmacological treatments, seem to lack in efficacy and/or safety, and only a limited number of approved therapies, such as Semaglutide, are available in the US and in Europe. Therefore, there is still a high medical need for more efficacious and safe treatment options. Future obesity treatments may benefit from targeting GLP1-R, GIPR and NPY2R simultaneously in view of the biological role of PYY, GIP, or GLP-1. Indeed, recent research efforts in the obesity field have aimed at developing hybrid polypeptides as triple agonists for these receptors (see e.g. EP3467106 A1). Hybrid polypeptides are highly desirable compared to combination therapy with individual polypeptides for a number of reasons. First, hybrid polypeptides are easier to formulate into a single dosing unit as compared to mixtures of different polypeptides. This is primarily because different peptides may possess different physiochemical properties, e.g., isoelectric point, solubility, or chemical stability at a given pH, which means that one formulation developed for one polypeptide may be less optimal for or incompatible with a different polypeptide. Therefore, combination therapy with individual polypeptides may require individual dosing units. Secondly, hybrid polypeptides are cheaper to manufacture compared to individual polypeptides and also reduce the burden of regulatory approval.

The present invention set out to provide hybrid polypeptides that agonize all of the receptors GLP1-R, GIPR and NPY2R, in order to provide improved treatments for e.g. obesity, diabetes, and/or metabolic syndrome.

Further, it is an aim of the present invention to provide triple agonists that are soluble at or around physiological pH (e.g pH 7).

A further aim of the present invention is to provide triple agonists that have a long duration of action in the body, i.e. a long in-vivo half life.

A further aim of the present invention is to provide triple agonists that agonize all the receptors GLP1-R, GIPR and NPY2R and are soluble at or around physiological pH (e.g pH 7).

A further aim of the present invention is to provide triple agonists that agonize all the receptors GLP1-R, GIPR and NPY2R, are soluble at or around physiological pH (e.g pH 7) and have a long duration of action in the body.

A further aim of the present invention is to provide triple agonists that show good selectivity for the GLP-1, GIP and NPY2 receptors against other incretin or related receptors. For example, the agonists of the invention might not activate other receptors from the NPY receptor family, such as NPY1, NPY4 or NPY5 receptor, and/or might not activate GLP-2 or glucagon receptors.

### SUMMARY OF THE INVENTION

In a first aspect, the invention provides a polypeptide according to the general structure of Formula (I) or a pharmaceutically acceptable salt thereof,

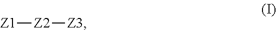

wherein,
Z1 is a hybrid polypeptide providing GIPR and GLP1R agonism comprising the amino acid sequence,
Y-Aib-E-G-T-F-T-S-D-Y-S-I-Aib-L-E-K-Q-A-Q-Aib-A-F-V-E-W-L-I-K (SEQ ID NO: 306),
or a derivative thereof having 1, 2, or 3 substitution(s);
Z2 is a linker;
Z3 is a polypeptide providing hY2R agonism comprising the amino acid sequence,
A-S-L-R-H-Y-Y-N-W-L-T-R-Q-R-Y (SEQ ID NO: 307) or a derivative thereof having 1, 2, 3, 4, or 5 substitution(s).

In a second aspect, the invention provides a polypeptide comprising the general structure of Formula (I) or a pharmaceutically acceptable salt thereof,

(I),
(SEQ ID NO: 647)
Z₁-Z₂-Z₃

wherein,
Z₁ is a GIP/GLP1 hybrid polypeptide comprising the amino acid sequence,
Y-Aib-X₃-G-T-F-T-S-D-X₁₀-S-I-X₁₃-L-X₁₅-X₁₆-X₁₇-A-X₁₉-X₂₀-X₂₁-F-X₂₃-X₂₄-X₂₅-L-X₂₇-K (SEQ ID NO: 646),
wherein X₃ is selected as E or D; X₁₀ is selected as Y or L; X₁₃ is selected as Aib or L; X₁₅ is selected as E, D, or A; X₁₆ is selected as K, E, D, Aib, G, LysAc, A, L, S, Q, or R; X₁₇ is selected as Q, E, K, or A; X₁₉ is selected as Q or E; X₂₀ is selected as Aib, D-Asp, or D-Arg; X₂₁ is selected as A, E, or K; X₂₃ is selected as V or I; X₂₄ is selected as E or Q; X₂₅ is selected as W or Y; X₂₇ is I or L;
Z₂ is a linker consisting of the amino acid sequence G-G-X₃₁-X₃₂-X₃₃-X₃₄,
wherein X₃₁ is selected as P, G, or Q; X₃₂ is selected as S, E, or R; X₃₃ is selected as S, E, Y, or T; X₃₄ is selected as G, P, Y, or A;
Z₃ is a polypeptide comprising the amino acid sequence,
X₃₅-X₃₆-X₃₇-X₃₈-X₃₉-Y-X₄₁-X₄₂-X₄₃-X₄₄-T-X₄₆-X₄₇-X₄₈-X₄₉,
wherein X₃₅ is selected as A, Q, I, V, E, or L; X₃₆ is selected as S, E, T, D, or L; X₃₇ is selected as L, Aib, V, D-Leu, I, or Tle; X₃₈ is selected as R, L, or Aib; X₃₉ is selected as H, Aib, D-His, or Tle; X₄₁ is selected as L, Y, Aib, I, or Tle; X₄₂ is selected as N or Aib; X₄₃ is selected as W, H, L, R, or Aib; X₄₄ is selected as L, Aib, D-Arg, D-Asp, or Tle; X₄₆ is selected as R, hArg, or D-Arg; X₄₇ is selected as Q, bh-Gln, or NMeQ; X₄₈ is selected as R or NMeR; X₄₉ is selected as Y, Tle, Chg, D-Tyr, Phg.

In a third aspect, the invention relates to a polypeptide according to the first or second aspect, for use as a medicament.

In a fourth aspect, the invention relates to a method for the treatment of a disease, disorder and/or condition selected from the list consisting of excessive weight, obesity, type 1 and type 2 diabetes, eating disorders, hyperlipidemia, metabolic syndrome, NAFLD/NASH, and/or cardiovascular diseases, said method comprising administering a therapeutic effective amount of a polypeptide or a pharmaceutically acceptable salt thereof, according to first or second aspect, to an individual in need thereof.

### Terms, Definitions and Conventions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

### Amino Acids

According to the present invention, unless otherwise stated, the amino acids are all L-amino acids (L-stereoisomer, natural amino acids). In the present context, substitutions in an analogue/derivative may be substitutions to natural amino acids as well as unnatural amino acids, including L- and D-stereoisomers. A substitution in a derivative may be a conservative substitution with a conservative amino acid. The groups of conservative amino acids may be defined as:
G, A, V, L, I, P (aliphatic or cyclic),
S, C, T, M (hydroxyl or sulphur containing)
F, Y, W (aromatic)
H, K, R (basic)
D, E, N, Q (acidic or amide)

Common unnatural amino acids include NMeG, which denotes the amino acid methylglycine (also referred to as N-Methylglycine, NMeGly, MeGly or sarcosine); NMeP, which denotes the amino acid methyl-L-proline (also referred to as N-Methyl-L-proline, NMePro or (S)-1-methylpyrrolidine-2-carboxylic acid); Dpr, which denotes the amino acid (S)-2,3-diaminopropionic acid (also referred to as L-2,3-diaminopropionic acid); Aib, which denotes the amino acid 2-amino-2-methylpropanoic acid (also referred to as 2-aminoisobutyric acid); NMeQ, which denotes the amino acid N-methyl-L-glutamine (also referred to as N-Methylglutamine, NMeGIn, or MeGln); NMeAla, which denotes the amino acid N-methyl-L-alanine (also referred to as N-Methylalanine, NMeA, or MeAla); Cha, which denotes the amino acid (S)-2-amino-3-cyclohexylpropanoic acid (also referred to as L-cyclohexylalanine); Tle, which denotes the amino acid (S)-2-amino-3,3-dimethylbutanoic acid (also referred to as L-2-(tert-butyl)glycine); 1-Nal denotes the amino acid (S)-2-amino-3-(naphthalen-1-yl)propanoic acid (also referred to as 3-(1-napthyl)-L-alanine); Pip, which denotes the amino acid (S)-piperidine-2-carboxylic acid (also referred to as L-pipecolic acid); Nip, which denotes the amino acid (S)-piperidine-3-carboxylic acid (also referred to as L-nipecotic acid); Nle, which denotes the amino acid (2S)-2-aminohexanoic acid (also referred to as L-norleucine); Phe(4F), which denotes the amino acid (S)-2-amino-3-(4-fluorophenyl)propanoic acid (also referred to as 4-fluoro-L-phenylalanine).

### Salts and Pharmaceutically Acceptable Salts

According to the present invention, a polypeptide or a derivative thereof may be in the form of a salt. According to the present invention, a polypeptide or a derivative thereof may be in the form of a pharmaceutically acceptable salt. Thus, pharmaceutically acceptable salts are intended to include any salts that are commonly used in formulations of peptides. Such salts include both acid addition salts and basic salts, and examples may be found in e.g., Remington's pharmaceutical sciences, 17^{th} edition. Likewise, a polypeptide or a pharmaceutically acceptable salt may be in the form of a solvate (e.g., a hydrate).

### Nomenclature of Compounds:

Compounds are represented in the Boehringer Ingelheim Line Notation (BILN), which describes complex peptides in a human-readable format (Fox et al, J. Chem. Inf Model. 2022, 62, 17, 3942-3947,). In BILN, chains are built up of monomers, e.g. A represents alanine, Aib represents isobutyric acid, and two monomers are linked by a hyphen “-”. Different chains are separated by a dot “.” and connections between chains are explicitly defined in brackets following the monomer, which includes the link ID number and the R-group number, e.g. K(1,3) represents a lysine residue that is linked to another monomer carrying the same link ID “1” by its R″3″-group (E-amino group). As an example, Y-Aib-E-G-T-F-T-S-D-Y-S-I-Aib-L-E-K-Q-A-Q-Aib-A-F-V-E-W-L-I-K(1,3)-G-G-P—S-S-G-A-S-L-R-H-Y-L-N-W-L-T-R-Q-R—Y-NH₂.C20DA-gGlu-gGlu-gGlu-gGlu-gGlu-gGlu-eLys(1,2) (SEQ ID NO: 412), wherein C20DA represents 19-carboxynonadecanoyl, gGlu ([γE]) represents L-y-glutamyl, connected via its amino-group to C20DA and via its y-carboxy-group to the amino-group of the next gGlu, which is repeated 4 times resulting in 6 gGlu residues total, connected via the final y-carboxy-group to the E-amino-group of lysine (eLys), connected via is carboxy-group to the E-amino-group of lysine (K) in the peptide backbone, completely defines the following structure:

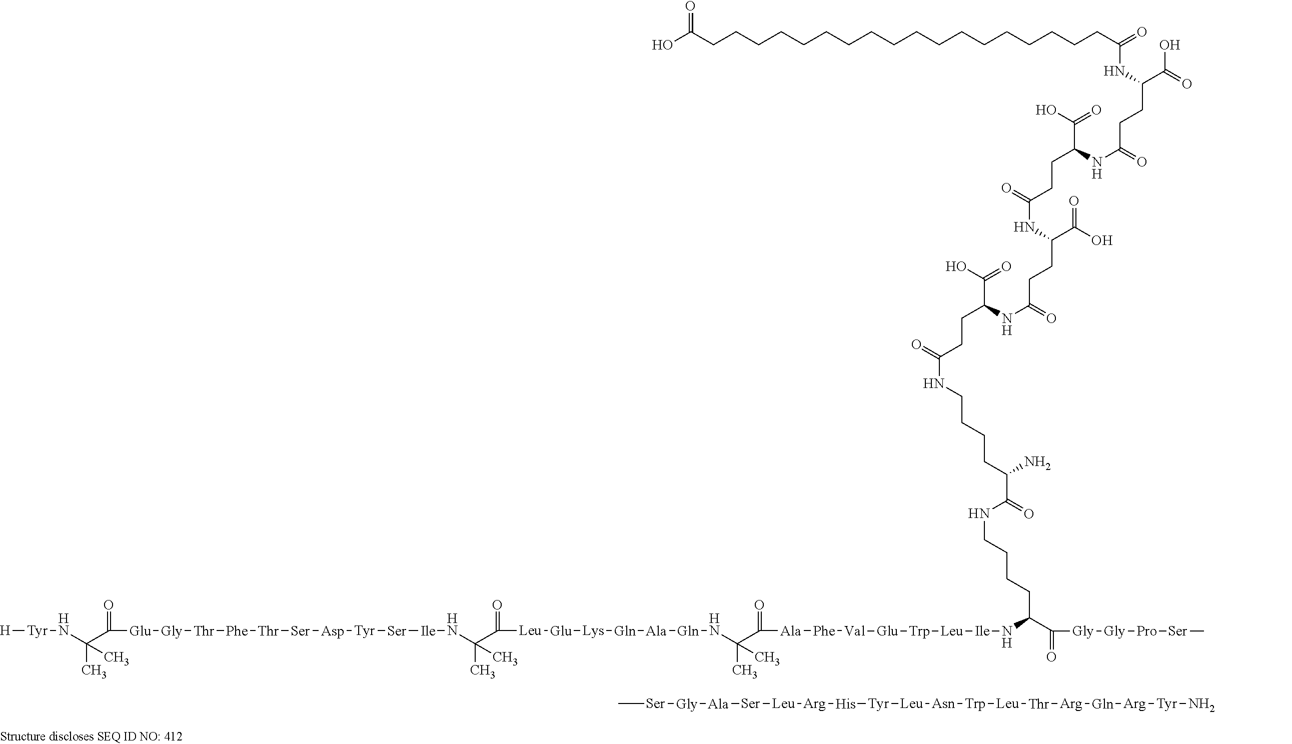

### Lipidation

In the present context, lipidation refers to the covalent attachment of a lipid (L), such as C18DA (octadecanedioic acid), C20DA (icosanedioic acid) or other half-life extending moieties, to a hybrid polypeptide according to the invention, optionally through a linker/spacer (—(Y—)₁₋₈). A linker/spacer may consist of one or more covalently connected monomers commonly used in the art, such as [γE], [OEG] or [AHX] illustrated below.

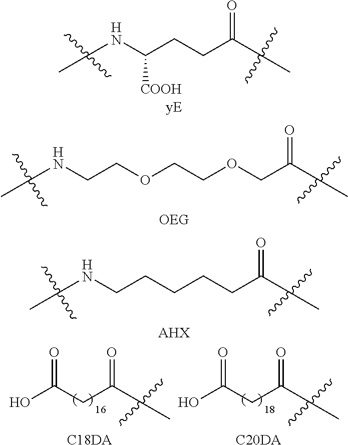

Lipidation may be performed at a lysine residue (i.e. at the E (epsilon) amino group) in the polypeptide or at the N-terminal, preferably at a lysine residue as exemplified herein. Without wishing to be bound by any theory, it is thought that such lipophilic substituents bind albumin and other plasma components in the blood stream, thereby shielding the compound of the invention from renal filtration as well as enzymatic degradation. Thus, lipidation is typically performed to improve the pharmacokinetic profile of a polypeptide by e.g. improving metabolic stability, reducing enzymatic degradation, lowering excretion and metabolism, all in all resulting in a prolonged in vivo half-life (t_{1/2}). The polypeptides according to the invention may be lipidated or non-lipidated depending on the desired half-life. If a linker/spacer is present, the lipid (L) is covalently attached to linker/spacer (—(Y—)₁₋₈) in one end and the polypeptide is covalently attached to the linker/spacer (—(Y—)₁₋₈) in the opposite end (i.e. lipid-linker-peptide). Alternatively, when no linker/spacer is present, the lipid (L) is directly covalently attached to polypeptide (i.e. Lipid-polypeptide). The linker/spacer may consist of 1 up to and including 8 covalently connected monomers (i.e. —Y¹—; —Y¹—Y²—; —Y¹—Y²—Y³—; —Y¹—Y²—Y³—Y⁴—; —Y¹—Y²—Y³—Y⁴—Y⁵—; —Y¹-Y²—Y³—Y⁴—Y⁵—Y⁶—; —Y¹—Y²—Y³—Y⁴—Y⁵—Y⁶—Y⁷—; or —Y¹—Y²—Y³—Y⁴—Y⁵—Y⁶—Y⁷—Y⁸-), wherein each unit is independently selected from e.g. [γE], [OEG] or [AHX], or, alternatively, from e.g. [γE], [E], [OEG], [eLys], or [AHX]. Most preferably, the polypeptide is lipidated with a structure of general formula L-Y¹—Y²—Y³—Y⁴—Y⁵—Y⁶—Y⁷—Y⁸-, wherein L is a lipid selected from C18DA or C20DA and further wherein each of Y¹—Y⁸ are independently selected from being, not present, [γE], [OEG], [eLys], or [AHX] or, alternatively, from being, not present, [γE], [E], [OEG], [eLys], or [AHX].

The lipid (L) may be attached to the linker/spacer (—(Y—)₁₋₈) via an ester, ether, a sulfonyl ester, a thioester, an amide, an amine, triazole or a sulfonamide, preferably via an amide or ester. Accordingly, it will be understood that preferably the lipid (L) includes an acyl group, a sulfonyl group, an alkyne, an azide, an N atom, an O atom or an S atom, which forms part of the ester, sulfonyl ester, thioester, triazole, amide, amine or sulfonamide. Preferably, an acyl group, or an 0 or N atom in the lipophilic substituent (L) forms part of an amide or ester with the linker/spacer (—(Y—)₁₋₈).

Likewise, the linker/spacer (—(Y—)₁₋₈) (if present) is attached to an amino acid residue of the hybrid polypeptides according to the invention via an ester, a sulfonyl ester, a thioester, an amide, an amine or a sulfonamide. Accordingly, it will be understood that preferably the linker/spacer (if present) includes an acyl group, a sulfonyl group, an N atom, an O atom or an S atom which forms part of the ester, sulfonyl ester, thioester, amide, amine or sulfonamide. Preferably, an acyl group, or an O or N atom in the linker (—(Y—)₁₋₈) forms part of an amide or ester with the amino acid residue.

The lipophilic substituent (L) may comprise a hydrocarbon chain having from 10 to 24 carbon atoms, e.g. from 14 to 22 carbon atoms, e.g. from 16 to 20 carbon atoms. Preferably, it has at least 14 carbon atoms, and preferably has 20 carbon atoms or fewer. For example, the hydrocarbon chain may contain 14, 15, 16, 17, 18, 19 or 20 carbon atoms. The hydrocarbon chain may be linear or branched, and may be saturated or unsaturated. Furthermore, it can include a functional group at the end of the hydrocarbon chain, e.g. a carboxylic acid group, a sulphonic acid group, or a tetrazole group. From the discussion above it will also be understood that the hydrocarbon chain is preferably substituted with a moiety, which forms part of the attachment to an amino acid residue of the hybrid polypeptides according to the invention or to the linker (—(Y—)₁₋₈), for example an acyl group, a sulfonyl group, an N atom, an O atom or an S atom. Most preferably, the hydrocarbon chain is substituted with an acyl group (for the attachment to the linker/spacer), and accordingly the hydrocarbon chain may be part of an alkanoyl group, for example a dodecanoyl, 2-butyloctanoyl, tetradecanoyl, hexadecanoyl, heptadecanoyl, octadecanoyl, nonadecanoyl or eicosanoyl group. These hydrocarbon chains substituted with an acyl group at one end may further be functionalized with a carboxylic acid group at the other end of the chain. Examples of functionalized hydrocarbon chains (e.g. lipophilic substituents L) are 15-carboxy-pentadecanoyl (briefly C16DA), 17-carboxy-heptadecanoyl (briefly C18DA) and 19-carboxy-nonadecanoyl (briefly C20DA). Preferred lipid or lipid/linkers in the present disclosure are C18DA, C18DA[E][E][E][E]- (SEQ ID NO: 640), C18DA[E][E][E][E][E]- (SEQ ID NO:636), C18DA[E][E][E][E][E][E]-(SEQ ID NO: 637), C18DA[γE]-, C18DA[γE][γE]-, C18DA[γE][γE][γE]-, C18DA[γE][γE][γE][γE]-, C18DA[γE][γE][γE][γE][γE]-, C18DA[γE][γE][γE][γE][γE][γE]-, C18DA[γE][γE][γE][γE][γE][γE][γE]-, C20DA, C20DA[γE]-, C20DA[γE][γE]-, C20DA[γE][γE][γE]-, C20DA[γE][γE][γE][γE]-, C20DA[γE][γE][γE][γE][γE]-, C20DA[γE][γE][γE][γE][γE][γE]-, C20DA[γE][γE][γE][γE][γE][γE][γE]-, C18DA[eLys], C18DA[E][E][E][E][E][eLys]-(SEQ ID NO: 635), C18DA[E][E][E][E][E][E][eLys]- (SEQ ID NO: 643), C18DA[γE][eLys]-, C18DA[γE][γE][eLys]-C18DA[γE][γE][γE][eLys]-, C18DA[γE][γE][γE][γE][eLys]-, C18DA[γE][γE][γE][γE][γE][eLys]-, C18DA[γE][γE][γE][γE][γE][γE][eLys]-, C20DA[eLys], C20DA[E][E][E][E][E][eLys]- (SEQ ID NO: 639), C20DA[γE][eLys]-, C20DA[γE][γE][eLys]-, C20DA[γE][γE][γE][eLys]-, C20DA[γE][γE][γE][γE][eLys]-, C20DA[γE][γE][γE][γE][γE][eLys]-, C20DA[γE][γE][γE][γE][γE][γE][eLys]-, C20DA[γE][γE][γE][γE][γE][γE][γE][eLys]-, C20DA[γE][γE][γE][γE][γE][eLys][eLys]-, C18DA[OEG][OEG]-, C18DA[E][E][E][OEG][OEG]-, C18DA[E][E][E][E][OEG][OEG]- (SEQ ID NO: 634), C18DA[γE][OEG][OEG]-, C18DA[γE][γE][OEG][OEG]-, C18DA[γE][γE][γE][OEG][OEG]-, C18DA[γE][γE][γE][γE][OEG][OEG]-, C18DA[γE][γE][γE][γE][γE][OEG][OEG]-, C20DA[OEG][OEG]-, C20DA[E][E][E][E][OEG][OEG]- (SEQ ID NO: 638), C20DA[γE][OEG][OEG]-, C20DA[γE][γE][OEG][OEG]-, C20DA[γE][γE][γE][OEG][OEG]-, C20DA[γE][γE][γE][γE][OEG][OEG]-, C20DA[γE][γE][γE][γE][γE][OEG][OEG]-, C18DA[OEG]-, C18DA[γE][OEG]-, C18DA[γE][γE][OEG]-, C18DA[γE][γE][γE][OEG]-, C18DA[γE][γE][γE][γE][OEG]-, C18DA[γE][γE][γE][γE][γE][OEG]-, C18DA[γE][γE][γE][γE][γE][γE][OEG]-, C20DA[OEG]-, C20DA[γE][OEG]-, C20DA[γE][γE][OEG]-, C20DA[γE][γE][γE][OEG]-, C20DA[γE][γE][γE][γE][OEG]-, C20DA[γE][γE][γE][γE][γE][OEG]-, C20DA[γE][γE][γE][γE][γE][γE][OEG]-, C18DA[OEG][eLys]-, C18DA[γE][OEG][eLys]-, C18DA[γE][γE][OEG][eLys]-, C18DA[γE][γE][γE][OEG][eLys]-, C18DA[γE][γE][γE][γE][OEG][eLys]-, C18DA[γE][γE][γE][γE][γE][OEG][eLys]-, C20DA[OEG][eLys]-, C20DA[γE][OEG][eLys]-, C20DA[γE][γE][OEG][eLys]-, C20DA[γE][γE][γE][OEG][eLys]-, C20DA[γE][γE][γE][γE][OEG][eLys]-, C20DA[γE][γE][γE][γE][γE][OEG][eLys]-, C18DA[OEG][OEG][eLys]-, C18DA[γE][OEG][OEG][eLys]-, C18DA[γE][γE][OEG][OEG][eLys]-, C18DA[γE][γE][γE][OEG][OEG][eLys]-, C18DA[γE][γE][γE][γE][OEG][OEG][eLys]-, C20DA[OEG][OEG][eLys]-, C20DA[γE][OEG][OEG][eLys]-, C20DA[γE][γE][OEG][OEG][eLys]-, C20DA[γE][γE][γE][OEG][OEG][eLys]-, C20DA[γE][γE][γE][γE][OEG][OEG][eLys]-, C18DA[AHX], C18DA[γE][AHX]-, C18DA[γE][γE][AHX]-, C18DA[γE][γE][γE][AHX]-, C18DA[γE][γE][γE][γE][AHX]-, C18DA[γE][γE][γE][γE][γE][AHX]-, C18DA[γE][γE][γE][γE][γE][γE][AHX]-, C20DA[AHX], C20DA[γE][AHX]-, C20DA[γE][γE][AHX]-, C20DA[γE][γE][γE][AHX]-, C20DA[γE][γE][γE][γE][AHX]-, C20DA[γE][γE][γE][γE][γE][AHX]-, or C20DA[γE][γE][γE][γE][γE][γE][AHX]-. Preferably the polypeptide is lipidated at the lysine (K) residue in amino acid position X₂₈.

### N- and C-Terminal

In the present context, the polypeptides are generally amidated at the C-terminal (—CONH₂), like the native peptides. However, the polypeptides of the present invention may also have either a free carboxylic acid (—COOH) or another post-translational modification, such as a methyl ester (—COOMe). In a highly preferred embodiment of the invention, the polypeptides are amidated at the C-terminal. The polypeptides according to the present invention may have a free amine (—NH₂), be N-acylated (—NHCOR), N-methylated (—NHCH₃ or -N(CH₃)₂), deaminated at the N-terminal, or N-lipidated.

### Pharmaceutical Compositions

In the present context it should be understood that the polypeptides according to the invention, or pharmaceutically acceptable salts thereof, may be in the form of a pharmaceutical composition. A pharmaceutical composition may comprise a pharmaceutically acceptable carrier and/or one or more excipients. The pharmaceutical composition (i.e. formulation) includes but are not limited to tablets, pills, capsules, emulsions, suspensions, sustained release formulations, solutions, or freeze-dried powder intended for dissolution prior to administration. In some embodiments, the formulation may be a depot formulation providing slow release. It should be appreciated that different routes of administration may be used depending on the choice of formulation and chemical and/or metabolic stability of the polypeptides. Such administration routes may include but are not limited to oral administration, parenteral administration (intravenous (IV), subcutaneous (SC), intradermal (ID) and intramuscular (IM)), or inhalation. In a preferred embodiment of the invention, the administration route is parenteral administration. In an even more preferred embodiment, the administration route is subcutaneous.

Peptide therapeutics are usually provided as pharmaceutical liquid formulation in a pre-filled ready-to-use injection device. These peptide formulations for subcutaneous administration have limited application volumes. Therefore, good solubility of the peptides at or around physiological pH (e.g pH 7) is a requirement for the application in a ready-to-use injection device.

### DETAILED DESCRIPTION OF THE INVENTION

In a first aspect, the invention provides a polypeptide according to the general structure of Formula (I) or a salt or a pharmaceutically acceptable salt thereof,

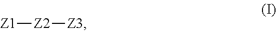

wherein,
Z1 is a GIP/GLP1 hybrid polypeptide comprising or consisting of the amino acid sequence, Y-Aib-E-G-T-F-T-S-D-Y-S-I-Aib-L-E-K-Q-A-Q-Aib-A-F-V-E-W-L-I-K (SEQ ID NO: 306), or a derivative thereof having 1, 2, or 3 amino acid substitution(s);
Z2 is a linker;
Z3 is a polypeptide comprising or consisting of the amino acid sequence,
A-S-L-R-H-Y-Y-N-W-L-T-R-Q-R-Y (SEQ ID NO: 307) or a derivative thereof having 1, 2, 3, 4, or 5 amino acid substitution(s).

The compounds of the present invention bind to and/or activate the GLP-1, the GIP and the hY2 (NPY2) receptors.

### Linker Z2

The peptide fragments Z1 and Z3 are linked through a linker Z2. It should be appreciated that Z1 and Z3 may be linked by various linkers commonly used in the art of e.g. fusion protein. Preferably the linker comprises or consists of a short peptide consisting of 1-10 amino acid residues, such as 2-9 amino acids, preferably 3-8 amino acids, more preferably, 4-7 amino acids, even more preferably 5-7 amino acids, most preferably 6 amino acid residues. Thus, in a preferred embodiment, the linker Z2 consists of 6 amino acid residues (i.e. amino acids X₂₉-X₃₄). In a more preferred embodiment, Z2 has or comprises the amino acid sequence GGPSEG (SEQ ID NO: 311, i.e. amino acids X₂₉-X₃₄) or is a derivative thereof having 1, 2, 3, or 4 amino acid substitution(s). Preferably, the 1, 2, 3, or 4 amino acid substitution(s) in Z2 is/are present in any of the positions X₃₁, X₃₂, X₃₃, or X₃₄ (i.e. in the amino acid sequence PSEG (SEQ ID NO: 644)). Therefore, said Z2 derivatives are Z2 peptide analogues of the amino acid sequence SEQ ID NO: 311 having one or more amino acid substitution(s) compared to SEQ ID NO: 311.

In a highly preferred embodiment (Z2-Emb1), Z2 (i.e. amino acids X₂₉-X₃₄) has or comprises the amino acid sequence GGX₃₁X₃₂X₃₃X₃₄, wherein X₃₁ is selected as P, G, or Q; X₃₂ is selected as S, E, or R; X₃₃ is selected as S, E, or Y, or, preferably, as S, E, Y, or T; X₃₄ is selected as G, P, Y, or A; or is a derivative thereof having 1 amino acid substitution.

In further highly preferred embodiments, Z2 has the amino acid sequence of any one of the Z2 embodiments as disclosed hereinafter in the second aspect of the present invention (e.g. Z2-Emb2, Z2-Emb3).

### Hybrid Polyoeotide Z1

Z1 (i.e. amino acids X₁-X₂₈) is a GIP/GLP1 hybrid polypeptide comprising or consisting of the amino acid sequence, Y-Aib-E-G-T-F-T-S-D-Y-S-I-Aib-L-E-K-Q-A-Q-Aib-A-F-V-E-W-L-I-K (SEQ ID NO: 306), or is a derivative thereof having 1, 2, or 3 amino acid substitution(s). The sequence Y-Aib-E-G-T-F-T-S-D-Y-S-I-Aib-L-E-K-Q-A-Q-Aib-A-F-V-E-W-L-I-K (SEQ ID NO: 306) (i.e. Z1 in SEQ ID NO: 286) was chosen as reference sequence. Table 1 summarizes (see page 21ff), the number of substitutions in Z1, the position(s) of Z1 that was/were substituted, and the exemplified amino acids in the substitutions in comparison with the reference (i.e. SEQ ID NO: 286). As shown in table 1, the amino acid positions X₃, X₁₀, X₁₃, X₁₅, X₁₆, X₁₇, X₁₉, X₂₀, X₂₁, X₂₃, X₂₄, X₂₅, or X₂₇ were found to tolerate substitutions. Thus, in a preferred embodiment, the 1, 2 or 3 substitution(s) in the derivate of Z1 is/are present in any of the amino acid positions X₃, X₁₀, X₁₃, X₁₅, X₁₆, X₁₇, X₁₉, X₂₀, X₂₁, X₂₃, X₂₄, X₂₅, or X₂₇. Preferably, the derivate of Z1 has 1, or 2 substitution(s). Most preferably, the derivate of Z1 has 1 substitution. Therefore, said Z1 derivatives are Z1 peptide analogues of the amino acid sequence SEQ ID NO: 306 (i.e. Z1 in SEQ ID NO: 286) having one or more amino acid substitution(s) compared to SEQ ID NO: 306.

In a highly preferred embodiment, the substitution(s), in the derivate of Z1, is/are selected as follows: X₃ is selected as D; X₁₀ is selected as L; X₁₃ is selected as L; X₁₅ is selected as D or A; X₁₆ is selected as E, D, Aib, G, LysAc, A, L, S, Q, or R; X₁₇ is selected as E, K, or A; X₁₉ is selected as E; X₂₀ is selected as D-Asp, or D-Arg; X₂₁ is selected as E or, preferably, as E or K; X₂₃ is selected as I; X₂₄ is selected as Q; X₂₅ is selected as Y; X₂₇ is selected as L.

Z1 may consist of or comprise the amino acid sequence Y-Aib-E-G-T-F-T-S-D-Y-S-I-Aib-L-E-K-Q-A-Q-Aib-A-F-V-E-W-L-I-K (SEQ ID NO: 306) or may be a derivative thereof having 1, 2 or 3 amino acid substitution(s) at any of the amino acid positions X₃, X₁₀, X₁₃, X₁₅, X₁₆, X₁₇, X₁₉, X₂₁, X₂₃, X₂₄, X₂₅, or X₂₇, wherein the substitution(s) is/are selected as follows: X₃ is selected as D; X₁₀ is selected as L; X₁₃ is selected as L; X₁₅ is selected as D; X₁₆ is selected as E, D, G, A, S, Q, or R; X₁₇ is selected as E, or A; X₁₉ is selected as E; X₂₁ is selected as E or K; X₂₃ is selected as I; X₂₄ is selected as Q; X₂₅ is selected as Y; X₂₇ is selected as L.

Z1 may consist of or comprise the amino acid sequence Y-Aib-E-G-T-F-T-S-D-Y-S-I-Aib-L-E-K-Q-A-Q-Aib-A-F-V-E-W-L-I-K (SEQ ID NO: 306) or may be a derivative thereof having 1, 2 or 3 amino acid substitution(s) at any of the amino acid positions X₃, X₁₀, X₁₃, X₁₅, X₁₆, X₁₇, X₂₁, X₂₃, X₂₄, or X₂₅, wherein the substitution(s) is/are selected as follows: X₃ is selected as D; X₁₀ is selected as L; X₁₃ is selected as L; X₁₅ is selected as D; X₁₆ is selected as E, D, G, A, S, Q, or R; X₁₇ is selected as E, or A; X₂₁ is selected as E; X₂₃ is selected as I; X₂₄ is selected as Q; X₂₅ is selected as Y.

Z1 may consist of or comprise the amino acid sequence Y-Aib-E-G-T-F-T-S-D-Y-S-I-Aib-L-E-K-Q-A-Q-Aib-A-F-V-E-W-L-I-K (SEQ ID NO: 306) or may be a derivative thereof having 1 or 2 amino acid substitution(s), preferably 1, at any of the amino acid positions X₂₁, or X₂₄, wherein the substitution(s) is/are selected as follows: X₂₁ is selected as A or E; X₂₄ is selected as E or Q.

### Polypeptide Z3

Z3 (i.e. amino acids X₃₅-X₄₉) is a polypeptide providing hY2R agonism comprising or consisting of the amino acid sequence, A-S-L-R-H-Y-Y-N-W-L-T-R-Q-R-Y (SEQ ID NO: 307) or a derivative thereof having 1, 2, 3, 4, or 5 amino acid substitution(s). The sequence A-S-L-R-H-Y-Y-N-W-L-T-R-Q-R-Y (SEQ ID NO: 307) (i.e. Z3 in SEQ ID NO: 286) was chosen as reference sequence. Table 1 summarizes (see page 39ff) the number of substitutions in Z3, the positions of Z3 that can be substituted, and the exemplified amino acids in the substitutions in comparison with the reference (i.e. SEQ ID NO: 286). As shown in table 1, the amino acid positions X₃₅, X₃₆, X₃₇, X₃₈, X₃₉, X₄₁, X₄₂, X₄₃, X44, X₄₆, X₄₇, X₄₈, or X₄₉ were found to tolerate substitutions. Thus, in a preferred embodiment, the 1, 2, 3, 4, or 5 substitution(s) in the derivate of Z3 is/are present in any of the amino acid positions X₃₅, X₃₆, X₃₇, X₃₈, X₃₉, X₄₁, X₄₂, X₄₃, X44, X₄₆, X₄₇, X₄₈, or X₄₉. In a preferred embodiment, Z3 has 1, 2, or 3 substitution(s). More preferably, Z3 has 1 or 2 substitution(s). Most preferably Z3 has 1 substitution. Therefore, said Z3 derivatives are Z3 peptide analogues of the amino acid sequence SEQ ID NO: 307 (i.e. Z3 in SEQ ID NO: 286) having one or more amino acid substitution(s) compared to SEQ ID NO: 307.

In a highly preferred embodiment, the substitution(s), in the derivate of Z3, is selected as follows: X₃₅ is selected as Q, I, V, E, or L; X₃₆ is selected as E, T, D, or L; X₃₇ is selected as Aib, V, D-Leu, or I, or, preferably, as Aib, V, D-Leu, I, or Tle; X₃₈ is selected as L or Aib; X₃₉ is selected as Aib, D-His, or Tle; X₄₁ is selected as L, Aib, I, or Tle; X₄₂ is selected as Aib; X₄₃ is selected as H, L, R, or Aib; X44 is selected as Aib, D-Arg, or D-Asp, or, preferably, as Aib, D-Arg, D-Asp, or Tle; X₄₆ is selected as hArg or D-Arg; X₄₇ is selected as bh-Gln or NMeQ; X₄₈ is selected as NMeR; X₄₉ is selected as Tle, Chg, D-Tyr, Phg.

Z3 may consist of or comprise the amino acid sequence A-S-L-R-H-Y-Y-N-W-L-T-R-Q-R-Y (SEQ ID NO: 307) or may be a derivative thereof having 1, 2 or 3 amino acid substitution(s) at any of the amino acid positions X₃₅, X₃₆, X₃₇, X₃₈, X₃₉, X₄₁, X₄₄, X₄₇, or X₄₈, wherein the substitution(s) is/are selected as follows: X₃₅ is selected as Q, I, V, E, or L; X₃₆ is selected as T; X₃₇ is selected as Aib, V, I or Tle; X₃₈ is selected as Aib; X₃₉ is selected as Tle; X₄₁ is selected as I, L, or Tle; X₄₄ is selected as Tle; X₄₇ is selected as NMeQ; X₄₈ is selected as NMeR.

Z3 may consist of or comprise the amino acid sequence A-S-L-R-H-Y-Y-N-W-L-T-R-Q-R-Y (SEQ ID NO: 307) or may be a derivative thereof having 1, or 2 amino acid substitution(s), preferably 1, at any of the amino acid positions X₃₅, X₃₇, X₄₇, or X₄₈, wherein the substitution(s) is/are selected as follows: X₃₅ is selected as Q, V; X₃₇ is selected as I; X₄₇ is selected as NMeQ; X₄₈ is selected as NMeR.

Overall, the polypeptides Z1-Z2-Z3 according to the first aspect of the invention, and according to any or more embodiments disclosed in the present invention, preferably have 0, 1, 2, 3, 4, 5, or 6 amino acid substitutions, more preferably 0, 1, 2, 3, or 4 amino acid substitutions, highly preferably 0, 1, 2, or 3 substitutions, compared to SEQ ID NO: 286.

In a second aspect, the invention provides a polypeptide according to the general structure of Formula (I) or a salt or a pharmaceutically acceptable salt thereof,

(SEQ ID NO: 647)
Z1-Z2-Z3
(I),

wherein,
Z1 is a GIP/GLP1 hybrid polypeptide comprising or consisting of the amino acid sequence,
Y-Aib-X₃-G-T-F-T-S-D-X₁₀-S-I-X₁₃-L-X₁₅-X₁₆-X₁₇-A-X₁₉-X₂₀-X₂₁-F-X₂₃-X₂₄-X₂₅-L-X₂₇-K (SEQ ID NO: 646), wherein X₃ is selected as E or D; X₁₀ is selected as Y or L; X₁₃ is selected as Aib or L; X₁₅ is selected as E, D, or A; X₁₆ is selected as K, E, D, Aib, G, LysAc, A, L, S, Q, or R; X₁₇ is selected as Q, E, K, or A; X₁₉ is selected as Q or E; X₂₀ is selected as Aib, D-Asp, or D-Arg; X₂₁ is selected as A, E, or K; X₂₃ is selected as V or I; X₂₄ is selected as E or Q; X₂₅ is selected as W or Y; X₂₇ is I or L; or Z1 is a derivative thereof having 1 amino acid substitution;
Z2 is a linker comprising or consisting of the amino acid sequence G-G-X₃₁-X₃₂-X₃₃-X₃₄, wherein X₃₁ is selected as P, G, or Q; X₃₂ is selected as S, E, or R; X₃₃ is selected as S, E, Y, or T; X₃₄ is selected as G, P, Y, or A; or Z2 is a derivative thereof having 1 amino acid substitution.
Z3 is a polypeptide comprising or consisting of the amino acid sequence,
X₃₅-X₃₆-X₃₇-X₃₈-X₃₉-Y-X₄₁-X₄₂-X₄₃-X₄₄-T-X₄₆-X₄₇-X₄₈-X₄₉,
wherein X₃₅ is selected as A, Q, I, V, E, or L; X₃₆ is selected as S, E, T, D, or L; X₃₇ is selected as L, Aib, V, D-Leu, I, or Tle; X₃₈ is selected as R, L, or Aib; X₃₉ is selected as H, Aib, D-His, or Tle; X₄₁ is selected as Y, L, Aib, I, or Tle; X₄₂ is selected as N or Aib; X₄₃ is selected as W, H, L, R, or Aib; X₄₄ is selected as L, Aib, D-Arg, D-Asp, or Tle; X₄₆ is selected as R, hArg, or D-Arg; X₄₇ is selected as Q, bh-Gln, or NMeQ; X₄₈ is selected as R or NMeR; X₄₉ is selected as Y, Tle, Chg, D-Tyr, Phg; or Z3 is a derivative thereof having 1 amino acid substitution.

### Preferred Amino Acid in Z1

In a preferred embodiment (Z1-Emb1), X₃ is selected as E or D;
X₁₀ is selected as Y or L;
X₁₃ is selected as Aib or L;
X₁₅ is selected as E or D;
X₁₆ is selected as K, E, D, G, A, S, Q, or R;
X₁₇ is selected as Q, E, or A;
X₁₉ is selected as E or Q;
X₂₀ is selected as Aib;
X₂₁ is selected as A, E or K;
X₂₃ is selected as V or I;
X₂₄ is selected as E or Q;
X₂₅ is selected as W or Y;
X₂₇ is selected as I or L;
or Z1 is a derivative thereof having 1 amino acid substitution;

In a preferred embodiment (Z1-Emb2), X₃ is selected as E or D;
X₁₀ is selected as Y or L;
X₁₃ is selected as Aib or L;
X₁₅ is selected as E or D;
X₁₆ is selected as K, E, D, G, A, S, Q, or R;
X₁₇ is selected as Q, E, or A;
X₁₉ is selected as Q;
X₂₀ is selected as Aib;
X₂₁ is selected as A or E;
X₂₃ is selected as V or I;
X₂₄ is selected as E or Q;
X₂₅ is selected as W or Y;
X₂₇ is I;
or Z1 is a derivative thereof having 1 amino acid substitution;

In a preferred embodiment (Z1-Emb3), X₃ is selected as E;
X₁₀ is selected as Y;
X₁₃ is selected as Aib;
X₁₅ is selected as E;
X₁₆ is selected as K;
X₁₇ is selected as Q;
X₁₉ is selected as Q;
X₂₀ is selected as Aib;
X₂₁ is selected as A or E;
X₂₃ is selected as V;
X₂₄ is selected as E or Q;
X₂₅ is selected as W;
X₂₇ is I;
or Z1 is a derivative thereof having 1 amino acid substitution.

In a preferred embodiment, X₃ in Z1 is selected as E. In a preferred embodiment, X₁₀ in Z1 is selected as Y. In a preferred embodiment, X₁₃ in Z1 is selected as Aib. In a preferred embodiment, X₁₅ in Z1 is selected as E. In a preferred embodiment, X₁₆ in Z1 is selected as K. In a preferred embodiment, X₁₇ in Z1 is selected as Q. In a preferred embodiment, X₁₉ in Z1 is selected as Q. In a preferred embodiment, X₂₀ in Z1 is selected as Aib. In a preferred embodiment, X₂₁ in Z1 is selected as A. In a preferred embodiment, X₂₃ in Z1 is selected as V. In a preferred embodiment, X₂₄ in Z1 is selected as E. In a preferred embodiment, X₂₅ in Z1 is selected as W. In a preferred embodiment, X₂₇ in Z1 is selected as I.

In a highly preferred embodiment, X₃ in Z1 is selected as E; X₁₀ in Z1 is selected as Y; and X₁₃ in Z1 is selected as Aib. In a highly preferred embodiment, X₁₅ in Z1 is selected as E; X₁₆ in Z1 is selected as K; and X₁₇ in Z1 is selected as Q. In a highly preferred embodiment, X₁₉ in Z1 is selected as Q; X₂₀ in Z1 is selected as Aib; and X₂₁ in Z1 is selected as A. In a highly preferred embodiment, X₂₃ in Z1 is selected as V; X₂₄ in Z1 is selected as E; X₂₅ in Z1 is selected as W; and X₂₇ in Z1 is selected as I.

Preferred amino acids in Z2 In a preferred embodiment (Z2-Emb1), Z2 comprises or consist of the amino acid sequence GGX₃₁X₃₂X₃₃X₃₄, wherein X₃₁ is selected as P, G, or Q; X₃₂ is selected as S, E, or R; X₃₃ is selected as S, E, Y, or T; X₃₄ is selected as G, P, Y, or A; or Z2 is a derivative thereof having 1 amino acid substitution.

In a preferred embodiment (Z2-Emb2), Z2 comprises or consists of the amino acid sequence G-G-X₃₁-X₃₂-X₃₃—X₃₄ wherein X₃₁ is selected as P or Q; X₃₂ is selected as S or E; X₃₃ is selected as S, E, Y or T; X₃₄ is selected as G, P, Y, or A; or Z2 is a derivative thereof having 1 amino acid substitution.

In a highly preferred embodiment (Z2-Emb3), Z2 comprises or consists of the amino acid sequence GGPSEG (SEQ ID NO: 311) or GGPSSG (SEQ ID NO: 320); or Z2 is a derivative thereof having 1 amino acid substitution.

In a preferred embodiment, X₃₁ in Z2 is selected as P. In a preferred embodiment, X₃₂ in Z2 is selected as S. In a preferred embodiment, X₃₃ in Z2 is selected as S. In a preferred embodiment, X₃₄ in Z2 is selected as G.

In a further embodiment (Z2-Emb4) Z2 is selected from the group consisting of GGPEEG (SEQ ID NO: 313), GGPEEP (SEQ ID NO: 314), GGPESG (SEQ ID NO: 315), GGPESP (SEQ ID NO: 316), GGPSEG (SEQ ID NO: 311), GGPSEP (SEQ ID NO: 317), GGPSEY (SEQ ID NO: 318), GGPSSA (SEQ ID NO: 319), GGPSSG (SEQ ID NO: 320), GGPSSP (SEQ ID NO: 321), GGPSSY (SEQ ID NO: 322), GGPSTG (SEQ ID NO: 323), GGPSYG (SEQ ID NO: 324), GGQSSG (SEQ ID NO: 325), GGGSEY (SEQ ID NO:648), GGPEYY (SEQ ID NO: 649), GGPRSG (SEQ ID NO: 650), GGPRYY (SEQ ID NO: 651), GGPSYY (SEQ ID NO: 652), GGQRSY (SEQ ID NO: 653), GGQRYY (SEQ ID NO: 654), GGQSSY (SEQ ID NO: 655).

In a further embodiment (Z2-Emb5) Z2 is selected from the group consisting of GGPEEG (SEQ ID NO: 313), GGPEEP (SEQ ID NO: 314), GGPESG (SEQ ID NO: 315), GGPESP (SEQ ID NO: 316), GGPSEG (SEQ ID NO: 311), GGPSEP (SEQ ID NO: 317), GGPSEY (SEQ ID NO: 318), GGPSSA (SEQ ID NO: 319), GGPSSG (SEQ ID NO: 320), GGPSSP (SEQ ID NO: 321), GGPSSY (SEQ ID NO: 322), GGPSTG (SEQ ID NO: 323), GGPSYG (SEQ ID NO: 324), GGQSSG (SEQ ID NO: 325), GGPRSG (SEQ ID NO: 650), GGPRYY (SEQ ID NO: 651), GGPSYY (SEQ ID NO: 652).

In a further embodiment (Z2-Emb6) Z2 is selected from the group consisting of GGPEEG (SEQ ID NO: 313), GGPEEP (SEQ ID NO: 314), GGPESG (SEQ ID NO: 315), GGPESP (SEQ ID NO: 316), GGPSEG (SEQ ID NO: 311), GGPSEP (SEQ ID NO: 317), GGPSEY (SEQ ID NO: 318), GGPSSA (SEQ ID NO: 319), GGPSSG (SEQ ID NO: 320), GGPSSP (SEQ ID NO: 321), GGPSSY (SEQ ID NO: 322), GGPSTG (SEQ ID NO: 323), GGPSYG (SEQ ID NO: 324), GGQSSG (SEQ ID NO: 325).

In a further embodiment (Z2-Emb7) Z2 is selected from the group consisting of GGPEEG (SEQ ID NO: 313), GGPESG (SEQ ID NO: 315), GGPESP (SEQ ID NO: 316), GGPSEG (SEQ ID NO: 311), GGPSEP (SEQ ID NO: 317), GGPSEY (SEQ ID NO: 318), GGPSSA (SEQ ID NO: 319), GGPSSG (SEQ ID NO: 320), GGPSSP (SEQ ID NO: 321), GGPSSY (SEQ ID NO: 322), GGPSTG (SEQ ID NO: 323), GGPSYG (SEQ ID NO: 324), GGQSSG (SEQ ID NO: 325).

### Preferred Amino Acids in Z3

In a further embodiment (Z3-Emb1) X₃₅ is selected as A, E, I, L, Q, and V;
X₃₆ is selected as S or T;
X₃₇ is selected as Aib, I, L, Tle and V;
X₃₈ is selected as Aib or R;
X₃₉ is selected as H or Tle;
X₄₁ is selected as I, L, Tle or Y;
X₄₂ is selected as N;
X₄₃ is selected as W;
X₄₄ is selected as L or Tle;
X₄₆ is selected as R;
X₄₇ is selected as NMeQ or Q;
X₄₈ is selected as NMeR or R;
X₄₉ is selected as Y;
or Z3 is a derivative thereof having 1 amino acid substitution.

In a preferred embodiment (Z3-Emb2) X₃₅ is selected as A, Q or V;
X₃₆ is selected as S;
X₃₇ is selected as I or L;
X₃₈ is selected as R;
X₃₉ is selected as H;
X₄₁ is selected as Y;
X₄₂ is selected as N;
X₄₃ is selected as W;
X₄₄ is selected as L;
X₄₆ is selected as R;
X₄₇ is selected as Q or NMeQ;
X₄₈ is selected as R or NMeR;
X₄₉ is selected as Y;
or Z3 is a derivative thereof having 1 amino acid substitution.

In a preferred embodiment, X₃₅ in Z3 is selected as A. In a preferred embodiment, X₃₆ in Z3 is selected as S. In a preferred embodiment, X₃₇ in Z3 is selected as L. In a preferred embodiment, X₃₈ in Z3 is selected as R. In a preferred embodiment, X₃₉ in Z3 is selected as H. In a preferred embodiment, X₄₁ in Z3 is selected as Y. In a preferred embodiment, X₄₂ in Z3 is selected as N. In a preferred embodiment, X₄₃ in Z3 is selected as W. In a preferred embodiment, X44 in Z3 is selected as L. In a preferred embodiment, X₄₆ in Z3 is selected as R. In a preferred embodiment, X₄₇ in Z3 is selected as Q. In a preferred embodiment, X₄₈ in Z3 is selected as R. In a preferred embodiment, X₄₉ in Z3 is selected as Y.

In a highly preferred embodiment, X₃₅X₃₆X₃₇X₃₈X₃₉ is selected as ASLRH (SEQ ID NO: 645). In a highly preferred embodiment, X₄₁X₄₂X₄₃X₄₄ are selected as YNWL (SEQ ID NO: 326). In a highly preferred embodiment, X₄₆X₄₇X₄₈X₄₉ are selected as RQRY (SEQ ID NO: 327).

It should be appreciated that the second aspect of the invention and/or any relative embodiment hereinbefore and hereinafter disclosed may be dependent from the first aspect of the invention and/or any relative embodiment hereinbefore and hereinafter disclosed.

It should be appreciated that one or more of the embodiments, according to either the first or second aspect of the invention, describing preferred amino acids in the positions of Z1 may be combined with one or more of the embodiments describing preferred amino acids in the positions of Z2 and/or Z3 and vice versa. Thus, any combination of one or more of the embodiments mentioned under “Preferred amino acids in Z1” may be combined with one or more of the embodiments mentioned under “Preferred amino acids in Z2” and/or “Preferred amino acids in Z3” and vice versa. Any such combination of embodiments is to be understood as a directly and unambiguously part of the disclosure.

With reference to the second aspect of the invention, examples of combinations of embodiments mentioned hereinbefore are:
Z1-Z2-Z3 according to the second aspect, wherein Z2 is Z2-Emb4;
Z1-Emb1, Z2-Emb2, and Z3-Emb1; Z1-Emb1, Z2-Emb5, and Z3-Emb1; Z1-Emb1, Z2-Emb6 and Z3-Emb1;
Z1-Emb2, Z2-Emb2, and Z3-Emb1; Z1-Emb2, Z2-Emb7, and Z3-Emb1;
Z1-Emb3, Z2-Emb3, and Z3-Emb2.

### Preferred Lipid/Linker

The polypeptide according to the first and/or second aspect of the invention, may be lipidated at a lysine (K) residue, preferably at the lysine (K) residue in amino acid position X₂₈, with a structure (i.e lipid/linker) of general formula L-Y¹—Y²—Y³—Y⁴—Y⁵—Y⁶—Y⁷—Y⁸, wherein L is a lipid selected from 17-carboxy-heptadecanoyl (briefly C18DA) and 19-carboxy-nonadecanoyl (briefly C20DA) and further wherein each of Y¹—Y⁸ are independently selected from: not present, [γE], [E], [OEG], [eLys], or [AHX].

Preferably, the lipid/linker has a structure of general formula L-Y¹—Y²—Y³—Y⁴—Y⁵—Y⁶—Y⁷—Y⁸, wherein L is a lipid selected from C18DA or C20DA, wherein each of Y¹—Y⁸ are independently selected from: not present, [γE], [E], [OEG], [eLys], or [AHX], wherein each of Y¹—Y³ are [γE] or each of Y¹—Y³ are [E], wherein Y⁴ is selected from: [γE], [E], or [OEG], and wherein each of Y⁵—Y⁸ are independently selected from: not present, [γE], [E], [OEG], [eLys], or [AHX].

Preferably, the lipid/linker has a structure of general formula L-Y¹—Y²—Y³—Y⁴—Y⁵—Y⁶—Y⁷—Y⁸, wherein L is C20DA, wherein each of Y¹—Y⁸ are independently selected from: not present, [γE], [E], [OEG], or [eLys], wherein each of Y¹—Y⁴ are [γE] or each of Y¹—Y⁴ are [E], wherein Y⁵ is selected from: [γE], [E], [OEG], or [eLys], and wherein each Y⁶—Y⁸ are independently selected from: not present, [γE], [E], [OEG], or [eLys].

The polypeptide of the invention, for example, is lipidated at the lysine (K) residue in amino acid position X₂₈, and the lipid/linker is selected from the group consisting of:

(SEQ ID NO: 640)
C18DA[E][E][E][E]-,
(SEQ ID NO: 636)
C18DA[E][E][E][E][E]-,
(SEQ ID NO: 637)
C18DA[E][E][E][E][E][E]-,
C18DA[γE][γE][γE][γE]-,
C18DA[γE][γE][γE][γE][γE]-,
C18DA[γE][γE][γE][γE][γE][γE]-,
C18DA[γE][γE][γE][γE][γE][γE][γE]-,
C20DA[γE][γE][γE][γE]-,
C20DA[γE][γE][γE][γE][γE]-,
C20DA[γE][γE][γE][γE][γE][γE]-,
C20DA[γE][γE][γE][γE][γE][γE][γE]-,
(SEQ ID NO: 635)
C18DA[E][E][E][E][E][eLys]-,
(SEQ ID NO: 643)
C18DA[E][E][E][E][E][E][eLys]-,
C18DA[γE][γE][γE][γE][eLys]-,
C18DA[γE][γE][γE][γE][γE][eLys]-,
C18DA[γE][γE][γE][γE][γE][γE][eLys]-,
(SEQ ID NO: 639)
C20DA[E][E][E][E][E][eLys]-,
C20DA[γE][γE][γE][γE][eLys]-,
C20DA[γE][γE][γE][γE][γE][eLys]-,
C20DA[γE][γE][γE][γE][γE][γE][eLys]-,
C20DA[γE][γE][γE][γE][γE][γE][γE][eLys]-,
C20DA[γE][γE][γE][γE][γE][eLys][eLys]-,
C18DA[E][E][E][OEG][OEG]-,
(SEQ ID NO: 634)
C18DA[E][E][E][E][OEG][OEG]-,
C18DA[γE][γE][γE][OEG][OEG]-,
C18DA[γE][γE][γE][γE][OEG][OEG]-,
C18DA[γE][γE][γE][γE][γE][OEG][OEG]-,
(SEQ ID NO: 638)
C20DA[E][E][E][E][OEG][OEG]-,
C20DA[γE][γE][γE][OEG][OEG]-,
C20DA[γE][γE][γE][γE][OEG][OEG]-,
C20DA[γE][γE][γE][γE][γE][OEG][OEG]-,
C18DA[γE][γE][γE][OEG]-,
C18DA[γE][γE][γE][γE][OEG]-,
C18DA[γE][γE][γE][γE][γE][OEG]-,
C18DA[γE][γE][γE][γE][γE][γE][OEG]-,
C20DA[γE][γE][γE][OEG]-,
C20DA[γE][γE][γE][γE][OEG]-,
C20DA[γE][γE][γE][γE][γE][OEG]-,
C20DA[γE][γE][γE][γE][γE][γE][OEG]-,
C18DA[γE][γE][γE][OEG][eLys]-,
C18DA[γE][γE][γE][γE][OEG][eLys]-,
C18DA[γE][γE][γE][γE][γE][OEG][eLys]-,
C20DA[γE][γE][γE][OEG][eLys]-,
C20DA[γE][γE][γE][γE][OEG][eLys]-,
C20DA[γE][γE][γE][γE][γE][OEG][eLys]-,
C18DA[γE][γE][γE][OEG][OEG][eLys]-,
C18DA[γE][γE][γE][γE][OEG][OEG][eLys]-,
C20DA[γE][γE][γE][OEG][OEG][eLys]-,
C20DA[γE][γE][γE][γE][OEG][OEG][eLys]-,
C18DA[γE][γE][γE][γE][AHX]-,
C18DA[γE][γE][γE][γE][γE][AHX]-,
C18DA[γE][γE][γE][γE][γE][γE][AHX]-,
C20DA[γE][γE][γE][γE][AHX]-,
C20DA[γE][γE][γE][γE][γE][AHX]-,
or
C20DA[γE][γE][γE][γE][γE][γE][AHX]-.

The polypeptide of the invention, for example, is lipidated at the lysine (K) residue in amino acid position X₂₈, and the lipid/linker is selected from the group consisting of:

C20DA[γE][γE][γE][γE][γE]-,
C20DA[γE][γE][γE][γE][γE][γE]-,
C20DA[γE][γE][γE][γE][γE][γE][γE]-,
(SEQ ID NO: 635)
C18DA[E][E][E][E][E][eLys]-,
(SEQ ID NO: 639)
C20DA[E][E][E][E][E][eLys]-,
C20DA[γE][γE][γE][γE][eLys]-,
C20DA[γE][γE][γE][γE][γE][eLys]-,
C20DA[γE][γE][γE][γE][γE][γE][eLys]-,
C20DA[γE][γE][γE][γE][γE][γE][γE][eLys]-,
C20DA[γE][γE][γE][γE][γE][eLys][eLys]-,
(SEQ ID NO: 638)
C20DA[E][E][E][E][OEG][OEG]-,
C20DA[γE][γE][γE][γE][OEG][OEG]-,
C20DA[γE][γE][γE][γE][γE][OEG][OEG]-,
C20DA[γE][γE][γE][γE][OEG]-,
C20DA[γE][γE][γE][γE][γE][OEG]-,
C20DA[γE][γE][γE][γE][γE][γE][OEG]-,
C20DA[γE][γE][γE][γE][OEG][eLys]-,
C20DA[γE][γE][γE][γE][γE][OEG][eLys]-,
C20DA[E][γE][γE][γE][OEG][OEG][eLys]-.

The triple agonists of the invention are generally soluble around pH 7. There are several techniques known to the skilled person in the art how to determine solubility. Preferably the solubility of the peptide of the invention is determined as disclosed in Example 2 hereinafter.

In some embodiments, the solubility of the compounds of the invention is greater than or equal to 1.0 mg/ml around pH 7.

In some embodiments, the solubility of the compounds of the invention is greater than 3.0 mg/ml around pH 7.

In some embodiments, the solubility of the compounds of the invention is greater than 5.0 mg/ml around pH 7.

In some embodiments, the solubility of the compounds of the invention is equal or greater than 6.0 mg/ml, 7.0 mg/ml, 8.0 mg/ml or 9.0 mg/ml around pH 7.

The hybrid polypeptides of the present invention are able to agonize the hGLP1R, the GIPR and the hNPY2R in the presence of human serum albumin (see table 3), the main protein present in human plasma. Activity of peptides of the invention in the presence of human plasma is an important prerequisite for viable compounds useful for the treatment of the disclosed diseases. The skilled person will be aware of suitable assay formats, and an example is provided hereinafter. For instance, for peptides of the present invention the EC₅₀ values to the hGLP1R, the GIPR and the hNPY2R in the presence of 100% human plasma (100% hP) was evaluated by the assay as described in Example 1 hereinafter.

In some embodiments of peptides of the present invention, the EC₅₀ towards hGLP1R 100% hP and GIPR 100% hP is below 100 nM (e.g. 0.01 nM to 100 nM), and towards hNPY2R 100% hP is below 1000 nM (e.g. 0.01 nM to 1000 nM).

In some embodiments of peptides of the present invention, the EC₅₀ towards hGLP1R 100% hP and GIPR 100% hP is below or equal to 30 nM (e.g. 0.01 nM to 50 nM), and towards hNPY2R 100% hP is below 300 nM (e.g. 0.01 nM to 300 nM).

In some embodiments of peptides of the present invention, the EC₅₀ towards hGLP1R 100% hP and GIPR 100% hP is below or equal to 10 nM (e.g. 0.01 nM to 10 nM), and towards hNPY2R 100% hP is below 100 nM (e.g. 0.01 nM to 100 nM).

In a preferred embodiment of the peptides of the present invention, the EC₅₀ towards hGLP1R 100% hP and GIPR 100% hP is below or equal to 10 nM (e.g. 0.01 nM to 10 nM), and towards hNPY2R 100% hP is below 100 nM (e.g. 0.01 nM to 100 nM), and the solubility is greater than or equal to 1.0 mg/ml around pH 7.

In some embodiments the peptides of the invention have favourable pharmacokinetic properties. In this regard, the in-vivo half-life of the peptides of the invention may be greater than 6 hours or greater than 8 hours or greater than 10 hours in the mouse (NMRI mice, see measurement described in Example 3).

In a preferred embodiment of the peptides of the present invention, the EC₅₀ towards hGLP1R 100% hP and GIPR 100% hP is below or equal to 10 nM (e.g. 0.01 nM to 10 nM), and towards hNPY2R 100% hP is below 100 nM (e.g. 0.01 nM to 100 nM), the solubility is greater than or equal to 1.0 mg/ml around pH 7, and the in-vivo half-life is greater than 8 hours.

In a third aspect, the invention provides a polypeptide for use as a medicament. Since Y² receptors have a feeding suppressive action, a polypeptide having Y² agonistic activity may be useful in the treatment of symptoms associated with eating disorder, obesity (e.g. simple obesity or symptomatic obesity) and/or diabetes. Furthermore, the gut hormones GLP-1 and GIP (called incretins), promote insulin secretion from the pancreas. Since incretins are closely related to glucose metabolism, a polypeptide having GLP-1 receptor agonistic activity and GIP receptor agonistic activity may be useful in the treatment of symptoms associated with a glucose metabolism disorder, including diabetes and obesity. Thus, the polypeptides of the present invention may have a feeding suppressive action, and/or a body weight-lowering action. In a preferred embodiment the invention provides a polypeptide for use in the prevention, treatment, and/or remission of a disease, disorder, or condition selected from the list consisting of excessive weight (overweight), obesity (e.g. simple obesity (chronic weight management) or symptomatic obesity), insulin resistance, diabetes (e.g. type 1 diabetes or type 2 diabetes) or prediabetes, eating disorders, hyperlipidemia (e.g., hypertriglyceridemia, hypercholesterolemia, high LDL-cholesterolemia, low HDL-cholesterolemia, postprandial hyperlipemia), metabolic syndrome (three of the following five medical conditions: abdominal obesity, high blood pressure, high blood sugar, high serum triglycerides, and low serum high-density lipoprotein (HDL)), liver diseases such as metabolic associated fatty liver disease (MAFLD), non-alcoholic fatty liver disease (NAFLD), non-alcoholic steato-hepatitis (NASH), portal hypertension; cardiovascular diseases (e.g. hypertension, atherosclerosis, stroke or cardiac failure); kidney diseases such as diabetic kidney disease (DKD), and chronic kidney disease (CKD); and neurodegenerative diseases (Alzheimer's or Parkinson's disease), but also other obesity related diseases such as osteoporosis, sleep apnoe, obesity associated cancers, and obesity associated asthma. Examples of the symptomatic obesity include endocrine obesity (e.g., Cushing syndrome, hypothyroidism, insulinoma, obese type 2 diabetes, pseudohypoparathyroidism, hypogonadism and associated endocrine disorders, such as polycystic ovary syndrome (PCOS)), central obesity (e.g., hypothalamic obesity, frontal lobe syndrome, Kleine-Levin syndrome), hereditary obesity (e.g., Prader-Willi syndrome, Laurence-Moon-Biedl syndrome), drug-induced obesity (e.g., steroid, phenothiazine, insulin, sulfonylurea agent, P-blocker-induced obesity).

The polypeptides of the invention may also be used for the prevention of obesity, or prevention or reversal of co-morbidities of obesity and/or overweight, such as type 2 diabetes, high blood pressure, NAFLD, NASH, DKD, CKD, sleep apnoe, obesity associated cancers, or obesity associated asthma.

In a fourth aspect, the invention provides a method for the treatment of a disease, disorder and/or condition said method comprising administering a therapeutic effective amount of a polypeptide or a pharmaceutically acceptable salt thereof, according to any of the aspects and embodiments disclosed herein, to an individual in need thereof. Preferably, the disease, disorder and/or condition is selected from the list consisting of excessive weight, obesity, diabetes (type 1 or type 2) or prediabetes, eating disorders, hyperlipidemia, metabolic syndrome, NAFLD, NASH, and/or cardiovascular diseases. Most preferably the disease, disorder and/or condition is obesity, prediabetes and/or diabetes (type 1 or type 2).

Furthermore, the invention provides a method of binding to and/or activating the GLP-1, the GIP and the hNPY2 receptors in an individual in need thereof, said method comprising administering a therapeutic effective amount of a polypeptide or a pharmaceutically acceptable salt thereof, according to any of the aspects and embodiments disclosed herein.

Likewise, the present invention relates to the use of a polypeptide or a pharmaceutically acceptable salt thereof in the manufacture of a medicament for the treatment of a disease, disorder and/or condition, according to any of the aspects and embodiments disclosed herein.

The dose range of the polypeptide according to the general structure of Formula (I) applicable per week is usually from 0.01 to 100 mg for humans (subcutaneous administration).

The polypeptide of the invention may be administered subcutaneously using a suitable device such as a pre-filled ready-to-use injection device, a syringe, a pen injector, or an auto-injector.

The actual pharmaceutically effective amount or therapeutic dosage will usually depend on factors known by those skilled in the art such as age and weight of the patient, route of administration and severity of disease.

In any case, the compounds will be administered at dosages and in a manner, which allows a pharmaceutically effective amount to be delivered based upon patient's unique condition.

### EXAMPLES

TABLE 1
GIP/GLP-1 part (Z1 of Formula (I))

| | | | | | | | | | | | | | | | | | | | | | | | | | | | | | Nr of |
|---|---|---|---|---|---|---|---|---|---|---|---|---|---|---|---|---|---|---|---|---|---|---|---|---|---|---|---|---|---|
| | | | | | | | | | | | | | | | | | | | | | | | | | | | | | substi- |
| | | | | | | | | | | | | | | | | | | | | | | | | | | | | | tutions |
| | | | | | | | | | | | | | | | | | | | | | | | | | | | | | to Z1 |
| | | | | | | | | | | | | | | | | | | | | | | | | | | | | | of SEQ |
| SEQ | | | | | | | | | | | | | | | | | | | | | | | | | | | | | ID |
| ID | | | | | | | | | | | | | | | | | | | | | | | | | | | | | NO: |
| NO: | X₁ | X₂ | X₃ | X₄ | X₅ | X₆ | X₇ | X₈ | X₉ | X₁₀ | X₁₁ | X₁₂ | X₁₃ | X₁₄ | X₁₅ | X₁₆ | X₁₇ | X₁₈ | X₁₉ | X₂₀ | X₂₁ | X₂₂ | X₂₃ | X₂₄ | X₂₅ | X₂₆ | X₂₇ | X₂₈ | 286 |
| 2 | Y | Aib | E | G | T | F | T | S | D | Y | S | I | Aib | L | E | K | Q | A | Q | Aib | A | F | V | E | W | L | I | K | 0 |
| 3 | Y | Aib | E | G | T | F | T | S | D | Y | S | I | Aib | L | E | K | Q | A | Q | Aib | A | F | V | E | W | L | I | K | 1 |
| 4 | Y | Aib | E | G | T | F | T | S | D | Y | S | I | Aib | L | E | K | Q | A | Q | Aib | A | F | V | E | W | L | I | K | 0 |
| 5 | Y | Aib | E | G | T | F | T | S | D | Y | S | I | Aib | L | E | K | Q | A | Q | Aib | A | F | V | E | W | L | I | K | 0 |
| 6 | Y | Aib | E | G | T | F | T | S | D | Y | S | I | Aib | L | E | K | Q | A | Q | Aib | A | F | V | Q | W | L | I | K | 1 |
| 7 | Y | Aib | E | G | T | F | T | S | D | Y | S | I | Aib | L | E | E | E | A | Q | Aib | A | F | V | Q | W | L | I | K | 3 |
| 8 | Y | Aib | E | G | T | F | T | S | D | Y | S | I | Aib | L | E | E | Q | A | Q | Aib | A | F | V | Q | W | L | I | K | 2 |
| 9 | Y | Aib | E | G | T | F | T | S | D | Y | S | I | Aib | L | E | K | Q | A | Q | Aib | A | F | V | E | W | L | I | K | 0 |
| 10 | Y | Aib | E | G | T | F | T | S | D | Y | S | I | Aib | L | E | K | Q | A | Q | Aib | A | F | V | E | W | L | I | K | 0 |
| 11 | Y | Aib | E | G | T | F | T | S | D | Y | S | I | Aib | L | E | K | Q | A | Q | Aib | A | F | V | Q | W | L | I | K | 1 |
| 12 | Y | Aib | E | G | T | F | T | S | D | Y | S | I | Aib | L | E | K | Q | A | Q | Aib | A | F | V | Q | W | L | I | K | 1 |
| 13 | Y | Aib | E | G | T | F | T | S | D | Y | S | I | Aib | L | E | K | Q | A | Q | Aib | A | F | V | E | W | L | I | K | 0 |
| 14 | Y | Aib | E | G | T | F | T | S | D | Y | S | I | Aib | L | E | K | Q | A | Q | Aib | A | F | V | Q | W | L | I | K | 1 |
| 15 | Y | Aib | E | F | T | F | T | S | D | Y | S | I | Aib | L | E | K | Q | A | Q | Aib | A | F | V | Q | W | L | I | K | 1 |
| 16 | Y | Aib | E | G | T | F | T | S | D | Y | S | I | Aib | L | E | Aib | E | A | Q | Aib | K | F | V | E | W | L | I | K | 3 |
| 17 | Y | Aib | E | G | T | F | T | S | D | Y | S | I | Aib | L | E | K | Q | A | Q | Aib | A | F | V | E | W | L | I | K | 0 |
| 18 | Y | Aib | E | G | T | F | T | S | D | Y | S | I | Aib | L | E | K | Q | A | Q | Aib | A | F | V | E | W | L | I | K | 0 |
| 19 | Y | Aib | E | G | T | F | T | S | D | Y | S | I | Aib | L | E | K | Q | A | Q | Aib | A | F | V | Q | W | L | I | K | 1 |
| 20 | Y | Aib | E | G | T | F | T | S | D | Y | S | I | Aib | L | A | A | A | A | Q | Aib | A | F | V | E | W | L | I | K | 3 |
| 21 | Y | Aib | E | G | T | F | T | S | D | Y | S | I | Aib | L | E | K | Q | A | Q | Aib | A | F | V | E | W | L | I | K | 0 |
| 22 | Y | Aib | E | G | T | F | T | S | D | Y | S | I | Aib | L | E | K | Q | A | Q | Aib | A | F | V | Q | W | L | I | K | 1 |
| 23 | Y | Aib | E | G | T | F | T | S | D | Y | S | I | Aib | L | E | K | Q | A | Q | Aib | A | F | V | V | W | L | I | K | 1 |
| 24 | Y | Aib | E | G | T | F | T | S | D | Y | S | I | Aib | L | E | Aib | K | A | Q | Aib | E | F | V | E | W | L | I | K | 3 |
| 25 | Y | Aib | E | G | T | F | T | S | D | Y | S | I | Aib | L | E | A | A | K | Q | Aib | A | F | V | E | W | L | I | K | 3 |
| 26 | Y | Aib | E | G | T | F | T | S | D | Y | S | I | Aib | L | E | K | Q | A | Q | Aib | A | F | V | E | W | L | I | K | 0 |
| 27 | Y | Aib | E | G | T | F | T | S | D | Y | S | I | Aib | L | E | K | Q | A | Q | Aib | A | F | V | E | W | L | I | K | 0 |
| 28 | Y | Aib | E | G | T | F | T | S | D | Y | S | I | Aib | L | E | K | Q | A | Q | Aib | E | F | V | Q | W | L | I | K | 2 |
| 29 | Y | Aib | E | G | T | F | T | S | D | Y | S | I | Aib | L | E | K | Q | A | Q | Aib | A | F | V | Q | W | L | I | K | 1 |
| 30 | Y | Aib | E | G | T | F | T | S | D | Y | S | I | Aib | L | E | E | Q | A | Q | Aib | A | F | V | Q | W | L | I | K | 2 |
| 31 | Y | Aib | E | G | T | F | T | S | S | Y | S | I | Aib | L | E | E | Q | A | Q | Aib | A | F | V | E | W | L | I | K | 1 |
| 32 | Y | Aib | E | G | T | F | T | S | D | Y | S | I | Aib | L | E | K | Q | A | Q | Aib | A | F | V | Q | W | L | I | K | 1 |
| 33 | Y | Aib | E | G | T | F | T | S | D | Y | S | I | Aib | L | E | K | Q | A | Q | Aib | A | F | V | E | W | L | I | K | 0 |
| 34 | Y | Aib | E | G | T | F | T | S | D | Y | S | I | Aib | L | E | E | Q | A | Q | Aib | A | F | V | Q | W | L | I | K | 2 |
| 35 | Y | Aib | E | G | T | F | T | S | D | Y | S | I | Aib | L | E | K | Q | A | Q | Aib | A | F | V | Q | W | L | I | K | 1 |
| 36 | Y | Aib | E | G | T | F | T | S | D | Y | S | I | Aib | L | E | K | Q | A | Q | Aib | E | F | V | Q | W | L | I | K | 2 |
| 37 | Y | Aib | E | G | T | F | T | S | S | Y | S | I | Aib | L | E | K | Q | A | Q | Aib | A | F | V | Q | W | L | I | K | 1 |
| 38 | Y | Aib | E | G | T | F | T | S | D | Y | S | I | Aib | L | E | K | Q | A | Q | Aib | A | F | V | E | W | L | I | K | 0 |
| 39 | Y | Aib | E | G | T | F | T | S | D | Y | S | I | Aib | L | E | K | Q | A | Q | Aib | A | F | V | E | W | L | I | K | 0 |
| 40 | Y | Aib | E | G | T | F | T | S | S | Y | S | I | Aib | L | E | K | Q | A | Q | Aib | A | F | V | E | W | L | I | K | 0 |
| 41 | Y | Aib | E | G | T | F | T | S | D | Y | S | I | Aib | L | E | K | Q | A | Q | Aib | A | F | V | E | W | L | I | K | 0 |
| 42 | Y | Aib | E | G | T | F | T | S | D | Y | S | I | Aib | L | E | K | Q | A | Q | Aib | A | F | V | E | W | L | I | K | 0 |
| 43 | Y | Aib | E | G | T | F | T | S | D | Y | S | I | Aib | L | E | K | Q | A | Q | Aib | E | F | V | Q | W | L | I | K | 2 |
| 44 | Y | Aib | E | G | T | F | T | S | D | Y | S | I | Aib | L | E | K | Q | A | Q | Aib | A | F | V | E | W | L | I | K | 0 |
| 45 | Y | Aib | E | G | T | F | T | S | D | Y | S | I | Aib | L | E | E | Q | A | Q | Aib | A | F | V | Q | W | L | I | K | 2 |
| 46 | Y | Aib | E | G | T | F | T | S | D | Y | S | I | Aib | L | E | K | E | A | Q | Aib | K | F | V | E | W | L | I | K | 2 |
| 47 | Y | Aib | E | G | T | F | T | S | D | Y | S | I | Aib | L | E | K | Q | A | Q | Aib | A | F | V | Q | W | L | I | K | 1 |
| 48 | Y | Aib | E | G | T | F | T | S | D | Y | S | I | Aib | L | E | K | Q | A | Q | Aib | A | F | V | E | W | L | I | K | 0 |
| 49 | Y | Aib | E | G | T | F | T | S | S | Y | S | I | Aib | L | E | K | Q | A | Q | Aib | A | F | V | E | W | L | I | K | 0 |
| 50 | Y | Aib | E | G | T | F | T | S | D | Y | S | I | Aib | L | E | K | Q | A | Q | Aib | A | F | V | E | W | L | I | K | 0 |
| 51 | Y | Aib | E | G | T | F | T | S | S | Y | S | I | Aib | L | E | K | Q | A | E | Aib | A | F | V | Q | W | L | I | K | 2 |
| 52 | Y | Aib | E | G | T | F | T | S | S | Y | S | I | Aib | L | E | K | Q | A | Q | Aib | A | F | V | Q | W | L | I | K | 1 |
| 53 | Y | Aib | E | G | T | F | T | S | D | Y | S | I | Aib | L | E | K | Q | A | Q | Aib | A | F | V | Q | W | L | I | K | 1 |
| 54 | Y | Aib | E | G | T | F | T | S | D | Y | S | I | Aib | L | E | K | Q | A | E | Aib | A | F | V | Q | W | L | I | K | 2 |
| 55 | Y | Aib | E | G | T | F | T | S | D | Y | S | I | Aib | L | E | K | Q | A | Q | Aib | A | F | V | Q | W | L | I | K | 1 |
| 56 | Y | Aib | E | G | T | F | T | S | D | Y | S | I | Aib | L | E | K | Q | A | Q | Aib | E | F | V | E | W | L | I | K | 1 |
| 57 | Y | Aib | E | G | T | F | T | S | D | Y | S | I | Aib | L | E | K | Q | A | Q | Aib | A | F | V | Q | W | L | I | K | 1 |
| 58 | Y | Aib | E | G | T | F | T | S | D | Y | S | I | Aib | L | E | K | Q | A | Q | Aib | A | F | V | Q | W | L | I | K | 1 |
| 59 | Y | Aib | E | G | T | F | T | S | D | Y | S | I | Aib | L | E | K | Q | A | Q | Aib | A | F | V | Q | W | L | I | K | 1 |
| 60 | Y | Aib | E | G | T | F | T | S | D | Y | S | I | Aib | L | E | K | Q | A | Q | Aib | A | F | V | Q | W | L | I | K | 1 |
| 61 | Y | Aib | E | G | T | F | T | S | D | Y | S | I | Aib | L | E | K | Q | A | Q | Aib | A | F | V | Q | W | L | I | K | 1 |
| 62 | Y | Aib | E | G | T | F | T | S | D | Y | S | I | Aib | L | E | K | Q | A | Q | Aib | A | F | V | E | W | L | I | K | 0 |
| 63 | Y | Aib | E | G | T | F | T | S | D | Y | S | I | Aib | L | E | K | Q | A | Q | Aib | A | F | V | Q | W | L | I | K | 1 |
| 64 | Y | Aib | E | G | T | F | T | S | D | Y | S | I | Aib | L | E | K | Q | A | Q | Aib | A | F | V | E | W | L | I | K | 0 |
| 65 | Y | Aib | E | G | T | F | T | S | D | Y | S | I | Aib | L | E | K | Q | A | Q | Aib | A | F | V | E | W | L | I | K | 0 |
| 66 | Y | Aib | E | G | T | F | T | S | D | Y | S | I | Aib | L | E | E | E | A | Q | Aib | A | F | V | E | W | L | I | K | 2 |
| 67 | Y | Aib | E | G | T | F | T | S | D | Y | S | I | Aib | L | E | K | Q | D | Q | Aib | A | F | V | Q | W | L | I | K | 1 |
| 68 | Y | Aib | E | G | T | F | T | S | D | Y | S | I | Aib | L | E | K | Q | D | Q | Aib | A | F | V | E | W | L | I | K | 0 |
| 69 | Y | Aib | E | G | T | F | T | S | D | Y | S | I | Aib | L | E | K | E | K | Q | Aib | A | F | V | E | W | L | I | K | 2 |
| 70 | Y | Aib | E | G | T | F | T | S | D | Y | S | I | Aib | L | E | K | Q | A | Q | Aib | A | F | V | E | W | L | I | K | 0 |
| 71 | Y | Aib | E | G | T | F | T | S | D | Y | S | I | Aib | L | E | K | Q | A | Q | Aib | A | F | V | E | W | L | I | K | 0 |
| 72 | Y | Aib | E | G | T | F | T | S | D | Y | S | I | Aib | L | E | K | Q | A | Q | Aib | A | F | V | E | W | L | I | K | 0 |
| 73 | Y | Aib | E | G | T | F | T | S | D | Y | S | I | Aib | L | E | K | Q | A | Q | Aib | A | F | V | E | W | L | I | K | 0 |
| 74 | Y | Aib | E | G | T | F | T | S | D | Y | S | I | Aib | L | E | K | Q | A | Q | Aib | A | F | V | E | W | L | I | K | 0 |
| 75 | Y | Aib | E | G | T | F | T | S | D | Y | S | I | Aib | L | E | K | Q | A | Q | Aib | A | F | V | E | W | L | I | K | 0 |
| 76 | Y | Aib | E | G | T | F | T | S | D | Y | S | I | Aib | L | E | K | Q | A | Q | Aib | A | F | V | E | W | L | I | K | 0 |
| 77 | Y | Aib | E | G | T | F | T | S | D | Y | S | I | Aib | L | E | K | Q | A | Q | Aib | A | F | V | Q | W | L | I | K | 1 |
| 78 | Y | Aib | E | G | T | F | T | S | D | Y | S | I | Aib | L | E | K | Q | A | Q | Aib | A | F | V | Q | W | L | I | K | 1 |
| 79 | Y | Aib | E | G | T | F | T | S | D | Y | S | I | Aib | L | E | K | Q | A | Q | Aib | A | F | V | E | W | L | I | K | 0 |
| 80 | Y | Aib | E | G | T | F | T | S | D | Y | S | I | Aib | L | E | K | Q | A | Q | Aib | A | F | V | E | W | L | I | K | 0 |
| 81 | Y | Aib | E | G | T | F | T | S | D | Y | S | I | Aib | L | E | K | Q | A | Q | Aib | E | F | V | E | W | L | I | K | 1 |
| 82 | Y | Aib | E | G | T | F | T | S | D | Y | S | I | Aib | L | E | K | Q | A | Q | Aib | A | F | V | E | W | L | I | K | 0 |
| 83 | Y | Aib | E | G | T | F | T | S | D | Y | S | I | Aib | L | E | K | Q | A | Q | Aib | A | F | V | E | W | L | I | K | 0 |
| 84 | Y | Aib | E | G | T | F | T | S | D | Y | S | I | Aib | L | E | K | Q | A | Q | Aib | A | F | V | E | W | L | I | K | 0 |
| 85 | Y | Aib | E | G | T | F | T | S | D | Y | S | I | Aib | L | E | K | Q | A | Q | Aib | A | F | V | E | W | L | I | K | 0 |
| 86 | Y | Aib | E | G | T | F | T | S | D | Y | S | I | Aib | L | E | K | Q | A | Q | Aib | A | F | V | E | W | L | I | K | 0 |
| 87 | Y | Aib | D | G | T | F | T | S | D | Y | S | I | Aib | L | E | K | Q | A | Q | Aib | A | F | V | E | W | L | I | K | 1 |
| 88 | Y | Aib | E | G | T | F | T | S | D | Y | S | I | Aib | L | E | K | Q | A | Q | Aib | E | F | V | E | W | L | I | K | 1 |
| 89 | Y | Aib | E | G | T | F | T | S | D | Y | S | I | Aib | L | E | K | Q | A | Q | Aib | A | F | V | Q | W | L | I | K | 1 |
| 90 | Y | Aib | E | G | T | F | T | S | D | Y | S | I | Aib | L | E | D | Q | A | Q | Aib | A | F | V | E | W | L | I | K | 1 |
| 91 | Y | Aib | E | G | T | F | T | S | D | Y | S | I | Aib | L | E | K | Q | A | Q | Aib | A | F | V | E | W | L | I | K | 0 |
| 92 | Y | Aib | E | G | T | F | T | S | D | Y | S | I | Aib | L | E | K | Q | A | Q | Aib | A | F | V | Q | W | L | I | K | 1 |
| 93 | Y | Aib | E | G | T | F | T | S | D | Y | S | I | Aib | L | E | K | Q | A | Q | Aib | A | F | V | E | W | L | I | K | 0 |
| 94 | Y | Aib | E | G | T | F | T | S | D | Y | S | I | Aib | L | E | K | Q | A | Q | Aib | A | F | V | E | W | L | I | K | 0 |
| 95 | Y | Aib | E | G | T | F | T | S | D | Y | S | I | Aib | L | E | K | Q | A | Q | Aib | A | F | V | E | W | L | I | K | 0 |
| 96 | Y | Aib | E | G | T | F | T | S | S | Y | S | I | Aib | L | E | K | Q | A | Q | Aib | A | F | V | E | W | L | I | K | 0 |
| 97 | Y | Aib | E | G | T | F | T | S | D | Y | S | I | Aib | L | E | E | Q | A | Q | Aib | A | F | V | E | W | L | I | K | 1 |
| 98 | Y | Aib | E | G | T | F | T | S | D | Y | S | I | Aib | L | E | K | Q | A | Q | Aib | A | F | V | E | W | L | I | K | 0 |
| 99 | Y | Aib | E | G | T | F | T | S | D | Y | S | I | Aib | L | E | G | Q | A | Q | Aib | A | F | V | E | W | L | I | K | 1 |
| 100 | Y | Aib | E | G | T | F | T | S | D | Y | S | I | Aib | L | E | E | Q | A | Q | Aib | A | F | V | E | W | L | I | K | 1 |
| 101 | Y | Aib | E | G | T | F | T | S | D | Y | S | I | Aib | L | E | K | Q | A | Q | Aib | A | F | V | Q | W | L | I | K | 1 |
| 102 | Y | Aib | E | G | T | F | T | S | D | Y | S | I | Aib | L | E | A | Q | A | Q | Aib | A | F | V | E | W | L | I | K | 1 |
| 103 | Y | Aib | E | G | T | F | T | S | D | Y | S | I | Aib | L | E | K | Q | A | Q | Aib | A | F | V | E | W | L | I | K | 0 |
| 104 | Y | Aib | E | G | T | F | T | S | D | Y | S | I | Aib | L | E | K | Q | A | Q | Aib | A | F | V | E | W | L | I | K | 1 |
| 105 | Y | Aib | E | G | T | F | T | S | D | Y | S | I | Aib | L | E | E | Q | A | Q | Aib | A | F | V | E | W | L | I | K | 1 |
| 106 | Y | Aib | E | G | T | F | T | S | D | Y | S | I | Aib | L | E | K | Q | A | Q | Aib | A | F | V | E | W | L | I | K | 0 |
| 107 | Y | Aib | E | G | T | F | T | S | D | Y | S | I | Aib | L | E | K | Q | A | Q | Aib | A | F | V | E | W | L | I | K | 0 |
| 108 | Y | Aib | E | G | T | F | T | S | D | Y | S | I | Aib | L | E | K | Q | A | Q | Aib | A | F | V | Q | W | L | I | K | 1 |
| 109 | Y | Aib | E | G | T | F | T | S | D | Y | S | I | Aib | L | E | K | Q | A | Q | Aib | A | F | V | E | W | L | I | K | 1 |
| 110 | Y | Aib | E | G | T | F | T | S | D | Y | S | I | Aib | L | E | E | Q | A | Q | Aib | A | F | V | E | W | L | I | K | 1 |
| 111 | Y | Aib | E | G | T | F | T | S | D | Y | S | I | Aib | L | E | K | Q | A | Q | Aib | A | F | V | E | W | L | I | K | 1 |
| 112 | Y | Aib | E | G | T | F | T | S | S | Y | S | I | Aib | L | E | K | Q | A | Q | Aib | A | F | V | E | W | L | I | K | 1 |
| 113 | Y | Aib | E | G | T | F | T | S | D | L | S | I | Aib | L | E | K | Q | A | Q | Aib | A | F | V | E | W | L | I | K | 2 |
| 114 | Y | Aib | E | G | T | F | T | S | D | Y | S | I | Aib | L | E | K | Q | A | Q | Aib | A | F | V | Q | W | L | I | K | 1 |
| 115 | Y | Aib | E | G | T | F | T | S | D | Y | S | I | Aib | L | E | K | Q | A | Q | Aib | A | F | V | Q | W | L | I | K | 1 |
| 116 | Y | Aib | E | G | T | F | T | S | D | Y | S | I | Aib | L | E | E | Q | A | Q | Aib | A | F | V | E | W | L | I | K | 1 |
| 117 | Y | Aib | E | G | T | F | T | S | D | Y | S | I | Aib | L | E | K | Q | A | Q | Aib | A | F | V | E | W | L | I | K | 0 |
| 118 | Y | Aib | E | G | T | F | T | S | D | Y | S | I | Aib | L | E | K | Q | A | Q | Aib | A | F | V | E | W | L | I | K | 0 |
| 119 | Y | Aib | E | G | T | F | T | S | D | Y | S | I | Aib | L | E | K | Q | A | Q | Aib | A | F | V | E | W | L | I | K | 0 |
| 120 | Y | Aib | E | G | T | F | T | S | D | Y | S | I | Aib | L | E | K | Q | A | Q | Aib | A | F | V | E | W | L | I | K | 0 |
| 121 | Y | Aib | E | G | T | F | T | S | D | Y | S | I | Aib | L | E | S | Q | A | Q | Aib | A | F | V | E | W | L | I | K | 1 |
| 122 | Y | Aib | E | G | T | F | T | S | D | Y | S | I | Aib | L | E | K | Q | A | Q | Aib | A | F | V | E | W | L | I | K | 0 |
| 123 | Y | Aib | E | G | T | F | T | S | D | Y | S | I | Aib | L | E | K | Q | A | Q | Aib | A | F | V | E | W | L | I | K | 0 |
| 124 | Y | Aib | E | G | T | F | T | S | D | Y | S | I | Aib | L | E | Q | Q | A | Q | Aib | A | F | V | E | W | L | I | K | 1 |
| 125 | Y | Aib | E | G | T | F | T | S | D | Y | S | I | Aib | L | E | K | Q | A | Q | Aib | A | F | V | Q | W | L | I | K | 1 |
| 126 | Y | Aib | E | G | T | F | T | S | D | Y | S | I | Aib | L | E | K | Q | A | Q | Aib | A | F | V | E | W | L | I | K | 0 |
| 127 | Y | Aib | E | G | T | F | T | S | D | Y | S | I | Aib | L | E | K | Q | A | Q | Aib | A | F | V | E | W | L | I | K | 0 |
| 128 | Y | Aib | E | G | T | F | T | S | D | Y | S | I | Aib | L | D | K | Q | A | Q | Aib | A | F | V | E | W | L | I | K | 1 |
| 129 | Y | Aib | E | G | T | F | T | S | D | Y | S | I | Aib | L | E | K | Q | A | Q | Aib | A | F | V | E | W | L | I | K | 0 |
| 130 | Y | Aib | E | G | T | F | T | S | D | Y | S | I | Aib | L | E | K | Q | A | Q | Aib | A | F | I | E | W | L | I | K | 1 |
| 131 | Y | Aib | E | G | T | F | T | S | D | Y | S | I | Aib | L | E | R | Q | A | Q | Aib | A | F | V | E | W | L | I | K | 1 |
| 132 | Y | Aib | E | G | T | F | T | S | D | Y | S | I | Aib | L | E | K | Q | A | Q | Aib | A | F | V | E | W | L | I | K | 0 |
| 133 | Y | Aib | E | G | T | F | T | S | D | Y | S | I | Aib | L | E | K | Q | A | Q | Aib | A | F | V | E | W | L | I | K | 0 |
| 134 | Y | Aib | E | G | T | F | T | S | D | Y | S | I | Aib | L | E | K | Q | A | Q | Aib | A | F | V | E | W | L | I | K | 0 |
| 135 | Y | Aib | E | G | T | F | T | S | D | Y | S | I | Aib | L | E | K | Q | A | Q | Aib | A | F | V | E | Y | L | I | K | 1 |
| 136 | Y | Aib | E | G | T | F | T | S | D | Y | S | I | Aib | L | E | I | A | Q | Q | Aib | A | F | V | E | W | L | I | K | 1 |
| 137 | Y | Aib | E | G | T | F | T | S | D | Y | S | I | Aib | L | E | K | Q | A | Q | Aib | A | F | V | E | W | L | I | K | 0 |
| 138 | Y | Aib | E | G | T | F | T | S | D | Y | S | I | Aib | L | E | K | Q | A | E | Aib | A | F | V | Q | W | L | I | K | 2 |
| 139 | Y | Aib | E | G | T | F | T | S | D | Y | S | I | Aib | L | E | K | Q | A | Q | Aib | A | F | V | Q | W | L | I | K | 1 |
| 140 | Y | Aib | E | G | T | F | T | S | D | Y | S | I | Aib | L | E | K | Q | A | Q | Aib | A | F | V | E | W | L | I | K | 0 |
| 141 | Y | Aib | E | G | T | F | T | S | S | Y | S | I | Aib | L | E | K | Q | A | Q | Aib | E | F | V | Q | W | L | I | K | 2 |
| 142 | Y | Aib | E | G | T | F | T | S | D | Y | S | I | Aib | L | E | K | Q | A | Q | Aib | A | F | V | Q | W | L | I | K | 1 |
| 143 | Y | Aib | E | G | T | F | T | S | D | Y | S | I | Aib | L | E | K | Q | A | Q | Aib | A | F | V | Q | W | L | I | K | 1 |
| 144 | Y | Aib | E | G | T | F | T | S | D | Y | S | I | Aib | L | E | K | Q | A | Q | Aib | A | F | V | Q | W | L | I | K | 1 |
| 145 | Y | Aib | E | G | T | F | T | S | D | Y | S | I | Aib | L | E | K | Q | A | Q | Aib | E | F | V | E | W | L | I | K | 1 |
| 146 | Y | Aib | E | G | T | F | T | S | D | Y | S | I | Aib | L | E | K | Q | A | Q | Aib | A | F | V | Q | W | L | I | K | 1 |
| 147 | Y | Aib | E | G | T | F | T | S | D | Y | S | I | Aib | L | E | K | Q | A | Q | Aib | A | F | V | Q | W | L | I | K | 1 |
| 148 | Y | Aib | E | G | T | F | T | S | D | Y | S | I | Aib | L | E | K | Q | A | Q | Aib | A | F | V | Q | W | L | I | K | 1 |
| 149 | Y | Aib | E | G | T | F | T | S | D | Y | S | I | Aib | L | E | K | Q | A | Q | Aib | A | F | V | Q | W | L | I | K | 1 |
| 150 | Y | Aib | E | G | T | F | T | S | D | Y | S | I | Aib | L | E | K | Q | A | E | Aib | A | F | V | E | W | L | I | K | 1 |
| 151 | Y | Aib | E | G | T | F | T | S | D | Y | S | I | Aib | L | E | K | Q | A | Q | Aib | E | F | V | Q | W | L | I | K | 2 |
| 152 | Y | Aib | E | G | T | F | T | S | D | Y | S | I | Aib | L | E | K | Q | A | Q | Aib | A | F | V | E | W | L | I | K | 0 |
| 153 | Y | Aib | E | G | T | F | T | S | D | Y | S | I | Aib | L | E | K | Q | A | Q | Aib | E | F | V | Q | W | L | I | K | 2 |
| 154 | Y | Aib | E | G | F | T | S | D | Y | Y | S | I | Aib | L | E | K | Q | A | Q | Aib | E | F | V | Q | W | L | I | K | 2 |
| 155 | Y | Aib | E | G | T | F | T | S | D | Y | S | I | Aib | L | E | K | Q | A | Q | Aib | A | F | V | Q | W | L | I | K | 1 |
| 156 | Y | Aib | E | G | T | F | T | S | D | Y | S | I | Aib | L | E | K | Q | A | E | Aib | A | F | V | Q | W | L | I | K | 2 |
| 157 | Y | Aib | E | G | T | F | T | S | D | Y | S | I | Aib | L | E | K | Q | A | Q | Aib | E | F | V | Q | W | L | I | K | 2 |
| 158 | Y | Aib | E | G | T | F | T | S | D | Y | S | I | Aib | L | E | K | Q | A | Q | Aib | A | F | V | E | W | L | I | K | 0 |
| 159 | Y | Aib | E | G | T | F | T | S | D | Y | S | I | Aib | L | E | K | Q | A | Q | Aib | E | F | V | E | W | L | I | K | 1 |
| 160 | Y | Aib | E | G | T | F | T | S | D | Y | S | I | Aib | L | E | K | Q | A | Q | Aib | E | F | V | Q | W | L | I | K | 2 |
| 161 | Y | Aib | E | G | T | F | T | S | D | Y | S | I | Aib | L | E | K | Q | A | Q | Aib | A | F | V | E | W | L | I | K | 0 |
| 162 | Y | Aib | E | G | T | F | T | S | D | Y | S | I | Aib | L | E | K | Q | A | Q | Aib | A | F | V | Q | W | L | I | K | 1 |
| 163 | Y | Aib | E | G | T | F | T | S | D | Y | S | I | Aib | L | E | K | Q | A | Q | Aib | A | F | V | E | W | L | I | K | 0 |
| 164 | Y | Aib | E | G | T | F | T | S | D | Y | S | I | Aib | L | E | K | Q | A | Q | Aib | E | F | V | Q | W | L | I | K | 2 |
| 165 | Y | Aib | E | G | T | F | T | S | D | Y | S | I | Aib | L | E | K | Q | A | Q | Aib | A | F | V | Q | W | L | I | K | 1 |
| 166 | Y | Aib | E | G | T | F | T | S | D | Y | S | I | Aib | L | E | K | Q | A | Q | Aib | A | F | V | E | W | L | I | K | 0 |
| 167 | Y | Aib | E | G | T | F | T | S | D | Y | S | I | Aib | L | E | K | Q | A | Q | Aib | A | F | V | Q | W | L | I | K | 1 |
| 168 | Y | Aib | E | G | T | F | T | S | D | Y | S | I | Aib | L | E | K | Q | A | Q | Aib | A | F | V | E | W | L | I | K | 0 |
| 169 | Y | Aib | E | G | T | F | T | S | D | Y | S | I | Aib | L | E | K | Q | A | Q | Aib | A | F | V | Q | W | L | I | K | 1 |
| 170 | Y | Aib | E | G | T | F | T | S | D | Y | S | I | Aib | L | E | K | Q | A | Q | Aib | A | F | V | Q | W | L | I | K | 1 |
| 171 | Y | Aib | E | G | T | F | T | S | D | Y | S | I | Aib | L | E | K | Q | A | Q | Aib | E | F | V | E | W | L | I | K | 1 |
| 172 | Y | Aib | E | G | T | F | T | S | D | Y | S | I | Aib | L | E | K | Q | A | Q | Aib | A | F | V | E | W | L | I | K | 1 |
| 173 | Y | Aib | E | T | T | F | T | S | D | Y | S | I | Aib | L | E | K | Q | A | Q | Aib | A | F | V | Q | W | L | I | K | 1 |
| 174 | Y | Aib | E | G | T | F | T | S | D | Y | S | I | Aib | L | E | K | Q | A | E | Aib | A | F | V | Q | W | L | I | K | 2 |
| 175 | Y | Aib | E | G | T | F | T | S | D | Y | S | I | Aib | L | E | K | Q | A | Q | Aib | A | F | V | E | W | L | I | K | 0 |
| 176 | Y | Aib | E | G | T | F | T | S | D | Y | S | I | Aib | L | E | K | Q | A | Q | Aib | E | F | V | Q | W | L | I | K | 2 |
| 177 | Y | Aib | E | G | T | F | T | S | D | Y | S | I | Aib | L | E | K | Q | A | Q | Aib | A | F | V | Q | W | L | I | K | 1 |
| 178 | Y | Aib | E | G | T | F | T | S | D | Y | S | I | Aib | L | E | K | Q | A | Q | Aib | A | F | V | E | W | L | I | K | 0 |
| 179 | Y | Aib | E | G | T | F | T | S | D | Y | S | I | Aib | L | E | K | Q | A | Q | Aib | E | F | V | Q | W | L | I | K | 2 |
| 180 | Y | Aib | E | G | T | F | T | S | D | Y | S | I | Aib | L | E | K | Q | A | Q | Aib | A | F | V | E | W | L | I | K | 0 |
| 181 | Y | Aib | E | G | T | F | T | S | D | Y | S | I | Aib | L | E | E | Q | A | Q | Aib | A | F | V | E | W | L | I | K | 1 |
| 182 | Y | Aib | E | G | T | F | T | S | D | Y | S | I | Aib | L | E | K | Q | A | Q | Aib | A | F | V | E | W | L | I | K | 0 |
| 183 | Y | Aib | E | G | T | F | T | S | D | Y | S | I | Aib | L | E | K | Q | A | Q | Aib | A | F | V | E | W | L | I | K | 0 |
| 184 | Y | Aib | E | G | T | F | T | S | D | Y | S | I | Aib | L | E | K | Q | A | Q | Aib | A | F | V | E | W | L | I | K | 0 |
| 185 | Y | Aib | E | G | T | F | T | S | D | Y | S | I | Aib | L | E | K | Q | A | Q | Aib | E | F | V | Q | W | L | I | K | 2 |
| 186 | Y | Aib | E | G | T | F | T | S | D | Y | S | I | Aib | L | E | K | Q | A | Q | Aib | A | F | V | Q | W | L | I | K | 1 |
| 187 | Y | Aib | E | G | T | F | T | S | D | Y | S | I | Aib | L | E | K | Q | A | Q | Aib | E | F | V | E | W | L | I | K | 1 |
| 188 | Y | Aib | E | G | T | F | T | S | D | Y | S | I | Aib | L | E | K | Q | A | Q | Aib | A | F | V | Q | W | L | I | K | 1 |
| 189 | Y | Aib | E | G | T | F | T | S | D | Y | S | I | Aib | L | E | K | Q | A | Q | Aib | A | F | V | Q | W | L | I | K | 1 |
| 190 | Y | Aib | E | G | T | F | T | S | D | Y | S | I | Aib | L | E | K | Q | A | Q | Aib | A | F | V | E | W | L | I | K | 0 |
| 191 | Y | Aib | E | G | T | F | T | S | D | Y | S | I | Aib | L | E | K | Q | A | Q | Aib | A | F | V | E | W | L | I | K | 0 |
| 192 | Y | Aib | E | G | T | F | T | S | D | Y | S | I | Aib | L | E | Lys | Q | A | Q | Aib | A | F | V | E | W | L | I | K | 1 |
| | | | | | | | | | | | | | | | | Ac | | | | | | | | | | | | | |
| 193 | Y | Aib | E | G | T | F | T | S | D | Y | S | I | Aib | L | E | K | Q | A | Q | Aib | A | F | V | Q | W | L | I | K | 1 |
| 194 | Y | Aib | E | G | T | F | T | S | D | Y | S | I | Aib | L | E | K | Q | A | Q | Aib | E | F | V | Q | W | L | I | K | 2 |
| 195 | Y | Aib | E | G | T | F | T | S | D | Y | S | I | Aib | L | E | K | Q | A | Q | Aib | A | F | V | E | W | L | I | K | 0 |
| 196 | Y | Aib | E | G | T | F | T | S | D | Y | S | I | Aib | L | E | K | Q | A | Q | Aib | A | F | V | Q | W | L | I | K | 1 |
| 197 | Y | Aib | E | G | T | F | T | S | D | Y | S | I | Aib | L | E | K | Q | A | Q | Aib | A | F | V | E | W | L | I | K | 0 |
| 198 | Y | Aib | E | G | T | F | T | S | D | Y | S | I | Aib | L | E | K | Q | A | Q | Aib | E | F | V | E | W | L | I | K | 1 |
| 199 | Y | Aib | E | G | T | F | T | S | D | Y | S | I | Aib | L | E | Aib | Q | A | Q | Aib | A | F | V | E | W | L | I | K | 1 |
| 200 | Y | Aib | E | G | T | F | T | S | D | Y | S | I | Aib | L | E | K | Q | A | E | Aib | A | F | V | Q | W | L | I | K | 2 |
| 201 | Y | Aib | E | G | T | F | T | S | D | Y | S | I | Aib | L | E | K | Q | A | Q | Aib | E | F | V | E | W | L | I | K | 1 |
| 202 | Y | Aib | E | G | T | F | T | S | D | Y | S | I | Aib | L | E | K | Q | A | Q | Aib | E | F | V | Q | W | L | I | K | 2 |
| 203 | Y | Aib | E | G | T | F | T | S | D | Y | S | I | Aib | L | E | K | Q | A | Q | Aib | A | F | V | E | W | L | I | K | 0 |
| 204 | Y | Aib | E | G | T | F | T | S | D | Y | S | I | Aib | L | E | K | Q | A | Q | Aib | A | F | V | E | W | L | I | K | 0 |
| 205 | Y | Aib | E | G | T | F | T | S | D | Y | S | I | Aib | L | E | K | Q | A | Q | Aib | E | F | V | E | W | L | I | K | 1 |
| 206 | Y | Aib | E | G | T | F | T | S | D | Y | S | I | Aib | L | E | K | Q | A | Q | Aib | A | F | V | E | W | L | I | K | 0 |
| 207 | Y | Aib | E | G | T | F | T | S | D | Y | S | I | Aib | L | E | K | Q | A | Q | Aib | E | F | V | Q | W | L | I | K | 2 |
| 208 | Y | Aib | E | G | T | F | T | S | D | Y | S | I | Aib | L | E | K | Q | A | Q | Aib | A | F | V | E | W | L | I | K | 0 |
| 209 | Y | Aib | E | G | T | F | T | S | D | Y | S | I | Aib | L | E | L | Q | A | Q | Aib | A | F | V | E | W | L | I | K | 1 |
| 210 | Y | Aib | E | G | T | F | T | S | D | Y | S | I | Aib | L | E | K | Q | A | Q | Aib | A | F | V | E | W | L | I | K | 0 |
| 211 | Y | Aib | E | G | T | F | T | S | D | Y | S | I | Aib | L | E | K | Q | A | Q | D | A | F | V | E | W | L | I | K | 1 |
| | | | | | | | | | | | | | | | | | | | | Asp | | | | | | | | | |
| 212 | Y | Aib | E | G | T | F | T | S | D | Y | S | I | Aib | L | E | K | Q | A | Q | Aib | A | F | V | E | W | L | I | K | 0 |
| 213 | Y | Aib | E | G | T | F | T | S | D | Y | S | I | Aib | L | E | K | Q | A | Q | Aib | A | F | V | E | W | L | I | K | 0 |
| 214 | Y | Aib | E | G | T | F | T | S | D | Y | S | I | Aib | L | E | K | Q | A | Q | Aib | A | F | V | E | W | L | I | K | 0 |
| 215 | Y | Aib | E | G | T | F | T | S | D | Y | S | I | Aib | L | E | K | Q | A | Q | Aib | E | F | V | E | W | L | I | K | 1 |
| 216 | Y | Aib | E | G | T | F | T | S | D | Y | S | I | Aib | L E | K | Q | A | Q | D | A | F | V | E | W | L | I | K | 1 | |
| | | | | | | | | | | | | | | | | | | | | Arg | | | | | | | | | |
| 217 | Y | Aib | E | G | T | F | T | S | D | Y | S | I | Aib | L | E | K | Q | A | Q | Aib | A | F | V | E | W | L | I | K | 0 |
| 218 | Y | Aib | E | G | T | F | T | S | D | Y | S | I | Aib | L | E | K | Q | A | Q | Aib | A | F | V | E | W | L | I | K | 0 |
| 219 | Y | Aib | E | G | T | F | T | S | D | Y | S | I | Aib | L | E | K | Q | A | E | Aib | A | F | V | Q | W | L | I | K | 2 |
| 220 | Y | Aib | E | G | T | F | T | S | D | Y | S | I | Aib | L | E | K | Q | A | Q | Aib | A | F | V | Q | W | L | I | K | 1 |
| 221 | Y | Aib | E | G | T | F | T | S | D | Y | S | I | Aib | L | E | K | Q | A | Q | Aib | A | F | V | E | W | L | I | K | 0 |
| 222 | Y | Aib | E | G | T | F | T | S | D | Y | S | I | Aib | L | E | K | Q | A | Q | Aib | A | F | V | E | W | L | I | K | 0 |
| 223 | Y | Aib | E | G | T | F | T | S | D | Y | S | I | Aib | L | E | K | Q | A | Q | Aib | A | F | V | Q | W | L | I | K | 1 |
| 224 | Y | Aib | E | G | T | F | T | S | D | Y | S | I | Aib | L | E | K | Q | A | Q | Aib | A | F | V | Q | W | L | I | K | 1 |
| 225 | Y | Aib | E | G | T | F | T | S | D | Y | S | I | Aib | L | E | K | Q | A | Q | Aib | A | F | V | E | W | L | I | K | 0 |
| 226 | Y | Aib | E | G | T | F | T | S | D | Y | S | I | Aib | L | E | K | Q | A | Q | Aib | A | F | V | E | W | L | I | K | 0 |
| 227 | Y | Aib | E | G | T | F | T | S | D | Y | S | I | Aib | L | E | K | Q | A | Q | Aib | A | F | V | E | W | L | I | K | 0 |
| 228 | Y | Aib | E | G | T | F | T | S | D | Y | S | I | Aib | L | E | K | Q | A | Q | Aib | A | F | V | Q | W | L | I | K | 1 |
| 229 | Y | Aib | E | G | T | F | T | S | D | Y | S | I | Aib | L | E | K | Q | A | Q | Aib | A | F | V | E | W | L | I | K | 0 |
| 230 | Y | Aib | E | G | T | F | T | S | D | Y | S | I | Aib | L | E | K | Q | A | Q | Aib | A | F | V | E | W | L | I | K | 0 |
| 231 | Y | Aib | E | G | T | F | T | S | D | Y | S | I | Aib | L | E | K | Q | A | Q | Aib | A | F | I | E | W | L | I | K | 1 |
| 232 | Y | Aib | E | G | T | F | T | S | D | Y | S | I | Aib | L | E | K | Q | A | Q | Aib | A | F | V | E | W | L | I | K | 0 |
| 233 | Y | Aib | E | G | T | F | T | S | D | Y | S | I | Aib | L | E | K | Q | A | Q | Aib | A | F | V | E | W | L | I | K | 0 |
| 234 | Y | Aib | E | G | T | F | T | S | D | L | S | I | Aib | L | E | K | Q | A | Q | Aib | A | F | V | E | W | L | I | K | 1 |
| 235 | Y | Aib | E | G | T | F | T | S | D | Y | S | I | Aib | L | E | K | Q | A | Q | Aib | A | F | V | E | W | L | I | K | 0 |
| 236 | Y | Aib | E | G | T | F | T | S | D | Y | S | I | Aib | L | E | K | Q | A | Q | Aib | A | F | V | E | W | L | I | K | 0 |
| 237 | Y | Aib | E | G | T | F | T | S | D | Y | S | I | Aib | L | E | K | Q | A | Q | Aib | A | F | V | E | W | L | I | K | 0 |
| 238 | Y | Aib | E | G | T | F | T | S | D | Y | S | I | Aib | L | E | K | Q | A | Q | Aib | A | F | V | E | W | L | I | K | 0 |
| 239 | Y | Aib | E | G | T | F | T | S | D | Y | S | I | Aib | L | E | K | Q | A | Q | Aib | A | F | V | Q | W | L | I | K | 1 |
| 240 | Y | Aib | E | G | T | F | T | S | D | Y | S | I | Aib | L | E | K | Q | A | Q | Aib | A | F | V | Q | W | L | I | K | 1 |
| 241 | Y | Aib | E | G | T | F | T | S | D | Y | S | I | Aib | L | E | K | Q | A | Q | Aib | A | F | V | E | W | L | I | K | 0 |
| 242 | Y | Aib | E | G | T | F | T | S | D | Y | S | I | Aib | L | E | K | Q | A | Q | Aib | A | F | V | Q | W | L | I | K | 1 |
| 243 | Y | Aib | E | G | T | F | T | S | D | Y | S | I | Aib | L | E | K | Q | A | Q | Aib | A | F | V | Q | W | L | I | K | 1 |
| 244 | Y | Aib | E | G | T | F | T | S | D | Y | S | I | Aib | L | E | K | Q | A | Q | Aib | A | F | V | E | W | L | I | K | 0 |
| 245 | Y | Aib | E | G | T | F | T | S | D | Y | S | I | Aib | L | E | K | Q | A | Q | Aib | A | F | V | Q | W | L | I | K | 1 |
| 246 | Y | Aib | E | G | T | F | T | S | D | Y | S | I | Aib | L | E | K | Q | A | Q | Aib | A | F | V | Q | W | L | I | K | 1 |
| 247 | Y | Aib | E | G | T | F | T | S | D | Y | S | I | Aib | L | E | K | Q | A | Q | Aib | A | F | I | E | W | L | I | K | 1 |
| 248 | Y | Aib | E | G | T | F | T | S | D | Y | S | I | Aib | L | E | K | Q | A | Q | Aib | A | F | V | E | W | L | I | K | 0 |
| 249 | Y | Aib | E | G | T | F | T | S | D | Y | S | I | Aib | L | E | K | Q | A | Q | Aib | A | F | V | Q | W | L | I | K | 1 |
| 250 | Y | Aib | E | G | T | F | T | S | D | Y | S | I | Aib | L | E | K | Q | A | Q | Aib | A | F | V | Q | W | L | I | K | 1 |
| 251 | Y | Aib | E | G | T | F | T | S | D | Y | S | I | Aib | L | E | K | Q | A | E | Aib | A | F | V | E | W | L | I | K | 1 |
| 252 | Y | Aib | E | G | T | F | T | S | D | Y | S | I | Aib | L | E | K | Q | A | Q | Aib | A | F | V | Q | W | L | I | K | 1 |
| 253 | Y | Aib | E | G | T | F | T | S | D | Y | S | I | Aib | L | E | K | Q | A | Q | Aib | A | F | V | E | W | L | I | K | 0 |
| 254 | Y | Aib | E | G | T | F | T | S | D | Y | S | I | Aib | L | E | K | Q | A | Q | Aib | A | F | V | E | W | L | I | K | 0 |
| 255 | Y | Aib | E | G | T | F | T | S | D | Y | S | I | Aib | L | E | K | Q | A | Q | Aib | E | F | V | E | W | L | I | K | 1 |
| 256 | Y | Aib | E | G | T | F | T | S | D | Y | S | I | Aib | L | E | K | Q | A | Q | Aib | A | F | V | E | W | L | I | K | 0 |
| 257 | Y | Aib | E | G | T | F | T | S | D | Y | S | I | Aib | L | E | K | Q | A | Q | Aib | A | F | V | Q | W | L | I | K | 1 |
| 258 | Y | Aib | E | G | T | F | T | S | D | Y | S | I | Aib | L | E | K | Q | A | Q | Aib | A | F | I | E | W | L | I | K | 1 |
| 259 | Y | Aib | E | G | T | F | T | S | D | Y | S | I | Aib | L | E | K | Q | A | Q | Aib | A | F | V | E | W | L | I | K | 0 |
| 260 | Y | Aib | E | G | T | F | T | S | D | Y | S | I | Aib | L | E | K | Q | A | Q | Aib | A | F | V | E | W | L | I | K | 0 |
| 261 | Y | Aib | E | G | T | F | T | S | D | Y | S | I | Aib | L | E | K | Q | A | Q | Aib | A | F | V | E | W | L | I | K | 0 |
| 262 | Y | Aib | E | G | T | F | T | S | D | Y | S | I | Aib | L | E | K | Q | A | E | Aib | A | F | V | E | W | L | I | K | 1 |
| 263 | Y | Aib | E | G | T | F | T | S | D | Y | S | I | Aib | L | E | K | Q | A | Q | Aib | A | F | V | E | W | L | I | K | 0 |
| 264 | Y | Aib | E | G | T | F | T | S | D | Y | S | I | Aib | L | E | K | Q | A | Q | Aib | A | F | V | Q | W | L | I | K | 1 |
| 265 | Y | Aib | E | G | T | F | T | S | D | Y | S | I | Aib | L | E | K | Q | A | Q | Aib | A | F | V | Q | W | L | I | K | 1 |
| 266 | Y | Aib | E | G | T | F | T | S | D | Y | S | I | Aib | L | E | K | Q | A | Q | Aib | A | F | V | Q | W | L | I | K | 1 |
| 267 | Y | Aib | E | G | T | F | T | S | D | Y | S | I | Aib | L | E | K | Q | A | Q | Aib | A | F | V | Q | W | L | I | K | 1 |
| 268 | Y | Aib | E | G | T | F | T | S | D | Y | S | I | Aib | L | E | K | Q | A | Q | Aib | A | F | V | Q | W | L | I | K | 1 |
| 269 | Y | Aib | E | G | T | F | T | S | D | Y | S | I | Aib | L | E | K | Q | D | Q | Aib | A | F | V | Q | W | L | I | K | 1 |
| 270 | Y | Aib | E | G | T | F | T | S | D | Y | S | I | Aib | L | E | K | Q | A | Q | Aib | A | F | V | Q | W | L | I | K | 1 |
| 271 | Y | Aib | E | G | T | F | T | S | D | Y | S | I | Aib | L | E | K | Q | A | Q | Aib | E | F | V | E | W | L | I | K | 1 |
| 272 | Y | Aib | E | G | T | F | T | S | D | Y | S | I | Aib | L | E | K | Q | A | Q | Aib | A | F | V | E | W | L | I | K | 0 |
| 273 | Y | Aib | E | G | T | F | T | S | D | Y | S | I | Aib | L | E | K | Q | A | Q | Aib | E | F | V | E | W | L | I | K | 1 |
| 274 | Y | Aib | E | G | T | F | T | S | D | Y | S | I | Aib | L | E | K | Q | A | Q | Aib | E | F | V | E | W | L | I | K | 1 |
| 275 | Y | Aib | E | G | T | F | T | S | D | Y | S | I | Aib | L | E | K | Q | A | Q | Aib | E | F | V | E | W | L | I | K | 1 |
| 276 | Y | Aib | E | G | T | F | T | S | D | Y | S | I | Aib | L | E | K | Q | A | Q | Aib | E | F | V | E | W | L | I | K | 1 |
| 277 | Y | Aib | E | G | T | F | T | S | D | Y | S | I | Aib | L | E | K | Q | A | Q | Aib | A | F | V | E | W | L | I | K | 0 |
| 278 | Y | Aib | E | G | T | F | T | S | D | Y | S | I | Aib | L | E | K | Q | D | E | Aib | A | F | V | E | W | L | I | K | 1 |
| 279 | Y | Aib | E | G | T | F | T | S | D | Y | S | I | Aib | L | E | K | Q | A | Q | Aib | E | F | V | E | W | L | I | K | 1 |
| 280 | Y | Aib | E | G | T | F | T | S | D | Y | S | I | Aib | L | E | K | Q | A | E | Aib | A | F | V | E | W | L | I | K | 1 |
| 281 | Y | Aib | E | G | T | F | T | S | D | Y | S | I | Aib | L | E | K | Q | A | Q | Aib | A | F | V | Q | W | L | I | K | 1 |
| 282 | Y | Aib | E | G | T | F | T | S | D | Y | S | I | Aib | L | E | K | Q | A | Q | Aib | E | F | V | E | W | L | I | K | 1 |
| 283 | Y | Aib | E | G | T | F | T | S | D | Y | S | I | Aib | L | E | K | Q | A | Q | Aib | A | F | I | E | W | L | I | K | 1 |
| 284 | Y | Aib | E | G | T | F | T | S | D | Y | S | I | Aib | L | E | K | Q | A | Q | Aib | E | F | V | E | W | L | I | K | 1 |
| 285 | Y | Aib | E | G | T | F | T | S | D | Y | S | I | Aib | L | E | K | Q | A | Q | Aib | A | F | V | E | W | L | I | K | 0 |
| 286 | Y | Aib | E | G | T | F | T | S | D | Y | S | I | Aib | L | E | K | Q | A | Q | Aib | A | F | V | E | W | L | I | K | 0 |
| 287 | Y | Aib | E | G | T | F | T | S | D | Y | S | I | Aib | L | E | K | Q | A | Q | Aib | A | F | V | E | W | L | I | K | 0 |
| 288 | Y | Aib | E | G | T | F | T | S | D | Y | S | I | Aib | L | E | K | Q | A | Q | Aib | A | F | V | E | W | L | I | K | 0 |
| 289 | Y | Aib | E | G | T | F | T | S | D | Y | S | I | Aib | L | E | K | Q | A | Q | Aib | A | F | V | E | W | L | I | K | 0 |
| 290 | Y | Aib | E | G | T | F | T | S | D | Y | S | I | Aib | L | E | K | Q | A | Q | Aib | A | F | V | E | W | L | I | K | 0 |
| 291 | Y | Aib | E | G | T | F | T | S | D | Y | S | I | Aib | L | E | K | Q | A | Q | Aib | A | F | V | E | W | L | I | K | 0 |
| 292 | Y | Aib | E | G | T | F | T | S | D | Y | S | I | Aib | L | E | K | Q | A | Q | Aib | A | F | V | E | W | L | I | K | 0 |
| 293 | Y | Aib | E | G | T | F | T | S | D | Y | S | I | Aib | L | E | K | Q | A | Q | Aib | A | F | V | E | W | L | I | K | 0 |
| 294 | Y | Aib | E | G | T | F | T | S | D | Y | S | I | Aib | L | E | K | Q | A | Q | Aib | A | F | V | E | W | L | I | K | 0 |
| 295 | Y | Aib | E | G | T | F | T | S | D | Y | S | I | Aib | L | E | K | Q | A | Q | Aib | A | F | V | E | W | L | I | K | 0 |
| 296 | Y | Aib | E | G | T | F | T | S | D | Y | S | I | Aib | L | E | K | Q | A | Q | Aib | A | F | V | E | W | L | I | K | 0 |
| 297 | Y | Aib | E | G | T | F | T | S | D | Y | S | I | Aib | L | E | K | Q | A | Q | Aib | A | F | V | E | W | L | I | K | 0 |
| 298 | Y | Aib | E | G | T | F | T | S | D | Y | S | I | Aib | L | E | K | Q | A | Q | Aib | A | F | V | E | W | L | I | K | 0 |
| 299 | Y | Aib | E | G | T | F | T | S | D | Y | S | I | Aib | L | E | K | Q | A | Q | Aib | A | F | V | E | W | L | I | K | 0 |
| 300 | Y | Aib | E | G | T | F | T | S | D | Y | S | I | Aib | L | E | K | Q | A | Q | Aib | A | F | V | E | W | L | I | K | 0 |
| 301 | Y | Aib | E | G | T | F | T | S | D | Y | S | I | Aib | L | E | K | Q | A | Q | Aib | A | F | V | E | W | L | I | K | 0 |
| 302 | Y | Aib | E | G | T | F | T | S | D | Y | S | I | Aib | L | E | K | Q | A | Q | Aib | A | F | V | E | W | L | I | K | 0 |
| 303 | Y | Aib | E | G | T | F | T | S | D | Y | S | I | Aib | L | E | K | Q | A | Q | Aib | A | F | V | E | W | L | I | K | 0 |
| 304 | Y | Aib | E | G | T | F | T | S | D | Y | S | I | Aib | L | E | K | Q | A | Q | Aib | A | F | V | E | W | L | I | K | 0 |
| 305 | Y | Aib | E | G | T | F | T | S | D | Y | S | I | Aib | L | E | K | Q | A | Q | Aib | A | F | V | E | W | L | I | K | 0 |

| | | | | | | | | | | | | | | | | | | | | | | Nr of |
|---|---|---|---|---|---|---|---|---|---|---|---|---|---|---|---|---|---|---|---|---|---|---|
| | | | | | | | | | | | | | | | | | | | | | | substitutions |
| | | | | | | | | | | | | | | | | | | | | | | to Z1 |
| | | | | | | | | | | | | | | | | | | | | | | of SEQ |
| | | | | | | | | | | | | | | | | | | | | | SEQ | ID |
| | | | | | | | | | | | Exemplified substitutions vs. | | | | | | | | | | ID | NO: |
| Exemplified amino acids | | | | | | | | | | | SEQ ID NO: 286 | | | | | | | | | | NO: | 286 |
| Y | | | | | | | | | | | None | | | | | | | | | | X₁ | |
| Aib | | | | | | | | | | | None | | | | | | | | | | X₂ | |
| E, D | | | | | | | | | | | D | | | | | | | | | | X₃ | |
| G | | | | | | | | | | | None | | | | | | | | | | X₄ | |
| T | | | | | | | | | | | None | | | | | | | | | | X₅ | |
| F | | | | | | | | | | | None | | | | | | | | | | X₆ | |
| T | | | | | | | | | | | None | | | | | | | | | | X₇ | |
| S | | | | | | | | | | | None | | | | | | | | | | X₈ | |
| D | | | | | | | | | | | None | | | | | | | | | | X₉ | |
| Y, L | | | | | | | | | | | L | | | | | | | | | | X₁₀ | |
| S | | | | | | | | | | | None | | | | | | | | | | X₁₁ | |
| I | | | | | | | | | | | None | | | | | | | | | | X₁₂ | |
| Aib, L | | | | | | | | | | | L | | | | | | | | | | X₁₃ | |
| L | | | | | | | | | | | None | | | | | | | | | | X₁₄ | |
| E | | | | | | | | | | | D or A | | | | | | | | | | X₁₅ | |
| K, E, D, Aib, G,LysAc, A, L, S, Q, or R | | | | | | | | | | | E, D, Aib, G, LysAc, A, L, S, | | | | | | | | | | X₁₆ | |
| | | | | | | | | | | | Q, or R | | | | | | | | | | | |
| Q, E, K, or A | | | | | | | | | | | E, K, or A | | | | | | | | | | X₁₇ | |
| A | | | | | | | | | | | None | | | | | | | | | | X₁₈ | |
| Q, E | | | | | | | | | | | E | | | | | | | | | | X₁₉ | |
| Aib, D-Asp, or D-Arg | | | | | | | | | | | D-Asp, or D-Arg | | | | | | | | | | X₂₀ | |
| A or E | | | | | | | | | | | E | | | | | | | | | | X₂₁ | |
| F | | | | | | | | | | | None | | | | | | | | | | X₂₂ | |
| V, I | | | | | | | | | | | I | | | | | | | | | | X₂₃ | |
| Q, E | | | | | | | | | | | Q | | | | | | | | | | X₂₄ | |
| W, Y | | | | | | | | | | | Y | | | | | | | | | | X₂₅ | |
| L | | | | | | | | | | | None | | | | | | | | | | X₂₆ | |
| I, L | | | | | | | | | | | L | | | | | | | | | | X₂₇ | |
| K | | | | | | | | | | | None | | | | | | | | | | X₂₈ | |
| | | | | | | | | | | | | | | | | | | | | | | Nr of |
| | | | | | | | | | | | | | | | | | | | | | | substitutions |
| | | | | | | | | | | | | | | | | | | | | | | to Z3 |
| SEQ | | | | | | | | | | | | | | | | | | | | | | of SEQ |
| ID | Linker (Z2 of Formula (I)) | | | | | | PPY part (Z3 of Formula (I)) | | | | | | | | | | | | | | | ID NO: |
| NO: | X₂₉ | X₃₀ | X₃₁ | X₃₂ | X₃₃ | X₃₄ | X₃₅ | X₃₆ | X₃₇ | X₃₈ | X₃₉ | X₄₀ | X₄₁ | X₄₂ | X₄₃ | X₄₄ | X₄₅ | X₄₆ | X₄₇ | X₄₈ | X₄₉ | 286 |
| 2 | G | G | P | S | S | G | I | E | L | R | H | F | L | N | H | L | T | R | Q | R | Y | 5 |
| 3 | G | G | Q | R | S | Y | A | S | L | R | H | Y | Y | N | W | L | T | R | Q | R | Y | 0 |
| 4 | G | G | P | S | S | G | A | S | Aib | R | H | Y | Aib | N | R | Aib | T | R | Q | R | Y | 4 |
| 5 | G | G | P | S | S | G | A | S | D | R | D | Y | Y | N | W | L | T | D | Q | R | D | 4 |
| | | | | | | | | | Leu | | His | | | | | | | Arg | | | Tyr | |
| 6 | G | G | Q | R | Y | Y | A | S | L | R | H | Y | Y | N | W | L | T | R | Q | R | Y | 0 |
| 7 | G | G | P | S | S | G | A | S | L | R | H | Y | Y | N | W | L | T | R | Q | R | Y | 0 |
| 8 | G | G | P | S | S | G | Q | S | L | R | H | Y | Y | N | W | L | T | R | Q | R | Y | 1 |
| 9 | G | G | P | S | S | Y | V | S | I | R | H | Y | Y | N | W | L | T | R | Q | R | Y | 2 |
| 10 | G | G | P | S | S | P | E | S | Aib | R | H | Y | Y | N | W | L | T | R | Q | R | Y | 2 |
| 11 | G | G | P | E | S | P | A | S | L | R | H | Y | Y | N | W | L | T | R | Q | R | Y | 0 |
| 12 | G | G | P | E | S | P | A | S | L | R | H | Y | Y | N | W | L | T | R | Q | R | Y | 0 |
| 13 | G | G | P | S | S | G | A | S | Aib | R | H | Y | Y | N | R | Aib | T | R | Q | R | Y | 3 |
| 14 | G | G | P | R | Y | Y | A | S | L | R | H | Y | Y | N | W | L | T | R | Q | R | Y | 0 |
| 15 | G | G | P | S | S | Y | V | S | L | R | H | Y | Y | N | W | L | T | R | Q | R | Y | 1 |
| 16 | G | G | P | S | S | G | A | S | L | R | H | Y | Y | N | W | L | T | R | Q | R | Y | 0 |
| 17 | G | G | P | S | S | G | A | S | L | R | H | Y | L | N | L | Aib | T | R | Q | R | Y | 3 |
| 18 | G | G | P | S | S | G | A | S | L | R | H | Y | Aib | N | R | Aib | T | R | Q | R | Y | 3 |
| 19 | G | G | P | S | Y | Y | V | S | L | R | H | Y | Y | N | W | L | T | R | Q | R | Y | 1 |
| 20 | G | G | P | S | S | G | A | S | L | R | H | Y | Y | N | W | L | T | R | Q | R | Y | 0 |
| 21 | G | G | P | S | S | G | A | S | L | R | D | Y | Y | N | W | L | T | D | Q | R | D | 3 |
| | | | | | | | | | | | His | | | | | | | Arg | | | Tyr | |
| 22 | G | G | Q | S | S | Y | A | S | L | R | H | Y | Y | 2 | W | L | T | R | Q | R | Y | 0 |
| 23 | G | G | P | E | Y | Y | A | S | L | R | H | Y | Y | N | W | L | T | R | Q | R | Y | 0 |
| 24 | G | G | P | S | S | G | A | S | L | R | H | Y | Y | N | W | L | T | R | Q | R | Y | 0 |
| 25 | G | G | P | S | S | G | A | S | L | R | H | Y | Y | N | W | L | T | R | Q | R | Y | 0 |
| 26 | G | G | P | S | S | G | A | S | Aib | R | H | Y | Y | N | W | L | T | h | Q | R | D | 3 |
| | | | | | | | | | | | | | | | | | | Arg | | | Tyr | |
| 27 | G | G | P | S | S | G | V | S | I | R | H | Y | Y | N | W | L | T | R | Q | R | Y | 2 |
| 28 | G | G | P | S | S | G | A | S | L | R | H | Y | Y | N | W | L | T | R | Q | R | Y | 0 |
| 29 | G | G | P | E | S | G | A | S | L | R | H | Y | Y | N | W | L | T | R | Q | R | Y | 0 |
| 30 | G | G | P | S | S | G | A | S | L | R | H | Y | Y | N | W | L | T | R | Q | R | Y | 0 |
| 31 | G | G | P | S | S | G | Q | S | L | R | H | Y | Y | N | W | L | T | R | Q | R | Y | 1 |
| 32 | G | G | P | E | S | G | A | S | L | R | H | Y | Y | N | W | L | T | R | Q | R | Y | 0 |
| 33 | G | G | P | S | S | A | V | S | L | R | H | Y | Y | N | W | L | T | R | Q | R | Y | 1 |
| 34 | G | G | P | S | S | G | A | S | L | R | H | Y | Y | N | W | L | T | R | Q | R | Y | 0 |
| 35 | G | G | P | S | S | G | E | S | L | R | H | Y | Y | N | W | L | T | R | Q | R | Y | 1 |
| 36 | G | G | P | S | S | G | A | S | L | R | H | Y | Y | N | W | L | T | R | Q | R | Y | 0 |
| 37 | G | G | P | E | S | G | A | S | L | R | H | Y | Y | N | W | L | T | R | Q | R | Y | 0 |
| 38 | G | G | P | S | S | G | V | S | Aib | R | H | Y | Y | N | W | L | T | R | Q | R | Y | 2 |
| 39 | G | G | P | S | S | G | A | S | Aib | R | H | Y | Y | N | W | L | T | R | Q | NMe | Y | 2 |
| | | | | | | | | | | | | | | | | | | | | R | | |
| 40 | G | G | P | S | S | Y | V | S | L | R | H | Y | Y | N | W | L | T | R | Q | R | Y | 1 |
| 41 | G | G | P | S | S | P | Q | S | L | R | H | Y | Y | N | W | L | T | R | Q | R | Y | 1 |
| 42 | G | G | P | S | E | Y | V | S | I | R | H | Y | Y | N | W | L | T | R | Q | R | Y | 2 |
| 43 | G | G | P | S | S | G | A | S | L | R | H | Y | Y | N | W | L | T | R | Q | R | Y | 0 |
| 44 | G | G | P | S | S | G | Q | S | Aib | R | H | Y | Y | N | W | L | T | R | Q | R | Y | 2 |
| 45 | G | G | P | S | S | G | A | S | L | R | H | Y | Y | N | W | L | T | R | Q | R | Y | 0 |
| 46 | G | G | P | S | S | G | A | S | L | R | H | Y | Y | N | W | L | T | R | Q | R | Y | 0 |
| 47 | G | G | P | S | S | G | V | S | L | R | H | Y | Y | N | W | L | T | R | Q | R | Y | 1 |
| 48 | G | G | P | S | E | P | Q | S | I | R | H | Y | Y | N | W | L | T | R | Q | R | Y | 2 |
| 49 | G | G | P | S | S | G | E | S | Aib | R | H | Y | Y | N | W | L | T | R | Q | R | Y | 2 |
| 50 | G | G | P | S | S | Y | E | S | L | R | H | Y | Y | N | W | L | T | R | Q | R | Y | 1 |
| 51 | G | G | P | S | S | G | A | S | L | R | H | Y | Y | N | W | L | T | R | Q | R | Y | 0 |
| 52 | G | G | P | E | S | G | A | S | L | R | H | Y | Y | N | W | L | T | R | Q | R | Y | 0 |
| 53 | G | G | P | E | S | G | A | S | L | R | H | Y | Y | N | W | L | T | R | Q | R | Y | 0 |
| 54 | G | G | P | S | E | P | A | S | L | R | H | Y | Y | N | W | L | T | R | Q | R | Y | 0 |
| 55 | G | G | P | E | S | G | A | S | L | R | H | Y | Y | N | W | L | T | R | Q | R | Y | 0 |
| 56 | G | G | P | S | S | P | A | S | L | R | H | Y | Y | N | W | L | T | R | Q | R | Y | 0 |
| 57 | G | G | P | E | E | P | A | S | L | R | H | Y | Y | N | W | L | T | R | Q | R | Y | 0 |
| 58 | G | G | P | E | S | G | A | S | L | R | H | Y | Y | N | W | L | T | R | Q | R | Y | 0 |
| 59 | G | G | P | S | Y | Y | A | S | L | R | H | Y | Y | N | W | L | T | R | Q | R | Y | 0 |
| 60 | G | G | P | E | S | G | A | S | L | R | H | Y | Y | N | W | L | T | R | Q | R | Y | 0 |
| 61 | G | G | P | E | S | G | A | S | L | R | H | Y | Y | N | W | L | T | R | Q | R | Y | 0 |
| 62 | G | G | P | R | Y | Y | A | S | L | R | H | Y | Y | N | W | L | T | R | Q | R | Y | 0 |
| 63 | G | G | P | E | S | G | A | S | L | R | H | Y | Y | N | W | L | T | R | Q | R | Y | 0 |
| 64 | G | G | P | E | S | P | A | S | L | R | H | Y | Y | N | W | L | T | R | Q | R | Y | 0 |
| 65 | G | G | G | S | E | Y | V | S | L | R | H | Y | Y | N | W | L | T | R | Q | R | Y | 1 |
| 66 | G | G | P | S | S | G | A | S | L | R | H | Y | Y | N | W | L | T | R | Q | R | Y | 0 |
| 67 | G | G | P | E | E | P | A | S | L | R | H | Y | Y | N | W | L | T | R | Q | R | Y | 0 |
| 68 | G | G | P | S | S | G | A | S | L | R | H | Y | Y | N | R | Aib | T | R | Q | R | Y | 2 |
| 69 | G | G | P | S | S | G | A | S | L | R | H | Y | Y | N | W | L | T | R | Q | R | Y | 0 |
| 70 | G | G | P | E | S | P | A | S | L | R | H | Y | Y | N | W | L | T | R | Q | R | Y | 0 |
| 71 | G | G | P | S | S | G | A | S | L | R | H | Y | Y | N | W | L | T | h | Q | R | D | 2 |
| | | | | | | | | | | | | | | | | | | Arg | | | Tyr | |
| 72 | G | G | P | S | S | G | A | D | L | R | H | Y | L | N | W | L | T | R | Q | R | Y | 2 |
| 73 | G | G | P | S | S | P | E | S | L | R | H | Y | Y | N | W | L | T | R | Q | R | Y | 1 |
| 74 | G | G | P | S | S | G | A | S | Aib | R | H | Y | Y | N | W | L | T | R | Q | R | D | 2 |
| | | | | | | | | | | | | | | | | | | | | | Tyr | |
| 75 | G | G | P | S | Y | Y | V | S | L | R | H | Y | Y | N | W | L | T | R | Q | R | Y | 1 |
| 76 | G | G | P | S | S | G | A | S | Aib | R | H | Y | Aib | N | W | L | T | R | Q | R | Y | 2 |
| 77 | G | G | P | E | E | P | A | S | L | R | H | Y | Y | N | W | L | T | R | Q | R | Y | 0 |
| 78 | G | G | P | S | E | Y | V | S | L | R | H | Y | Y | N | W | L | T | R | Q | R | Y | 1 |
| 79 | G | G | P | S | S | G | A | S | L | R | H | Y | Y | N | L | Aib | T | R | Q | R | Y | 2 |
| 80 | G | G | P | S | Y | Y | V | S | L | R | H | Y | Y | N | W | L | T | R | Q | R | Y | 1 |
| 81 | G | G | P | E | S | G | A | S | L | R | H | Y | Y | N | W | L | T | R | Q | R | Y | 0 |
| 82 | G | G | P | E | S | P | A | S | L | R | H | Y | Y | N | W | L | T | R | Q | R | Y | 0 |
| 83 | G | G | P | S | S | G | A | S | L | R | H | Y | Y | N | W | L | T | R | Q | NMe | Y | 1 |
| | | | | | | | | | | | | | | | | | | | | R | | |
| 84 | G | G | P | S | S | G | Q | S | L | R | H | Y | Y | N | W | L | T | R | Q | R | Y | 1 |
| 85 | G | G | P | S | S | G | A | S | L | R | H | Y | Y | N | W | L | T | R | NMe | R | Y | 1 |
| | | | | | | | | | | | | | | | | | | | Q | | | |
| 86 | G | G | P | S | S | G | A | S | L | R | H | Y | L | N | W | L | T | R | Q | R | Y | 1 |
| 87 | G | G | P | S | S | G | A | S | L | R | H | Y | Y | N | W | L | T | R | Q | R | Y | 0 |
| 88 | G | G | P | S | S | G | A | S | L | R | H | Y | Y | N | W | L | T | R | Q | R | Y | 0 |
| 89 | G | G | P | E | E | G | A | S | L | R | H | Y | Y | N | W | L | T | R | Q | R | Y | 0 |
| 90 | G | G | P | S | S | G | A | S | L | R | H | Y | Y | N | W | L | T | R | Q | R | Y | 0 |
| 91 | G | G | P | S | S | G | A | T | L | R | H | Y | Y | N | W | L | T | R | Q | R | Y | 1 |
| 92 | G | G | P | S | S | G | A | S | L | R | H | Y | Y | N | W | L | T | R | Q | R | Y | 0 |
| 93 | G | G | P | S | E | Y | E | S | L | R | H | Y | Y | N | W | L | T | R | Q | R | Y | 1 |
| 94 | G | G | P | S | S | G | I | S | L | R | H | Y | Y | N | W | L | T | R | Q | R | Y | 1 |
| 95 | G | G | P | S | S | P | A | S | L | R | H | Y | Y | N | W | L | T | R | Q | R | Y | 0 |
| 96 | G | G | P | S | S | G | V | S | L | R | H | Y | Y | N | W | L | T | R | Q | R | Y | 1 |
| 97 | G | G | P | S | S | G | A | S | L | R | H | Y | Y | N | W | L | T | R | Q | R | Y | 0 |
| 98 | G | G | P | S | S | A | A | S | L | R | H | Y | Y | N | W | L | T | R | Q | R | Y | 0 |
| 99 | G | G | P | S | S | G | A | S | L | R | H | Y | Y | N | W | L | T | R | Q | R | Y | 0 |
| 100 | G | G | P | S | S | G | A | S | L | R | H | Y | Y | N | W | L | T | R | Q | R | Y | 0 |
| 101 | G | G | P | S | E | P | A | S | L | R | H | Y | Y | N | W | L | T | R | Q | R | Y | 0 |
| 102 | G | G | P | S | S | G | A | S | L | R | H | Y | Y | N | W | L | T | R | Q | R | Y | 0 |
| 103 | G | G | P | S | S | G | A | S | Tle | R | H | Y | Y | N | W | L | T | R | Q | R | Y | 1 |
| 104 | G | G | P | S | S | G | A | S | L | R | H | Y | Y | N | W | L | T | R | Q | R | Y | 0 |
| 105 | G | G | P | S | S | G | A | S | L | R | H | Y | Y | N | W | L | T | R | Q | R | Y | 0 |
| 106 | G | G | P | S | S | G | A | S | L | R | H | Y | I | N | W | L | T | R | Q | R | Y | 1 |
| 107 | G | G | P | S | S | G | L | S | L | R | H | Y | Y | N | W | L | T | R | Q | R | Y | 1 |
| 108 | G | G | P | E | E | G | A | S | L | R | H | Y | Y | N | W | L | T | R | Q | R | Y | 0 |
| 109 | G | G | P | S | S | G | A | S | L | R | H | Y | Y | N | W | L | T | R | Q | R | Y | 0 |
| 110 | G | G | P | S | S | G | A | S | L | R | H | Y | Y | N | W | L | T | R | Q | R | Y | 0 |
| 111 | G | G | P | S | E | P | A | S | L | R | H | Y | Y | N | W | L | T | R | Q | R | Y | 0 |
| 112 | G | G | P | S | S | G | A | S | L | R | H | Y | Y | N | W | L | T | R | Q | R | Y | 0 |
| 113 | G | G | P | S | E | G | A | S | L | R | H | Y | Y | N | W | L | T | R | Q | R | Y | 0 |
| 114 | G | G | P | S | E | G | V | S | L | R | H | Y | Y | N | W | L | T | R | Q | R | Y | 1 |
| 115 | G | G | P | S | E | G | A | S | L | R | H | Y | Y | N | W | L | T | R | NMe | R | Y | 1 |
| | | | | | | | | | | | | | | | | | | | Q | | | |
| 116 | G | G | P | S | S | G | A | S | L | R | H | Y | Y | N | W | L | T | R | Q | R | Y | 0 |
| 117 | G | G | P | S | S | G | A | S | V | R | H | Y | Y | N | W | L | T | R | Q | R | Y | 1 |
| 118 | G | G | P | S | S | G | A | S | Aib | R | H | Y | Y | N | W | L | R | Q | R | Y | 1 | |
| 119 | G | G | P | S | S | G | A | S | L | R | Tle | Y | Y | N | W | L | T | R | Q | R | Y | 1 |
| 120 | G | G | P | S | S | P | A | S | L | R | H | Y | Y | N | W | L | T | R | Q | R | Y | 0 |
| 121 | G | G | P | S | S | G | A | S | L | R | H | Y | Y | N | W | L | T | R | Q | R | Y | 0 |
| 122 | G | G | P | S | E | P | E | S | L | R | H | Y | Y | N | W | L | T | R | Q | R | Y | 1 |
| 123 | G | G | Q | S | S | G | A | S | L | R | H | Y | Y | N | W | L | T | R | Q | R | Y | 0 |
| 124 | G | G | P | S | S | G | A | S | L | R | H | Y | Y | N | W | L | T | R | Q | R | Y | 0 |
| 125 | G | G | P | S | E | G | A | S | L | R | H | Y | Y | N | W | L | T | R | Q | NMe | Y | 1 |
| | | | | | | | | | | | | | | | | | | | | R | | |
| 126 | G | G | P | S | S | Y | A | S | L | R | H | Y | Y | N | W | L | T | R | Q | R | Y | 0 |
| 127 | G | G | P | S | S | G | E | S | L | R | H | Y | Y | N | W | L | T | R | Q | R | Y | 1 |
| 128 | G | G | P | S | S | G | A | S | L | R | H | Y | Y | N | W | L | T | R | Q | R | Y | 0 |
| 129 | G | G | P | S | S | G | A | S | L | Aib | H | Y | Y | N | W | L | T | R | Q | R | Y | 1 |
| 130 | G | G | P | S | S | G | A | S | L | R | H | Y | Y | N | W | L | T | R | Q | R | Y | 0 |
| 131 | G | G | P | S | S | G | A | S | L | R | H | Y | Y | N | W | L | T | R | Q | R | Y | 0 |
| 132 | G | G | P | S | S | G | A | S | I | R | H | Y | Y | N | W | L | T | R | Q | R | Y | 1 |
| 133 | G | G | P | S | S | G | A | S | L | R | H | Y | Tle | N | W | L | T | R | Q | R | Y | 1 |
| 134 | G | G | P | S | E | G | Q | S | I | R | H | Y | Y | N | W | L | T | R | Q | R | Y | 2 |
| 135 | G | G | P | S | S | G | A | S | L | R | H | Y | Y | N | W | L | T | R | Q | R | Y | 0 |
| 136 | G | G | P | S | S | G | A | S | L | R | H | Y | Y | N | W | L | T | R | Q | R | Y | 0 |
| 137 | G | G | P | S | S | G | A | S | L | R | H | Y | Y | N | W | Tle | T | R | Q | R | Y | 1 |
| 138 | G | G | P | S | E | G | A | S | L | R | H | Y | Y | N | W | L | T | R | Q | R | Y | 0 |
| 139 | G | G | P | S | S | G | A | S | L | R | H | Y | Y | N | W | L | T | R | Q | R | Y | 0 |
| 140 | G | G | P | S | S | P | A | S | L | R | H | Y | Y | N | W | L | T | R | Q | R | Y | 0 |
| 141 | G | G | P | S | E | G | A | S | L | R | H | Y | Y | N | W | L | T | R | Q | R | Y | 0 |
| 142 | G | G | P | S | E | P | A | S | L | R | H | Y | Y | N | W | L | T | R | Q | R | Y | 0 |
| 143 | G | G | P | E | E | G | A | S | L | R | H | Y | Y | N | W | L | T | R | Q | R | Y | 0 |
| 144 | G | G | P | E | E | G | A | S | L | R | H | Y | Y | N | W | L | T | R | Q | R | Y | 0 |
| 145 | G | G | P | S | S | G | A | S | L | R | H | Y | Y | N | W | L | T | R | Q | R | Y | 0 |
| 146 | G | G | P | E | E | G | A | S | L | R | H | Y | Y | N | W | L | T | R | Q | T | Y | 0 |
| 147 | G | G | P | S | S | G | A | S | L | R | H | Y | Y | N | W | L | T | R | Q | R | Y | 0 |
| 148 | G | G | P | S | E | P | A | S | L | R | H | Y | Y | N | W | L | T | R | Q | R | Y | 0 |
| 149 | G | G | P | E | E | G | A | S | L | R | H | Y | Y | N | W | L | T | R | Q | R | Y | 0 |
| 150 | G | G | P | S | S | G | A | S | L | R | H | Y | Y | N | W | L | T | R | Q | R | Y | 0 |
| 151 | G | G | P | S | E | G | A | S | L | R | H | Y | Y | N | W | L | T | R | Q | R | Y | 0 |
| 152 | G | G | P | E | S | G | A | S | L | R | H | Y | Y | N | W | L | T | R | Q | R | Y | 0 |
| 153 | G | G | P | S | E | G | A | S | L | R | H | Y | Y | N | W | L | T | R | Q | R | Y | 0 |
| 154 | G | G | P | S | E | G | A | S | L | R | H | Y | Y | N | W | L | T | R | Q | R | Y | 0 |
| 155 | G | G | P | E | E | G | A | S | L | R | H | Y | Y | N | W | L | T | R | Q | R | Y | 0 |
| 156 | G | G | P | S | E | G | A | S | L | R | H | Y | Y | N | W | L | T | R | Q | R | Y | 0 |
| 157 | G | G | P | S | E | G | A | S | L | R | H | Y | Y | N | W | L | T | R | Q | R | Y | 0 |
| 158 | G | G | P | S | S | P | A | S | L | R | H | Y | Y | A | W | L | T | R | Q | R | Y | 0 |
| 159 | G | G | P | S | S | G | A | S | L | R | H | Y | Y | N | W | L | T | R | Q | R | Y | 0 |
| 160 | G | G | P | S | E | G | A | S | L | R | H | Y | Y | N | W | L | T | R | Q | R | Y | 0 |
| 161 | G | G | P | E | E | P | A | S | L | R | H | Y | Y | N | W | L | T | R | Q | R | Y | 0 |
| 162 | G | G | P | E | E | G | A | S | L | R | H | Y | Y | N | W | L | T | R | Q | R | Y | 0 |
| 163 | G | G | P | E | S | G | A | S | L | R | H | Y | Y | N | W | L | T | R | Q | R | Y | 0 |
| 164 | G | G | P | S | E | G | A | S | L | R | H | Y | Y | N | W | L | T | R | Q | R | Y | 0 |
| 165 | G | G | P | S | E | P | A | S | L | R | H | Y | Y | N | W | L | T | R | Q | R | Y | 0 |
| 166 | G | G | P | E | S | G | A | S | L | R | H | Y | Y | N | W | L | T | R | Q | R | Y | 0 |
| 167 | G | G | P | S | S | G | A | S | L | R | H | Y | Y | N | W | L | T | R | Q | R | Y | 0 |
| 168 | G | G | P | R | S | G | A | S | L | R | H | Y | Y | N | W | L | T | R | Q | R | Y | 0 |
| 169 | G | G | P | S | E | P | A | S | L | R | H | Y | Y | N | W | L | T | R | Q | R | Y | 0 |
| 170 | G | G | P | S | S | G | A | S | L | R | H | Y | Y | N | W | L | T | R | Q | R | Y | 0 |
| 171 | G | G | P | S | S | G | A | S | L | R | H | Y | Y | N | W | L | T | R | Q | R | Y | 0 |
| 172 | G | G | P | S | S | G | A | S | L | R | H | Y | Y | N | W | L | T | R | Q | R | Y | 0 |
| 173 | G | G | P | S | E | P | A | S | L | R | H | Y | Y | N | W | L | T | R | Q | R | Y | 0 |
| 174 | G | G | P | S | E | G | A | S | L | R | H | Y | Y | Z | W | L | T | R | Q | R | Y | 0 |
| 175 | G | G | P | S | S | P | A | S | L | R | H | Y | Y | N | W | L | T | R | Q | R | Y | 0 |
| 176 | G | G | P | S | E | G | A | S | L | R | H | Y | Y | N | W | L | T | R | Q | R | Y | 0 |
| 177 | G | G | P | E | E | G | A | S | L | R | H | Y | Y | N | W | L | T | R | Q | R | Y | 0 |
| 178 | G | G | P | S | S | P | A | S | L | R | H | Y | Y | N | W | L | T | R | Q | R | Y | 0 |
| 179 | G | G | P | S | E | G | A | S | L | R | H | Y | Y | N | W | L | T | R | Q | R | Y | 0 |
| 180 | G | G | P | S | S | P | A | S | L | R | H | Y | Y | N | W | L | T | R | Q | R | Y | 0 |
| 181 | G | G | P | S | S | G | A | S | L | R | H | Y | Y | N | W | L | T | R | Q | R | Y | 0 |
| 182 | G | G | P | S | S | G | A | S | L | R | Aib | Y | Y | N | W | L | T | R | Q | R | Y | 1 |
| 183 | G | G | P | S | S | Y | A | S | L | R | H | Y | Y | N | W | L | T | R | Q | R | Y | 0 |
| 184 | G | G | P | S | S | G | A | S | L | R | H | Y | Aib | N | W | L | T | R | Q | R | Y | 1 |
| 185 | G | G | P | S | E | G | A | S | L | R | H | Y | Y | N | W | L | T | R | Q | R | Y | 0 |
| 186 | G | G | P | S | E | G | A | S | L | R | H | Y | Y | N | W | L | T | R | bh | R | Y | 1 |
| | | | | | | | | | | | | | | | | | | | Gln | | | |
| 187 | G | G | P | S | S | G | A | S | L | R | H | Y | Y | N | W | L | T | R | Q | R | Y | 0 |
| 188 | G | G | P | E | E | G | A | S | L | R | H | Y | Y | N | W | L | T | R | Q | R | Y | 0 |
| 189 | G | G | P | S | E | Y | A | S | L | R | H | Y | Y | N | W | L | T | R | Q | R | Y | 0 |
| 190 | G | G | P | S | S | G | A | S | L | R | H | Y | Y | N | W | D | T | R | Q | R | Y | 1 |
| | | | | | | | | | | | | | | | | Arg | | | | | | |
| 191 | G | G | P | S | S | G | A | S | L | R | H | Y | Y | N | W | L | T | R | Q | R | Tle | 1 |
| 192 | G | G | P | S | S | G | A | S | L | R | H | Y | Y | N | W | L | T | R | Q | R | Y | 0 |
| 193 | G | G | P | E | E | G | A | S | L | R | H | Y | Y | N | W | L | T | R | Q | R | Y | 0 |
| 194 | G | G | P | S | E | G | A | S | L | R | H | Y | Y | N | W | L | T | R | Q | R | Y | 0 |
| 195 | G | G | P | S | S | G | A | S | L | R | H | Y | Y | N | L | L | T | R | Q | R | Y | 1 |
| 196 | G | G | P | E | E | G | A | S | L | R | H | Y | Y | N | W | L | T | R | Q | R | Y | 0 |
| 197 | G | G | P | S | S | G | A | L | L | R | H | Y | Y | N | W | L | T | R | Q | R | Y | 1 |
| 198 | G | G | P | S | S | G | A | S | L | R | H | Y | Y | N | W | L | T | R | Q | R | Y | 0 |
| 199 | G | G | P | S | S | G | A | S | L | R | H | Y | Y | N | W | L | T | R | Q | R | Y | 0 |
| 200 | G | G | P | S | E | G | A | S | L | R | H | Y | Y | N | W | L | T | R | Q | R | Y | 0 |
| 201 | G | G | P | S | S | G | A | S | L | R | H | Y | Y | N | W | L | T | R | Q | R | Y | 0 |
| 202 | G | G | P | S | E | G | A | S | L | R | H | Y | Y | N | W | L | T | R | Q | R | Y | 0 |
| 203 | G | G | P | S | Y | Y | A | S | L | R | H | Y | Y | N | W | L | T | R | Q | R | Y | 0 |
| 204 | G | G | P | S | S | G | A | S | L | R | H | Y | Y | N | W | D | T | R | Q | R | Y | 1 |
| | | | | | | | | | | | | | | | | Asp | | | | | | |
| 205 | G | G | P | S | S | G | A | S | L | R | H | Y | Y | N | W | L | T | R | Q | R | Y | 0 |
| 206 | G | G | P | E | E | P | A | S | L | R | H | Y | Y | N | W | L | T | R | Q | R | Y | 0 |
| 207 | G | G | P | S | E | G | A | S | L | R | H | Y | Y | N | W | L | T | R | Q | R | Y | 0 |
| 208 | G | G | P | S | S | G | A | S | L | R | H | Y | Y | N | W | L | T | R | bh | R | Y | 1 |
| | | | | | | | | | | | | | | | | | | | Gln | | | |
| 209 | G | G | P | S | S | G | A | S | L | R | H | Y | Y | N | W | L | T | R | Q | R | Y | 0 |
| 210 | G | G | P | S | S | G | A | S | L | L | H | Y | Y | N | W | L | T | R | Q | R | Y | 1 |
| 211 | G | G | P | S | S | G | A | S | L | R | H | Y | Y | N | W | L | T | R | Q | R | Y | 0 |
| 212 | G | G | P | S | S | G | V | S | L | R | H | Y | Y | N | W | L | T | R | Q | R | Y | 1 |
| 213 | G | G | P | S | S | G | A | S | L | R | H | Y | Y | Aib | W | L | T | R | Q | R | Y | 1 |
| 214 | G | G | P | S | S | G | A | S | L | R | H | Y | Y | N | W | L | T | R | Q | R | D | 1 |
| | | | | | | | | | | | | | | | | | | | | | Tyr | |
| 215 | G | G | P | S | S | G | A | S | L | R | H | Y | Y | N | W | L | T | R | Q | R | Y | 0 |
| 216 | G | G | P | S | S | G | A | S | L | R | H | Y | Y | N | W | L | T | R | Q | R | Y | 0 |
| 217 | G | G | P | S | E | Y | V | S | L | R | H | Y | Y | N | W | L | T | R | Q | R | Y | 1 |
| 218 | G | G | P | S | S | G | A | S | L | R | H | Y | Y | N | Aib | L | T | R | Q | R | Y | 1 |
| 219 | G | G | P | S | E | G | A | S | L | R | H | Y | Y | N | W | L | T | R | Q | R | Y | 0 |
| 220 | G | G | P | S | E | G | A | S | L | R | H | Y | Y | N | W | L | T | R | Q | R | Y | 0 |
| 221 | G | G | P | S | S | G | A | S | L | R | H | Y | Y | N | W | L | T | R | Q | R | Y | 0 |
| 222 | G | G | P | S | E | G | A | S | L | R | H | Y | Y | N | W | L | T | R | NMe | R | Y | 1 |
| | | | | | | | | | | | | | | | | | | | Q | | | |
| 223 | G | G | P | S | E | G | A | S | L | R | H | Y | Y | N | W | L | T | R | Q | R | Y | 0 |
| 224 | G | G | P | S | E | G | A | S | L | R | H | Y | Y | N | W | L | T | R | Q | R | Y | 0 |
| 225 | G | G | P | S | S | G | A | S | L | R | H | Y | Y | N | W | L | T | R | Q | R | Y | 0 |
| 226 | G | G | P | S | E | P | A | S | L | R | H | Y | Y | N | W | L | T | R | Q | R | Y | 0 |
| 227 | G | G | P | S | E | G | Q | S | L | R | H | Y | Y | N | W | L | T | R | Q | R | Y | 1 |
| 228 | G | G | P | S | E | G | A | S | L | R | H | Y | Y | N | W | L | T | R | Q | R | Y | 0 |
| 229 | G | G | P | S | S | G | A | S | L | R | H | Y | Y | N | W | L | T | R | Q | R | Y | 0 |
| 230 | G | G | P | E | E | G | A | S | L | R | H | Y | Y | N | W | L | T | R | Q | R | Y | 0 |
| 231 | G | G | P | S | E | G | A | S | L | R | H | Y | Y | N | W | L | T | R | Q | R | Y | 0 |
| 232 | G | G | P | S | Y | G | A | S | L | R | H | Y | Y | N | W | L | T | R | Q | R | Y | 0 |
| 233 | G | G | P | S | E | P | A | S | L | R | H | Y | Y | N | W | L | T | R | Q | R | Y | 0 |
| 234 | G | G | P | S | E | G | A | S | L | R | H | Y | Y | N | W | L | T | R | Q | R | Y | 0 |
| 235 | G | G | P | S | S | G | A | S | L | R | H | Y | Y | N | W | L | T | R | Q | R | Y | 0 |
| 236 | G | G | P | S | E | G | A | S | L | R | H | Y | Y | N | W | L | T | R | Q | NMe | Y | 1 |
| | | | | | | | | | | | | | | | | | | | | R | | |
| 237 | G | G | P | S | E | G | E | S | L | R | H | Y | Y | N | W | L | T | R | Q | R | Y | 1 |
| 238 | G | G | P | S | S | G | A | S | L | R | H | Y | Y | N | W | L | T | R | Q | R | Y | 0 |
| 239 | G | G | P | S | E | G | A | S | L | R | H | Y | Y | N | W | L | T | R | Q | R | Y | 0 |
| 240 | G | G | P | S | E | G | A | S | L | R | H | Y | Y | N | W | L | T | R | Q | R | Y | 0 |
| 241 | G | G | P | S | E | G | V | S | L | R | H | Y | Y | N | W | L | T | R | Q | R | Y | 1 |
| 242 | G | G | P | S | E | G | A | S | L | R | H | Y | Y | N | W | L | T | R | Q | R | Y | 0 |
| 243 | G | G | P | S | E | G | A | S | L | R | H | Y | Y | N | W | L | T | R | Q | R | Y | 0 |
| 244 | G | G | P | S | T | G | A | S | L | R | H | Y | Y | N | W | L | T | R | Q | R | Y | 0 |
| 245 | G | G | P | S | E | G | A | S | L | R | H | Y | Y | N | W | L | T | R | Q | R | Y | 0 |
| 246 | G | G | P | S | E | G | A | S | L | R | H | Y | Y | N | W | L | T | R | Q | R | Y | 0 |
| 247 | G | G | P | S | E | G | A | S | L | R | H | Y | Y | N | W | L | T | R | Q | R | Y | 0 |
| 248 | G | G | P | E | E | G | A | S | L | R | H | Y | Y | N | W | L | T | R | Q | R | Y | 0 |
| 249 | G | G | P | S | E | G | A | S | L | R | H | Y | Y | N | W | L | T | R | Q | R | Y | 0 |
| 250 | G | G | P | S | E | G | A | S | L | R | H | Y | Y | N | W | L | T | R | Q | R | Y | 0 |
| 251 | G | G | P | S | E | G | A | S | L | R | H | Y | Y | N | W | L | T | R | Q | R | Y | 0 |
| 252 | G | G | P | S | E | G | A | S | L | R | H | Y | Y | N | W | L | T | R | Q | R | Y | 0 |
| 253 | G | G | P | S | S | G | A | S | L | R | H | Y | Y | N | W | L | T | R | Q | R | Y | 0 |
| 254 | G | G | P | S | S | G | A | S | L | R | H | Y | Y | N | W | L | T | R | Q | R | Y | 0 |
| 255 | G | G | P | S | E | G | A | S | L | R | H | Y | Y | N | W | L | T | R | Q | R | Y | 0 |
| 256 | G | G | P | S | E | P | A | S | L | R | H | Y | Y | N | W | L | T | R | Q | R | Y | 0 |
| 257 | G | G | P | S | E | G | A | S | L | R | H | Y | Y | N | W | L | T | R | Q | R | Y | 0 |
| 258 | G | G | P | S | E | G | A | S | L | R | H | Y | Y | N | W | L | T | R | Q | R | Y | 0 |
| 259 | G | G | P | S | S | G | A | S | L | R | H | Y | Y | N | W | L | T | R | Q | R | Y | 0 |
| 260 | G | G | P | S | S | G | A | S | L | R | H | Y | Y | N | W | L | T | R | Q | R | Y | 0 |
| 261 | G | G | P | S | S | G | A | S | L | R | H | Y | Y | N | W | L | T | R | Q | R | Y | 0 |
| 262 | G | G | P | S | E | G | A | S | L | R | H | Y | Y | N | W | L | T | R | Q | R | Y | 0 |
| 263 | G | G | P | S | E | P | A | S | L | R | H | Y | Y | N | W | L | T | R | Q | R | Y | 0 |
| 264 | G | G | P | S | E | G | A | S | L | R | H | Y | Y | N | W | L | T | R | Q | R | Y | 0 |
| 265 | G | G | P | S | E | G | A | S | L | R | H | Y | Y | N | W | L | T | R | Q | R | Y | 0 |
| 266 | G | G | P | S | E | G | A | S | L | R | H | Y | Y | N | W | L | T | R | Q | R | Y | 0 |
| 267 | G | G | P | S | E | G | A | S | L | R | H | Y | Y | N | W | L | T | R | Q | R | Y | 0 |
| 268 | G | G | P | S | E | G | A | S | L | R | H | Y | Y | N | W | L | T | R | Q | R | Y | 0 |
| 269 | G | G | P | S | E | G | A | S | L | R | H | Y | Y | N | W | L | T | R | Q | R | Y | 0 |
| 270 | G | G | P | S | E | G | A | S | L | R | H | Y | Y | N | W | L | T | R | Q | R | Y | 0 |
| 271 | G | G | P | S | E | G | A | S | L | R | H | Y | Y | N | W | L | T | R | Q | R | Y | 0 |
| 272 | G | G | P | E | E | G | A | S | L | R | H | Y | Y | N | W | L | T | R | Q | R | Y | 0 |
| 273 | G | G | P | S | E | G | A | S | L | R | H | Y | Y | N | W | L | T | R | Q | R | Y | 0 |
| 274 | G | G | P | S | E | G | A | S | L | R | H | Y | Y | N | W | L | T | R | Q | R | Y | 0 |
| 275 | G | G | P | S | E | G | A | S | L | R | H | Y | Y | N | W | L | T | R | Q | R | Y | 0 |
| 276 | G | G | P | S | E | G | A | S | L | R | H | Y | Y | N | W | L | T | R | Q | R | Y | 0 |
| 277 | G | G | P | S | E | G | A | S | L | R | H | Y | Y | N | W | L | T | R | bh | R | Y | 1 |
| | | | | | | | | | | | | | | | | | | | Gln | | | |
| 278 | G | G | P | S | E | G | A | S | L | R | H | Y | Y | N | W | L | T | R | Q | R | Y | 0 |
| 279 | G | G | P | S | E | G | A | S | L | R | H | Y | Y | N | W | L | T | R | Q | R | Y | 0 |
| 280 | G | G | P | S | E | G | A | S | L | R | H | Y | Y | N | W | L | T | R | Q | R | Y | 0 |
| 281 | G | G | P | S | E | G | A | S | L | R | H | Y | Y | N | W | L | T | R | Q | R | Y | 0 |
| 282 | G | G | P | S | E | G | A | S | L | R | H | Y | Y | N | W | L | T | R | Q | R | Y | 0 |
| 283 | G | G | P | S | E | G | A | S | L | R | H | Y | Y | N | W | L | T | R | Q | R | Y | 0 |
| 284 | G | G | P | S | E | G | A | S | L | R | H | Y | Y | N | W | L | T | R | Q | R | Y | 0 |
| 285 | G | G | P | E | E | G | A | S | L | R | H | Y | Y | N | W | L | T | R | Q | R | Y | 0 |
| 286 | G | G | P | S | E | G | A | S | L | R | H | Y | Y | N | W | L | T | R | Q | R | Y | 0 |
| 287 | G | G | P | S | E | G | A | S | L | R | H | Y | Y | N | W | L | T | R | Q | R | Y | 0 |
| 288 | G | G | P | S | E | G | A | S | L | R | H | Y | Y | N | W | L | T | R | Q | R | Y | 0 |
| 289 | G | G | P | S | E | G | A | S | L | R | H | Y | Y | N | W | L | T | R | Q | R | Y | 0 |
| 290 | G | G | P | S | E | G | A | S | L | R | H | Y | Y | N | W | L | T | R | Q | R | Y | 0 |
| 291 | G | G | P | S | E | G | A | S | L | R | H | Y | Y | N | W | L | T | R | Q | R | Y | 0 |
| 292 | G | G | P | S | E | G | A | S | L | R | H | Y | Y | N | W | L | T | R | Q | R | Y | 0 |
| 293 | G | G | P | S | E | G | A | S | L | R | H | Y | Y | N | W | L | T | R | Q | R | Y | 0 |
| 294 | G | G | P | S | E | G | A | S | L | R | H | Y | Y | N | W | L | T | R | Q | R | Y | 0 |
| 295 | G | G | P | S | E | G | A | S | L | R | H | Y | Y | N | W | L | T | R | Q | R | Y | 0 |
| 296 | G | G | P | S | E | G | A | S | L | R | H | Y | Y | N | W | L | T | R | Q | R | Y | 0 |
| 297 | G | G | P | S | E | G | A | S | L | R | H | Y | Y | N | W | L | T | R | Q | R | Y | 0 |
| 298 | G | G | P | S | E | G | A | S | L | R | H | Y | Y | N | W | L | T | R | Q | R | Y | 0 |
| 299 | G | G | P | S | E | G | A | S | L | R | H | Y | Y | N | W | L | T | R | Q | R | Y | 0 |
| 300 | G | G | P | S | E | G | A | S | L | R | H | Y | Y | N | W | L | T | R | Q | R | Y | 0 |
| 301 | G | G | P | S | E | G | A | S | L | R | H | Y | Y | N | W | L | T | R | Q | R | Y | 0 |
| 302 | G | G | P | S | E | G | A | S | L | R | H | Y | Y | N | W | L | T | R | Q | R | Y | 0 |
| 303 | G | G | P | S | E | G | A | S | L | R | H | Y | Y | N | W | L | T | R | Q | R | Y | 0 |
| 304 | G | G | P | S | E | G | A | S | L | R | H | Y | Y | N | W | L | T | R | Q | R | Y | 0 |
| 305 | G | G | P | S | E | G | A | S | L | R | H | Y | Y | N | W | L | T | R | Q | R | Y | 0 |

| Exemplified amino acids | Exemplified substitutions vs. SEQ ID NO: 286 |
|---|---|
| G | None |
| G | None |
| P, G, or Q | G, or Q |
| S, E, or R; | E, or R |
| S, E, or Y | S, or Y |
| G, P, Y, or A | P, Y, or A |
| A, Q, I, V, E, or L | Q, I, V, E, or L |
| S, E, T, D, or L | E, T, D, or L |
| L, Aib, V, D-Leu, or I | Aib, V, D-Leu, or I |
| R, L, or Aib | L, or Aib |
| H, Aib, D-His, or Tle | Aib, D-His, or Tle |
| Y | None |
| L, Y, Aib, I, or Tle | L, Aib, I, or Tle |
| N or Aib | Aib |
| W, H, L, R, or Aib | H, L, R, or Aib |
| L, Aib, D-Arg, or D-Asp | Aib, D-Arg, or D-Asp |
| T | None |
| R, hArg, or D-Arg | hArg, or D-Arg |
| Q, bh-Gln, or NMeQ | bh-Gln, or NMeQ |
| R or NMeR | NMeR |
| Y, Tle, Chg, D-Tyr, Phg. | Tle, Chg, D-TyR, Phg. |

### General Procedure for Solid Phase Synthesis of Peptides

All peptides were synthesized by standard Fmoc-based solid phase peptide chemistry on a Tentagel S RAM resin (loading 0.23-0.25 mmol/g, bead size 90 μm) supplied by Iris Biotech GmbH or Rapp Polymere GmbH.

The following protected amino acids were used: Fmoc-Ala-OH, Fmoc-Aib-OH, Fmoc-Arg(Pbf)-OH, Fmoc-NMeArg(Pbf)-OH, Fmoc-Asn(Trt)-OH, Fmoc-Asp(tBu)-OH, Fmoc-Gln(Trt)-OH, Fmoc-NMeGln(Trt)-OH, Fmoc-Glu(tBu)-OH, Fmoc-Glu-OtBu, Fmoc-Gly-OH, Fmoc-His(Trt)-OH, Fmoc-Ile-OH, Fmoc-Leu-OH, Fmoc-Lys(Boc)-OH, Fmoc-Lys(Dde)-OH, Fmoc-Lys(Mtt)-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, Boc-Tyr(tBu)-OH, Fmoc-D-Tyr(tBu)-OH, Fmoc-Val-OH. The L-form of the amino acid building blocks was utilized if not specified otherwise.

The modular half-life extending group was built up by solid-phase peptide synthesis (SPPS) using protected building blocks such as, but not limited to, C18DA(tBu), C20DA(tBu), Fmoc-OEG-OEG-OH, Fmoc-OEG-OH, Boc-Lys(Fmoc)-OH, Fmoc-Glu-OtBu, Fmoc-Glu(tBu)-OH, Fmoc-Ahx-OH, Fmoc-Trx-OH and Fmoc-Sar-OH.

The amino acids, Fmoc-Glu-OtBu, Oxyma and DIC, were purchased from standard suppliers, e.g. Bachem, Novabiochem, ABCR GmbH & CO. KG., Iris Biotech GmbH, Sigma-Aldrich. C18DA(tBu) and C20DA(tBu) were supplied by Cool Pharm Ltd. or AstraTech, Fmoc-OEG-OEG-OH was supplied by ABCR GmbH & CO. KG or Iris Biotech GmbH, Fmoc-OEG-OH was supplied by Angene International Limited, Combi Blocks Inc., Iris Biotech GmbH or Hangzhou APIChem Technology Co., Ltd. Fmoc-Ahx-OH was purchased from Activate Scientific GmbH, Fmoc-Trx-OH was purchased from ABCR GmbH & CO. KG.

Assembly of peptides started from the C-terminus by stepwise chain elongation towards the N-terminus according to the respective sequences until the N-terminal amino acid was reached. Deprotection of the side chain of the branching amino acid, e.g. Lys(Dde), was followed by assembly of the half-life extending group. The peptides were obtained as TFA salts from cleavage/deprotection or from HPLC purification. The trifluoroacetate can be exchanged by common procedures, such as resin-ion exchange procedures, e.g. as disclosed in Roux, St. et al. J. Pept. Sci. 2008; 14: 354-359.

Synthesis Method 1 (501) Peptides were synthesized by microwave-assisted solid-phase peptide synthesis (SPPS) on a CEM Liberty Blue Peptide Synthesizer at 0.25 mmol scale on Tentagel S RAM resin using the Fmoc strategy. The stoichiometry and concentrations for peptide coupling reactions were 4 eq of suitably protected amino acid in DMF (0.2 mol/l, 5 ml), 4 eq of Oxyma in DMF (1 mol/l, 1 ml) and 8 eq DIC in DMF (1 mol/l, 2 ml).

The reaction times and temperatures were different for the amino acids. Single coupling 4 min at 90° C. was used for all standard amino acids except those mentioned below. Single coupling 20 min at 75° C. was used for Fmoc-Aib-OH, the amino acid after Aib was coupled twice. Double coupling 4 min at 90° C. was used for Fmoc-Arg(Pbf)-OH, C18DA(tBu), C20DA(tBu), Fmoc-OEG-OH, FmocNMeArg(Pbf)-OH and Fmoc-NMeGln(Trt)-OH. The amino acid after NMeArg and NMeGln was coupled twice. The last 8 standard amino acids in the main chain were coupled at 90° C. for 8 min.

A capping step with 20% acetic anhydride in DMF (10 ml) for 5 min at 65° C. was performed prior and after the coupling of Fmoc-Glu-OtBu and prior the coupling of C18DA(tBu) and C20DA(tBu). N^{a} Fmoc deprotection was performed with 10% piperidine/DMF (10 ml) for 1 min at 90° C. Deprotection of the Lys(Dde)-group was carried out 2 times with 5% hydrazine hydrate in DMF (10 ml) for 3 min at 90° C. Raw products were washed on resin with DCM and dried prior to cleavage. Cleavage from resin and deprotection was performed with a mixture of 95% TFA/water (10 ml) and triisopropylsilane (250 μl) for 45 min at 40° C. Crude peptides were precipitated with cold tert.butyl-methyl ether, dissolved in 50% acetonitrile/water and purified by preparative HPLC.

### Synthesis Method 2 (S02)

Peptides were synthesized by SPPS on a MultiSynTech SYRO II at 0.2 mmol scale. Standard coupling of amino acids was achieved by using 4 eq of suitably protected amino acids dissolved in 0.5 mol/l Oxyma-DMF solution (0.5 mol/1) and 4.5 eq DIC (1 mol/1) in DMF. Fmoc-Phe-OH was dissolved in 0.5 mol/l Oxyma-NMP and 4 eq were used for coupling (0.5 mol/1). Coupling time of the first 23 amino acids starting from the C-terminus was 15 min at 75° C. (Cys and His were coupled at 50° C.). Further elongation was done by double coupling (2×15 min at 75° C.). Deprotection of Lys(Mtt)-group was carried out selectively using hexafluoroisopropanol (10×10 min at RT, 10 ml). Derivatisation of the deprotected lysine intermediate was done by double coupling 4 eq. of the relevant linker building blocks (Fmoc-OEG-OEG-OH, Fmoc-Glu-OtBu, Boc-Lys(Fmoc)-OH, C18DA(tBu), C20DA(tBu) for 15 min at 75° C. Na Fmoc deprotections were performed using 40% piperidine in NMP (4 ml) for 3 min followed by 20% piperidine in NMP (4 ml) for 15 min at 45° C. Suitably protected Fmoc-Asp, Fmoc-Cys and Fmoc-His intermediates were deprotected at room temperature. Peptides were cleaved from resin and side-chains deprotected by adding 15 ml 95:2:1:2 TFA/DODT/TES/water for 4 h at RT or 60 min at 45° C. The peptides were precipitated with cold diethyl ether, dissolved in acetonitrile/water and purified by preparative HPLC (Purification Method 3, P02).

### Purification Method 1 (P01)

Crude peptides were dissolved in DMF/acetonitrile/water and purified by reversed phase chromatography using an Agilent preparative HPLC-MS System with preparative pumps G7161B, G7111B and G7110B, a diode array detector G7115A, a mass-spectrometer G6135B and a fraction collector G7158B. A Waters Luna Prep C8(3) column (100 Å, 10 μm, 300 g, self-filled steel column) served as stationary phase. The mobile phase was run with a gradient of buffer A (ACN) and buffer (H₂O+0.1% TFA) as described in the table below at a flow rate of 150 ml/min at 40° C. The relevant fractions were pooled and lyophilized. The final product was characterized by analytical HPLC-MS (U046_001 and U046_006).

Purification method 1 standard gradient

| Time in Min | Flow[Pump A + B] | Pump A % | Pump B % |
|---|---|---|---|
| 0.0 | 150 ml/min | 20 | 80 |
| 2.0 | 150 ml/min | 20 | 80 |
| 4.0 | 120 ml/min | 28 | 72 |
| 8.0 | 120 ml/min | 28 | 72 |
| 9.0 | 120 ml/min | 33 | 67 |
| 17.0 | 120 ml/min | 34 | 66 |
| 18.0 | 120 ml/min | 38 | 62 |
| 21.5 | 120 ml/min | 39 | 61 |
| 22.0 | 150 ml/min | 100 | 0 |
| 24.0 | 150 ml/min | 100 | 0 |

### Purification Method 2 (P02)

Crude peptides were dissolved in DMF/acetonitrile/water and purified by reversed phase chromatography using a GILSON preparative HPLC System with preparative pumps AP-MOD (max. flow rate: 200 ml/min), a diode array detector ECOM Flash 10 and a fraction collector GILSON GX 281. Stationary phase was a Phenomenex LUNA C8 10 μm Prep Column (50×250 mm). The peptides were eluted with a focused gradient using water (eluent A) and acetonitrile (eluent B) at a flow rate of 120 ml/min and 40° C.; modifier solution was added in ‘at-column dilution’ mode to maintain a constant amount of 0.1% TFA within the mobile phase. Homogeneous fractions were pooled and lyophilized. The final product was characterized by HPLC-MS (U046_001 and U046_006).

Purification method 2 standard gradient

| Time in Min | Flow[Pump A + B] | Pump A % | Pump B % |
|---|---|---|---|
| 0.0 | 120 mL/min | 20 | 80 |
| 6.0 | 120 mL/min | 20 | 80 |
| 45.0 | 120 mL/min | 40 | 60 |
| 52.0 | 120 mL/min | 40 | 60 |
| 55.0 | 120 mL/min | 45 | 55 |
| 62.0 | 120 mL/min | 45 | 55 |
| 63.0 | 120 mL/min | 100 | 0 |
| 70.0 | 120 mL/min | 100 | 0 |
| 70.1 | 0 mL/min | 100 | 0 |

### Purification Method 3 (P03)

Crude peptides were purified by reverse phase HPLC using a Waters preparative HPLC system with C8 column (Reprosil Gold 200 Å, 5 μm, 40 mm×250 mm), preparative pumps (waters 2545), UV/VIS detector (Waters 2489) and a Waters fraction collector III. The mobile phase was run with a gradient of buffer A (0.1% TFA in H₂O) and buffer B (0.1% TFA in ACN, gradient: 35-45% B over 20 min) at a flow rate of 50 ml/min at RT. Relevant fractions were analysed, pooled and lyophilized. The final product was characterized by analytical UPLC-MS (A02).

### Analytical Method 1 (A01-A/B)

Peptide purity and mass were estimated by analytical HPLC-MS on a Kinetex C8 column (4.6 mm×150 mm, 2.6 um, Phenomenex) using a Agilent 1260 HPLC system equipped with Mass Detector G6135. Analysis was performed by gradient elution with buffer A (0.3% TFA in H₂O) and buffer B (0.24% TFA in ACN) at a temperature of 40° C. Details of the gradient and flow rates are summarized in the tables below. Retention times and masses were recorded.

Peptide purities (relative peak areas @214 nm) were in the range from 80 to 99%, preferably greater than 95%.

| | | Gradient/ | % Sol | % Sol | | |
|---|---|---|---|---|---|---|
| Method | A01-A | Solvent | [Water 0.3% | [ACN 0.24% | Flow | Temp |
| name | (U046_001) | Time [min] | TFA (v/v)] | TFA (v/v)] | ml/min | [° C.] |
| Device | Agilent 1260 with MS | 0.0 | 65.0 | 35.0 | 0.5 | 40.0 |
| | G6135 | | | | | |
| Column | Kinetex C8_4.6 × 150 | 15.0 | 45.0 | 55.0 | 0.5 | 40.0 |
| | mm_2.6 μm | | | | | |
| Column | Phenomenex | 16.0 | 10.0 | 90.0 | 1.0 | 40.0 |
| producer | | | | | | |
| | | 17.0 | 65.0 | 35.0 | 1.0 | 40.0 |
| | | 19.0 | 65.0 | 35.0 | 1.0 | 40.0 |

| | | Gradient/ | % Sol | % Sol | | |
|---|---|---|---|---|---|---|
| Method | A01-B | Solvent | [Water 0.3% | [ACN 0.24% | Flow | Temp |
| Name | (U046_006) | Time [min] | TFA (v/v)] | TFA (v/v)] | ml/min | [° C.] |
| Device | Agilent 1260 with MS | 0.0 | 85.0 | 15.0 | 0.5 | 40.0 |
| | G6135 | | | | | |
| Column | Kinetex C8_4.6 × 150 | 50.0 | 35.0 | 65.0 | 0.5 | 40.0 |
| | mm_2.6 μm | | | | | |
| Column | Phenomenex | 52.0 | 10.0 | 90.0 | 1.0 | 40.0 |
| producer | | | | | | |
| | | 85.0 | 65.0 | 15.0 | 1.0 | 40.0 |
| | | 85.0 | 65.0 | 15.0 | 1.0 | 40.0 |

### Analytical Method 2 (A02-A/B) SPG

Peptide purity and mass were determined by analytical HPLC-MS on a Kinetex C8 column (Phenomenex, 100 2.6 μm, 4.6 mm×150 mm) using a Waters Acquity HPLC System equipped with 3100 Mass Detector. Analysis was performed by gradient elution with buffer A (0.3% TFA in H₂O) and buffer B (0.3% TFA in ACN) at a temperature of 40° C. Details of the gradient and flow rates are summarized in the table below. Retention times and masses were recorded.

| | | Gradient/ | % Sol | % Sol | | |
|---|---|---|---|---|---|---|
| Method | | Solvent | [Water 0.3% | [ACN 0.3% | Flow | Temp |
| Name | A02-A | Time [min] | TFA (v/v)] | TFA (v/v)] | [ml/min] | [° C.] |
| Device | Waters Acquity | 0.0 | 90.0 | 10.0 | 0.3 | 40.0 |
| | UPLC peptide | | | | | |
| | C18 2.1 × 100 | | | | | |
| | mm_1.7 μm | | | | | |
| Column | Kinetex C8_4.6 × | 2.0 | 60.0 | 40.0 | 0.3 | 40.0 |
| | 150 mm_2.6 μm | | | | | |
| Column | Phenomenex | 8.5 | 40.0 | 60.0 | 0.3 | 40.0 |
| producer | | | | | | |
| | | 10.5 | 10.0 | 90.0 | 0.3 | 40.0 |
| | | 12.0 | 10.0 | 90.0 | 0.3 | 40.0 |
| | | Gradient/ | % Sol | % Sol | | |
| Method | | Solvent | [Water 0.3% | [ACN 0.3% | Flow | Temp |
| Name | A02-B | Time [min] | TFA (v/v)] | TFA (v/v)] | [ml/min] | [° C.] |
| Device | Waters Acquity | 0.0 | 90.0 | 10.0 | 0.3 | 40.0 |
| | UPLC peptide | | | | | |
| | C18 2.1 × 100 | | | | | |
| | mm_1.7 μm | | | | | |
| Column | Kinetex C8_4.6 × | 2.0 | 60.0 | 40.0 | 0.3 | 40.0 |
| | 150 mm_2.6 μm | | | | | |
| Column | Phenomenex | 16.5 | 40.0 | 60.0 | 0.3 | 40.0 |
| producer | | | | | | |
| | | 18.5 | 10.0 | 90.0 | 0.3 | 40.0 |
| | | 21.0 | 10.0 | 90.0 | 0.3 | 40.0 |

### List of Abbreviations

ACN: acetonitrile
AHX: 6-aminohexanoic acid
Aib: amino-isobutyric acid
aMeF: alpha-methyl-L-phenylalanine
bh-Gln: beta-homo-L-glutamine
Boc: tert-butyloxycarbonyl
Chg: cyclohexyl-glycine
C18DA(tBu): 18-(tert-butoxy)-18-oxooctadecanoic acid
C20DA(tBu): 20-(tert-butoxy)-20-oxoicosanoic acid
D-Arg: D-arginine
D-Asp: D-aspartic acid
DCM: dichloromethane
DIC: diisopropylcarbodiimide
DIPEA: diisopropylethylamine
Dde: (4,4-dimethyl-2,6-dioxocyclohex-1-ylidene)ethyl
D-His: D-histidine
D-Leu: D-leucin
DMF: N,N-dimethylformamid
DODT: 3,6-dioxa-1,8-octanedithiol
DPBS: Dulbecco's phosphate-buffered saline
D-Tyr: D-tyrosine
eLys: N-epsilon-L-lysine
Fmoc: 9H-fluoren-9-ylmethoxycarbonyl
Fmoc-Ahx: 6-{[(9H-fluoren-9-ylmethoxy)carbonyl]amino}hexanoic acid
Fmoc-OEG-OH: 2-[2-[2-(9H-fluoren-9-ylmethoxycarbonylamino)ethoxy]ethoxy] acetic acid
Fmoc-OEG-OEG-OH:2-[2-[2-[[2-[2-[2-(9H-fluoren-9-ylmethoxycarbonyl-
amino)ethoxy]ethoxy]acetyl]amino]ethoxy]ethoxy]acetic acid
gGlu: C-gamma-L-glutamate
hArg: homo-L-arginine
HTRF: homogeneous time resolved fluorescence
IBMX: 3-isobutyl-1-methylxanthine
Iva: 2-amino-2-methylbutanoic acid
iVal: 3-methylbutanoyl (isovalerianoyl)
MRT: mean residence time
Mtt: 4-Methyl-trityl
NMeR: N-methyl-L-arginine
NMeQ: N-methyl-L-glutamine
NMeY: N-methyl-L-tyrosine
NMP: 1-methyl-pyrrolidine-2-one
Oxyma: 2-cyano-2-(hydroxyimino)acetic acid ethyl ester
OEG: 2-[2-(2-aminoethoxy)ethoxy]acetic acid
Phg: S-phenylglycine
Pbf: 2,2,4,6,7-pentamethyldihydrobenzofuran-5-sulfonyl
Rt: retention time
RT: room temperature
Sarcosine: N-Methyl-glycine
SPPS: solid-phase peptide synthesis
tBu: tert-butyl
Tle: L-tert-butylglycine
Trt: trityl
TRX: tranexamic acid, trans-4-(aminomethyl)cyclohexane-1-carboxylic
acid
TES: triethylsilane
TFA: trifluoroacetic acid

The following compounds were synthesised. All compounds were obtained as TFA salts:

Compound 2
(SEQ ID NO: 328)
Y-Aib-E-G-T-F-T-S-D-Y-S-I-Aib-L-E-K-Q-A-Q-Aib-A-F-
V-E-W-L-I-K(1,3)-G-G-P-S-S-G-I-E-L-R-H-F-L-N-H-L-
T-R-Q-R-Y-NH2.C20DA-gGlu-gGlu-gGlu-gGlu-gGlu-gGlu-
eLys(1,2)

MW (calculated): 6869.8 Da
Synthesis and purification methods: 501; P02
LCMS: A01-B; Rt: 35.8 min; m/3: m/4: 1718.1 m/5:

Compound 3
(SEQ ID NO: 329)
Y-Aib-E-G-T-F-T-S-D-Y-S-I-Aib-L-E-K-Q-A-Q-Aib-A-F-V-Q-W-L-I-K(1,3)-G-G-Q-R-S-Y-A-S-L-R-H-
Y-Y-N-W-L-T-R-Q-R-Y-NH2.C20DA-gGlu-gGlu-gGlu-gGlu-gGlu-gGlu-eLys(1,2)

MW (calculated): 7106.0 Da
Synthesis and purification methods: S02; P03
LCMS: A02-A; Rt: 7.7 min; m/3: 2370.2 m/4: 1777.9 m/5:

Compound 4
(SEQ ID NO: 330)
Y-Aib-E-G-T-F-T-S-D-Y-S-I-Aib-L-E-K-Q-A-Q-Aib-A-F-V-E-W-L-I-K(1,3)-G-G-P-S-S-G-A-S-Aib-R-H-
Y-Aib-N-R-Aib-T-R-Q-R-Y-NH2.C20DA-gGlu-gGlu-gGlu-gGlu-gGlu-gGlu-eLys(1,2)

MW (calculated): 6736.5 Da
Synthesis and purification methods: S01; P02
LCMS: A01-B; Rt: 34.0 min; m/3: m/4: 1684.9 m/5:

Compound 5
(SEQ ID NO: 331)
Y-Aib-E-G-T-F-T-S-D-Y-S-I-Aib-L-E-K-Q-A-Q-Aib-A-F-V-E-W-L-I-K(1,3)-G-G-P-S-S-G-A-S-DLeu-R-
DHis-Y-Y-N-W-L-T-DArg-Q-R-DTyr-NH2.C20DA-gGlu-gGlu-gGlu-gGlu-gGlu-gGlu-eLys(1,2)

MW (calculated): 6900.7 Da
Synthesis and purification methods: S01; P02
LCMS: A01-B; Rt: 34.8 min; m/3: m/4: 1726.0 m/5:

Compound 6
(SEQ ID NO: 332)
Y-Aib-E-G-T-F-T-S-D-Y-S-I-Aib-L-E-K-Q-A-Q-Aib-A-F-V-Q-W-L-I-K(1,3)-G-G-Q-R-Y-Y-A-S-L-R-H-Y-
Y-N-W-L-T-R-Q-R-Y-NH2.C20DA-gGlu-gGlu-gGlu-Glu-gGlu-Glu-eLys(1,2)

MW (calculated): 7182.1 Da
Synthesis and purification methods: S02; P03
LCMS: A02-A; Rt: 8.0 min; m/3: 2395.5 m/4: 1797.0 m/5:

Compound 7
(SEQ ID NO: 333)
Y-Aib-E-G-T-F-T-S-D-Y-S-I-Aib-L-E-E-E-A-Q-Aib-A-F-V-Q-W-L-I-K(1,3)-G-G-P-S-S-G-A-S-L-R-H-Y-
Y-N-W-L-T-R-Q-R-Y-NH2.C20DA-gGlu-gGlu-gGlu-gGlu-gGlu-eLys-eLys(1,2)

MW (calculated): 6900.7 Da
Synthesis and purification methods: S01; P02
LCMS: A01-B; Rt: 35.9 min; m/3: m/4: 1725.9 m/5:

Compound 8
(SEQ ID NO: 334)
Y-Aib-E-G-T-F-T-S-D-Y-S-I-Aib-L-E-E-Q-A-Q-Aib-A-F-V-Q-W-L-I-K(1,3)-G-G-P-S-S-G-Q-S-L-R-H-Y-Y-
N-W-L-T-R-Q-R-Y-NH2.C20DA-gGlu-Glu-gGlu-gGlu-gGlu-gGlu-eLys(1,2)

MW (calculated): 6957.7 Da
Synthesis and purification methods: S02; P03
LCMS: A02-A; Rt: 7.5 min; m/3: 2320.3 m/4: 1740.7 m/5:

Compound 9
(SEQ ID NO: 335)
Y-Aib-E-G-T-F-T-S-D-Y-S-I-Aib-L-E-K-Q-A-Q-Aib-A-F-V-E-W-L-I-K(1,3)-G-G-P-S-S-Y-V-S-I-R-H-Y-
Y-N-W-L-T-R-Q-R-Y-NH2.C20DA-gGlu-gGlu-gGlu-gGlu-gGlu-gGlu-eLys(1,2)

MW (calculated): 7034.9 Da
Synthesis and purification methods: S02; P03
LCMS: A02-A; Rt: 7.7 min; m/3: 2346.7 m/4: 1760.1 m/5:

Compound 10
(SEQ ID NO: 336)
Y-Aib-E-G-T-F-T-S-D-Y-S-I-Aib-L-E-K-Q-A-Q-Aib-A-F-V-E-W-L-I-K(1,3)-G-G-P-S-S-P-E-S-Aib-R-H-Y-
Y-N-W-L-T-R-Q-R-Y-NH2.C20DA-gGlu-gGlu-gGlu-gGlu-gGlu-gGlu-eK

MW (calculated): 6970.8 Da
Synthesis and purification methods: S02; P03
LCMS: A02-A; Rt: 6.1 min; m/3: 2324.9 m/4: 1743.9 m/5:

Compound 11
(SEQ ID NO: 337)
Y-Aib-E-G-T-F-T-S-D-Y-S-I-Aib-L-E-K-Q-A-Q-Aib-A-F-V-Q-W-L-I-K(1,3)-G-G-P-E-S-P-A-S-L-R-H-
Y-Y-N-W-L-T-R-Q-R-Y-NH2.C20DA-gGlu-gGlu-gGlu-gGlu-gGlu(1,2)

MW (calculated): 6724.6 Da
Synthesis and purification methods: S02; P03
LCMS: A02-A; Rt: 7.8 min; m/3: 2242.2 m/4: 1681.8 m/5:

Compound 12
(SEQ ID NO: 338)
Y-Aib-E-G-T-F-T-S-D-Y-S-I-Aib-L-E-K-Q-A-Q-Aib-A-F-V-Q-W-L-I-K(1,3)-G-G-P-E-S-P-A-S-L-R-H-Y-Y-
N-W-L-T-R-Q-R-Y-NH2.C18DA-gGlu-gGlu-gGlu-gGlu-gGlu(1,2)

MW (calculated): 6696.5 Da
Synthesis and purification methods: S02; P03
LCMS: A02-A; Rt: 7.1 min; m/3: 2232.7 m/4: 1674.9 m/5:

Compound 13
(SEQ ID NO: 339)
Y-Aib-E-G-T-F-T-S-D-Y-S-I-Aib-L-E-K-Q-A-Q-Aib-A-F-V-E-W-L-I-K(1,3)-G-G-P-S-S-G-A-S-
Aib-R-H-Y-Y-N-R-Aib-T-R-Q-R-Y-NH2.C20DA-gGlu-gGlu-gGlu-gGlu-gGlu-Glu-eLys(1,2)

MW (calculated): 6814.6 Da
Synthesis and purification methods: 501; P02
LCMS: A01-B; Rt: 33.6 min; m/3: m/4: 1704.5 m/5:

Compound 14
(SEQ ID NO: 340)
Y-Aib-E-G-T-F-T-S-D-Y-S-I-Aib-L-E-K-Q-A-Q-Aib-A-F-V-Q-W-L-I-K(1,3)-G-G-P-R-Y-Y-A-S-L-R-H-Y-
Y-N-W-L-T-R-Q-R-Y-NH2.C20DA-gGlu-gGlu-gGlu-gGlu-gGlu-gGlu-eLys(1,2)

MW (calculated): 7151.1 Da
Synthesis and purification methods: S02; P03
LCMS: A02-A; Rt: 7.2 min; m/3: 2385.6 m/4: 1789.2 m/5:

Compound 15
(SEQ ID NO: 341)
Y-Aib-E-G-T-F-T-S-D-Y-S-I-Aib-L-E-K-Q-A-Q-Aib-A-F-V-Q-W-L-I-K(1,3)-G-G-P-S-S-Y-V-S-L-R-H-Y-
Y-N-W-L-T-R-Q-R-Y-NH2.C20DA-gGlu-gGlu-gGlu-gGlu-gGlu-gGlu-eLys(1,2)

Synthesis and purification methods: S02; P03
LCMS: A02-A; Rt: 7.6 min; m/3: 2346.2 m/4: 1759.9 m/5:

Compound 16
(SEQ ID NO: 342)
Y-Aib-E-G-T-F-T-S-D-Y-S-I-Aib-L-E-Aib-E-A-Q-Aib-K-F-V-E-W-L-I-K(1,3)-G-G-P-S-S-G-A-S-L-
R-H-Y-Y-N-W-L-T-R-Q-R-Y-NH2.C20DA-gGlu-gGlu-gGlu-gGlu-gGlu-gGlu-eLys(1,2)

MW (calculated): 6915.7 Da
Synthesis and purification methods: S01; P02
LCMS: A01-B; Rt: 38.2 min; m/3: m/4: 1729.7 m/5:

Compound 17
(SEQ ID NO: 343)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-H-
Y-L-N-L-Aib-T-R-Q-R-Y-NH2.C20DA-gGlu-gGlu-gGlu-gGlu-gGlu-gGlu-eLys(1,2)

MW (calculated): 6749.6 Da
Synthesis and purification methods: S01; P02
LCMS: A01-B; Rt: 35.1 min; m/3: m/4: 1688.1 m/5:

Compound 18
(SEQ ID NO: 344)
Y-Aib-E-G-T-F-T-S-D-Y-S-I-Aib-L-E-K-Q-A-Q-Aib-A-F-V-E-W-L-I-K(1,3)-G-G-P-S-S-G-A-S-
L-R-H-Y-Aib-N-R-Aib-T-R-Q-R-Y-NH2.C20DA-gGlu-gGlu-gGlu-gGlu-gGlu-gGlu-eLys(1,2)

MW (calculated): 6764.6 Da
Synthesis and purification methods: S01; P02
LCMS: A01-B; Rt: 34.3 min; m/3: m/4: 1691.9 m/5:

Compound 19
(SEQ ID NO: 345)
Y-Aib-E-G-T-F-T-S-D-Y-S-I-Aib-L-E-K-Q-A-Q-Aib-A-F-V-Q-W-L-I-K(1,3)-G-G-P-S-Y-Y-V-S-L-R-
H-Y-Y-N-W-L-T-R-Q-R-Y-NH2.C20DA-gGlu-gGlu-gGlu-gGlu-gGlu-gGlu-eLys(1,2)

MW (calculated): 7110.0 Da
Synthesis and purification methods: S02; P03
LCMS: A02-A; Rt: 7.6 min; m/3: 2370.9 m/4: 1778.2 m/5:

Compound 20
(SEQ ID NO: 346)
Y-Aib-E-G-T-F-T-S-D-Y-S-I-Aib-L-A-A-A-A-Q-Aib-A-F-V-E-W-L-I-K(1,3)-G-G-P-S-S-G-A-S-L-R-H-
Y-Y-N-W-L-T-R-Q-R-Y-NH2.C20DA-gGlu-gGlu-gGlu-gGlu-gGlu-gGlu-eLys(1,2)

MW (calculated): 6728.5 Da
Synthesis and purification methods: S01; P02
LCMS: A01-B; Rt: 39.8 min; m/3: m/4: 1682.9 m/5:

Compound 21
(SEQ ID NO: 347)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-
DHis-Y-Y-N-W-L-T-DArg-Q-R-DTyr-NH2.C20DA-gGlu-gGlu-gGlu-gGlu-gGlu-gGlu-eLys(1,2)

MW (calculated): 6900.7 Da
Synthesis and purification methods: S01; P02
LCMS: A01-B; Rt: 35.0 min; m/3: m/4: 1725.9 m/5:

Compound 22
(SEQ ID NO: 348)
Y-Aib-E-G-T-F-T-S-D-Y-S-I-Aib-L-E-K-Q-A-Q-Aib-A-F-V-Q-W-L-I-K(1,3)-G-G-Q-S-S-Y-A-S-L-R-H-
Y-Y-N-W-L-T-R-Q-R-Y-NH2.C20DA-gGlu-gGlu-gGlu-gGlu-gGlu-Glu-eLys(1,2)

MW (calculated): 7036.9 Da
Synthesis and purification methods: S02; P03
LCMS: A02-A; Rt: 7.2 min; m/3: 2347.4 m/4: 1760.7 m/5:

Compound 23
(SEQ ID NO: 349)
Y-Aib-E-G-T-F-T-S-D-Y-S-I-Aib-L-E-K-Q-A-Q-Aib-A-F-V-Q-W-L-I-K(1,3)-G-G-P-E-Y-Y-A-S-L-R-H-Y-
Y-N-W-L-T-R-Q-R-Y-NH2.C20DA-gGlu-gGlu-gGlu-gGlu-Glu-eLys(1,2)

MW (calculated): 6994.9 Da
Synthesis and purification methods: S02; P03
LCMS: A02-A; Rt: 7.4 min; m/3: 2331.8 m/4: 1749.4 m/5:

Compound 24
(SEQ ID NO: 350)
Y-Aib-E-G-T-F-T-S-D-Y-S-I-Aib-L-E-Aib-K-A-Q-Aib-E-F-V-E-W-L-I-K(1,3)-G-G-P-S-S-G-A-S-L-
R-H-Y-Y-N-W-L-T-R-Q-R-Y-NH2.C20DA-gGlu-gGlu-gGlu-gGlu-gGlu-gGlu-eLys(1,2)

MW (calculated): 6915.7 Da
Synthesis and purification methods: S01; P02
LCMS: A01-B; Rt: 36.6 min; m/3: m/4: 1723.2 m/5:

Compound 25
(SEQ ID NO: 351)
Y-Aib-E-G-T-F-T-S-D-Y-S-I-Aib-L-E-A-A-K-Q-Aib-A-F-V-E-W-L-I-K(1,3)-G-G-P-S-S-G-A-S-
L-R-H-Y-Y-N-W-L-T-R-Q-R-Y-NH2.C20DA-gGlu-gGlu-gGlu-gGlu-gGlu-gGlu-eLys(1,2)

MW (calculated): 6843.7 Da
Synthesis and purification methods: S01; P02
LCMS: A01-B; Rt: 37.2 min; m/3: m/4: 1711.7 m/5:

Compound 26
(SEQ ID NO: 352)
Y-Aib-E-G-T-F-T-S-D-Y-S-I-Aib-L-E-K-Q-A-Q-Aib-A-F-V-E-W-L-I-K(1,3)-G-G-P-S-S-G-A-S-Aib-R-
H-Y-Y-N-W-L-T-hArg-Q-R-DTyr-NH2.C20DA-gGlu-gGlu-gGlu-gGlu-gGlu-gGlu-eLys(1,2)

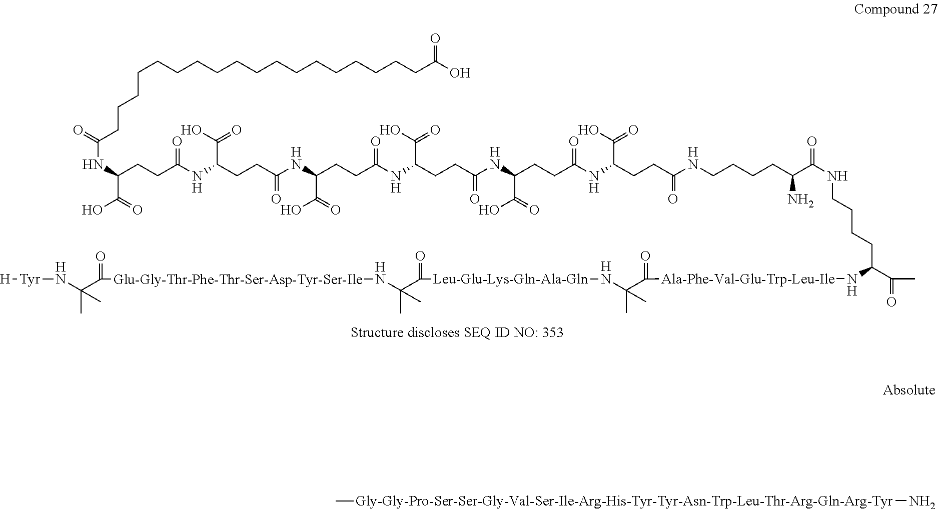

(SEQ ID NO: 353)
Y-Aib-E-G-T-F-T-S-D-Y-S-I-Aib-L-E-K-Q-A-Q-Aib-A-F-V-E-W-L-I-K(1,3)-G-G-P-S-S-G-V-S-I-R-H-
Y-Y-N-W-L-T-R-Q-R-Y-NH2.C20DA-gGlu-gGlu-gGlu-gGlu-gGlu-gGlu-eLys(1,2)

MW (calculated): 6928.8 Da
Synthesis and purification methods: S02; P03
LCMS: A02-A; Rt: 7.3 min; m/3: 2311.6 m/4: 1733.8 m/5:

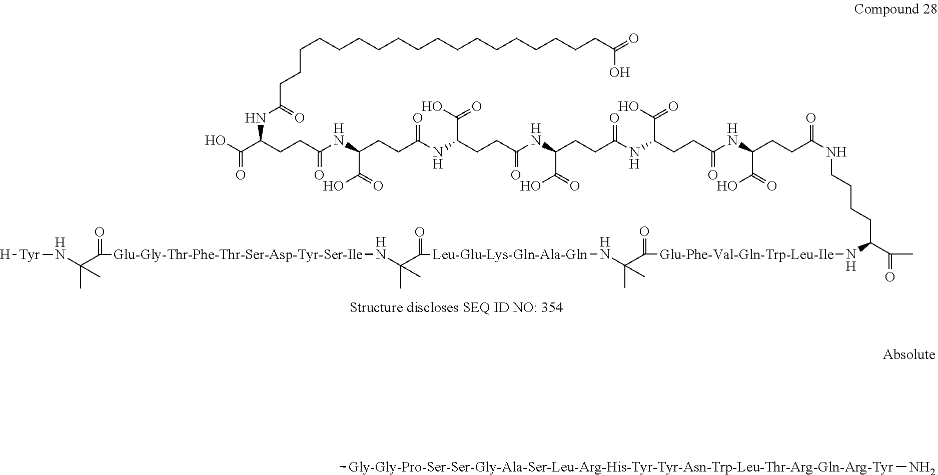

(SEQ ID NO: 354)
Y-Aib-E-G-T-F-T-S-D-Y-S-I-Aib-L-E-K-Q-A-Q-Aib-E-F-V-Q-W-L-I-K(1,3)-G-G-P-S-S-G-A-S-L-R-
H-Y-Y-N-W-L-T-R-Q-R-Y-NH2.C20DA-gGlu-Glu-gGlu-gGlu-gGlu-gGlu(1,2)

MW (calculated): 6829.6 Da
Synthesis and purification methods: 501; P02
LCMS: A01-A; Rt: 13.3 min; m/3: m/4: 1708.0 m/5:

Compound 29
(SEQ ID NO: 355)
Y-Aib-E-G-T-F-T-S-D-Y-S-I-Aib-L-E-K-Q-A-Q-Aib-A-F-V-Q-W-L-I-K(1,3)-G-G-P-E-S-G-A-S-L-R-H-
Y-Y-N-W-L-T-R-Q-R-Y-NH2.C20DA-gGlu-gGlu-gGlu-gGlu-gGlu(1,2)

MW (calculated): 6684.5 Da
Synthesis and purification methods: S02; P03
LCMS: A02-A; Rt: 7.7 min; m/3: 2228.8 m/4: 1672.0 m/5:

Compound 30
(SEQ ID NO: 356)
Y-Aib-E-G-T-F-T-S-D-Y-S-I-Aib-L-E-E-Q-A-Q-Aib-A-F-V-Q-W-L-I-K(1,3)-G-G-P-S-S-G-A-S-L-R-H-
Y-Y-N-W-L-T-R-Q-R-Y-NH2.C20DA-gGlu-gGlu-Glu-gGlu-gGlu-gGlu-eLys(1,2)

MW (calculated): 6900.7 Da
Synthesis and purification methods: 501; P02
LCMS: A01-B; Rt: 36.2 min; m/3: m/4: 1725.9 m/5:

Compound 31
(SEQ ID NO: 357)
Y-Aib-E-G-T-F-T-S-D-Y-S-I-Aib-L-E-E-Q-A-Q-Aib-A-F-V-E-W-L-I-K(1,3)-G-G-P-S-S-G-Q-S-L-R-
H-Y-Y-N-W-L-T-R-Q-R-Y-NH2.C20DA-gGlu-gGlu-gGlu-gGlu-gGlu-gGlu-eLys(1,2)

MW (calculated): 6958.7 Da
Synthesis and purification methods: S02; P03

Compound 32
(SEQ ID NO: 358)
Y-Aib-E-G-T-F-T-S-D-Y-S-I-Aib-L-E-K-Q-A-Q-Aib-A-F-V-Q-W-L-I-K(1,3)-G-G-P-E-S-G-A-S-L-
R-H-Y-Y-N-W-L-T-R-Q-R-Y-NH2.C20DA-gGlu-gGlu-gGlu-gGlu-gGlu-OEG-OEG(1,2)

LCMS: A02-A; Rt: 7.6 min; m/3: 2320.8 m/4: 1740.7 m/5:
MW (calculated): 6974.8 Da
Synthesis and purification methods: S02; P03
LCMS: A02-A; Rt: 7.7 min; m/3: 2325.7 m/4: 1744.4 m/5:

Compound 33
(SEQ ID NO: 359)
Y-Aib-E-G-T-F-T-S-D-Y-S-I-Aib-L-E-K-Q-A-Q-Aib-A-F-V-E-W-L-I-K(1,3)-G-G-P-S-S-A-V-S-L-R-H-
Y-Y-N-W-L-T-R-Q-R-Y-NH2.C20DA-gGlu-gGlu-gGlu-gGlu-gGlu-gGlu-eLys(1,2)

MW (calculated): 6942.8 Da
Synthesis and purification methods: S02; P03
LCMS: A02-A; Rt: 7.5 min; m/3: 2316.3 m/4: 1737.1 m/5:

Compound 34
(SEQ ID NO: 360)
Y-Aib-E-G-T-F-T-S-D-Y-S-I-Aib-L-E-E-Q-A-Q-Aib-A-F-V-Q-W-L-I-K(1,3)-G-G-P-S-S-G-A-S-L-R-
H-Y-Y-N-W-L-T-R-Q-R-Y-NH2.C20DA-gGlu-gGlu-gGlu-gGlu-gGlu-eLys(1,2)

MW (calculated): 6771.6 Da
Synthesis and purification methods: 501; P01
LCMS: A01-B; Rt: 36.4 min; m/3: m/4: 1693.6 m/5:

Compound 35
(SEQ ID NO: 361)
Y-Aib-E-G-T-F-T-S-D-Y-S-I-Aib-L-E-K-Q-A-Q-Aib-A-F-V-Q-W-L-I-K(1,3)-G-G-P-S-S-G-E-S-L-
R-H-Y-Y-N-W-L-T-R-Q-R-Y-NH2.C20DA-gGlu-gGlu-gGlu-gGlu-gGlu-gGlu-eK

MW (calculated): 6957.8 Da
Synthesis and purification methods: S02; P03
LCMS: A02-A; Rt: 6.2 min; m/3: 2320.5 m/4: 1740.4 m/5:

Compound 36
(SEQ ID NO: 362)
Y-Aib-E-G-T-F-T-S-D-Y-S-I-Aib-L-E-K-Q-A-Q-Aib-E-F-V-Q-W-L-I-K(1,3)-G-G-P-S-S-G-A-S-L-R-H-
Y-Y-N-W-L-T-R-Q-R-Y-NH2.C20DA-gGlu-gGlu-gGlu-gGlu-gGlu-OEG-OEG(1,2)

MW (calculated): 6990.8 Da
Synthesis and purification methods: S02; P03
LCMS: A02-A; Rt: 7.6 min; m/3: 2331.2 m/4: 1748.3 m/5:

Compound 37
(SEQ ID NO: 363)
Y-Aib-E-G-T-F-T-S-D-Y-S-I-Aib-L-E-K-Q-A-Q-Aib-A-F-V-Q-W-L-I-K(1,3)-G-G-P-E-S-G-A-S-L-R-H-
Y-Y-N-W-L-T-R-Q-R-Y-NH2.C20DA-gGlu-gGlu-gGlu-gGlu-gGlu-gGlu-eLys(1,2)

MW (calculated): 6941.8 Da
Synthesis and purification methods: 501; L20
LCMS: A01-A; Rt: 7.2 min; m/3: 2314.9 m/4: 1736.3 m/5:

Compound 38
(SEQ ID NO: 364)
Y-Aib-E-G-T-F-T-S-D-Y-S-I-Aib-L-E-K-Q-A-Q-Aib-A-F-V-E-W-L-I-K(1,3)-G-G-P-S-S-G-V-S-
Aib-R-H-Y-Y-N-W-L-T-R-Q-R-Y-NH2.C20DA-gGlu-gGlu-gGlu-gGlu-gGlu-gGlu-eLys(1,2)

MW (calculated): 6900.7 Da
Synthesis and purification methods: S02; P03
LCMS: A02-A; Rt: 6.1 min; m/3: 2301.4 m/4: 1726.2 m/5:

Compound 39
(SEQ ID NO: 365)
Y-Aib-E-G-T-F-T-S-D-Y-S-I-Aib-L-E-K-Q-A-Q-Aib-A-F-V-E-W-L-I-K(1,3)-G-G-P-S-S-G-A-S-Aib-
R-H-Y-Y-N-W-L-T-R-Q-NMeR-Y-NH2.C20DA-gGlu-gGlu-gGlu-gGlu-gGlu-gGlu-eLys(1,2)

MW (calculated): 6886.7 Da
Synthesis and purification methods: 501; P02
LCMS: A01-B; Rt: 34.4 min; m/3: m/4: 1722.3 m/5:

Compound 40
(SEQ ID NO: 366)
Y-Aib-E-G-T-F-T-S-D-Y-S-I-Aib-L-E-K-Q-A-Q-Aib-A-F-V-E-W-L-I-K(1,3)-G-G-P-S-S-Y-V-S-L-
R-H-Y-Y-N-W-L-T-R-Q-R-Y-NH2.C20DA-gGlu-gGlu-gGlu-gGlu-gGlu-gGlu-eLys(1,2)

MW (calculated): 7034.9 Da
Synthesis and purification methods: S02; P03
LCMS: A02-A; Rt: 7.5 min; m/3: 2345.5 m/4: 1759.4 m/5:

Compound 41
(SEQ ID NO: 367)
Y-Aib-E-G-T-F-T-S-D-Y-S-I-Aib-L-E-K-Q-A-Q-Aib-A-F-V-E-W-L-I-K(1,3)-G-G-P-S-S-P-Q-S-
L-R-H-Y-Y-N-W-L-T-R-Q-R-Y-NH2.C20DA-gGlu-gGlu-gGlu-gGlu-gGlu-gGlu-eLys(1,2)

MW (calculated): 6997.8 Da
Synthesis and purification methods: S02; P03
LCMS: A02-B; Rt: 10.4 min; m/3: 2333.6 m/4: 1750.6 m/5:

Compound 42
(SEQ ID NO: 368)
Y-Aib-E-G-T-F-T-S-D-Y-S-I-Aib-L-E-K-Q-A-Q-Aib-A-F-V-E-W-L-I-K(1,3)-G-G-P-S-E-Y-V-S-I-
R-H-Y-Y-N-W-L-T-R-Q-R-Y-NH2.C20DA-gGlu-gGlu-gGlu-gGlu-gGlu-gGlu-eLys(1,2)

MW (calculated): 7076.9 Da
Synthesis and purification methods: S02; P03
LCMS: A02-A; Rt: 7.7 min; m/3: 2360.0 m/4: 1770.6 m/5:

Compound 43
(SEQ ID NO: 369)
Y-Aib-E-G-T-F-T-S-D-Y-S-I-Aib-L-E-K-Q-A-Q-Aib-E-F-V-Q-W-L-I-K(1,3)-G-G-P-S-S-G-A-S-L-R-
H-Y-Y-N-W-L-T-R-Q-R-Y-NH2.C20DA-gGlu-gGlu-gGlu-gGlu-gGlu-gGlu-gGlu-eLys(1,2)

MW (calculated): 7086.9 Da
Synthesis and purification methods: S01; P02
LCMS: A01-A; Rt: 12.3 min; m/3: m/4: 1772.6 m/5:

Compound 44
(SEQ ID NO: 370)
Y-Aib-E-G-T-F-T-S-D-Y-S-I-Aib-L-E-K-Q-A-Q-Aib-A-F-V-E-W-L-I-K(1,3)-G-G-P-S-S-G-Q-S-Aib-
R-H-Y-Y-N-W-L-T-R-Q-R-Y-NH2.C20DA-gGlu-gGlu-gGlu-gGlu-gGlu-gGlu-eLys(1,2)

MW (calculated): 6929.7 Da
Synthesis and purification methods: S02; P03
LCMS: A02-A; Rt: 6.0 min; m/3: 2311.2 m/4: 1733.2 m/5:

Compound 45
(SEQ ID NO: 371)
Y-Aib-E-G-T-F-T-S-D-Y-S-I-Aib-L-E-E-Q-A-Q-Aib-A-F-V-Q-W-L-I-K(1,3)-G-G-P-S-S-G-A-S-
L-R-H-Y-Y-N-W-L-T-R-Q-R-Y-NH2.C20DA-gGlu-gGlu-gGlu-gGlu-gGlu-OEG-eLys(1,2)

MW (calculated): 6916.7 Da
Synthesis and purification methods: S01; P01
LCMS: A01-B; Rt: 36.2 min; m/3: m/4: 1729.8 m/5:

Compound 46
(SEQ ID NO: 372)
Y-Aib-E-G-T-F-T-S-D-Y-S-I-Aib-L-E-K-E-A-Q-Aib-K-F-V-E-W-L-I-K(1,3)-G-G-P-S-S-G-A-S-L-R-H-
Y-Y-N-W-L-T-R-Q-R-Y-NH2.C20DA-gGlu-gGlu-gGlu-gGlu-gGlu-gGlu-eLys(1,2)

MW (calculated): 6958.8 Da
Synthesis and purification methods: 501; P02
LCMS: A01-B; Rt: 34.6 min; m/3: m/4: 1740.5 m/5:

Compound 47
(SEQ ID NO: 373)
Y-Aib-E-G-T-F-T-S-D-Y-S-I-Aib-L-E-K-Q-A-Q-Aib-A-F-V-Q-W-L-I-K(1,3)-G-G-P-S-S-G-V-S-L-R-H-Y-
Y-N-W-L-T-R-Q-R-Y-NH2.C20DA-gGlu-gGlu-gGlu-gGlu-gGlu-gGlu-eLys(1,2)

MW (calculated): 6927.8 Da
Synthesis and purification methods: S02; P03
LCMS: A02-B; Rt: 10.7 min; m/3: 2310.9 m/4: 1733.2 m/5:

Compound 48
(SEQ ID NO: 374)
Y-Aib-E-G-T-F-T-S-D-Y-S-I-Aib-L-E-K-Q-A-Q-Aib-A-F-V-E-W-L-I-K(1,3)-G-
G-P-S-E-P-Q-S-I-R-H-Y-Y-N-W-L-T-R-Q-R-Y-NH2.C20DA-gGlu-gGlu-gGlu-gGlu-
gGlu-gGlu-eLys(1,2)

MW (calculated): 7039.9 Da
Synthesis and purification methods: S02; P03
LCMS: A02-B; Rt: 10.1 min; m/3: 2347.9 m/4: 1761.1 m/5:

Compound 49
(SEQ ID NO: 375)
Y-Aib-E-G-T-F-T-S-D-Y-S-I-Aib-L-E-K-Q-A-Q-Aib-A-F-V-E-W-L-I-K(1,3)-G-G-P-
S-S-G-E-S-Aib-R-H-Y-Y-N-W-L-T-R-Q-R-Y-NH2.C20DA-gGlu-gGlu-gGlu-gGlu-gGlu-
gGlu-eLys(1,2)

MW (calculated): 6930.7 Da
Synthesis and purification methods: S02; P03
LCMS: A02-A; Rt: 6.1 min; m/3: 2311.3 m/4: 1733.7 m/5:

Compound 50
(SEQ ID NO: 376)
Y-Aib-E-G-T-F-T-S-D-Y-S-I-Aib-L-E-K-Q-A-Q-Aib-A-F-V-E-W-L-I-K(1,3)-G-G-P-
S-S-Y-E-S-L-R-H-Y-Y-N-W-L-T-R-Q-R-Y-NH2.C20DA-gGlu-gGlu-gGlu-gGlu-gGlu-
gGlu-eLys(1,2)

MW (calculated): 7064.9 Da
Synthesis and purification methods: S02; P03
LCMS: A02-A; Rt: 7.4 min; m/3: 2356.2 m/4: 1767.5 m/5:

Compound 51
(SEQ ID NO: 377)
Y-Aib-E-G-T-F-T-S-D-Y-S-I-Aib-L-E-K-Q-A-E-Aib-A-F-V-Q-W-L-I-K(1,3)-G-G-P-S-S-G-
A-S-L-R-H-Y-Y-N-W-L-T-R-Q-R-Y-NH2.C18DA-gGlu-gGlu-gGlu-gGlu-gGlu-gGlu-eLys(1,2)

MW (calculated): 6872.7 Da
Synthesis and purification methods: S02; P03
LCMS: A02-A; Rt: 5.7 min; m/3: 2291.8 m/4: 1719.6 m/5:

Compound 52
(SEQ ID NO: 378)
Y-Aib-E-G-T-F-T-S-D-Y-S-I-Aib-L-E-K-Q-A-Q-Aib-A-F-V-Q-W-L-I-K(1,3)-G-
G-P-E-S-G-A-S-L-R-H-Y-Y-N-W-L-T-R-Q-R-Y-NH2.C18DA-E-E-E-E-OEG-OEG(1,2)

MW (calculated): 6817.6 Da
Synthesis and purification methods: S02; P03
LCMS: A02-B; Rt: 10.3 min; m/3: 2273.8 m/4: 1705.5 m/5:

Compound 53
(SEQ ID NO: 379)
Y-Aib-E-G-T-F-T-S-D-Y-S-I-Aib-L-E-K-Q-A-Q-Aib-A-F-V-Q-W-L-I-K(1,3)-G-
G-P-E-S-G-A-S-L-R-H-Y-Y-N-W-L-T-R-Q-R-Y-NH2.C18DA-gGlu-gGlu-gGlu-gGlu-
gGlu(1,2)

MW (calculated): 6656.4 Da
Synthesis and purification methods: S02; P03
LCMS: A02-A; Rt: 7.9 min; m/3: 2219.5 m/4: 1664.9 m/5:

Compound 54
(SEQ ID NO: 380)
Y-Aib-E-G-T-F-T-S-D-Y-S-I-Aib-L-E-K-Q-A-E-Aib-A-F-V-Q-W-L-I-K(1,3)-G-G-P-S-E-
P-A-S-L-R-H-Y-Y-N-W-L-T-R-Q-R-Y-NH2.C18DA-gGlu-gGlu-gGlu-gGlu-eLys(1,2)

MW (calculated): 6696.5 Da
Synthesis and purification methods: S02; P03
LCMS: A02-A; Rt: 5.9 min; m/3: 2232.8 m/4: 1675.3 m/5:

Compound 55
(SEQ ID NO: 381)
Y-Aib-E-G-T-F-T-S-D-Y-S-I-Aib-L-E-K-Q-A-Q-Aib-A-F-V-Q-W-L-I-K(1,3)-G-G-P-E-
S-G-A-S-L-R-H-Y-Y-N-W-L-T-R-Q-R-Y-NH2.C18DA-gGlu-gGlu-glu-gGlu-gGlu-OEG-
OEG(1,2)

MW (calculated): 6946.8 Da
Synthesis and purification methods: S02; P03
LCMS: A02-A; Rt: 7.1 min; m/3: 2316.4 m/4: 1737.6 m/5:

Compound 56
(SEQ ID NO: 382)
Y-Aib-E-G-T-F-T-S-D-Y-S-I-Aib-L-E-K-Q-A-Q-Aib-E-F-V-E-W-L-I-K(1,3)-G-G-P-
S-S-P-A-S-L-R-H-Y-Y-N-W-L-T-R-Q-R-Y-NH2.C18DA-gGlu-gGlu-gGlu-gGlu-OEG-
OEG(1,2)

MW (calculated): 6874.7 Da
Synthesis and purification methods: S02; P03
LCMS: A02-A; Rt: 7.2 min; m/3: 2292.4 m/4: 1719.8 m/5:

Compound 57
(SEQ ID NO: 383)
Y-Aib-E-G-T-F-T-S-D-Y-S-I-Aib-L-E-K-Q-A-Q-Aib-A-F-V-Q-W-L-I-K(1,3)-G-G-P-E-E-P-
A-S-L-R-H-Y-Y-N-W-L-T-R-Q-R-Y-NH2.C18DA-gGlu-gGlu-gGlu-gGlu-OEG-OEG(1,2)

MW (calculated): 6899.7 Da
Synthesis and purification methods: S02; P03
LCMS: A02-A; Rt: 7.1 min; m/3: 2300.8 m/4: 1725.9 m/5:

Compound 58
(SEQ ID NO: 384)
Y-Aib-E-G-T-F-T-S-D-Y-S-I-Aib-L-E-K-Q-A-Q-Aib-A-F-V-Q-W-L-I-K(1,3)-G-G-P-E-S-G-
A-S-L-R-H-Y-Y-N-W-L-T-R-Q-R-Y-NH2.C18DA-gGlu-gGlu-gGlu-gGlu-OEG-OEG(1,2)

MW (calculated): 6817.6 Da
Synthesis and purification methods: S02; P03
LCMS: A02-B; Rt: 10.0 min; m/3: 2273.4 m/4: 1705.5 m/5:

(SEQ ID NO: 385)
Y-Aib-E-G-T-F-T-S-D-Y-S-I-Aib-L-E-K-Q-A-Q-Aib-A-F-V-Q-W-L-I-K(1,3)-G-G-P-S-Y-Y-A-
S-L-R-H-Y-Y-N-W-L-T-R-Q-R-Y-NH2.C20DA-gGlu-gGlu-gGlu-Glu-gGlu-eLys(1,2)

MW (calculated): 6952.9 Da
Synthesis and purification methods: S02; P03
LCMS: A02-A; Rt: min; m/3: 2318.5 m/4: 1738.9 m/5:

Compound 60
(SEQ ID NO: 386)
Y-Aib-E-G-T-F-T-S-D-Y-S-I-Aib-L-E-K-Q-A-Q-Aib-A-F-V-Q-W-L-I-K(1,3)-G-G-P-E-S-G-A-
S-L-R-H-Y-Y-N-W-L-T-R-Q-R-Y-NH2.C18DA-gGlu-gGlu-gGlu-gGlu-gGlu-eLys(1,2)

MW (calculated): 6784.6 Da
Synthesis and purification methods: S02; P03
LCMS: A02-B; Rt: 9.2 min; m/3: 2262.5 m/4: 1697.3 m/5:

Compound 61
(SEQ ID NO: 387)
Y-Aib-E-G-T-F-T-S-D-Y-S-I-Aib-L-E-K-Q-A-Q-Aib-A-F-V-Q-W-L-I-K(1,3)-G-G-P-E-S-G-A-
S-L-R-H-Y-Y-N-W-L-T-R-Q-R-Y-NH2.C20DA-gGlu-gGlu-gGlu-gGlu-gGlu-eLys(1,2)

MW (calculated): 6812.7 Da
Synthesis and purification methods: S02; P03
LCMS: A02-A; Rt: 7.4 min; m/3: 2271.8 m/4: 1704.2 m/5:

Compound 62
(SEQ ID NO: 388)
Y-Aib-E-G-T-F-T-S-D-Y-S-I-Aib-L-E-K-Q-A-Q-Aib-A-F-V-E-W-L-I-K(1,3)-G-G-P-R-Y-Y-A-S-L-
R-H-Y-Y-N-W-L-T-R-Q-R-Y-NH2.C20DA-gGlu-gGlu-gGlu-gGlu-gGlu-gGlu-eLys(1,2)

MW (calculated): 7152.1 Da
Synthesis and purification methods: S01; P02
LCMS: A01-B; Rt: 35.2 min; m/3: m/4: 1788.8 m/5:

Compound 63
(SEQ ID NOS 389
Y-Aib-E-G-T-F-T-S-D-Y-S-I-Aib-L-E-K-Q-A-Q-Aib-A-F-V-Q-W-L-I-K(1,3)-G-G-P-E-S-G-A-S-L-
R-H-Y-Y-N-W-L-T-R-Q-R-Y-NH2.C18DA-E-E-E-E-E-eLys(1,2)

MW (calculated): 6784.6 Da
Synthesis and purification methods: S02; P03
LCMS: A02-B; Rt: 9.6 min; m/3: 2262.4 m/4: 1697.1 m/5:

Compound 64
(SEQ ID NO: 390)
Y-Aib-E-G-T-F-T-S-D-Y-S-I-Aib-L-E-K-Q-A-Q-Aib-A-F-V-E-W-L-I-K(1,3)-G-G-P-E-S-P-A-S-
L-R-H-Y-Y-N-W-L-T-R-Q-R-Y-NH2.C18DA-gGlu-gGlu-gGlu-Glu-OEG-OEG(1,2)

MW (calculated): 6858.7 Da
Synthesis and purification methods: S02; P03
LCMS: A02-A; Rt: 7.1 min; m/3: 2286.9 m/4: 1715.2 m/5:

Compound 65
(SEQ ID NO: 391)
Y-Aib-E-G-T-F-T-S-D-Y-S-I-Aib-L-E-K-Q-A-Q-Aib-A-F-V-E-W-L-I-K(1,3)-G-G-G-S-E-Y-V-
S-L-R-H-Y-Y-N-W-L-T-R-Q-R-Y-NH2.C20DA-gGlu-gGlu-gGlu-gGlu-gGlu-gGlu(1,2)

MW (calculated): 6908.7 Da
Synthesis and purification methods: S02; P03
LCMS: A02-A; Rt: 7.4 min; m/3: 2345.9 m/4: 1760.1 m/5:

Compound 66
(SEQ ID NO: 392)
Y-Aib-E-G-T-F-T-S-D-Y-S-I-Aib-L-E-E-E-A-Q-Aib-A-F-V-E-W-L-I-K(1,3)-G-G-P-S-S-G-
A-S-L-R-H-Y-Y-N-W-L-T-R-Q-R-Y-NH2.C20DA-gGlu-gGlu-gGlu-gGlu-gGlu-gGlu-eLys(1,2)

MW (calculated): 6902.7 Da
Synthesis and purification methods: 501; P02
LCMS: A01-A; Rt: 14.1 min; m/3: m/4: 1726.5 m/5:

Compound 67
(SEQ ID NO: 393)
Y-Aib-E-G-T-F-T-S-D-Y-S-I-Aib-L-E-K-Q-A-Q-Aib-A-F-V-Q-W-L-I-K(1,3)-G-G-P-E-E-P-A-
S-L-R-H-Y-Y-N-W-L-T-R-Q-R-Y-NH2.C20DA-gGlu-gGlu-gGlu-gGlu-gGlu-eLys(1,2)

MW (calculated): 6894.8 Da
Synthesis and purification methods: S02; P03
LCMS: A02-A; Rt: 7.1 min; m/3: 2298.9 m/4: 1724.2 m/5:

Compound 68
(SEQ ID NO: 394)
Y-Aib-E-G-T-F-T-S-D-Y-S-I-Aib-L-E-K-Q-A-Q-Aib-A-F-V-E-W-L-I-K(1,3)-G-G-P-S-S-G-A-S-
L-R-H-Y-Y-N-R-Aib-T-R-Q-R-Y-NH2.C20DA-gGlu-gGlu-gGlu-gGlu-gGlu-gGlu-eLys(1,2)

MW (calculated): 6842.7 Da
Synthesis and purification methods: 501; P02
LCMS: A01-B; Rt: 34.3 min; m/3: m/4: 1711.4 m/5:

Compound 69
(SEQ ID NO: 395)
Y-Aib-E-G-T-F-T-S-D-Y-S-I-Aib-L-E-K-E-K-Q-Aib-A-F-V-E-W-L-I-K(1,3)-G-G-P-S-S-G-A-S-L-R-
H-Y-Y-N-W-L-T-R-Q-R-Y-NH2.C20DA-gGlu-gGlu-gGlu-gGlu-gGlu-gGlu-eLys(1,2)

MW (calculated): 6958.8 Da
Synthesis and purification methods: S01; P02
LCMS: A01-B; Rt: 33.8 min; m/3: m/4: 1740.4 m/5:

Compound 70
(SEQ ID NO: 396)
Y-Aib-E-G-T-F-T-S-D-Y-S-I-Aib-L-E-K-Q-A-Q-Aib-A-F-V-E-W-L-I-K(1,3)-G-G-P-E-S-P-A-S-L-R-
H-Y-Y-N-W-L-T-R-Q-R-Y-NH2.C20DA-gGlu-gGlu-gGlu-gGlu-gGlu-eLys(1,2)

MW (calculated): 6853.7 Da
Synthesis and purification methods: S02; P03
LCMS: A02-A; Rt: 7.3 min; m/3: 2284.9 m/4: 1714.4 m/5:

Compound 71
(SEQ ID NO: 397)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-H-
Y-Y-N-W-L-T-hArg-Q-R-DTyr-NH2.C20DA-gGlu-gGlu-gGlu-gGlu-gGlu-gGlu-eLys(1,2)

MW (calculated): 6914.8 Da
Synthesis and purification methods: S01; P02
LCMS: A01-B; Rt: 34.7 min; m/3: m/4: 1729.4 m/5:

Compound 72
(SEQ ID NO: 398)
Y-Aib-E-G-T-F-T-S-D-Y-S-I-Aib-L-E-K-Q-A-Q-Aib-A-F-V-E-W-L-I-K(1,3)-G-G-P-S-S-G-A-D-L-R-H-
Y-L-N-W-L-T-R-Q-R-Y-NH2.C20DA-gGlu-gGlu-gGlu-gGlu-gGlu-gGlu-eLys(1,2)

MW (calculated): 6878.7 Da
Synthesis and purification methods: S01; P01
LCMS: A01-B; Rt: 36.2 min; m/3: m/4: 1720.4 m/5:

Compound 73
(SEQ ID NO: 399)
Y-Aib-E-G-T-F-T-S-D-Y-S-I-Aib-L-E-K-Q-A-Q-Aib-A-F-V-E-W-L-I-K(1,3)-G-G-P-S-S-P-E-S-L-
R-H-Y-Y-N-W-L-T-R-Q-R-Y-NH2.C20DA-gGlu-gGlu-gGlu-gGlu-gGlu-gGlu-eLys(1,2)

MW (calculated): 6998.8 Da
Synthesis and purification methods: S02; P03
LCMS: A02-A; Rt: 6.3 min; m/3: 2334.1 m/4: 1751.0 m/5:

Compound 74
(SEQ ID NO: 400)
Y-Aib-E-G-T-F-T-S-D-Y-S-I-Aib-L-E-K-Q-A-Q-Aib-A-F-V-E-W-L-I-K(1,3)-G-G-P-S-S-G-A-S-
Aib-R-H-Y-Y-N-W-L-T-R-Q-R-DTyr-NH2.C20DA-gGlu-gGlu-gGlu-gGlu-gGlu-gGlu-eLys(1,2)

MW (calculated): 6872.7 Da
Synthesis and purification methods: S01; P01
LCMS: A01-B; Rt: 34.3 min; m/3: m/4: 1718.8 m/5:

Compound 75
(SEQ ID NO: 401)
Y-Aib-E-G-T-F-T-S-D-Y-S-I-Aib-L-E-K-Q-A-Q-Aib-A-F-V-E-W-L-I-K(1,3)-G-G-P-S-Y-
Y-V-S-L-R-H-Y-Y-N-W-L-T-R-Q-R-Y-NH2.C20DA-gGlu-gGlu-gGlu-gGlu-Glu-eLys(1,2)

MW (calculated): 6981.9 Da
Synthesis and purification methods: S02; P03
LCMS: A02-A; Rt: 7.6 min; m/3: 2328.1 m/4: 1746.1 m/5:

Compound 76
(SEQ ID NO: 402)
Y-Aib-E-G-T-F-T-S-D-Y-S-I-Aib-L-E-K-Q-A-Q-Aib-A-F-V-E-W-L-I-K(1,3)-G-G-P-S-S-G-A-S-Aib-R-
H-Y-Aib-N-W-L-T-R-Q-R-Y-NH2.C20DA-gGlu-gGlu-Glu-gGlu-gGlu-gGlu-eLys(1,2)

MW (calculated): 6794.6 Da
Synthesis and purification methods: 501; P02
LCMS: A01-B; Rt: 34.9 min; m/3: m/4: 1699.4 m/5:

Compound 77
(SEQ ID NO: 403)
Y-Aib-E-G-T-F-T-S-D-Y-S-I-Aib-L-E-K-Q-A-Q-Aib-A-F-V-Q-W-L-I-K(1,3)-G-G-P-E-E-P-A-S-L-R-H-
Y-Y-N-W-L-T-R-Q-R-Y-NH2.C20DA-gGlu-gGlu-gGlu-gGlu-OEG-OEG(1,2)

MW (calculated): 6927.8 Da
Synthesis and purification methods: S02; P03
LCMS: A02-A; Rt: 7.8 min; m/3: 2310.2 m/4: 1733.1 m/5:

Compound 78
(SEQ ID NO: 404)
Y-Aib-E-G-T-F-T-S-D-Y-S-I-Aib-L-E-K-Q-A-Q-Aib-A-F-V-Q-W-L-I-K(1,3)-G-G-P-S-E-Y-V-S-L-R-H-
Y-Y-N-W-L-T-R-Q-R-Y-NH2.C20DA-gGlu-gGlu-gGlu-gGlu-gGlu-gGlu-eLys(1,2)

MW (calculated): 7076.0 Da
Synthesis and purification methods: S02; P03
LCMS: A02-A; Rt: 7.4 min; m/3: 2358.9 m/4: 1769.7 m/5:

Compound 79
(SEQ ID NO: 405)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-H-
Y-Y-N-L-Aib-T-R-Q-R-Y-NH2.C20DA-gGlu-gGlu-gGlu-gGlu-gGlu-gGlu-eLys(1,2)

MW (calculated): 6799.6 Da
Synthesis and purification methods: 501; P02
LCMS: A01-B; Rt: 34.9 min; m/3: m/4: 1700.6 m/5:

Compound 80
(SEQ ID NO: 406)
Y-Aib-E-G-T-F-T-S-D-Y-S-I-Aib-L-E-K-Q-A-Q-Aib-A-F-V-E-W-L-I-K(1,3)-G-G-P-S-Y-Y-V-S-L-R-H-
Y-Y-N-W-L-T-R-Q-R-Y-NH2.C20DA-gGlu-gGlu-gGlu-gGlu-gGlu-gGlu-eLys(1,2)

MW (calculated): 7111.0 Da
Synthesis and purification methods: S02; P03
LCMS: A02-B; Rt: 9.7 min; m/3: 2370.9 m/4: 1778.5 m/5:

Compound 81
(SEQ ID NO: 407)
Y-Aib-E-G-T-F-T-S-D-Y-S-I-Aib-L-E-K-Q-A-Q-Aib-E-F-V-E-W-L-I-K(1,3)-G-G-P-E-S-G-A-S-L-R-H-
Y-Y-N-W-L-T-R-Q-R-Y-NH2.C18DA-gGlu-gGlu-gGlu-OEG-OEG(1,2)

MW (calculated): 6747.5 Da
Synthesis and purification methods: S02; P03
LCMS: A02-A; Rt: 6.1 min; m/3: 2250.8 m/4: 1688.3 m/5:

Compound 82
(SEQ ID NO: 408)
Y-Aib-E-G-T-F-T-S-D-Y-S-I-Aib-L-E-K-Q-A-Q-Aib-A-F-V-E-W-L-I-K(1,3)-G-G-P-E-S-P-A-S-L-R-H-
Y-Y-N-W-L-T-R-Q-R-Y-NH2.C20DA-gGlu-gGlu-gGlu-gGlu-OEG-OEG(1,2)

MW (calculated): 6886.7 Da
Synthesis and purification methods: S02; P03
LCMS: A02-B; Rt: 11.4 min; m/3: 2296.2 m/4: 1722.4 m/5:

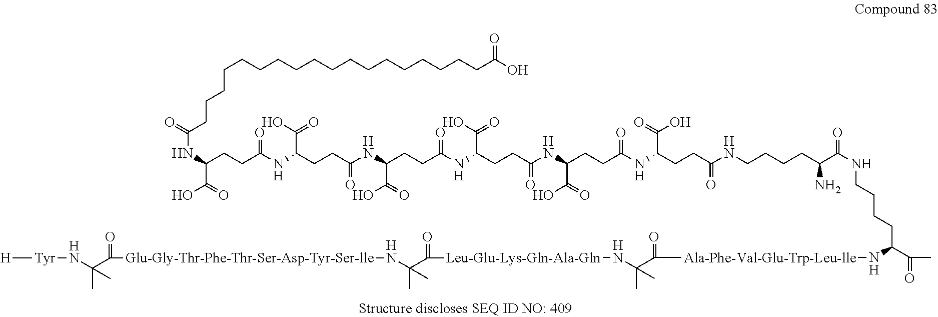

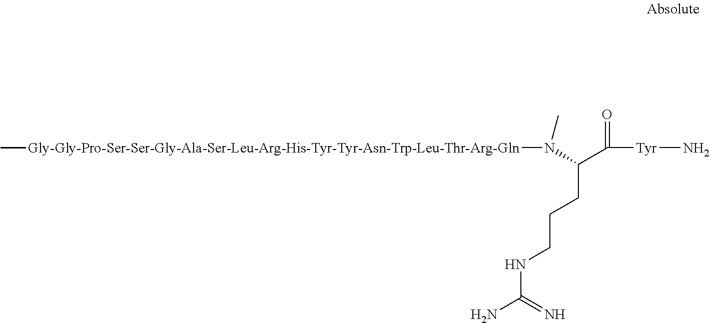

(SEQ ID NO: 409)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-H-
Y-Y-N-W-L-T-R-Q-NMeR-Y-NH2.C20DA-gGlu-gGlu-gGlu-gGlu-gGlu-gGlu-eLys(1,2)

MW (calculated): 6914.8 Da
Synthesis and purification methods: 501; P02
LCMS: A01-B; Rt: 35.0 min; m/3: m/4: 1729.4 m/5:

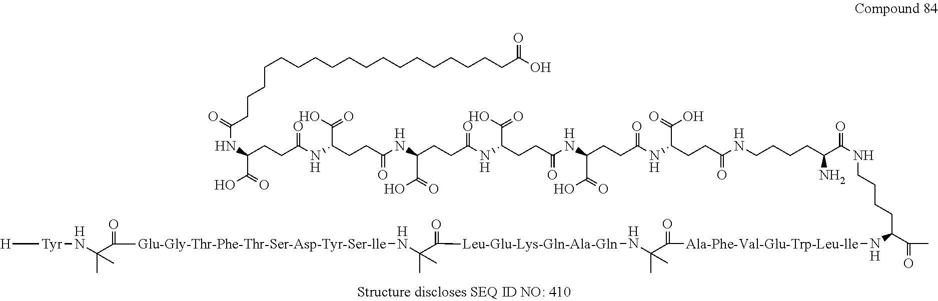

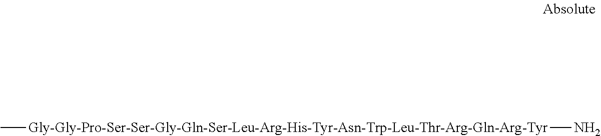

(SEQ ID NO: 410)
Y-Aib-E-G-T-F-T-S-D-Y-S-I-Aib-L-E-K-Q-A-Q-Aib-A-F-V-E-W-L-I-K(1,3)-G-G-P-S-S-G-Q-S-L-R-H-Y-
Y-N-W-L-T-R-Q-R-Y-NH2.C20DA-gGlu-gGlu-gGlu-gGlu-gGlu-gGlu-eLys(1,2)

MW (calculated): 6957.8 Da
Synthesis and purification methods: S02; P03
LCMS: A02-A; Rt: 7.1 min; m/3: 2320.5 m/4: 1740.5 m/5:

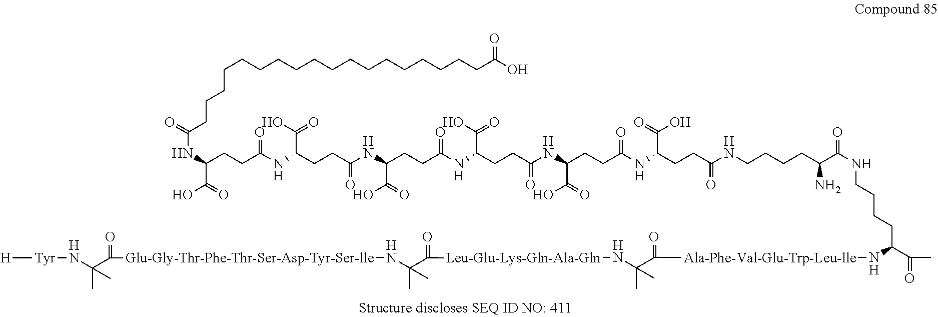

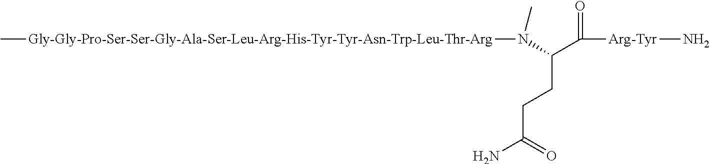

(SEQ ID NO: 411)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-H-Y-
Y-N-W-L-T-R-NMeQ-R-Y-NH2.C20DA-gGlu-gGlu-gGlu-gGlu-gGlu-gGlu-eLys(1,2)

MW (calculated): 6914.8 Da
Synthesis and purification methods: 501; P02
LCMS: A01-A; Rt: 12.7 min; m/3: m/4: m/5: 1383.8

Compound 86
(SEQ ID NO: 412)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-H-Y-
L-N-W-L-T-R-Q-R-Y-NH2.C20DA-gGlu-gGlu-gGlu-gGlu-gGlu-gGlu-eLys(1,2)

MW (calculated): 6850.7 Da
Synthesis and purification methods: 501; P02
LCMS: A01-B; Rt: 35.9 min; m/3: m/4: 1713.5 m/5:

Compound 87
(SEQ ID NO: 413)
Y-Aib-D-G-T-F-T-S-D-Y-S-I-Aib-L-E-K-Q-A-Q-Aib-A-F-V-E-W-L-I-K(1,3)-G-G-P-S-S-G-A-S-L-R-
H-Y-Y-N-W-L-T-R-Q-R-Y-NH2.C20DA-gGlu-gGlu-gGlu-gGlu-gGlu-gGlu-eLys(1,2)

MW (calculated): 6886.7 Da
Synthesis and purification methods: 501; P01
LCMS: A01-B; Rt: 35.4 min; m/3: m/4: 1722.4 m/5:

Compound 88
(SEQ ID NO: 414)
Y-Aib-E-G-T-F-T-S-D-Y-S-I-Aib-L-E-K-Q-A-Q-Aib-E-F-V-E-W-L-I-K(1,3)-G-G-P-S-S-G-A-S-L-R-
H-Y-Y-N-W-L-T-R-Q-R-Y-NH2.C20DA-gGlu-gGlu-gGlu-gGlu-gGlu-gGlu-gGlu-eLys(1,2)

MW (calculated): 7087.9 Da
Synthesis and purification methods: 501; P02
LCMS: A01-A; Rt: 12.4 min; m/3: m/4: 1772.7 m/5:

Compound 89
(SEQ ID NO: 415)
Y-Aib-E-G-T-F-T-S-D-Y-S-I-Aib-L-E-K-Q-A-Q-Aib-A-F-V-Q-W-L-I-K(1,3)-G-G-P-E-E-G-A-S-L-
R-H-Y-Y-N-W-L-T-R-Q-R-Y-NH2.C20DA-gGlu-gGlu-gGlu-gGlu-gGlu-gGlu-eLys(1,2)

MW (calculated): 6983.8 Da
Synthesis and purification methods: S02; P03
LCMS: A02-B; Rt: 10.6 min; m/3: 2328.9 m/4: 1746.9 m/5:

Compound 90
(SEQ ID NO: 416)
Y-Aib-E-G-T-F-T-S-D-Y-S-I-Aib-L-E-D-Q-A-Q-Aib-A-F-V-E-W-L-I-K(1,3)-G-G-P-S-S-G-A-S-L-
R-H-Y-Y-N-W-L-T-R-Q-R-Y-NH2.C20DA-gGlu-gGlu-gGlu-gGlu-gGlu-gGlu-eLys(1,2)

MW (calculated): 6887.6 Da
Synthesis and purification methods: 501; P02
LCMS: A01-B; Rt: 36.3 min; m/3: m/4: 1722.7 m/5:

Compound 91
(SEQ ID NO: 417)
Y-Aib-E-G-T-F-T-S-D-Y-S-I-Aib-L-E-K-Q-A-Q-Aib-A-F-V-E-W-L-I-K(1,3)-G-G-P-S-S-G-A-T-L-
R-H-Y-Y-N-W-L-T-R-Q-R-Y-NH2.C20DA-gGlu-gGlu-gGlu-gGlu-gGlu-gGlu-eLys(1,2)

MW (calculated): 6914.8 Da
Synthesis and purification methods: S02; P03
LCMS: A02-A; Rt: 7.4 min; m/3: 2306.6 m/4: 1730.1 m/5:

Compound 92
(SEQ ID NO: 418)
Y-Aib-E-G-T-F-T-S-D-Y-S-I-Aib-L-E-K-Q-A-Q-Aib-A-F-V-Q-W-L-I-K(1,3)-G-G-P-S-S-G-A-S-L-
R-H-Y-Y-N-W-L-T-R-Q-R-Y-NH2.C20DA-gGlu-gGlu-gGlu-Glu-gGlu-gGlu-gGlu-eLys(1,2)

MW (calculated): 7028.9 Da
Synthesis and purification methods: S02; P03
LCMS: A02-A; Rt: 7.3 min; m/3: 2343.6 m/4: 1758.1 m/5:

Compound 93
(SEQ ID NO: 419)
Y-Aib-E-G-T-F-T-S-D-Y-S-I-Aib-L-E-K-Q-A-Q-Aib-A-F-V-E-W-L-I-K(1,3)-G-G-P-S-E-Y-E-S-L-
R-H-Y-Y-N-W-L-T-R-Q-R-Y-NH2.C20DA-gGlu-gGlu-gGlu-gGlu-gGlu-gGlu-eLys(1,2)

MW (calculated): 7106.9 Da
Synthesis and purification methods: S02; P03
LCMS: A02-B; Rt: 10.8 min; m/3: 2370.5 m/4: 1777.8 m/5:

Compound 94
(SEQ ID NO: 420)
Y-Aib-E-G-T-F-T-S-D-Y-S-I-Aib-L-E-K-Q-A-Q-Aib-A-F-V-E-W-L-I-K(1,3)-G-G-P-S-S-G-I-S-L-R-
H-Y-Y-N-W-L-T-R-Q-R-Y-NH2.C20DA-gGlu-gGlu-gGlu-gGlu-gGlu-gGlu-eLys(1,2)

MW (calculated): 6942.8 Da
Synthesis and purification methods: S02; P03
LCMS: A02-B; Rt: 11.1 min; m/3: 2315.9 m/4: 1737.1 m/5:

Compound 95
(SEQ ID NO: 421)
Y-Aib-E-G-T-F-T-S-D-Y-S-I-Aib-L-E-K-Q-A-Q-Aib-A-F-V-E-W-L-I-K(1,3)-G-G-P-S-S-P-A-S-L-R-H-
Y-Y-N-W-L-T-R-Q-R-Y-NH2.C20DA-gGlu-gGlu-gGlu-gGlu-gGlu-gGlu-eLys(1,2)

MW (calculated): 6940.8 Da
Synthesis and purification methods: 501; P02
LCMS: A01-B; Rt: 34.8 min; m/3: m/4: 1735.9 m/5:

(SEQ ID NO: 422)
Compound 96
Y-Aib-E-G-T-F-T-S-D-Y-S-I-Aib-L-E-K-Q-A-Q-Aib-A-F-V-E-W-L-I-K(1,3)-G-G-P-S-S-G-V-S-L-R-H-
Y-Y-N-W-L-T-R-Q-R-Y-NH2.C20DA-gGlu-gGlu-gGlu-gGlu-gGlu-gGlu-eLys(1,2)

MW (calculated): 6928.8 Da
Synthesis and purification methods: S02; P03
LCMS: A02-A; Rt: 7.3 min; m/3: 2310.1 m/4: 1732.7 m/5:

Compound 97
(SEQ ID NO: 423)
Y-Aib-E-G-T-F-T-S-D-Y-S-I-Aib-L-E-E-Q-A-Q-Aib-A-F-V-E-W-L-I-K(1,3)-G-G-P-S-S-G-A-S-L-R-H-
Y-Y-N-W-L-T-R-Q-R-Y-NH2.C20DA-gGlu-gGlu-gGlu-gGlu-eLys(1,2)

MW (calculated): 6643.4 Da
Synthesis and purification methods: S01; P01
LCMS: A01-B; Rt: 36.9 min; m/3: m/4: 1661.5 m/5:

Compound 98
(SEQ ID NO: 424)
Y-Aib-E-G-T-F-T-S-D-Y-S-I-Aib-L-E-K-Q-A-Q-Aib-A-F-V-E-W-L-I-K(1,3)-G-G-P-S-S-A-A-S-L-R-
H-Y-Y-N-W-L-T-R-Q-R-Y-NH2.C20DA-gGlu-gGlu-gGlu-gGlu-gGlu-gGlu-eLys(1,2)

MW (calculated): 6914.8 Da
Synthesis and purification methods: S02; P03
LCMS: A02-B; Rt: 11.0 min; m/3: 2306.3 m/4: 1730.1 m/5:

Compound 99
(SEQ ID NO: 425)
Y-Aib-E-G-T-F-T-S-D-Y-S-I-Aib-L-E-G-Q-A-Q-Aib-A-F-V-E-W-L-I-K(1,3)-G-G-P-S-S-G-A-S-L-R-H-
Y-Y-N-W-L-T-R-Q-R-Y-NH2.C20DA-gGlu-gGlu-gGlu-gGlu-gGlu-gGlu-eLys(1,2)

MW (calculated): 6829.6 Da
Synthesis and purification methods: S01; P02
LCMS: A01-B; Rt: 36.8 min; m/3: m/4: 1708.1 m/5:

Compound 100
(SEQ ID NO: 426)
Y-Aib-E-G-T-F-T-S-D-Y-S-I-Aib-L-E-E-Q-A-Q-Aib-A-F-V-E-W-L-I-K(1,3)-G-G-P-S-S-G-A-S-L-R-
H-Y-Y-N-W-L-T-R-Q-R-Y-NH2.C20DA-gGlu-gGlu-glu-gGlu-OEG-OEG-eLys(1,2)

MW (calculated): 6933.8 Da
Synthesis and purification methods: S01; P01
LCMS: A01-B; Rt: 36.5 min; m/3: m/4: 1734.0 m/5:

Compound 101
(SEQ ID NO: 427)
Y-Aib-E-G-T-F-T-S-D-Y-S-I-Aib-L-E-K-Q-A-Q-Aib-A-F-V-Q-W-L-I-K(1,3)-G-G-P-S-E-P-A-S-L-R-
H-Y-Y-N-W-L-T-R-Q-R-Y-NH2.C20DA-gGlu-gGlu-gGlu-gGlu-gGlu-Glu-eLys(1,2)

MW (calculated): 6981.8 Da
Synthesis and purification methods: S01; L20
LCMS: A01-B; Rt: 7.4 min; m/3: 2328.2 m/4: 1746.3 m/5:

Compound 102
(SEQ ID NO: 428)
Y-Aib-E-G-T-F-T-S-D-Y-S-I-Aib-L-E-A-Q-A-Q-Aib-A-F-V-E-W-L-I-K(1,3)-G-G-P-S-S-G-A-S-L-R-
H-Y-Y-N-W-L-T-R-Q-R-Y-NH2.C20DA-gGlu-gGlu-gGlu-gGlu-gGlu-gGlu-eLys(1,2)

MW (calculated): 6843.6 Da
Synthesis and purification methods: S01; P02
LCMS: A01-B; Rt: 37.5 min; m/3: m/4: m/5: 1369.4

Compound 103
(SEQ ID NO: 429)
Y-Aib-E-G-T-F-T-S-D-Y-S-I-Aib-L-E-K-Q-A-Q-Aib-A-F-V-E-W-L-I-K(1,3)-G-G-P-S-S-G-A-S-Tle-
R-H-Y-Y-N-W-L-T-R-Q-R-Y-NH2.C20DA-gGlu-gGlu-gGlu-gGlu-gGlu-gGlu-eLys(1,2)

MW (calculated): 6900.7 Da
Synthesis and purification methods: 501; P01
LCMS: A01-B; Rt: 34.9 min; m/3: m/4: m/5: 1380.9

Compound 104
(SEQ ID NO: 430)
Y-Aib-E-G-T-F-T-S-D-Y-S-I-L-L-E-K-Q-A-Q-Aib-A-F-V-E-W-L-I-K(1,3)-G-G-P-S-S-G-A-S-L-R-H-Y-
Y-N-W-L-T-R-Q-R-Y-NH2.C20DA-gGlu-Glu-gGlu-gGlu-gGlu-gGlu-eLys(1,2)

MW (calculated): 6928.8 Da
Synthesis and purification methods: 501; P02
LCMS: A01-B; Rt: 35.4 min; m/3: m/4: 1732.9 m/5:

Compound 105
(SEQ ID NO: 431)
Y-Aib-E-G-T-F-T-S-D-Y-S-I-Aib-L-E-E-Q-A-Q-Aib-A-F-V-E-W-L-I-K(1,3)-G-G-P-S-S-G-A-S-L-R-H-
Y-Y-N-W-L-T-R-Q-R-Y-NH2.C20DA-gGlu-gGlu-gGlu-Glu-gGlu-eLys-eLys(1,2)

MW (calculated): 6900.7 Da
Synthesis and purification methods: 501; P02
LCMS: A01-B; Rt: 35.9 min; m/3: m/4: 1725.9 m/5:

Compound 106
(SEQ ID NO: 432)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-H-
Y-I-N-W-L-T-R-Q-R-Y-NH2.C20DA-gGlu-gGlu-gGlu-gGlu-gGlu-gGlu-eLys(1,2)

MW (calculated): 6850.7 Da
Synthesis and purification methods: 501; P01
LCMS: A01-B; Rt: 35.9 min; m/3: m/4: m/5: 1370.8

Compound 107
(SEQ ID NO: 433)
Y-Aib-E-G-T-F-T-S-D-Y-S-I-Aib-L-E-K-Q-A-Q-Aib-A-F-V-E-W-L-I-K(1,3)-G-G-P-S-S-G-L-S-L-R-
H-Y-Y-N-W-L-T-R-Q-R-Y-NH2.C20DA-gGlu-gGlu-gGlu-gGlu-gGlu-gGlu-eLys(1,2)

MW (calculated): 6942.8 Da
Synthesis and purification methods: 501; P02
LCMS: A01-B; Rt: 35.7 min; m/3: m/4: 1736.4 m/5:

Compound 108
(SEQ ID NO: 434)
Y-Aib-E-G-T-F-T-S-D-Y-S-I-Aib-L-E-K-Q-A-Q-Aib-A-F-V-Q-W-L-I-K(1,3)-G-G-P-E-E-G-A-S-L-R-H-Y-
Y-N-W-L-T-R-Q-R-Y-NH2.C20DA-gGlu-gGlu-gGlu-gGlu-Glu-eLys(1,2)

MW (calculated): 6854.7 Da
Synthesis and purification methods: S01; P02
LCMS: A01-A; Rt: 12.9 min; m/3: m/4: 1714.4 m/5:

Compound 109
(SEQ ID NO: 435)
Y-Aib-E-G-T-F-T-S-D-Y-S-I-Aib-L-E-K-Q-A-Q-Aib-A-F-V-E-W-L-L-K(1,3)-G-G-P-S-S-G-A-S-L-R-H-Y-
Y-N-W-L-T-R-Q-R-Y-NH2.C20DA-gGlu-gGlu-glu-gGlu-gGlu-Glu-eLys(1,2)

MW (calculated): 6900.7 Da
Synthesis and purification methods: S01; P01
LCMS: A01-B; Rt: 35.2 min; m/3: m/4: 1725.9 m/5:

Compound 110
(SEQ ID NO: 436)
Y-Aib-E-G-T-F-T-S-D-Y-S-I-Aib-L-E-E-Q-A-Q-Aib-A-F-V-E-W-L-I-K(1,3)-G-G-P-S-S-G-A-S-L-R-H-
Y-Y-N-W-L-T-R-Q-R-Y-NH2.C20DA-gGlu-gGlu-gGlu-gGlu-gGlu-OEG-eLys(1,2)

MW (calculated): 6917.7 Da
Synthesis and purification methods: S01; P01
LCMS: A01-B; Rt: 36.5 min; m/3: m/4: 1730.1 m/5:

Compound 111
(SEQ ID NO: 437)
Y-Aib-E-G-T-F-T-S-D-Y-S-I-Aib-L-E-K-Q-A-Q-Aib-A-F-V-Q-W-L-I-K(1,3)-G-G-P-S-E-P-A-S-L-R-H-Y-
Y-N-W-L-T-R-Q-R-Y-NH2.C20DA-gGlu-gGlu-gGlu-gGlu-gGlu-OEG-OEG(1,2)

MW (calculated): 7014.9 Da
Synthesis and purification methods: S02; P03
LCMS: A02-B; Rt: 9.8 min; m/3: 2339.5 m/4: 1754.9 m/5:

Compound 112
(SEQ ID NO: 438)
Y-Aib-E-G-T-F-T-S-D-Y-S-I-Aib-L-E-K-Q-A-Q-Aib-A-F-V-Q-W-L-I-K(1,3)-G-G-P-S-S-G-A-S-L-R-H-Y-
Y-N-W-L-T-R-Q-R-Y-NH2.C20DA-gGlu-gGlu-gGlu-gGlu-gGlu-OEG-OEG(1,2)

MW (calculated): 6932.8 Da
Synthesis and purification methods: S02; P03
LCMS: A02-A; Rt: 7.8 min; m/3: 2312.2 m/4: 1734.1 m/5:

Compound 113
(SEQ ID NO: 439)
Y-Aib-E-G-T-F-T-S-D-L-S-I-Aib-L-E-K-Q-A-Q-Aib-A-F-V-Q-W-L-I-K(1,3)-G-G-P-S-E-G-A-S-L-R-H-Y-Y-
N-W-L-T-R-Q-R-Y-NH2.C20DA-gGlu-gGlu-gGlu-Glu-gGlu-gGlu-eLys(1,2)

MW (calculated): 6891.8 Da
Synthesis and purification methods: S01; P02
LCMS: A01-A; Rt: 13.7 min; m/3: m/4: 1723.7 m/5:

Compound 114
(SEQ ID NO: 440)
Y-Aib-E-G-T-F-T-S-D-Y-S-I-Aib-L-E-K-Q-A-Q-Aib-A-F-V-Q-W-L-I-K(1,3)-G-G-P-S-E-G-V-S-L-R-H-Y-
Y-N-W-L-T-R-Q-R-Y-NH2.C20DA-gGlu-gGlu-gGlu-gGlu-gGlu-gGlu-eLys(1,2)

MW (calculated): 6969.8 Da
Synthesis and purification methods: S02; P03
LCMS: A02-A; Rt: 7.3 min; m/3: 2323.6 m/4: 1743.1 m/5:

Compound 115
(SEQ ID NO: 441)
Y-Aib-E-G-T-F-T-S-D-Y-S-I-Aib-L-E-K-Q-A-Q-Aib-A-F-V-Q-W-L-I-K(1,3)-G-G-P-S-E-G-A-S-L-R-H-Y-
Y-N-W-L-T-R-NMeQ-R-Y-NH2.C20DA-gGlu-gGlu-gGlu-gGlu-gGlu-gGlu-eLys(1,2)

MW (calculated): 6955.8 Da
Synthesis and purification methods: 501; P02
LCMS: A01-A; Rt: 12.8 min; m/3: m/4: 1739.6 m/5:

Compound 116
(SEQ ID NO: 442)
Y-Aib-E-G-T-F-T-S-D-Y-S-I-Aib-L-E-E-Q-A-Q-Aib-A-F-V-E-W-L-I-K(1,3)-G-G-P-S-S-G-A-S-L-R-H-Y-
Y-N-W-L-T-R-Q-R-Y-NH2.C20DA-gGlu-gGlu-gGlu-gGlu-gGlu-gGlu-eLys(1,2)

MW (calculated): 6901.7 Da
Synthesis and purification methods: 501; P02
LCMS: A01-B; Rt: 36.7 min; m/3: m/4: 1726.2 m/5:

Compound 117
(SEQ ID NO: 443)
Y-Aib-E-G-T-F-T-S-D-Y-S-I-Aib-L-E-K-Q-A-Q-Aib-A-F-V-E-W-L-I-K(1,3)-G-G-P-S-S-G-A-S-V-R-H-
Y-Y-N-W-L-T-R-Q-R-Y-NH2.C20DA-gGlu-gGlu-gGlu-gGlu-gGlu-gGlu-eLys(1,2)

MW (calculated): 6886.7 Da
Synthesis and purification methods: S02; P03
LCMS: A02-A; Rt: 7.2 min; m/3: 2297.3 m/4: 1722.9 m/5:

Compound 118
(SEQ ID NO: 444)
Y-Aib-E-G-T-F-T-S-D-Y-S-I-Aib-L-E-K-Q-A-Q-Aib-A-F-V-E-W-L-I-K(1,3)-G-G-P-S-S-G-A-S-Aib-
R-H-Y-Y-N-W-L-T-R-Q-R-Y-NH2.C20DA-gGlu-gGlu-gGlu-gGlu-gGlu-gGlu-eLys(1,2)

MW (calculated): 6872.7 Da
Synthesis and purification methods: 501; P02
LCMS: A01-B; Rt: 34.5 min; m/3: m/4: 1718.9 m/5:

Compound 119
(SEQ ID NO: 445)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-
Tle-Y-Y-N-W-L-T-R-Q-R-Y-NH2.C20DA-gGlu-gGlu-gGlu-gGlu-gGlu-gGlu-eLys(1,2)

MW (calculated): 6876.7 Da
Synthesis and purification methods: 501; P01
LCMS: A01-B; Rt: 36.0 min; m/3: m/4: m/5: 1376.1

Compound 120
(SEQ ID NO: 446)
Y-Aib-E-G-T-F-T-S-D-Y-S-I-Aib-L-E-K-Q-A-Q-Aib-A-F-V-E-W-L-I-K(1,3)-G-G-P-S-S-P-A-S-L-R-H-Y-
Y-N-W-L-T-R-Q-R-Y-NH2.C20DA-gGlu-gGlu-gGlu-gGlu-gGlu-OEG-OEG(1,2)

MW (calculated): 6973.8 Da
Synthesis and purification methods: S02; P03
LCMS: A02-A; Rt: 7.9 min; m/3: 2325.1 m/4: 1744.6 m/5:

Compound 121
(SEQ ID NO: 447)
Y-Aib-E-G-T-F-T-S-D-Y-S-I-Aib-L-E-S-Q-A-Q-Aib-A-F-V-E-W-L-I-K(1,3)-G-G-P-S-S-G-A-S-L-R-H-
Y-Y-N-W-L-T-R-Q-R-Y-NH2.C20DA-gGlu-gGlu-gGlu-gGlu-gGlu-gGlu-eLys(1,2)

MW (calculated): 6859.6 Da
Synthesis and purification methods: S01; P02
LCMS: A01-B; Rt: 39.8 min; m/3: m/4: 1715.6 m/5:

Compound 122
(SEQ ID NO: 448)
Y-Aib-E-G-T-F-T-S-D-Y-S-I-Aib-L-E-K-Q-A-Q-Aib-A-F-V-E-W-L-I-K(1,3)-G-G-P-S-E-P-E-S-L-R-H-
Y-Y-N-W-L-T-R-Q-R-Y-NH2.C20DA-gGlu-gGlu-gGlu-gGlu-gGlu-gGlu-eLys(1,2)

MW (calculated): 7040.9 Da
Synthesis and purification methods: S02; P03
LCMS: A02-B; Rt: 9.3 min; m/3: 2347.9 m/4: 1761.4 m/5:

(SEQ ID NO: 449)
Compound 123
Y-Aib-E-G-T-F-T-S-D-Y-S-I-Aib-L-E-K-Q-A-Q-Aib-A-F-V-E-W-L-I-K(1,3)-G-G-Q-S-S-G-A-S-L-R-H-
Y-Y-N-W-L-T-R-Q-R-Y-NH2.C20DA-gGlu-gGlu-gGlu-gGlu-gGlu-gGlu-eLys(1,2)

MW (calculated): 6931.7 Da
Synthesis and purification methods: S02; P03
LCMS: A02-B; Rt: 10.9 min; m/3: 2312.0 m/4: 1732.4 m/5:

Compound 124
(SEQ ID NO: 450)
Y-Aib-E-G-T-F-T-S-D-Y-S-I-Aib-L-E-Q-Q-A-Q-Aib-A-F-V-E-W-L-I-K(1,3)-G-G-P-S-S-G-A-S-L-R-H-
Y-Y-N-W-L-T-R-Q-R-Y-NH2.C20DA-gGlu-gGlu-gGlu-gGlu-Glu-gGlu-eLys(1,2)

MW (calculated): 6900.7 Da
Synthesis and purification methods: S01; P02
LCMS: A01-B; Rt: 36.0 min; m/3: m/4: m/5: 1381.0

Compound 125
(SEQ ID NO: 451)
Y-Aib-E-G-T-F-T-S-D-Y-S-I-Aib-L-E-K-Q-A-Q-Aib-A-F-V-Q-W-L-I-K(1,3)-G-G-P-S-E-G-A-S-L-R-
H-Y-Y-N-W-L-T-R-Q-NMeR-Y-NH2.C20DA-gGlu-gGlu-gGlu-gGlu-gGlu-gGlu-eLys(1,2)

MW (calculated): 6955.8 Da
Synthesis and purification methods: S01; P02
LCMS: A01-B; Rt: 35.5 min; m/3: m/4: 1739.8 m/5:

Compound 126
(SEQ ID NO: 452)
Y-Aib-E-G-T-F-T-S-D-Y-S-I-Aib-L-E-K-Q-A-Q-Aib-A-F-V-E-W-L-I-K(1,3)-G-G-P-S-S-Y-A-S-L-R-H-Y-Y-
N-W-L-T-R-Q-R-Y-NH2.C20DA-gGlu-gGlu-gGlu-gGlu-gGlu-Glu-eLys(1,2)

MW (calculated): 7006.9 Da
Synthesis and purification methods: S01; P02
LCMS: A01-B; Rt: 34.7 min; m/3: m/4: 1752.5 m/5:

Compound 127
(SEQ ID NO: 453)
Y-Aib-E-G-T-F-T-S-D-Y-S-I-Aib-L-E-K-Q-A-Q-Aib-A-F-V-E-W-L-I-K(1,3)-G-G-P-S-S-G-E-S-L-R-H-Y-Y-
N-W-L-T-R-Q-R-Y-NH2.C20DA-gGlu-gGlu-gGlu-gGlu-gGlu-gGlu-eLys(1,2)

MW (calculated): 6958.8 Da
Synthesis and purification methods: S02; P03
LCMS: A02-A; Rt: 7.3 min; m/3: 2320.8 m/4: 1740.8 m/5:

Compound 128
(SEQ ID NO: 454)
Y-Aib-E-G-T-F-T-S-D-Y-S-I-Aib-L-D-K-Q-A-Q-Aib-A-F-V-E-W-L-I-K(1,3)-G-G-P-S-S-G-A-S-L-R-H-Y-
Y-N-W-L-T-R-Q-R-Y-NH2.C20DA-gGlu-gGlu-gGlu-gGlu-gGlu-gGlu-eLys(1,2)

MW (calculated): 6886.7 Da
Synthesis and purification methods: S01; P02
LCMS: A01-B; Rt: 34.9 min; m/3: m/4: 1722.3 m/5:

Compound 129
(SEQ ID NO: 455)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-Aib-H-
Y-Y-N-W-L-T-R-Q-R-Y-NH2.C20DA-gGlu-gGlu-gGlu-gGlu-gGlu-gGlu-eLys(1,2)

MW (calculated): 6829.6 Da
Synthesis and purification methods: S01; P02
LCMS: A01-B; Rt: 36.0 min; m/3: m/4: 1708.2 m/5:

Compound 130
(SEQ ID NO: 456)
Y-Aib-E-G-T-F-T-S-D-Y-S-I-Aib-L-E-K-Q-A-Q-Aib-A-F-I-E-W-L-I-K(1,3)-G-G-P-S-S-G-A-S-L-R-H-Y-
Y-N-W-L-T-R-Q-R-Y-NH2.C20DA-gGlu-gGlu-gGlu-Glu-gGlu-gGlu-eLys(1,2)

MW (calculated): 6914.8 Da
Synthesis and purification methods: S01; P02
LCMS: A01-B; Rt: 35.4 min; m/3: m/4: 1729.3 m/5:

Compound 131
(SEQ ID NO: 457)
Y-Aib-E-G-T-F-T-S-D-Y-S-I-Aib-L-E-R-Q-A-Q-Aib-A-F-V-E-W-L-I-K(1,3)-G-G-P-S-S-G-A-S-L-R-H-Y-
Y-N-W-L-T-R-Q-R-Y-NH2.C20DA-gGlu-gGlu-gGlu-gGlu-gGlu-gGlu-eLys(1,2)

MW (calculated): 6928.7 Da
Synthesis and purification methods: S01; P01
LCMS: A01-B; Rt: 35.4 min; m/3: m/4: m/5: 1386.5

Compound 132
(SEQ ID NO: 458)
Y-Aib-E-G-T-F-T-S-D-Y-S-I-Aib-L-E-K-Q-A-Q-Aib-A-F-V-E-W-L-I-K(1,3)-G-G-P-S-S-G-A-S-I-R-H-
Y-Y-N-W-L-T-R-Q-R-Y-NH2.C20DA-gGlu-gGlu-gGlu-gGlu-gGlu-gGlu-eLys(1,2)

MW (calculated): 6900.7 Da
Synthesis and purification methods: S02; P03
LCMS: A02-A; Rt: 7.3 min; m/3: 2301.5 m/4: 1726.6 m/5:

Compound 133
(SEQ ID NO: 459)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-
R-H-Y-Tle-N-W-L-T-R-Q-R-Y-NH2.C20DA-gGlu-gGlu-gGlu-gGlu-gGlu-gGlu-eLys(1,2)

MW (calculated): 6851.7 Da
Synthesis and purification methods: 501; P02
LCMS: A01-B; Rt: 36.0 min; m/3: m/4: 1713.4 m/5:

Compound 134
(SEQ ID NO: 460)
Y-Aib-E-G-T-F-T-S-D-Y-S-I-Aib-L-E-K-Q-A-Q-Aib-A-F-V-E-W-L-I-K(1,3)-G-G-P-S-E-G-Q-S-I-R-
H-Y-Y-N-W-L-T-R-Q-R-Y-NH2.C20DA-gGlu-gGlu-gGlu-gGlu-gGlu-gGlu-eLys(1,2)

MW (calculated): 6999.8 Da
Synthesis and purification methods: S02; P03
LCMS: A02-A; Rt: 7.1 min; m/3: 2334.4 m/4: 1750.9 m/5:

Compound 135
(SEQ ID NO: 461)
Y-Aib-E-G-T-F-T-S-D-Y-S-I-Aib-L-E-K-Q-A-Q-Aib-A-F-V-E-Y-L-I-K(1,3)-G-G-P-S-S-G-A-S-L-R-
H-Y-Y-N-W-L-T-R-Q-R-Y-NH2.C20DA-gGlu-gGlu-gGlu-gGlu-gGlu-gGlu-eLys(1,2)

MW (calculated): 6877.7 Da
Synthesis and purification methods: 501; P02
LCMS: A01-B; Rt: 34.2 min; m/3: m/4: 1720.1 m/5:

Compound 136
(SEQ ID NO: 462)
Y-Aib-E-G-T-F-T-S-D-Y-S-I-Aib-L-E-K-A-A-Q-Aib-A-F-V-E-W-L-I-K(1,3)-G-G-P-S-S-G-A-S-L-R-H-
Y-Y-N-W-L-T-R-Q-R-Y-NH2.C20DA-gGlu-gGlu-gGlu-gGlu-gGlu-gGlu-eLys(1,2)

MW (calculated): 6843.7 Da
Synthesis and purification methods: 501; P02
LCMS: A01-B; Rt: 36.1 min; m/3: m/4: 1711.7 m/5:

Compound 137
(SEQ ID NO: 463)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-H-
Y-Y-N-W-Tle-T-R-Q-R-Y-NH2.C20DA-gGlu-gGlu-gGlu-gGlu-gGlu-gGlu-eLys(1,2)

MW (calculated): 6900.7 Da
Synthesis and purification methods: 501; P02
LCMS: A01-B; Rt: 35.4 min; m/3: m/4: 1725.9 m/5:

Compound 138
(SEQ ID NO: 464)
Y-Aib-E-G-T-F-T-S-D-Y-S-I-Aib-L-E-K-Q-A-E-Aib-A-F-V-Q-W-L-I-K(1,3)-G-G-P-S-E-G-A-S-L-R-
H-Y-Y-N-W-L-T-R-Q-R-Y-NH2.C18DA-gGlu-gGlu-gGlu-gGlu-gGlu-eLys(1,2)

MW (calculated): 6785.6 Da
Synthesis and purification methods: S02; P03
LCMS: A02-A; Rt: 7.0 min; m/3: 2262.8 m/4: 1697.6 m/5:

Compound 139
(SEQ ID NO: 465)
Y-Aib-E-G-T-F-T-S-D-Y-S-I-Aib-L-E-K-Q-A-Q-Aib-A-F-V-Q-W-L-I-K(1,3)-G-G-P-S-S-G-A-S-L-R-
H-Y-Y-N-W-L-T-R-Q-R-Y-NH2.C18DA-gGlu-gGlu-gGlu-gGlu-gGlu-gGlu-AHX(1,2)

MW (calculated): 6856.7 Da
Synthesis and purification methods: S02; P03
LCMS: A02-A; Rt: 7.1 min; m/3: 2286.8 m/4: 1713.0 m/5:

Compound 140
(SEQ ID NO: 466)
Y-Aib-E-G-T-F-T-S-D-Y-S-I-Aib-L-E-K-Q-A-Q-Aib-A-F-V-E-W-L-I-K(1,3)-G-G-P-S-S-P-A-S-L-R-H-Y-
Y-N-W-L-T-R-Q-R-Y-NH2.C18DA-gGlu-gGlu-gGlu-gGlu-gGlu-OEG-OEG(1,2)

MW (calculated): 6945.8 Da
Synthesis and purification methods: S02; P03
LCMS: A02-A; Rt: 7.3 min; m/3: 2316.6 m/4: 1737.6 m/5:

Compound 141
(SEQ ID NO: 467)
Y-Aib-E-G-T-F-T-S-D-Y-S-I-Aib-L-E-K-Q-A-Q-Aib-E-F-V-Q-W-L-I-K(1,3)-G-G-P-S-E-G-A-S-L-R-H-
Y-Y-N-W-L-T-R-Q-R-Y-NH2.C18DA-gGlu-gGlu-gGlu-gGlu-gGlu(1,2)

MW (calculated): 6714.5 Da
Synthesis and purification methods: S02; P03
LCMS: A02-B; Rt: 9.8 min; m/3: 2239.3 m/4: 1679.7 m/5:

Compound 142
(SEQ ID NO: 468)
Y-Aib-E-G-T-F-T-S-D-Y-S-I-Aib-L-E-K-Q-A-Q-Aib-A-F-V-Q-W-L-I-K(1,3)-G-G-P-S-E-P-A-S-L-R-H-Y-Y-
N-W-L-T-R-Q-R-Y-NH2.C18DA-gGlu-gGlu-gGlu-gGlu-gGlu-OEG-OEG(1,2)

MW (calculated): 6986.8 Da
Synthesis and purification methods: S02; P03
LCMS: A02-A; Rt: 7.0 min; m/3: 2329.8 m/4: 1747.6 m/5:

Compound 143
(SEQ ID NO: 469)
Y-Aib-E-G-T-F-T-S-D-Y-S-I-Aib-L-E-K-Q-A-Q-Aib-A-F-V-Q-W-L-I-K(1,3)-G-G-P-E-E-G-A-S-L-R-H-
Y-Y-N-W-L-T-R-Q-R-Y-NH2.C18DA-gGlu-gGlu-gGlu-gGlu-gGlu-eLys(1,2)

MW (calculated): 6826.7 Da
Synthesis and purification methods: S02; P03
LCMS: A02-A; Rt: 6.8 min; m/3: 2276.4 m/4: 1707.7 m/5:

Compound 144
(SEQ ID NO: 470)
Y-Aib-E-G-T-F-T-S-D-Y-S-I-Aib-L-E-K-Q-A-Q-Aib-A-F-V-Q-W-L-I-K(1,3)-G-G-P-E-E-G-A-S-L-R-
H-Y-Y-N-W-L-T-R-Q-R-Y-NH2.C18DA-gGlu-gGlu-gGlu-OEG-OEG(1,2)

MW (calculated): 6730.6 Da
Synthesis and purification methods: S02; P03
LCMS: A02-B; Rt: 10.2 min; m/3: 2244.5 m/4: 1683.6 m/5:

Compound 145
(SEQ ID NO: 471)
Y-Aib-E-G-T-F-T-S-D-Y-S-I-Aib-L-E-K-Q-A-Q-Aib-E-F-V-E-W-L-I-K(1,3)-G-G-P-S-S-G-A-S-L-R-H-Y-
Y-N-W-L-T-R-Q-R-Y-NH2.C18DA-gGlu-gGlu-gGlu-gGlu-OEG-OEG(1,2)

MW (calculated): 6834.6 Da
Synthesis and purification methods: S02; P03
LCMS: A02-B; Rt: 9.8 min; m/3: 2279.3 m/4: 1709.6 m/5:

Compound 146
(SEQ ID NO: 472)
Y-Aib-E-G-T-F-T-S-D-Y-S-I-Aib-L-E-K-Q-A-Q-Aib-A-F-V-Q-W-L-I-K(1,3)-G-G-P-E-E-G-A-S-L-R-
H-Y-Y-N-W-L-T-R-Q-R-Y-NH2.C18DA-gGlu-gGlu-gGlu-gGlu-OEG-OEG(1,2)

MW (calculated): 6859.7 Da
Synthesis and purification methods: S02; P03
LCMS: A02-B; Rt: 10.0 min; m/3: 2287.7 m/4: 1716.0 m/5:

Compound 147
(SEQ ID NO: 473)
Y-Aib-E-G-T-F-T-S-D-Y-S-I-Aib-L-E-K-Q-A-Q-Aib-A-F-V-Q-W-L-I-K(1,3)-G-G-P-S-S-G-A-S-L-R-H-
Y-Y-N-W-L-T-R-Q-R-Y-NH2.C18DA-gGlu-gGlu-gGlu-gGlu-Glu-gGlu(1,2)

MW (calculated): 6743.5 Da
Synthesis and purification methods: S02; P03
LCMS: A02-A; Rt: 7.1 min; m/3: 2248.8 m/4: 1686.9 m/5:

Compound 148
(SEQ ID NO: 474)
Y-Aib-E-G-T-F-T-S-D-Y-S-I-Aib-L-E-K-Q-A-Q-Aib-A-F-V-Q-W-L-I-K(1,3)-G-G-P-S-E-P-A-S-L-
R-H-Y-Y-N-W-L-T-R-Q-R-Y-NH2.C18DA-gGlu-gGlu-gGlu-gGlu-gGlu-eLys(1,2)

MW (calculated): 6824.7 Da
Synthesis and purification methods: S02; P03
LCMS: A02-A; Rt: 6.8 min; m/3: 2275.9 m/4: 1707.1 m/5:

Compound 149
(SEQ ID NO: 475)
Y-Aib-E-G-T-F-T-S-D-Y-S-I-Aib-L-E-K-Q-A-Q-Aib-A-F-V-Q-W-L-I-K(1,3)-G-G-P-E-E-G-A-S-
L-R-H-Y-Y-N-W-L-T-R-Q-R-Y-NH2.C18DA-gGlu-gGlu-gGlu-gGlu-gGlu-gGlu-eLys(1,2)

MW (calculated): 6955.8 Da
Synthesis and purification methods: S02; P03
LCMS: A02-B; Rt: 9.2 min; m/3: 2319.9 m/4: 1740.0 m/5:

Compound 150
(SEQ ID NO: 476)
Y-Aib-E-G-T-F-T-S-D-Y-S-I-Aib-L-E-K-Q-A-E-Aib-A-F-V-E-W-L-I-K(1,3)-G-G-P-S-S-G-A-S-L-R-
H-Y-Y-N-W-L-T-R-Q-R-Y-NH2.C18DA-gGlu-glu-Glu-Glu-OEG-OEG(1,2)

MW (calculated): 6777.6 Da
Synthesis and purification methods: S02; P03
LCMS: A02-A; Rt: 6.2 min; m/3: 2260.5 m/4: 1695.4 m/5:

Compound 151
(SEQ ID NO: 477)
Y-Aib-E-G-T-F-T-S-D-Y-S-I-Aib-L-E-K-Q-A-Q-Aib-E-F-V-Q-W-L-I-K(1,3)-G-G-P-S-E-G-A-S-
L-R-H-Y-Y-N-W-L-T-R-Q-R-Y-NH2.C18DA-gGlu-gGlu-gGlu-gGlu-gGlu-gGlu(1,2)

MW (calculated): 6843.6 Da
Synthesis and purification methods: S02; P03
LCMS: A02-A; Rt: 6.9 min; m/3: 2281.9 m/4: 1711.8 m/5:

Compound 152
(SEQ ID NO: 478)
Y-Aib-E-G-T-F-T-S-D-Y-S-I-Aib-L-E-K-Q-A-Q-Aib-A-F-V-E-W-L-I-K(1,3)-G-G-P-E-S-G-A-S-L-R-
H-Y-Y-N-W-L-T-R-Q-R-Y-NH2.C18DA-gGlu-gGlu-gGlu-gGlu-gGlu-eLys(1,2)

MW (calculated): 6785.6 Da
Synthesis and purification methods: S02; P03
LCMS: A02-A; Rt: 6.9 min; m/3: 2263.3 m/4: 1697.4 m/5:

Compound 153
(SEQ ID NO: 479)
Y-Aib-E-G-T-F-T-S-D-Y-S-I-Aib-L-E-K-Q-A-Q-Aib-E-F-V-Q-W-L-I-K(1,3)-G-G-P-S-E-G-A-
S-L-R-H-Y-Y-N-W-L-T-R-Q-R-Y-NH2.C18DA-gGlu-gGlu-gGlu-gGlu-gGlu-gGlu-eLys(1,2)

MW (calculated): 6971.8 Da
Synthesis and purification methods: S02; P03
LCMS: A02-B; Rt: 7.5 min; m/3: 2325.0 m/4: 1743.9 m/5:

Compound 154
(SEQ ID NO: 480)
Y-Aib-E-G-T-F-T-S-D-Y-S-I-Aib-L-E-K-Q-A-Q-Aib-E-F-V-Q-W-L-I-K(1,3)-G-G-P-S-E-G-A-S-L-R-H-
Y-Y-N-W-L-T-R-Q-R-Y-NH2.C18DA-gGlu-gGlu-gGlu-gGlu-gGlu-eLys(1,2)

MW (calculated): 6842.7 Da
Synthesis and purification methods: S02; P03
LCMS: A02-B; Rt: 8.9 min; m/3: 2282.1 m/4: 1711.6 m/5:

Compound 155
(SEQ ID NOS 481
Y-Aib-E-G-T-F-T-S-D-Y-S-I-Aib-L-E-K-Q-A-Q-Aib-A-F-V-Q-W-L-I-K(1,3)-G-G-P-E-E-G-A-S-L-
R-H-Y-Y-N-W-L-T-R-Q-R-Y-NH2.C18DA-E-E-E-E-E-eLys(1,2)

MW (calculated): 6826.7 Da
Synthesis and purification methods: S02; P03
LCMS: A02-B; Rt: 9.6 min; m/3: 2276.8 m/4: 1707.6 m/5:

Compound 156
(SEQ ID NO: 482)
Y-Aib-E-G-T-F-T-S-D-Y-S-I-Aib-L-E-K-Q-A-E-Aib-A-F-V-Q-W-L-I-K(1,3)-G-G-P-S-E-G-A-S-L-R-H-
Y-Y-N-W-L-T-R-Q-R-Y-NH2.C18DA-gGlu-gGlu-gGlu-gGlu-OEG-OEG(1,2)

MW (calculated): 6818.6 Da
Synthesis and purification methods: S02; P03
LCMS: A02-A; Rt: 7.3 min; m/3: 2273.6 m/4: 1705.4 m/5:

Compound 157
(SEQ ID NO: 483)
Y-Aib-E-G-T-F-T-S-D-Y-S-I-Aib-L-E-K-Q-A-Q-Aib-E-F-V-Q-W-L-I-K(1,3)-G-G-P-S-E-G-A-S-L-R-
H-Y-Y-N-W-L-T-R-Q-R-Y-NH2.C18DA-E-E-E-OEG-OEG(1,2)

MW (calculated): 6688.5 Da
Synthesis and purification methods: S02; P03
LCMS: A02-A; Rt: 7.1 min; m/3: 2249.8 m/4: 1687.8 m/5: 6746.5

Compound 158
(SEQ ID NO: 484)
Y-Aib-E-G-T-F-T-S-D-Y-S-I-Aib-L-E-K-Q-A-Q-Aib-A-F-V-E-W-L-I-K(1,3)-G-G-P-S-S-P-A-S-L-R-H-
Y-Y-N-W-L-T-R-Q-R-Y-NH2.C18DA-gGlu-gGlu-gGlu-gGlu-OEG-OEG(1,2)

MW (calculated): 6816.7 Da
Synthesis and purification methods: S02; P03
LCMS: A02-A; Rt: 7.4 min; m/3: 2273.2 m/4: 1705.2 m/5:

Compound 159
(SEQ ID NO: 485)
Y-Aib-E-G-T-F-T-S-D-Y-S-I-Aib-L-E-K-Q-A-Q-Aib-E-F-V-E-W-L-I-K(1,3)-G-G-P-S-S-G-A-S-L-R-
H-Y-Y-N-W-L-T-R-Q-R-Y-NH2.C18DA-gGlu-gGlu-gGlu-gGlu-gGlu-eLys(1,2)

MW (calculated): 6801.6 Da
Synthesis and purification methods: S02; P03
LCMS: A02-A; Rt: 6.6 min; m/3: 2268.2 m/4: 1701.4 m/5:

Compound 160
(SEQ ID NO: 486)
Y-Aib-E-G-T-F-T-S-D-Y-S-I-Aib-L-E-K-Q-A-Q-Aib-E-F-V-Q-W-L-I-K(1,3)-G-G-P-S-E-G-A-S-L-R-
H-Y-Y-N-W-L-T-R-Q-R-Y-NH2.C18DA-gGlu-gGlu-gGlu-OEG-OEG(1,2)

MW (calculated): 6746.6 Da
Synthesis and purification methods: S02; P03
LCMS: A02-B; Rt: 9.8 min; m/3: 2249.8 m/4: 1687.6 m/5:

Compound 161
(SEQ ID NO: 487)
Y-Aib-E-G-T-F-T-S-D-Y-S-I-Aib-L-E-K-Q-A-Q-Aib-A-F-V-E-W-L-I-K(1,3)-G-G-P-E-E-P-A-S-L-R-
H-Y-Y-N-W-L-T-R-Q-R-Y-NH2.C18DA-gGlu-gGlu-gGlu-gGlu-OEG-OEG(1,2)

MW (calculated): 6900.7 Da
Synthesis and purification methods: S02; P03
LCMS: A02-A; Rt: 7.2 min; m/3: 2301.2 m/4: 1726.2 m/5:

Compound 162
(SEQ ID NO: 488)
Y-Aib-E-G-T-F-T-S-D-Y-S-I-Aib-L-E-K-Q-A-Q-Aib-A-F-V-Q-W-L-I-K(1,3)-G-G-P-E-E-G-A-S-L-R-
H-Y-Y-N-W-L-T-R-Q-R-Y-NH2.C18DA-gGlu-gGlu-gGlu-gGlu-gGlu-AHX(1,2)

MW (calculated): 6811.6 Da
Synthesis and purification methods: S02; P03
LCMS: A02-B; Rt: 8.8 min; m/3: 2271.5 m/4: 1704.1 m/5:

Compound 163
(SEQ ID NO: 489)
Y-Aib-E-G-T-F-T-S-D-Y-S-I-Aib-L-E-K-Q-A-Q-Aib-A-F-V-E-W-L-I-K(1,3)-G-G-P-E-S-G-A-S-L-R-
H-Y-Y-N-W-L-T-R-Q-R-Y-NH2.C18DA-gGlu-gGlu-gGlu-gGlu-OEG-OEG(1,2)

MW (calculated): 6818.6 Da
Synthesis and purification methods: S02; P03
LCMS: A02-A; Rt: 7.2 min; m/3: 2273.8 m/4: 1705.6 m/5:

Compound 164
(SEQ ID NO: 490)
Y-Aib-E-G-T-F-T-S-D-Y-S-I-Aib-L-E-K-Q-A-Q-Aib-E-F-V-Q-W-L-I-K(1,3)-G-G-P-S-E-G-A-S-L-R-H-
Y-Y-N-W-L-T-R-Q-R-Y-NH2.C18DA-gGlu-gGlu-gGlu-Glu-gGlu-gGlu-AHX(1,2)

MW (calculated): 6956.8 Da
Synthesis and purification methods: S02; P03
LCMS: A02-B; Rt: 8.2 min; m/3: 2319.5 m/4: 1740.3 m/5:

Compound 165
(SEQ ID NO: 491)
Y-Aib-E-G-T-F-T-S-D-Y-S-I-Aib-L-E-K-Q-A-Q-Aib-A-F-V-Q-W-L-I-K(1,3)-G-G-P-S-E-P-A-S-L-
R-H-Y-Y-N-W-L-T-R-Q-R-Y-NH2.C20DA-gGlu-Glu-gGlu-gGlu-OEG-OEG(1,2)

MW (calculated): 6885.8 Da
Synthesis and purification methods: S02; P03
LCMS: A02-B; Rt: 10.8 min; m/3: 2295.9 m/4: 1722.1 m/5:

Compound 166
(SEQ ID NO: 492)
Y-Aib-E-G-T-F-T-S-D-Y-S-I-Aib-L-E-K-Q-A-Q-Aib-A-F-V-E-W-L-I-K(1,3)-G-G-P-E-S-G-A-S-L-R-H-
Y-Y-N-W-L-T-R-Q-R-Y-NH2.C18DA-gGlu-gGlu-gGlu-OEG-OEG(1,2)

MW (calculated): 6689.5 Da
Synthesis and purification methods: S02; P03
LCMS: A02-A; Rt: 6.2 min; m/3: 2231.0 m/4: 1673.6 m/5:

Compound 167
(SEQ ID NO: 493)
Y-Aib-E-G-T-F-T-S-D-Y-S-I-Aib-L-E-K-Q-A-Q-Aib-A-F-V-Q-W-L-I-K(1,3)-G-G-P-S-S-G-A-S-L-R-H-
Y-Y-N-W-L-T-R-Q-R-Y-NH2.C18DA-gGlu-gGlu-gGlu-gGlu-gGlu-OEG-OEG(1,2)

MW (calculated): 6904.7 Da
Synthesis and purification methods: S02; P03
LCMS: A02-A; Rt: 7.1 min; m/3: 2302.7 m/4: 1727.3 m/5:

Compound 168
(SEQ ID NO: 494)
Y-Aib-E-G-T-F-T-S-D-Y-S-I-Aib-L-E-K-Q-A-Q-Aib-A-F-V-E-W-L-I-K(1,3)-G-G-P-R-S-G-A-S-L-R-H-
Y-Y-N-W-L-T-R-Q-R-Y-NH2.C20DA-gGlu-gGlu-gGlu-gGlu-gGlu-gGlu-eLys(1,2)

MW (calculated): 6969.8 Da
Synthesis and purification methods: S02; P03
LCMS: A02-B; Rt: 9.9 min; m/3: 2324.9 m/4: 1743.5 m/5:

Compound 169
(SEQ ID NO: 495)
Y-Aib-E-G-T-F-T-S-D-Y-S-I-Aib-L-E-K-Q-A-Q-Aib-A-F-V-Q-W-L-I-K(1,3)-G-G-P-S-E-P-A-S-L-R-H-Y-
Y-N-W-L-T-R-Q-R-Y-NH2.C20DA-gGlu-gGlu-gGlu-gGlu-gGlu-eLys(1,2)

MW (calculated): 6853.7 Da
Synthesis and purification methods: S02; P03
LCMS: A02-A; Rt: 7.2 min; m/3: 2285.1 m/4: 1714.0 m/5:

Compound 170
(SEQ ID NO: 496)
Y-Aib-E-G-T-F-T-S-D-Y-S-I-Aib-L-E-K-Q-A-Q-Aib-A-F-V-Q-W-L-I-K(1,3)-G-G-P-S-S-G-A-S-L-R-H-
Y-Y-N-W-L-T-R-Q-R-Y-NH2.C18DA-gGlu-gGlu-gGlu-gGlu-gGlu-gGlu-eLys(1,2)

MW (calculated): 6871.7 Da
Synthesis and purification methods: S02; P03
LCMS: A02-A; Rt: 6.7 min; m/3: 2291.8 m/4: 1719.1 m/5:

Compound 171
(SEQ ID NO: 497)
Y-Aib-E-G-T-F-T-S-D-Y-S-I-Aib-L-E-K-Q-A-Q-Aib-E-F-V-E-W-L-I-K(1,3)-G-G-P-S-S-G-A-S-L-R-H-
Y-Y-N-W-L-T-R-Q-R-Y-NH2.C18DA-gGlu-gGlu-gGlu-OEG-OEG(1,2)

MW (calculated): 6705.5 Da
Synthesis and purification methods: S02; P03
LCMS: A02-B; Rt: 10.1 min; m/3: 2236.5 m/4: 1677.6 m/5:

Compound 172
(SEQ ID NO: 498)
Y-Aib-E-G-T-F-T-S-D-L-S-I-Aib-L-E-K-Q-A-Q-Aib-A-F-V-E-W-L-I-K(1,3)-G-G-P-S-S-G-A-S-L-R-H-Y-
Y-N-W-L-T-R-Q-R-Y-NH2.C20DA-gGlu-gGlu-gGlu-gGlu-gGlu-gGlu-eLys(1,2)

MW (calculated): 6850.7 Da
Synthesis and purification methods: S01; P02
LCMS: A01-B; Rt: 36.4 min; m/3: m/4: 1713.4 m/5:

Compound 173
(SEQ ID NO: 499)
Y-Aib-E-G-T-F-T-S-D-Y-S-I-Aib-L-E-K-Q-A-Q-Aib-A-F-V-Q-W-L-I-K(1,3)-G-G-P-S-E-P-A-S-L-R-H-
Y-Y-N-W-L-T-R-Q-R-Y-NH2.C18DA-gGlu-gGlu-gGlu-gGlu-OEG-OEG(1,2)

MW (calculated): 6857.7 Da
Synthesis and purification methods: S02; P03
LCMS: A02-B; Rt: 10.0 min; m/3: 2286.9 m/4: 1715.5 m/5:

Compound 174
(SEQ ID NO: 500)
Y-Aib-E-G-T-F-T-S-D-Y-S-I-Aib-L-E-K-Q-A-E-Aib-A-F-V-Q-W-L-I-K(1,3)-G-G-P-S-E-G-A-S-L-R-H-
Y-Y-N-W-L-T-R-Q-R-Y-NH2.C18DA-gGlu-gGlu-gGlu-gGlu-eLys(1,2)

MW (calculated): 6656.5 Da
Synthesis and purification methods: S02; P03
LCMS: A02-A; Rt: 5.9 min; m/3: 2220.1 m/4: 1665.7 m/5:

Compound 175
(SEQ ID NO: 501)
Y-Aib-E-G-T-F-T-S-D-Y-S-I-Aib-L-E-K-Q-A-Q-Aib-A-F-V-E-W-L-I-K(1,3)-G-G-P-S-S-P-A-S-L-R-H-Y-
Y-N-W-L-T-R-Q-R-Y-NH2.C20DA-gGlu-gGlu-gGlu-gGlu-OEG-OEG(1,2)

MW (calculated): 6844.7 Da
Synthesis and purification methods: S02; P03
LCMS: A02-A; Rt: 8.0 min; m/3: 2282.2 m/4: 1712.2 m/5:

Compound 176
(SEQ ID NOS 502
Y-Aib-E-G-T-F-T-S-D-Y-S-I-Aib-L-E-K-Q-A-Q-Aib-E-F-V-Q-W-L-I-K(1,3)-G-G-P-S-E-G-A-S-L-R-H-
Y-Y-N-W-L-T-R-Q-R-Y-NH2.C18DA-E-E-E-E-E(1,2)

MW (calculated): 6714.5 Da
Synthesis and purification methods: S02; P03
LCMS: A02-A; Rt: 7.1 min; m/3: 2239.9 m/4: 1679.8 m/5:

Compound 177
(SEQ ID NOS 503
Y-Aib-E-G-T-F-T-S-D-Y-S-I-Aib-L-E-K-Q-A-Q-Aib-A-F-V-Q-W-L-I-K(1,3)-G-G-P-E-E-G-A-S-L-R-H-
Y-Y-N-W-L-T-R-Q-R-Y-NH2.C18DA-E-E-E-E-OEG-OEG(1,2)

MW (calculated): 6859.7 Da
Synthesis and purification methods: S02; P03
LCMS: A02-B; Rt: 10.3 min; m/3: 2287.1 m/4: 1716.0 m/5:

Compound 178
(SEQ ID NO: 504)
Y-Aib-E-G-T-F-T-S-D-Y-S-I-Aib-L-E-K-Q-A-Q-Aib-A-F-V-E-W-L-I-K(1,3)-G-G-P-S-S-P-A-S-L-R-H-
Y-Y-N-W-L-T-R-Q-R-Y-NH2.C20DA-gGlu-Glu-gGlu-gGlu-gGlu(1,2)

MW (calculated): 6683.5 Da
Synthesis and purification methods: S02; P03
LCMS: A02-A; Rt: 7.7 min; m/3: 2228.3 m/4: 1671.5 m/5:

Compound 179
(SEQ ID NOS 505
Y-Aib-E-G-T-F-T-S-D-Y-S-I-Aib-L-E-K-Q-A-Q-Aib-E-F-V-Q-W-L-I-K(1,3)-G-G-P-S-E-G-A-S-L-R-H-Y-
Y-N-W-L-T-R-Q-R-Y-NH2.C18DA-E-E-E-E-E-E(1,2)

and 637, respectively)
MW (calculated): 6843.6 Da
Synthesis and purification methods: S02; P03
LCMS: A02-B; Rt: 10.0 min; m/3: 2282.2 m/4: 1711.8 m/5:

Compound 180
(SEQ ID NO: 506)
Y-Aib-E-G-T-F-T-S-D-Y-S-I-Aib-L-E-K-Q-A-Q-Aib-A-F-V-E-W-L-I-K(1,3)-G-G-P-S-S-P-A-S-L-R-H-
Y-Y-N-W-L-T-R-Q-R-Y-NH2.C18DA-gGlu-gGlu-gGlu-gGlu-gGlu(1,2)

MW (calculated): 6655.5 Da
Synthesis and purification methods: S02; P03
LCMS: A02-A; Rt: 7.1 min; m/3: 2218.6 m/4: 1664.3 m/5:

Compound 181
(SEQ ID NO: 507)
Y-Aib-E-G-T-F-T-S-D-Y-S-I-Aib-L-E-E-Q-A-Q-Aib-A-F-V-E-W-L-I-K(1,3)-G-G-P-S-S-G-A-S-L-R-H-
Y-Y-N-W-L-T-R-Q-R-Y-NH2.C20DA-gGlu-gGlu-gGlu-gGlu-gGlu-eLys(1,2)

MW (calculated): 6772.6 Da
Synthesis and purification methods: 501; P01
LCMS: A01-B; Rt: 36.8 min; m/3: m/4: 1693.8 m/5:

Compound 182
(SEQ ID NO: 508)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-
Aib-Y-Y-N-W-L-T-R-Q-R-Y-NH2.C20DA-gGlu-gGlu-gGlu-gGlu-gGlu-gGlu-eLys(1,2)

MW (calculated): 6848.7 Da
Synthesis and purification methods: 501; P02
LCMS: A01-B; Rt: 36.1 min; m/3: m/4: 1712.9 m/5:
MW (calculated): 6877.7 Da PGP-232DNA

Compound 183
(SEQ ID NO: 509)
Y-Aib-E-G-T-F-T-S-D-Y-S-I-Aib-L-E-K-Q-A-Q-Aib-A-F-V-E-W-L-I-K(1,3)-G-G-P-S-S-Y-A-S-L-R-H-Y-
Y-N-W-L-T-R-Q-R-Y-NH2.C20DA-gGlu-gGlu-gGlu-Glu-gGlu-eLys(1,2)

Synthesis and purification methods: S02; P03
LCMS: A02-A; Rt: 7.4 min; m/3: 2293.2 m/4: 1720.3 m/5:

Compound 184
(SEQ ID NO: 510)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-H-
Y-Aib-N-W-L-T-R-Q-R-Y-NH2.C20DA-gGlu-gGlu-gGlu-gGlu-gGlu-gGlu-eLys(1,2)

MW (calculated): 6822.7 Da
Synthesis and purification methods: 501; P02
LCMS: A01-B; Rt: 35.2 min; m/3: m/4: 1706.5 m/5:

Compound 185
(SEQ ID NO: 511)
Y-Aib-E-G-T-F-T-S-D-Y-S-I-Aib-L-E-K-Q-A-Q-Aib-E-F-V-Q-W-L-I-K(1,3)-G-G-P-S-E-G-A-S-L-R-H-
Y-Y-N-W-L-T-R-Q-R-Y-NH2.C20DA-gGlu-gGlu-gGlu-gGlu-gGlu-gGlu(1,2)

MW (calculated): 6871.6 Da
Synthesis and purification methods: 501; P02
LCMS: A01-A; Rt: 13.3 min; m/3: m/4: 1718.7 m/5:

Compound 186
(SEQ ID NO: 512)
Y-Aib-E-G-T-F-T-S-D-Y-S-I-Aib-L-E-K-Q-A-Q-Aib-A-F-V-Q-W-L-I-K(1,3)-G-G-P-S-E-G-A-S-L-R-H-Y-
Y-N-W-L-T-R-bHGln-R-Y-NH2.C20DA-gGlu-gGlu-gGlu-gGlu-gGlu-gGlu-eLys(1,2)

MW (calculated): 6955.8 Da
Synthesis and purification methods: 501; P02
LCMS: A01-B; Rt: 35.0 min; m/3: m/4: 1739.6 m/5:

Compound 187
(SEQ ID NO: 513)
Y-Aib-E-G-T-F-T-S-D-Y-S-I-Aib-L-E-K-Q-A-Q-Aib-E-F-V-E-W-L-I-K(1,3)-G-G-P-S-S-G-A-S-L-R-H-Y-
Y-N-W-L-T-R-Q-R-Y-NH2.C18DA-gGlu-gGlu-gGlu-gGlu-gGlu-gGlu-eLys(1,2)

MW (calculated): 6930.7 Da
Synthesis and purification methods: S02; P03
LCMS: A02-B; Rt: 8.6 min; m/3: 2311.2 m/4: 1733.4 m/5:

Compound 188
(SEQ ID NO: 514)
Y-Aib-E-G-T-F-T-S-D-Y-S-I-Aib-L-E-K-Q-A-Q-Aib-A-F-V-Q-W-L-I-K(1,3)-G-G-P-E-E-G-A-S-L-R-H-Y-
Y-N-W-L-T-R-Q-R-Y-NH2.C18DA-E-E-E-OEG-OEG(1,2)

MW (calculated): 6730.6 Da
Synthesis and purification methods: S02; P03
LCMS: A02-B; Rt: 10.5 min; m/3: 2243.9 m/4: 1683.6 m/5:

(SEQ ID NO: 515)
Compound 189
Y-Aib-E-G-T-F-T-S-D-Y-S-I-Aib-L-E-K-Q-A-Q-Aib-A-F-V-Q-W-L-I-K(1,3)-G-G-P-S-E-Y-A-S-L-R-H-Y-
Y-N-W-L-T-R-Q-R-Y-NH2.C20DA-gGlu-gGlu-gGlu-gGlu-gGlu-eLys(1,2)

MW (calculated): 6918.8 Da
Synthesis and purification methods: S02; P03
LCMS: A02-A; Rt: 7.3 min; m/3: 2306.7 m/4: 1730.5 m/5:

Compound 190
(SEQ ID NO: 516)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-H-
Y-Y-N-W-DArg-T-R-Q-R-Y-NH2.C20DA-gGlu-gGlu-gGlu-gGlu-Glu-gGlu-eLys(1,2)

MW (calculated): 6943.8 Da
Synthesis and purification methods: S01; P01
LCMS: A01-B; Rt: 34.1 min; m/3: m/4: 1736.6 m/5:

Compound 191
(SEQ ID NO: 517)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-H-
Y-Y-N-W-L-T-R-Q-R-Tle-NH2.C20DA-gGlu-gGlu-gGlu-gGlu-gGlu-gGlu-eLys(1,2)

MW (calculated): 6850.7 Da
Synthesis and purification methods: S01; P02
LCMS: A01-B; Rt: 35.4 min; m/3: m/4: 1713.4 m/5:

Compound 192
(SEQ ID NO: 518)
Y-Aib-E-G-T-F-T-S-D-Y-S-I-Aib-L-E-KAc-Q-A-Q-Aib-A-F-V-E-W-L-I-K(1,3)-G-G-P-S-S-G-A-S-L-R-
H-Y-Y-N-W-L-T-R-Q-R-Y-NH2.C20DA-gGlu-gGlu-gGlu-gGlu-gGlu-glu-eLys(1,2)

MW (calculated): 6942.8 Da
Synthesis and purification methods: S01; P01
LCMS: A01-B; Rt: 37.0 min; m/3: m/4: 1736.3 m/5:

Compound 193
(SEQ ID NO: 519)
Y-Aib-E-G-T-F-T-S-D-Y-S-I-Aib-L-E-K-Q-A-Q-Aib-A-F-V-Q-W-L-I-K(1,3)-G-G-P-E-E-G-A-S-L-R-
H-Y-Y-N-W-L-T-R-Q-R-Y-NH2.C20DA-gGlu-gGlu-gGlu-gGlu-OEG-OEG(1,2)

MW (calculated): 6887.7 Da
Synthesis and purification methods: S02; P03
LCMS: A02-A; Rt: 6.6 min; m/3: 2297.1 m/4: 1723.5 m/5:

Compound 194
(SEQ ID NO: 520)
Y-Aib-E-G-T-F-T-S-D-Y-S-I-Aib-L-E-K-Q-A-Q-Aib-E-F-V-Q-W-L-I-K(1,3)-G-G-P-S-E-G-A-S-L-R-H-
Y-Y-N-W-L-T-R-Q-R-Y-NH2.C20DA-gGlu-gGlu-gGlu-gGlu(1,2)

MW (calculated): 6613.4 Da
Synthesis and purification methods: S02; P03
LCMS: A02-A; Rt: 7.7 min; m/3: 2205.7 m/4: 1654.4 m/5:

Compound 195
(SEQ ID NO: 521)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-
H-Y-Y-N-L-L-T-R-Q-R-Y-NH2.C20DA-gGlu-gGlu-gGlu-gGlu-gGlu-gGlu-eLys(1,2)

MW (calculated): 6827.7 Da
Synthesis and purification methods: S01; P02
LCMS: A01-B; Rt: 35.0 min; m/3: m/4: 1707.7 m/5:

Compound 196
(SEQ ID NOS 522
Y-Aib-E-G-T-F-T-S-D-Y-S-I-Aib-L-E-K-Q-A-Q-Aib-A-F-V-Q-W-L-I-K(1,3)-G-G-P-E-E-G-A-S-L-R-
H-Y-Y-N-W-L-T-R-Q-R-Y-NH2.C18DA-E-E-E-E-E-E-eLys(1,2)

MW (calculated): 6955.8 Da
Synthesis and purification methods: S02; P03
LCMS: A02-B; Rt: 9.4 min; m/3: 2319.1 m/4: 1739.8 m/5:

Compound 197
(SEQ ID NO: 523)
Y-Aib-E-G-T-F-T-S-D-Y-S-I-Aib-L-E-K-Q-A-Q-Aib-A-F-V-E-W-L-I-K(1,3)-G-G-P-S-S-G-A-L-L-R-H-
Y-Y-N-W-L-T-R-Q-R-Y-NH2.C20DA-gGlu-gGlu-gGlu-gGlu-gGlu-gGlu-eLys(1,2)

MW (calculated): 6927.8 Da
Synthesis and purification methods: S01; P02
LCMS: A01-B; Rt: 36.7 min; m/3: m/4: 1732.4 m/5:

Compound 198
(SEQ ID NO: 524)
Y-Aib-E-G-T-F-T-S-D-Y-S-I-Aib-L-E-K-Q-A-Q-Aib-E-F-V-E-W-L-I-K(1,3)-G-G-P-S-S-G-A-S-L-R-H-
Y-Y-N-W-L-T-R-Q-R-Y-NH2.C20DA-gGlu-gGlu-gGlu-gGlu-gGlu-glu(1,2)

MW (calculated): 6830.6 Da
Synthesis and purification methods: S01; P02
LCMS: A01-A; Rt: 13.4 min; m/3: m/4: 1708.3 m/5:

Compound 199
(SEQ ID NO: 525)
Y-Aib-E-G-T-F-T-S-D-Y-S-I-Aib-L-E-Aib-Q-A-Q-Aib-A-F-V-E-W-L-I-K(1,3)-G-G-P-S-S-G-A-S-L-R-H-
Y-Y-N-W-L-T-R-Q-R-Y-NH2.C20DA-gGlu-gGlu-gGlu-glu-gGlu-gGlu-eLys(1,2)

MW (calculated): 6857.7 Da
Synthesis and purification methods: S01; P02
LCMS: A01-B; Rt: 38.5 min; m/3: m/4: 1715.2 m/5:

Compound 200
(SEQ ID NO: 526)
Y-Aib-E-G-T-F-T-S-D-Y-S-I-Aib-L-E-K-Q-A-E-Aib-A-F-V-Q-W-L-I-K(1,3)-G-G-P-S-E-G-A-S-L-R-H-Y-Y-
N-W-L-T-R-Q-R-Y-NH2.C20DA-gGlu-gGlu-gGlu-gGlu-gGlu-eLys(1,2)

MW (calculated): 6813.7 Da
Synthesis and purification methods: S02; P03
LCMS: A02-A; Rt: 6.5 min; m/3: 2272.0 m/4: 1704.7 m/5:

Compound 201
(SEQ ID NO: 527)
Y-Aib-E-G-T-F-T-S-D-Y-S-I-Aib-L-E-K-Q-A-Q-Aib-E-F-V-E-W-L-I-K(1,3)-G-G-P-S-S-G-A-S-L-R-H-Y-
Y-N-W-L-T-R-Q-R-Y-NH2.C18DA-gGlu-gGlu-gGlu-gGlu-Glu-OEG-OEG(1,2)

MW (calculated): 6963.7 Da
Synthesis and purification methods: S02; P03
LCMS: A02-A; Rt: 6.9 min; m/3: 2322.1 m/4: 1742.1 m/5:

Compound 202
(SEQ ID NO: 528)
Y-Aib-E-G-T-F-T-S-D-Y-S-I-Aib-L-E-K-Q-A-Q-Aib-E-F-V-Q-W-L-I-K(1,3)-G-G-P-S-E-G-A-S-L-R-H-
Y-Y-N-W-L-T-R-Q-R-Y-NH2.C18DA-gGlu-gGlu-gGlu-gGlu(1,2)

MW (calculated): 6585.4 Da
Synthesis and purification methods: S02; P03
LCMS: A02-B; Rt: 9.9 min; m/3: 2196.5 m/4: 1647.5 m/5:

Compound 203
(SEQ ID NO: 529)
Y-Aib-E-G-T-F-T-S-D-Y-S-I-Aib-L-E-K-Q-A-Q-Aib-A-F-V-E-W-L-I-K(1,3)-G-G-P-S-Y-Y-A-S-L-R-H-Y-
Y-N-W-L-T-R-Q-R-Y-NH2.C20DA-gGlu-gGlu-gGlu-gGlu-Glu-eLys(1,2)

MW (calculated): 6953.8 Da
Synthesis and purification methods: S02; P03
LCMS: A02-A; Rt: 7.5 min; m/3: 2318.6 m/4: 1739.0 m/5:

Compound 204
(SEQ ID NO: 530)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-
H-Y-Y-N-W-DAsp-T-R-Q-R-Y-NH2.C20DA-gGlu-gGlu-gGlu-Glu-gGlu-gGlu-eLys(1,2)

MW (calculated): 6902.7 Da
Synthesis and purification methods: 501; P01
LCMS: A01-B; Rt: 34.6 min; m/3: m/4: 1726.4 m/5:

Compound 205
(SEQ ID NO: 531)
Y-Aib-E-G-T-F-T-S-D-Y-S-I-Aib-L-E-K-Q-A-Q-Aib-E-F-V-E-W-L-I-K(1,3)-G-G-P-S-S-G-A-S-L-
R-H-Y-Y-N-W-L-T-R-Q-R-Y-NH2.C20DA-gGlu-gGlu-gGlu-gGlu-OEG-OEG(1,2)

MW (calculated): 6862.7 Da
Synthesis and purification methods: S02; P03
LCMS: A02-A; Rt: 7.8 min; m/3: 2288.4 m/4: 1716.7 m/5:

Compound 206
(SEQ ID NO: 532)
Y-Aib-E-G-T-F-T-S-D-Y-S-I-Aib-L-E-K-Q-A-Q-Aib-A-F-V-E-W-L-I-K(1,3)-G-G-P-E-E-P-A-S-L-
R-H-Y-Y-N-W-L-T-R-Q-R-Y-NH2.C20DA-gGlu-gGlu-gGlu-gGlu-OEG-OEG(1,2)

MW (calculated): 6928.8 Da
Synthesis and purification methods: S02; P03
LCMS: A02-A; Rt: 7.9 min; m/3: 2310.6 m/4: 1733.1 m/5:

Compound 207
(SEQ ID NO: 533)
Y-Aib-E-G-T-F-T-S-D-Y-S-I-Aib-L-E-K-Q-A-Q-Aib-E-F-V-Q-W-L-I-K(1,3)-G-G-P-S-E-G-A-S-L-R-
H-Y-Y-N-W-L-T-R-Q-R-Y-NH2.C20DA-gGlu-gGlu-gGlu-gGlu-gGlu-Glu-gGlu-eLys(1,2)

MW (calculated): 7128.9 Da
Synthesis and purification methods: 501; P02
LCMS: A01-A; Rt: 12.4 min; m/3: m/4: 1783.0 m/5:

Compound 208
(SEQ ID NO: 534)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-
H-Y-Y-N-W-L-T-R-bHGln-R-Y-NH2.C20DA-gGlu-gGlu-gGlu-gGlu-gGlu-gGlu-eLys(1,2)

MW (calculated): 6914.8 Da
Synthesis and purification methods: 501; P01
LCMS: A01-B; Rt: 35.1 min; m/3: m/4: 1729.5 m/5:

Compound 209
(SEQ ID NO: 535)
Y-Aib-E-G-T-F-T-S-D-Y-S-I-Aib-L-E-L-Q-A-Q-Aib-A-F-V-E-W-L-I-K(1,3)-G-G-P-S-S-G-A-S-L-R-H-
Y-Y-N-W-L-T-R-Q-R-Y-NH2.C20DA-gGlu-gGlu-gGlu-gGlu-gGlu-gGlu-eLys(1,2)

MW (calculated): 6885.7 Da
Synthesis and purification methods: 501; P02
LCMS: A01-B; Rt: 39.6 min; m/3: m/4: 1722.1 m/5:

Compound 210
(SEQ ID NO: 536)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-L-
H-Y-Y-N-W-L-T-R-Q-R-Y-NH2.C20DA-gGlu-gGlu-gGlu-gGlu-gGlu-gGlu-eLys(1,2)

MW (calculated): 6857.7 Da
Synthesis and purification methods: 501; P02
LCMS: A01-B; Rt: 37.1 min; m/3: m/4: 1715.2 m/5:

Compound 211
(SEQ ID NO: 537)
Y-Aib-E-G-T-F-T-S-D-Y-S-I-Aib-L-E-K-Q-A-Q-DAsp-A-F-V-E-W-L-I-K(1,3)-G-G-P-S-S-G-A-S-L-
R-H-Y-Y-N-W-L-T-R-Q-R-Y-NH2.C20DA-gGlu-gGlu-gGlu-gGlu-gGlu-gGlu-eLys(1,2)

MW (calculated): 6930.7 Da
Synthesis and purification methods: 501; P01
LCMS: A01-B; Rt: 35.1 min; m/3: m/4: 1733.4 m/5:

Compound 212
(SEQ ID NO: 538)
Y-Aib-E-G-T-F-T-S-D-Y-S-I-Aib-L-E-K-Q-A-Q-Aib-A-F-V-E-W-L-I-K(1,3)-G-G-P-S-S-G-V-S-L-R-
H-Y-Y-N-W-L-T-R-Q-R-Y-NH2.C20DA-gGlu-gGlu-gGlu-Glu-gGlu-gGlu(1,2)

MW (calculated): 6800.6 Da
Synthesis and purification methods: S02; P03
LCMS: A02-B; Rt: 11.7 min; m/3: 2268.3 m/4: 1701.3 m/5:

Compound 213
(SEQ ID NO: 539)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-
H-Y-Y-Aib-W-L-T-R-Q-R-Y-NH2.C20DA-gGlu-gGlu-gGlu-gGlu-gGlu-gGlu-eLys(1,2)

MW (calculated): 6871.7 Da
Synthesis and purification methods: 501; P02
LCMS: A01-B; Rt: 36.5 min; m/3: m/4: 1718.6 m/5:

Compound 214
(SEQ ID NO: 540)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-
H-Y-Y-N-W-L-T-R-Q-R-DTyr-NH2.C20DA-gGlu-gGlu-gGlu-gGlu-gGlu-gGlu-eLys(1,2)

MW (calculated): 6900.7 Da
Synthesis and purification methods: 501; P02
LCMS: A01-B; Rt: 34.8 min; m/3: m/4: 1725.8 m/5:

Compound 215
(SEQ ID NO: 541)
Y-Aib-E-G-T-F-T-S-D-Y-S-I-Aib-L-E-K-Q-A-Q-Aib-E-F-V-E-W-L-I-K(1,3)-G-G-P-S-S-G-A-S-L-R-
H-Y-Y-N-W-L-T-R-Q-R-Y-NH2.C20DA-gGlu-gGlu-gGlu-gGlu-gGlu-gGlu-eLys(1,2)

MW (calculated): 6958.8 Da
Synthesis and purification methods: S02; P03
LCMS: A02-A; Rt: 6.1 min; m/3: 2320.6 m/4: 1740.7 m/5:

Compound 216
(SEQ ID NO: 542)
Y-Aib-E-G-T-F-T-S-D-Y-S-I-Aib-L-E-K-Q-A-Q-DArg-A-F-V-E-W-L-I-K(1,3)-G-G-P-S-S-G-A-S-L-R-
H-Y-Y-N-W-L-T-R-Q-R-Y-NH2.C20DA-gGlu-gGlu-gGlu-gGlu-gGlu-gGlu-eLys(1,2)

MW (calculated): 6971.8 Da
Synthesis and purification methods: 501; P01
LCMS: A01-B; Rt: 34.0 min; m/3: m/4: 1743.7 m/5:

Compound 217
(SEQ ID NO: 543)
Y-Aib-E-G-T-F-T-S-D-Y-S-I-Aib-L-E-K-Q-A-Q-Aib-A-F-V-E-W-L-I-K(1,3)-G-G-P-S-E-Y-V-S-L-R-
H-Y-Y-N-W-L-T-R-Q-R-Y-NH2.C20DA-gGlu-gGlu-gGlu-gGlu-gGlu-gGlu-eLys(1,2)

MW (calculated): 7076.9 Da
Synthesis and purification methods: S02; P03
LCMS: A02-A; Rt: 7.5 min; m/3: 2359.6 m/4: 1769.8 m/5:

Compound 218
(SEQ ID NO: 544)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-H-
Y-Y-N-Aib-L-T-R-Q-R-Y-NH2.C20DA-gGlu-gGlu-gGlu-gGlu-gGlu-gGlu-eLys(1,2)

MW (calculated): 6799.6 Da
Synthesis and purification methods: 501; P02
LCMS: A01-B; Rt: 35.0 min; m/3: m/4: 1700.6 m/5:

Compound 219
(SEQ ID NO: 545)
Y-Aib-E-G-T-F-T-S-D-Y-S-I-Aib-L-E-K-Q-A-E-Aib-A-
F-V-Q-W-L-I-K(1,3)-G-G-P-S-E-G-A-S-L-R-H-Y-Y-N-W-
L-T-R-Q-R-Y-NH2.C20DA-gGlu-gGlu-gGlu-gGlu-OEG-
OEG(1,2)

MW (calculated): 6846.7 Da
Synthesis and purification methods: S02; P03
LCMS: A02-B; Rt: 11.7 min; m/3: 2283.3 m/4: 1712.5 m/5: Compound 220

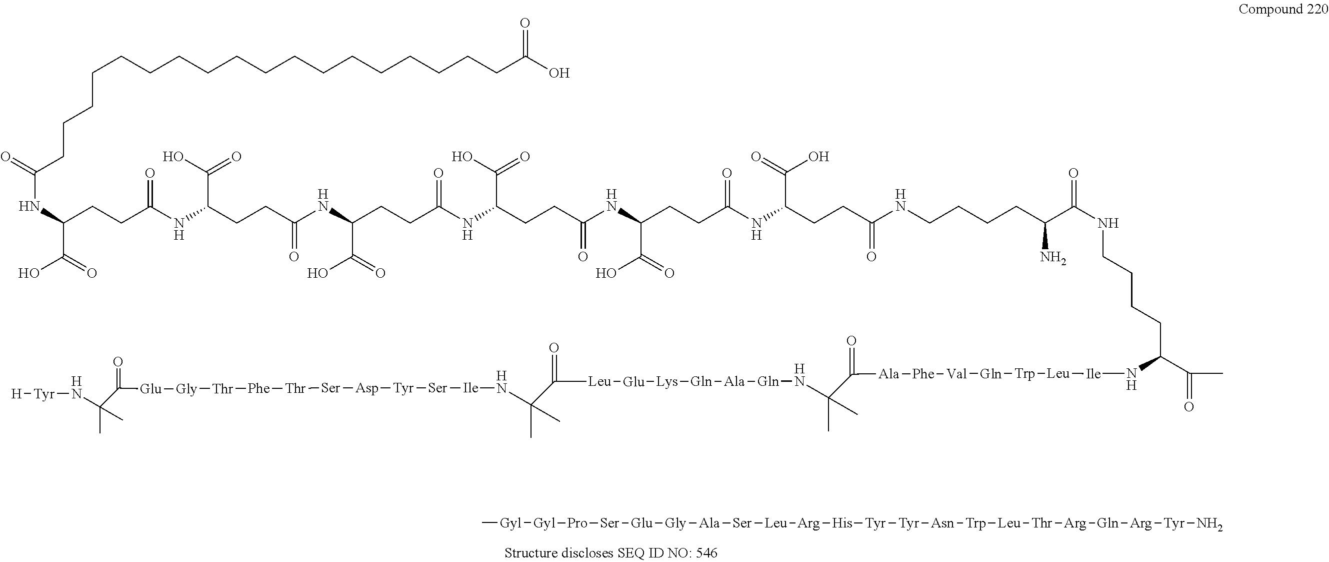

(SEQ ID NO: 546)
Y-Aib-E-G-T-F-T-S-D-Y-S-I-Aib-L-E-K-Q-A-Q-Aib-A-
F-V-Q-W-L-I-K(1,3)-G-G-P-S-E-G-A-S-L-R-H-Y-Y-N-W-
L-T-R-Q-R-Y-NH2.C20DA-gGlu-gGlu-gGlu-gGlu-gGlu-
gGlu-eLys(1,2)

MW (calculated): 6941.8 Da
Synthesis and purification methods: S02; P03
LCMS: A02-B; Rt: 14.5 min; m/3: 2314.5 m/4: 1736.1 m/5:

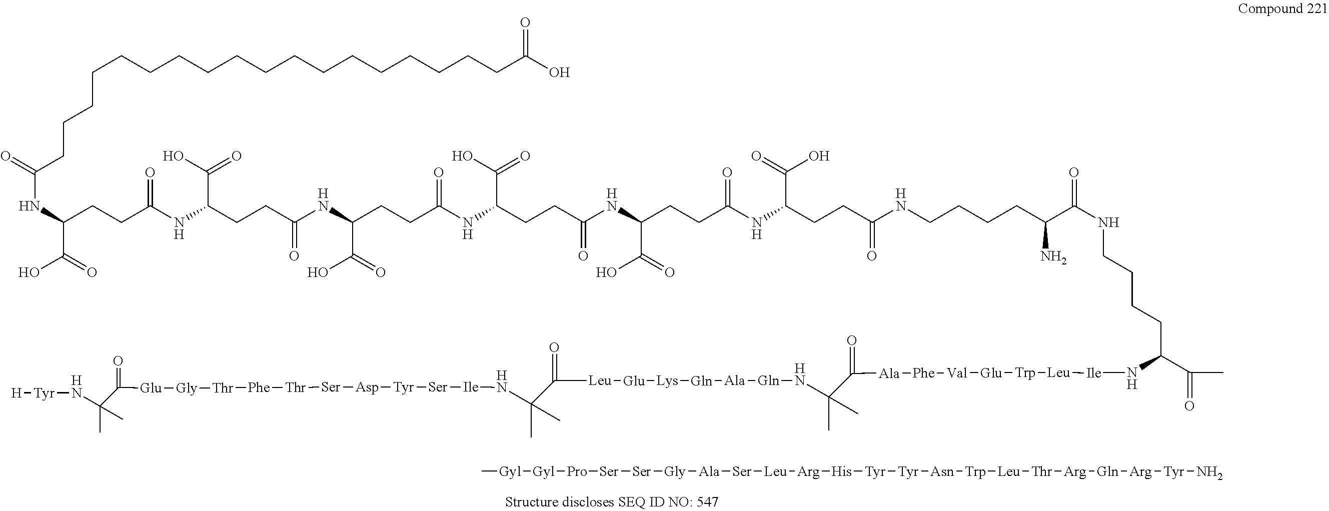

(SEQ ID NO: 547)
Y-Aib-E-G-T-F-T-S-D-Y-S-I-Aib-L-E-K-Q-A-Q-Aib-A-
F-V-E-W-L-I-K(1,3)-G-G-P-S-S-G-A-S-L-R-H-Y-Y-N-W-
L-T-R-Q-R-Y-NH2.C20DA-gGlu-gGlu-gGlu-gGlu-gGlu-
gGlu-eLys(1,2)

MW (calculated): 6900.7 Da
Synthesis and purification methods: 501; P02
LCMS: A01-A; Rt: 12.8 min; m/3: m/4: 1725.9 m/5:

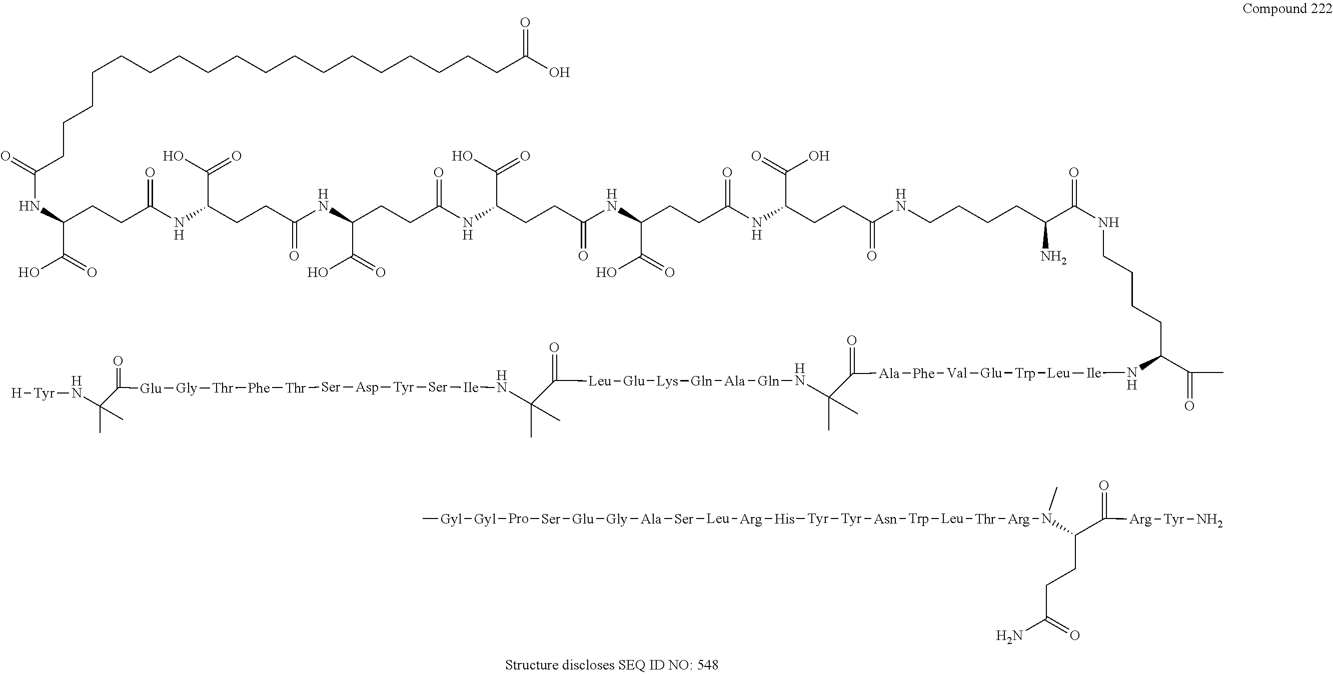

(SEQ ID NO: 548)
Y-Aib-E-G-T-F-T-S-D-Y-S-I-Aib-L-E-K-Q-A-Q-Aib-A-
F-V-E-W-L-I-K(1,3)-G-G-P-S-E-G-A-S-L-R-H-Y-Y-N-
W-L-T-R-NMeQ-R-Y-NH2.C20DA-gGlu-gGlu-gGlu-gGlu-
gGlu-gGlu-eLys(1,2)

MW (calculated): 6956.8 Da
Synthesis and purification methods: 501; P02
LCMS: A01-A; Rt: 12.7 min; m/3: m/4: 1740.0 m/5:

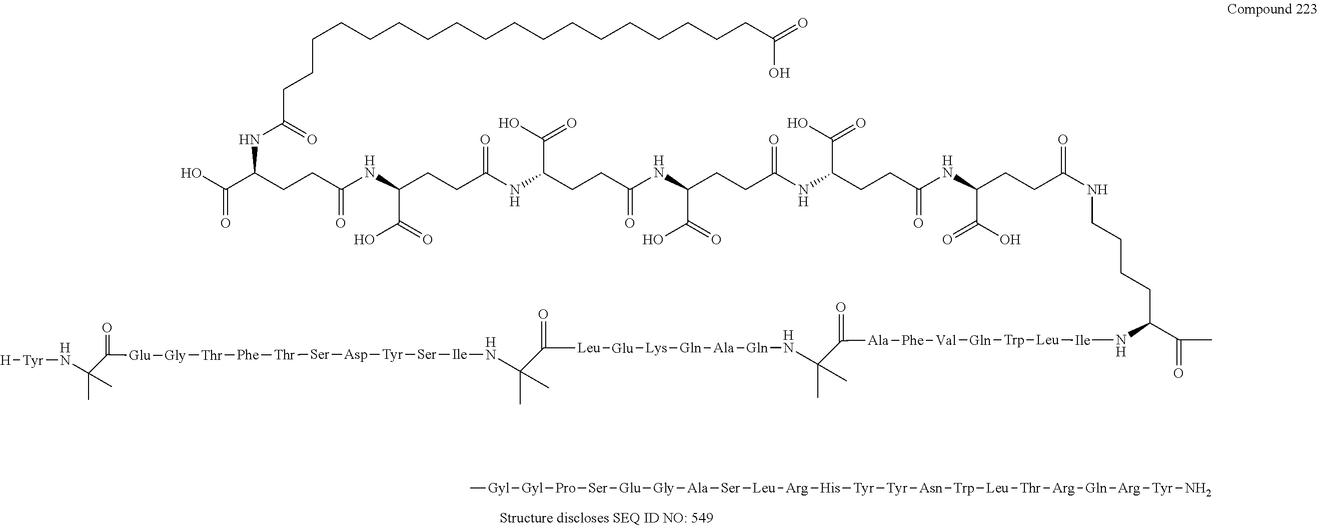

(SEQ ID NO: 549)
Y-Aib-E-G-T-F-T-S-D-Y-S-I-Aib-L-E-K-Q-A-Q-Aib-A-
F-V-Q-W-L-I-K(1,3)-G-G-P-S-E-G-A-S-L-R-H-Y-Y-N-
W-L-T-R-Q-R-Y-NH2.C20DA-gGlu-gGlu-gGlu-gGlu-
gGlu-gGlu(1,2)

MW (calculated): 6813.6 Da
Synthesis and purification methods: 501; L20
LCMS: A01-A; Rt: 6.7 min; m/3: 2272.1 m/4: 1704.5 m/5:

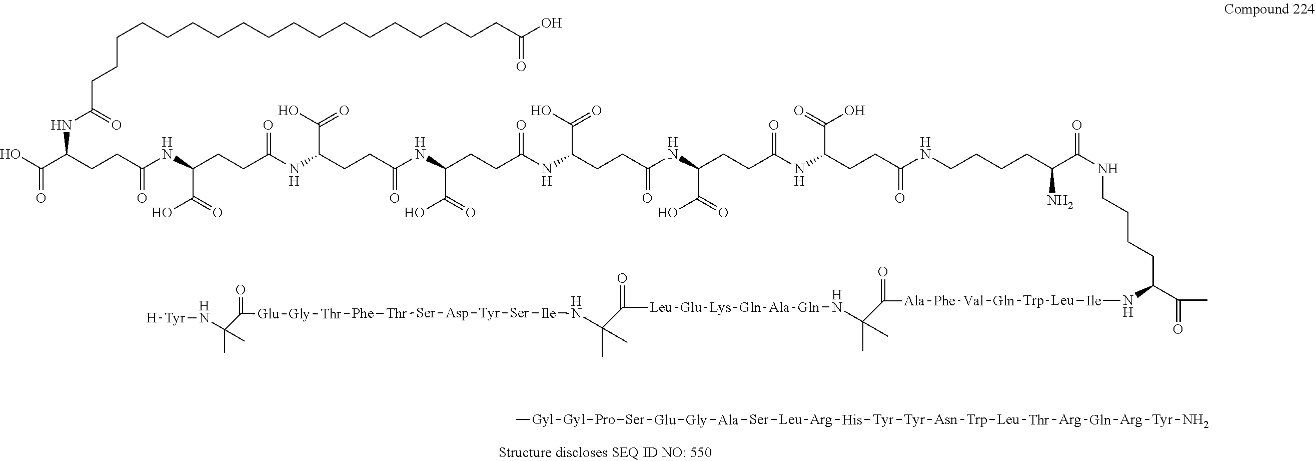

(SEQ ID NO: 550)
Y-Aib-E-G-T-F-T-S-D-Y-S-I-Aib-L-E-K-Q-A-Q-Aib-A-
F-V-Q-W-L-I-K(1,3)-G-G-P-S-E-G-A-S-L-R-H-Y-Y-N-
W-L-T-R-Q-R-Y-NH2.C20DA-gGlu-gGlu-gGlu-gGlu-gGlu-
gGlu-gGlu-eLys(1,2)

MW (calculated): 7070.9 Da
Synthesis and purification methods: 501; P02
LCMS: A01-B; Rt: 34.4 min; m/3: m/4: m/5: 1415.0

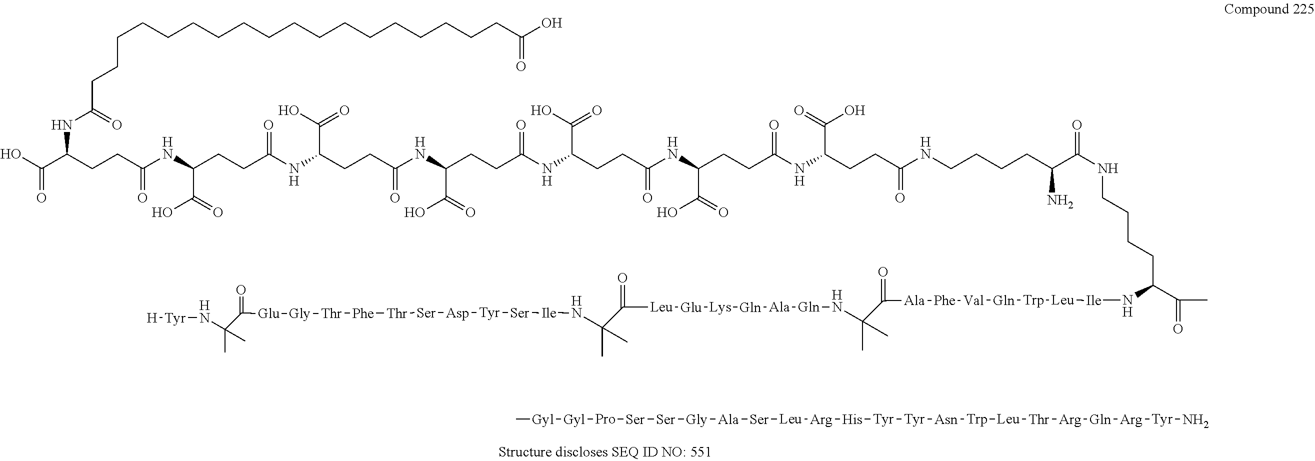

(SEQ ID NO: 551)
Y-Aib-E-G-T-F-T-S-D-Y-S-I-Aib-L-E-K-Q-A-Q-Aib-A-
F-V-E-W-L-I-K(1,3)-G-G-P-S-S-G-A-S-L-R-H-Y-Y-N-
W-L-T-R-Q-R-Y-NH2.C20DA-gGlu-gGlu-gGlu-gGlu-gGlu-
gGlu-gGlu-eLys(1,2)

MW (calculated): 7029.8 Da
Synthesis and purification methods: 501; P02
LCMS: A01-A; Rt: 12.6 min; m/3: m/4: 1758.2 m/5:

Compound 226
(SEQ ID NO: 552)
Y-Aib-E-G-T-F-T-S-D-Y-S-I-Aib-L-E-K-Q-A-Q-Aib-A-
F-V-E-W-L-I-K(1,3)-G-G-P-S-E-P-A-S-L-R-H-Y-Y-N-
W-L-T-R-Q-R-Y-NH2.C20DA-gGlu-gGlu-gGlu-
gGlu-OEG-OEG(1,2)

MW (calculated): 6886.7 Da
Synthesis and purification methods: S02; P03
LCMS: A02-B; Rt: 11.6 min; m/3: 2296.6 m/4: 1722.6 m/5:

Compound 227
(SEQ ID NO: 553)
Y-Aib-E-G-T-F-T-S-D-Y-S-I-Aib-L-E-K-Q-A-Q-Aib-A-
F-V-E-W-L-I-K(1,3)-G-G-P-S-E-G-Q-S-L-R-H-Y-Y-N-
W-L-T-R-Q-R-Y-NH2.C20DA-gGlu-gGlu-gGlu-gGlu-gGlu-
gGlu-eLys(1,2)

MW (calculated): 6999.8 Da
Synthesis and purification methods: S02; P03
LCMS: A02-B; Rt: 10.2 min; m/3: 2334.1 m/4: 1750.9 m/5:

Compound 228
(SEQ ID NO: 554)
Y-Aib-E-G-T-F-T-S-D-Y-S-I-Aib-L-E-K-Q-A-Q-Aib-A-
F-V-Q-W-L-I-K(1,3)-G-G-P-S-E-G-A-S-L-R-H-Y-Y-N-
W-L-T-R-Q-R-Y-NH2.C20DA-gGlu-gGlu-gGlu-gGlu-
gGlu-OEG-OEG(1,2)

MW (calculated): 6974.8 Da
Synthesis and purification methods: S02; P03
LCMS: A02-A; Rt: 7.7 min; m/3: 2326.1 m/4: 1744.6 m/5:

Compound 229
(SEQ ID NO: 555)
Y-Aib-E-G-T-F-T-S-D-Y-S-I-Aib-L-E-K-Q-A-Q-Aib-A-
F-V-E-W-L-I-K(1,3)-G-G-P-S-S-G-A-S-L-R-H-Y-Y-N-
W-L-T-R-Q-R-Y-NH2.C20DA-gGlu-gGlu-gGlu-gGlu-
gGlu-OEG-OEG(1,2)

MW (calculated): 6933.8 Da
Synthesis and purification methods: S02; P03
LCMS: A02-A; Rt: 7.8 min; m/3: 2312.1 m/4: 1734.4 m/5:

Compound 230
(SEQ ID NO: 556)
Y-Aib-E-G-T-F-T-S-D-Y-S-I-Aib-L-E-K-Q-A-Q-Aib-A-
F-V-E-W-L-I-K(1,3)-G-G-P-E-E-G-A-S-L-R-H-Y-Y-N-
W-L-T-R-Q-R-Y-NH2.C20DA-gGlu-gGlu-gGlu-gGlu-
gGlu-eLys(1,2)

MW (calculated): 6855.7 Da
Synthesis and purification methods: 501; P02
LCMS: A01-A; Rt: 13.0 min; m/3: m/4: 1714.6 m/5:

Compound 231
(SEQ ID NO: 557)
Y-Aib-E-G-T-F-T-S-D-Y-S-I-Aib-L-E-K-Q-A-Q-Aib-A-
F-I-E-W-L-I-K(1,3)-G-G-P-S-E-G-A-S-L-R-H-Y-Y-N-
W-L-T-R-Q-R-Y-NH2.C20DA-gGlu-gGlu-gGlu-gGlu-
OEG-OEG(1,2)

MW (calculated): 6860.7 Da
Synthesis and purification methods: S01; P02
LCMS: A01-A; Rt: 13.7 min; m/3: m/4: 1716.0 m/5:

Compound 232
(SEQ ID NO: 558)
Y-Aib-E-G-T-F-T-S-D-Y-S-I-Aib-L-E-K-Q-A-Q-Aib-A-
F-V-E-W-L-I-K(1,3)-G-G-P-S-Y-G-A-S-L-R-H-Y-Y-N-
W-L-T-R-Q-R-Y-NH2.C20DA-gGlu-gGlu-gGlu-gGlu-gGlu-
gGlu-eLys(1,2)

MW (calculated): 6976.8 Da
Synthesis and purification methods: S02; P03
LCMS: A02-A; Rt: 7.4 min; m/3: 2326.8 m/4: 1745.9 m/5:

Compound 233
(SEQ ID NO: 559)
Y-Aib-E-G-T-F-T-S-D-Y-S-I-Aib-L-E-K-Q-A-Q-Aib-A-
F-V-E-W-L-I-K(1,3)-G-G-P-S-E-P-A-S-L-R-H-Y-Y-N-
W-L-T-R-Q-R-Y-NH2.C20DA-gGlu-gGlu-gGlu-gGlu-gGlu-
gGlu-eLys(1,2)

MW (calculated): 6982.8 Da
Synthesis and purification methods: S01; P02
LCMS: A01-B; Rt: 34.6 min; m/3: m/4: 1746.4 m/5:

Compound 234
(SEQ ID NO: 560)
Y-Aib-E-G-T-F-T-S-D-L-S-I-Aib-L-E-K-Q-A-Q-Aib-A-F-V-E-W-L-I-K(1,3)-G-G-P-S-E-G-A-S-L-R-H-
Y-Y-N-W-L-T-R-Q-R-Y-NH2.C20DA-gGlu-gGlu-gGlu-gGlu-Glu-gGlu-eLys(1,2)

MW (calculated): 6892.8 Da
Synthesis and purification methods: S01; P02
LCMS: A01-B; Rt: 36.4 min; m/3: m/4: 1723.9 m/5:

Compound 235
(SEQ ID NO: 561)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-
H-Y-Y-N-W-L-T-R-Q-R-Y-NH2.C20DA-gGlu-gGlu-gGlu-gGlu-gGlu-gGlu(1,2)

MW (calculated): 6772.6 Da
Synthesis and purification methods: S01; L20
LCMS: A01-A; Rt: 6.7 min; m/3: 2259.1 m/4: 1694.4 m/5:

Compound 236
(SEQ ID NO: 562)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-R-
H-Y-Y-N-W-L-T-R-Q-NMeR-Y-NH2.C20DA-gGlu-gGlu-gGlu-gGlu-gGlu-gGlu-eLys(1,2)

MW (calculated): 6956.8 Da
Synthesis and purification methods: S01; P02
LCMS: A01-B; Rt: 35.7 min; m/3: m/4: 1739.9 m/5:

Compound 237
(SEQ ID NO: 563)
Y-Aib-E-G-T-F-T-S-D-Y-S-I-Aib-L-E-K-Q-A-Q-Aib-A-F-V-E-W-L-I-K(1,3)-G-G-P-S-E-G-E-S-L-
R-H-Y-Y-N-W-L-T-R-Q-R-Y-NH2.C20DA-gGlu-gGlu-gGlu-gGlu-gGlu-gGlu-eLys(1,2)

MW (calculated): 7000.8 Da
Synthesis and purification methods: S02; P03
LCMS: A02-A; Rt: 6.2 min; m/3: 2334.9 m/4: 1751.3 m/5:

Compound 238
(SEQ ID NO: 564)
Y-Aib-E-G-T-F-T-S-D-Y-S-I-Aib-L-E-K-Q-A-Q-Aib-A-F-V-E-W-L-I-K(1,3)-G-G-P-S-S-G-A-S-L-R-
H-Y-Y-N-W-L-T-R-Q-R-Y-NH2.C20DA-gGlu-gGlu-gGlu-gGlu-gGlu(1,2)

MW (calculated): 6643.4 Da
Synthesis and purification methods: 501; P02
LCMS: A01-A; Rt: 13.7 min; m/3: m/4: 1661.6 m/5:

Compound 239
(SEQ ID NOS 565 and 638, respectively)
Y-Aib-E-G-T-F-T-S-D-Y-S-I-Aib-L-E-K-Q-A-Q-Aib-A-F-V-Q-W-L-I-K(1,3)-G-G-P-S-E-G-A-S-L-R-H-
Y-Y-N-W-L-T-R-Q-R-Y-NH2.C20DA-E-E-E-E-OEG-OEG(1,2)

MW (calculated): 6845.7 Da
Synthesis and purification methods: S02; P03
LCMS: A02-A; Rt: 7.9 min; m/3: 2283.0 m/4: 1712.3 m/5:

Compound 240
(SEQ ID NOS 566
Y-Aib-E-G-T-F-T-S-D-Y-S-I-Aib-L-E-K-Q-A-Q-Aib-A-F-V-Q-W-L-I-K(1,3)-G-G-P-S-E-G-A-S-L-R-
H-Y-Y-N-W-L-T-R-Q-R-Y-NH2.C20DA-E-E-E-E-E-eLys(1,2)

MW (calculated): 6812.7 Da
Synthesis and purification methods: S02; P03
LCMS: A02-A; Rt: 7.5 min; m/3: 2272.1 m/4: 1704.2 m/5:

Compound 241
(SEQ ID NO: 567)
Y-Aib-E-G-T-F-T-S-D-Y-S-I-Aib-L-E-K-Q-A-Q-Aib-A-F-V-E-W-L-I-K(1,3)-G-G-P-S-E-G-V-S-L-R-
H-Y-Y-N-W-L-T-R-Q-R-Y-NH2.C20DA-gGlu-gGlu-gGlu-gGlu-gGlu-gGlu-eLys(1,2)

MW (calculated): 6970.8 Da
Synthesis and purification methods: S02; P03
LCMS: A02-A; Rt: 7.3 min; m/3: 2323.8 m/4: 1743.4 m/5:

Compound 242
(SEQ ID NO: 568)
Y-Aib-E-G-T-F-T-S-D-Y-S-I-Aib-L-E-K-Q-A-Q-Aib-A-F-V-Q-W-L-I-K(1,3)-G-G-P-S-E-G-A-S-L-R-
H-Y-Y-N-W-L-T-R-Q-R-Y-NH2.C20DA-gGlu-gGlu-gGlu-gGlu-gGlu-eLys(1,2)

MW (calculated): 6812.7 Da
Synthesis and purification methods: S02; P03
LCMS: A02-B; Rt: 10.6 min; m/3: 2272.0 m/4: 1704.2 m/5:

Compound 243
(SEQ ID NO: 569)
Y-Aib-E-G-T-F-T-S-D-Y-S-I-Aib-L-E-K-Q-A-Q-Aib-A-F-V-Q-W-L-I-K(1,3)-G-G-P-S-E-G-A-S-L-R-
H-Y-Y-N-W-L-T-R-Q-R-Y-NH2.C20DA-gGlu-gGlu-gGlu-gGlu-Glu(1,2)

MW (calculated): 6684.5 Da
Synthesis and purification methods: S02; P03
LCMS: A02-A; Rt: 7.8 min; m/3: 2228.7 m/4: 1672.1 m/5:

Compound 244
(SEQ ID NO: 570)
Y-Aib-E-G-T-F-T-S-D-Y-S-I-Aib-L-E-K-Q-A-Q-Aib-A-F-V-E-W-L-I-K(1,3)-G-G-P-S-T-G-A-S-L-R-
H-Y-Y-N-W-L-T-R-Q-R-Y-NH2.C20DA-gGlu-gGlu-gGlu-gGlu-gGlu-gGlu-eLys(1,2)

MW (calculated): 6914.8 Da
Synthesis and purification methods: S02; P03
LCMS: A02-B; Rt: 10.8 min; m/3: 2306.2 m/4: 1729.9 m/5: Compound 245

Compound 245
(SEQ ID NO: 571)
Y-Aib-E-G-T-F-T-S-D-Y-S-I-Aib-L-E-K-Q-A-Q-Aib-A-F-V-Q-W-L-I-K(1,3)-G-G-P-S-E-G-A-S-L-R-H-
Y-Y-N-W-L-T-R-Q-R-Y-NH2.C18DA-gGlu-gGlu-gGlu-glu-gGlu-Glu-AHX(1,2)

MW (calculated): 6898.7 Da
Synthesis and purification methods: S02; P03
LCMS: A02-B; Rt: 8.6 min; m/3: 2300.5 m/4: 1725.9 m/5:

Compound 246
(SEQ ID NO: 572)
Y-Aib-E-G-T-F-T-S-D-Y-S-I-Aib-L-E-K-Q-A-Q-Aib-A-F-V-Q-W-L-I-K(1,3)-G-G-P-S-E-G-A-S-L-R-
H-Y-Y-N-W-L-T-R-Q-R-Y-NH2.C18DA-gGlu-gGlu-gGlu-gGlu-gGlu-OEG-OEG(1,2)

MW (calculated): 6946.8 Da
Synthesis and purification methods: S02; P03
LCMS: A02-A; Rt: 7.0 min; m/3: 2316.7 m/4: 1737.6 m/5:
MW (calculated): 6542.3 Da
Synthesis and purification methods: S01; P02
LCMS: A01-A; Rt: 12.7 min; m/3: m/4: 1636.4 m/5:

Compound 247
(SEQ ID NO: 573)
Y-Aib-E-G-T-F-T-S-D-Y-S-I-Aib-L-E-K-Q-A-Q-Aib-A-F-I-E-W-L-I-K(1,3)-G-G-P-S-E-G-A-S-L-R-
H-Y-Y-N-W-L-T-R-Q-R-Y-NH2.C18DA-gGlu-gGlu-gGlu-gGlu(1,2)

MW (calculated): 6731.5 Da

Compound 248
(SEQ ID NO: 574)
Y-Aib-E-G-T-F-T-S-D-Y-S-I-Aib-L-E-K-Q-A-Q-Aib-A-F-V-E-W-L-I-K(1,3)-G-G-P-E-E-G-A-S-L-R-H-
Y-Y-N-W-L-T-R-Q-R-Y-NH2.C18DA-gGlu-gGlu-gGlu-OEG-OEG(1,2)

Synthesis and purification methods: S02; P03
LCMS: A02-A; Rt: 7.3 min; m/3: 2245.0 m/4: 1684.0 m/5:

Compound 249
(SEQ ID NOS 575 and 643, respectively)
Y-Aib-E-G-T-F-T-S-D-Y-S-I-Aib-L-E-K-Q-A-Q-Aib-A-
F-V-Q-W-L-I-K(1,3)-G-G-P-S-E-G-A-S-L-R-H-Y-Y-N-
W-L-T-R-Q-R-Y-NH2.C18DA-E-E-E-E-E-E-eLys(1,2)

MW (calculated): 6913.7 Da
Synthesis and purification methods: S02; P03
LCMS: A02-A; Rt: 6.8 min; m/3: 2305.4 m/4: 1729.5 m/5:

Compound 250
(SEQ ID NO: 576)
Y-Aib-E-G-T-F-T-S-D-Y-S-I-Aib-L-E-K-Q-A-Q-Aib-A-
F-V-Q-W-L-I-K(1,3)-G-G-P-S-E-G-A-S-L-R-H-Y-Y-N-
W-L-T-R-Q-R-Y-NH2.C18DA-gGlu-gGlu-gGlu-gGlu-
gGlu(1,2)

MW (calculated): 6656.4 Da
Synthesis and purification methods: S02; P03
LCMS: A02-A; Rt: 7.0 min; m/3: 2219.5 m/4: 1664.9 m/5:

Compound 251
(SEQ ID NO: 577)
Y-Aib-E-G-T-F-T-S-D-Y-S-I-Aib-L-E-K-Q-A-E-Aib-A-
F-V-E-W-L-I-K(1,3)-G-G-P-S-E-G-A-S-L-R-H-Y-Y-N-
W-L-T-R-Q-R-Y-NH2.C18DA-gGlu-gGlu-gGlu-gGlu-OEG-
OEG(1,2)

MW (calculated): 6819.6 Da
Synthesis and purification methods: S01; L20
LCMS: A01-A; Rt: 6.2 min; m/3: 2274.4 m/4: 1705.9 m/5:

Compound 252
(SEQ ID NO: 578)
Y-Aib-E-G-T-F-T-S-D-Y-S-I-Aib-L-E-K-Q-A-Q-Aib-A-
F-V-Q-W-L-I-K(1,3)-G-G-P-S-E-G-A-S-L-R-H-Y-Y-N-
W-L-T-R-Q-R-Y-NH2.C18DA-gGlu-gGlu-gGlu-gGlu-gGlu-
gGlu(1,2)

MW (calculated): 6785.6 Da
Synthesis and purification methods: S02; P03
LCMS: A02-B; Rt: 9.8 min; m/3: 2263.0 m/4: 1697.4 m/5:

Compound 253
(SEQ ID NO: 579)
Y-Aib-E-G-T-F-T-S-D-Y-S-I-Aib-L-E-K-Q-A-Q-Aib-A-
F-V-E-W-L-I-K(1,3)-G-G-P-S-S-G-A-S-L-R-H-Y-Y-N-
W-L-T-R-Q-R-Y-NH2.C18DA-gGlu-gGlu-gGlu-gGlu-
gGlu(1,2)

MW (calculated): 6615.4 Da
Synthesis and purification methods: S01; P02
LCMS: A01-A; Rt: 12.4 min; m/3: m/4: 1654.7 m/5:

Compound 254
(SEQ ID NO: 580)
Y-Aib-E-G-T-F-T-S-D-Y-S-I-Aib-L-E-K-Q-A-Q-Aib-A-
F-V-E-W-L-I-K(1,3)-G-G-P-S-S-G-A-S-L-R-H-Y-Y-N-
W-L-T-R-Q-R-Y-NH2.C18DA-gGlu-gGlu-gGlu-gGlu-
OEG-OEG(1,2)

MW (calculated): 6776.6 Da
Synthesis and purification methods: S02; P03
LCMS: A02-A; Rt: 7.2 min; m/3: 2259.7 m/4: 1695.0 m/5:

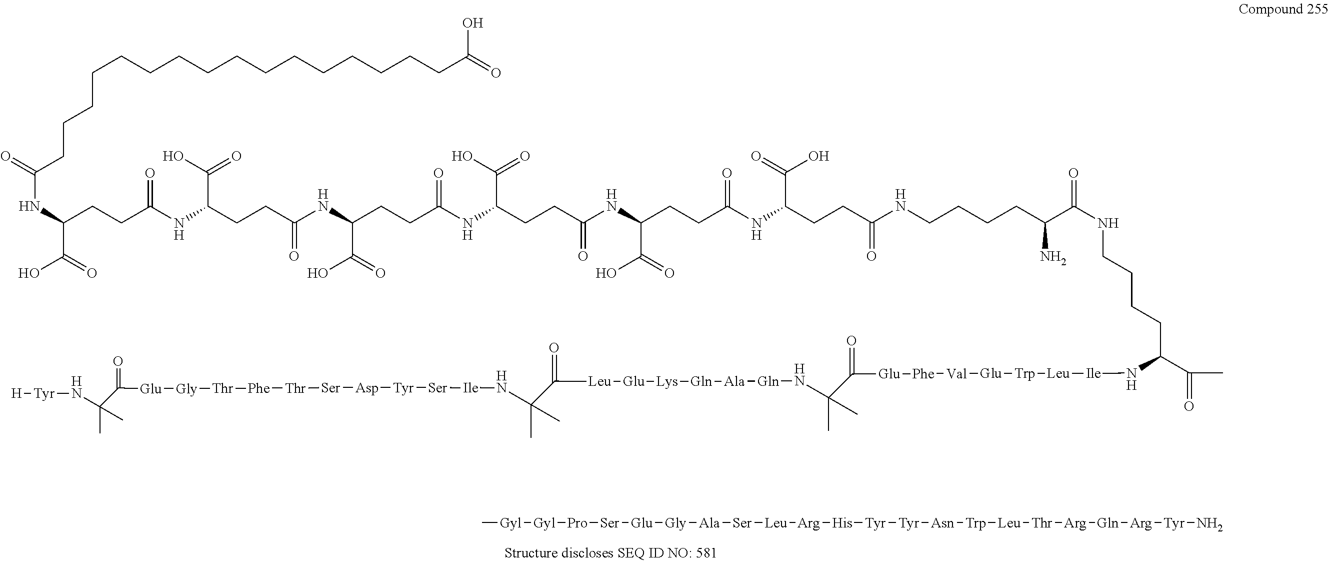

(SEQ ID NO: 581)
Y-Aib-E-G-T-F-T-S-D-Y-S-I-Aib-L-E-K-Q-A-Q-Aib-E-
F-V-E-W-L-I-K(1,3)-G-G-P-S-E-G-A-S-L-R-H-Y-Y-N-
W-L-T-R-Q-R-Y-NH2.C18DA-gGlu-gGlu-gGlu-gGlu-gGlu-
gGlu-eLys(1,2)

MW (calculated): 6972.7 Da
Synthesis and purification methods: S02; P03
LCMS: A02-A; Rt: 6.6 min; m/3: 2325.1 m/4: 1744.1 m/5:

Compound 256
(SEQ ID NO: 582)
Y-Aib-E-G-T-F-T-S-D-Y-S-I-Aib-L-E-K-Q-A-Q-Aib-A-
F-V-E-W-L-I-K(1,3)-G-G-P-S-E-P-A-S-L-R-H-Y-Y-N-
W-L-T-R-Q-R-Y-NH2.C18DA-gGlu-gGlu-gGlu-gGlu-OEG-
OEG(1,2)

MW (calculated): 6858.7 Da
Synthesis and purification methods: S02; P03
LCMS: A02-B; Rt: 10.2 min; m/3: 2286.9 m/4: 1715.7 m/5:

Compound 257
(SEQ ID NOS 583 and 636, respectively)
Y-Aib-E-G-T-F-T-S-D-Y-S-I-Aib-L-E-K-Q-A-Q-Aib-A-
F-V-Q-W-L-I-K(1,3)-G-G-P-S-E-G-A-S-L-R-H-Y-Y-N-
W-L-T-R-Q-R-Y-NH2.C18DA-E-E-E-E-E(1,2)

Synthesis and purification methods: S02; P03
LCMS: A02-B; Rt: 10.4 min; m/3: 2220.0 m/4: 1665.3 m/5:

Compound 258
(SEQ ID NO: 584)
Y-Aib-E-G-T-F-T-S-D-Y-S-I-Aib-L-E-K-Q-A-Q-Aib-A-
F-I-E-W-L-I-K(1,3)-G-G-P-S-E-G-A-S-L-R-H-Y-Y-N-
W-L-T-R-Q-R-Y-NH2.C18DA-gGlu-gGlu-gGlu-glu-OEG-
OEG(1,2)

MW (calculated): 6832.7 Da
Synthesis and purification methods: 501; P02
LCMS: A01-A; Rt: 12.5 min; m/3: m/4: 1708.9 m/5:

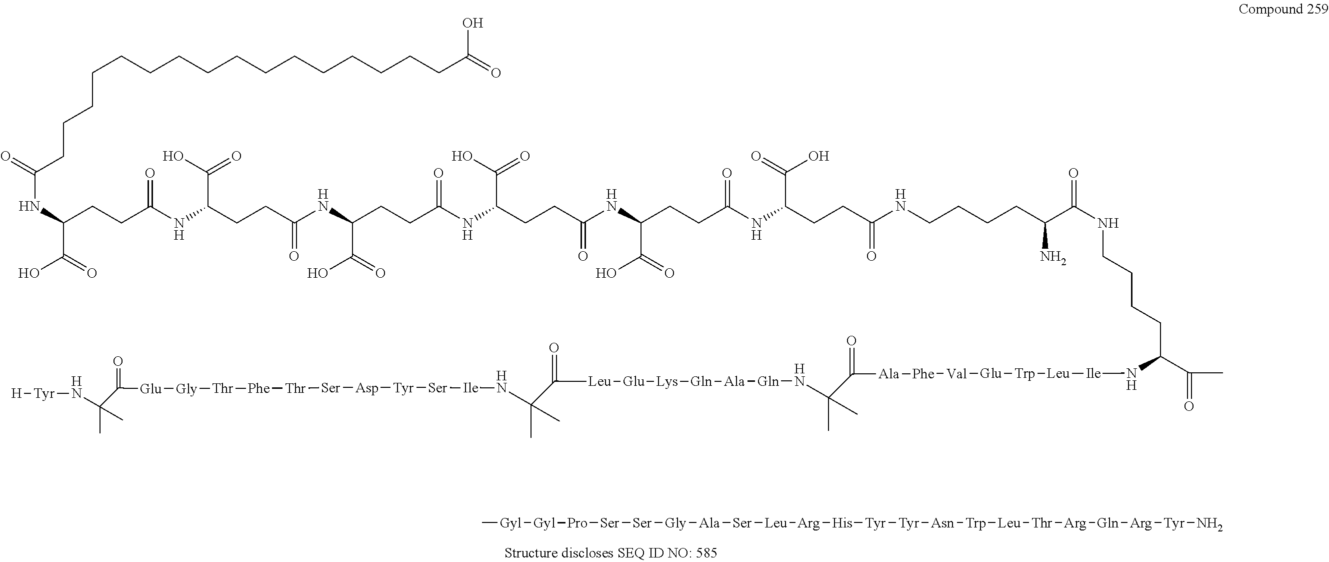

(SEQ ID NO: 585)
Y-Aib-E-G-T-F-T-S-D-Y-S-I-Aib-L-E-K-Q-A-Q-Aib-A-
F-V-E-W-L-I-K(1,3)-G-G-P-S-S-G-A-S-L-R-H-Y-Y-N-
W-L-T-R-Q-R-Y-NH2.C18DA-gGlu-gGlu-gGlu-gGlu-gGlu-
gGlu-eLys(1,2)

MW (calculated): 6872.7 Da
Synthesis and purification methods: S02; P03
LCMS: A02-A; Rt: 6.8 min; m/3: 2291.5 m/4: 1719.2 m/5:

Compound 260
(SEQ ID NO: 586)
Y-Aib-E-G-T-F-T-S-D-Y-S-I-Aib-L-E-K-Q-A-Q-Aib-A-
F-V-E-W-L-I-K(1,3)-G-G-P-S-S-G-A-S-L-R-H-Y-Y-N-
W-L-T-R-Q-R-Y-NH2.C18DA-gGlu-gGlu-gGlu-gGlu-gGlu-
AHX(1,2)

MW (calculated): 6728.5 Da
Synthesis and purification methods: S02; P03
LCMS: A02-A; Rt: 7.3 min; m/3: 2243.7 m/4: 1683.2 m/5:

Compound 261
(SEQ ID NO: 587)
Y-Aib-E-G-T-F-T-S-D-Y-S-I-Aib-L-E-K-Q-A-Q-Aib-A-
F-V-E-W-L-I-K(1,3)-G-G-P-S-S-G-A-S-L-R-H-Y-Y-N-
W-L-T-R-Q-R-Y-NH2.C18DA-gGlu-gGlu-gGlu-gGlu-gGlu-
OEG-OEG(1,2)

MW (calculated): 6905.7 Da
Synthesis and purification methods: S02; P03
LCMS: A02-A; Rt: 7.1 min; m/3: 2303.4 m/4: 1727.7 m/5:

Compound 262
(SEQ ID NO: 588)
Y-Aib-E-G-T-F-T-S-D-Y-S-I-Aib-L-E-K-Q-A-E-Aib-A-
F-V-E-W-L-I-K(1,3)-G-G-P-S-E-G-A-S-L-R-H-Y-Y-N-
W-L-T-R-Q-R-Y-NH2.C18DA-gGlu-gGlu-gGlu-
gGlu(1,2)

MW (calculated): 6529.3 Da
Synthesis and purification methods: S01; P02
LCMS: A01-A; Rt: 12.9 min; m/3: m/4: 1633.1 m/5:

Compound 263
(SEQ ID NO: 589)
Y-Aib-E-G-T-F-T-S-D-Y-S-I-Aib-L-E-K-Q-A-Q-Aib-A-
F-V-E-W-L-I-K(1,3)-G-G-P-S-E-P-A-S-L-R-H-Y-Y-N-
W-L-T-R-Q-R-Y-NH2.C18DA-gGlu-gGlu-gGlu-gGlu-gGlu-
eLys(1,2)

MW (calculated): 6825.7 Da
Synthesis and purification methods: S02; P03
LCMS: A02-B; Rt: 12.5 min; m/3: 2275.8 m/4: 1707.2 m/5:

Compound 264
(SEQ ID NOS 590
Y-Aib-E-G-T-F-T-S-D-Y-S-I-Aib-L-E-K-Q-A-Q-Aib-A-F-V-Q-W-L-I-K(1,3)-G-G-P-S-E-G-A-S-L-R-
H-Y-Y-N-W-L-T-R-Q-R-Y-NH2.C18DA-E-E-E-E-E-E(1,2)

MW (calculated): 6785.6 Da
Synthesis and purification methods: S02; P03
LCMS: A02-B; Rt: 10.3 min; m/3: 2263.0 m/4: 1697.4 m/5:

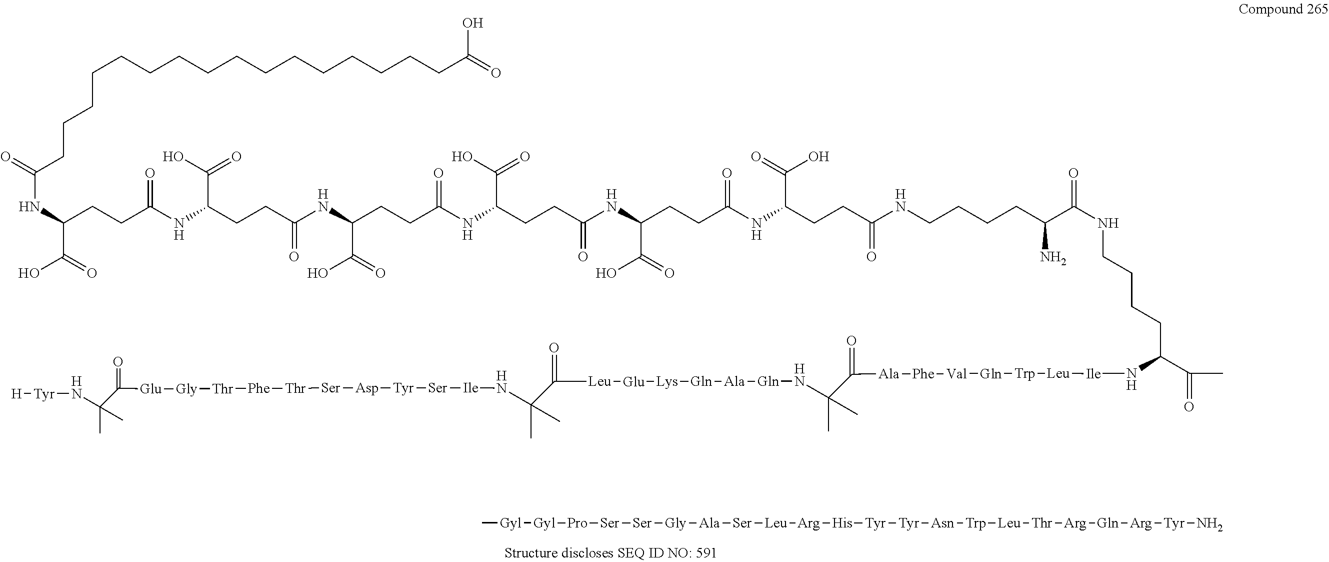

(SEQ ID NO: 591)
Y-Aib-E-G-T-F-T-S-D-Y-S-I-Aib-L-E-K-Q-A-Q-Aib-A-F-V-Q-W-L-I-K(1,3)-G-G-P-S-E-G-A-S-L-R-
H-Y-Y-N-W-L-T-R-Q-R-Y-NH2.C18DA-gGlu-gGlu-gGlu-gGlu-gGlu-gGlu-eLys(1,2)

MW (calculated): 6913.7 Da
Synthesis and purification methods: S02; P03
LCMS: A02-B; Rt: 8.9 min; m/3: 2305.4 m/4: 1729.3 m/5:

Compound 266
(SEQ ID NO: 592)
Y-Aib-E-G-T-F-T-S-D-Y-S-I-Aib-L-E-K-Q-A-Q-Aib-A-F-V-Q-W-L-I-K(1,3)-G-G-P-S-E-G-A-S-L-R-
H-Y-Y-N-W-L-T-R-Q-R-Y-NH2.C18DA-gGlu-gGlu-gGlu-OEG-OEG(1,2)

MW (calculated): 6688.5 Da
Synthesis and purification methods: S02; P03
LCMS: A02-B; Rt: 10.2 min; m/3: 2230.3 m/4: 1673.1 m/5:

Compound 267
(SEQ ID NO: 593)
Y-Aib-E-G-T-F-T-S-D-Y-S-I-Aib-L-E-K-Q-A-Q-Aib-A-F-V-Q-W-L-I-K(1,3)-G-G-P-S-E-G-A-S-L-R-
H-Y-Y-N-W-L-T-R-Q-R-Y-NH2.C18DA-gGlu-gGlu-gGlu-gGlu-gGlu-eLys(1,2)

MW (calculated): 6784.6 Da
Synthesis and purification methods: S02; P03
LCMS: A02-B; Rt: 9.3 min; m/3: 2263.0 m/4: 1697.2 m/5:

Compound 268
(SEQ ID NO: 594)
Y-Aib-E-G-T-F-T-S-D-Y-S-I-Aib-L-E-K-Q-A-Q-Aib-A-F-V-Q-W-L-I-K(1,3)-G-G-P-S-E-G-A-S-L-
R-H-Y-Y-N-W-L-T-R-Q-R-Y-NH2.C18DA-gGlu-gGlu-gGlu-gGlu(1,2)

MW (calculated): 6527.3 Da
Synthesis and purification methods: S02; P03
LCMS: A02-B; Rt: 10.3 min; m/3: 2176.8 m/4: 1632.9 m/5:

Compound 269
(SEQ ID NO: 595)
Y-Aib-E-G-T-F-T-S-D-Y-S-I-Aib-L-E-K-Q-A-Q-Aib-A-F-V-Q-W-L-I-K(1,3)-G-G-P-S-E-G-A-S-L-
R-H-Y-Y-N-W-L-T-R-Q-R-Y-NH2.C20DA-gGlu-gGlu-gGlu-gGlu-OEG-OEG(1,2)

MW (calculated): 6845.7 Da
Synthesis and purification methods: S02; P03
LCMS: A02-A; Rt: 7.7 min; m/3: 2282.5 m/4: 1712.3 m/5:

Compound 270
(SEQ ID NO: 596)
Y-Aib-E-G-T-F-T-S-D-Y-S-I-Aib-L-E-K-Q-A-Q-Aib-A-F-V-Q-W-L-I-K(1,3)-G-G-P-S-E-G-A-S-L-R-
H-Y-Y-N-W-L-T-R-Q-R-Y-NH2.C18DA-gGlu-gGlu-gGlu-gGlu-OEG-OEG(1,2)

MW (calculated): 6817.6 Da
Synthesis and purification methods: S02; P03
LCMS: A02-B; Rt: 10.1 min; m/3: 2273.8 m/4: 1705.2 m/5:

Compound 271
(SEQ ID NO: 597)
Y-Aib-E-G-T-F-T-S-D-Y-S-I-Aib-L-E-K-Q-A-Q-Aib-E-F-V-E-W-L-I-K(1,3)-G-G-P-S-E-G-A-S-L-
R-H-Y-Y-N-W-L-T-R-Q-R-Y-NH2.C20DA-gGlu-gGlu-gGlu-gGlu(1,2)

MW (calculated): 6614.4 Da
Synthesis and purification methods: S02; P03
LCMS: A02-A; Rt: 7.8 min; m/3: 2205.7 m/4: 1654.6 m/5:

Compound 272
(SEQ ID NO: 598)
Y-Aib-E-G-T-F-T-S-D-Y-S-I-Aib-L-E-K-Q-A-Q-Aib-A-F-V-E-W-L-I-K(1,3)-G-G-P-E-E-G-A-S-L-R-
H-Y-Y-N-W-L-T-R-Q-R-Y-NH2.C18DA-gGlu-gGlu-gGlu-gGlu(1,2)

MW (calculated): 6570.3 Da
Synthesis and purification methods: S02; P03
LCMS: A02-A; Rt: 7.4 min; m/3: 2191.6 m/4: 1644.1 m/5:

Compound 273
(SEQ ID NO: 599)
Y-Aib-E-G-T-F-T-S-D-Y-S-I-Aib-L-E-K-Q-A-Q-Aib-E-F-V-E-W-L-I-K(1,3)-G-G-P-S-E-G-A-S-L-R-H-
Y-Y-N-W-L-T-R-Q-R-Y-NH2.C20DA-gGlu-gGlu-gGlu-OEG-OEG(1,2)

MW (calculated): 6775.6 Da
Synthesis and purification methods: S02; P03
LCMS: A02-A; Rt: 7.8 min; m/3: 2259.7 m/4: 1694.9 m/5:

Compound 274
(SEQ ID NO: 600)
Y-Aib-E-G-T-F-T-S-D-Y-S-I-Aib-L-E-K-Q-A-Q-Aib-E-F-V-E-W-L-I-K(1,3)-G-G-P-S-E-G-A-S-L-R-
H-Y-Y-N-W-L-T-R-Q-R-Y-NH2.C18DA-gGlu-gGlu-gGlu-OEG(1,2)

MW (calculated): 6602.4 Da
Synthesis and purification methods: S02; P03
LCMS: A02-B; Rt: 8.9 min; m/3: 2201.8 m/4: 1651.7 m/5:

Compound 275
(SEQ ID NO: 601)
Y-Aib-E-G-T-F-T-S-D-Y-S-I-Aib-L-E-K-Q-A-Q-Aib-E-F-V-E-W-L-I-K(1,3)-G-G-P-S-E-G-A-S-L-R-
H-Y-Y-N-W-L-T-R-Q-R-Y-NH2.C20DA-gGlu-gGlu-gGlu-gGlu-gGlu-gGlu-eLys(1,2)

MW (calculated): 7000.8 Da
Synthesis and purification methods: S02; P03
LCMS: A02-A; Rt: 7.3 min; m/3: 2335.4 m/4: 1751.1 m/5:

Compound 276
(SEQ ID NO: 602)
Y-Aib-E-G-T-F-T-S-D-Y-S-I-Aib-L-E-K-Q-A-Q-Aib-E-F-V-E-W-L-I-K(1,3)-G-G-P-S-E-G-A-S-L-R-H-Y-
Y-N-W-L-T-R-Q-R-Y-NH2.C18DA-gGlu-gGlu-gGlu-gGlu(1,2)

MW (calculated): 6586.3 Da
Synthesis and purification methods: S02; P03
LCMS: A02-A; Rt: 7.1 min; m/3: 2196.1 m/4: 1647.5 m/5:

Compound 277
(SEQ ID NO: 603)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-R-H-
Y-Y-N-W-L-T-R-bHGln-R-Y-NH2.C20DA-gGlu-gGlu-gGlu-gGlu-gGlu-gGlu-eLys(1,2)

MW (calculated): 6956.8 Da
Synthesis and purification methods: S01; P02
LCMS: A01-B; Rt: 35.1 min; m/3: m/4: 1740.0 m/5:

Compound 278
(SEQ ID NO: 604)
Y-Aib-E-G-T-F-T-S-D-Y-S-I-Aib-L-E-K-Q-A-E-Aib-A-F-V-E-W-L-I-K(1,3)-G-G-P-S-E-G-A-S-L-R-
H-Y-Y-N-W-L-T-R-Q-R-Y-NH2.C20DA-gGlu-gGlu-gGlu-Glu-OEG-OEG(1,2)

MW (calculated): 6847.7 Da
Synthesis and purification methods: S01; P02
LCMS: A01-A; Rt: 13.8 min; m/3: m/4: 1712.7 m/5:

Compound 279
(SEQ ID NO: 605)
Y-Aib-E-G-T-F-T-S-D-Y-S-I-Aib-L-E-K-Q-A-Q-Aib-E-F-V-E-W-L-I-K(1,3)-G-G-P-S-E-G-A-S-L-R-H-Y-
Y-N-W-L-T-R-Q-R-Y-NH2.C18DA-gGlu-gGlu-gGlu-gGlu-gGlu-gGlu-AHX(1,2)

MW (calculated): 6957.7 Da
Synthesis and purification methods: S02; P03
LCMS: A02-B; Rt: 8.4 min; m/3: 2320.4 m/4: 1741.0 m/5:

Compound 280
(SEQ ID NO: 606)
Y-Aib-E-G-T-F-T-S-D-Y-S-I-Aib-L-E-K-Q-A-E-Aib-A-F-V-E-W-L-I-K(1,3)-G-G-P-S-E-G-A-S-L-R-H-Y-
Y-N-W-L-T-R-Q-R-Y-NH2.C20DA-gGlu-gGlu-gGlu-gGlu(1,2)

MW (calculated): 6557.3 Da
Synthesis and purification methods: S01; P02
LCMS: A01-A; Rt: 14.1 min; m/3: m/4: 1640.2 m/5:

Compound 281
(SEQ ID NOS 607
Y-Aib-E-G-T-F-T-S-D-Y-S-I-Aib-L-E-K-Q-A-Q-Aib-A-F-V-Q-W-L-I-K(1,3)-G-G-P-S-E-G-A-S-L-R-
H-Y-Y-N-W-L-T-R-Q-R-Y-NH2.C18DA-E-E-E-E(1,2)

MW (calculated): 6527.3 Da
Synthesis and purification methods: S02; P03
LCMS: A02-A; Rt: 7.3 min; m/3: 2177.4 m/4: 1632.6 m/5:

Compound 282
(SEQ ID NO: 608)
Y-Aib-E-G-T-F-T-S-D-Y-S-I-Aib-L-E-K-Q-A-Q-Aib-E-F-V-E-W-L-I-K(1,3)-G-G-P-S-E-G-A-S-L-R-
H-Y-Y-N-W-L-T-R-Q-R-Y-NH2.C18DA-gGlu-OEG-OEG(1,2)

MW (calculated): 6489.3 Da
Synthesis and purification methods: S02; P03
LCMS: A02-A; Rt: 7.5 min; m/3: 2163.1 m/4: 1623.1 m/5:

Compound 283
(SEQ ID NO: 609)
Y-Aib-E-G-T-F-T-S-D-Y-S-I-Aib-L-E-K-Q-A-Q-Aib-A-F-I-E-W-L-I-K(1,3)-G-G-P-S-E-G-A-S-L-R-H-
Y-Y-N-W-L-T-R-Q-R-Y-NH2.C20DA-gGlu-gGlu-gGlu-gGlu(1,2)

MW (calculated): 6570.4 Da
Synthesis and purification methods: 501; P02
LCMS: A01-A; Rt: 14.0 min; m/3: m/4: 1643.5 m/5:

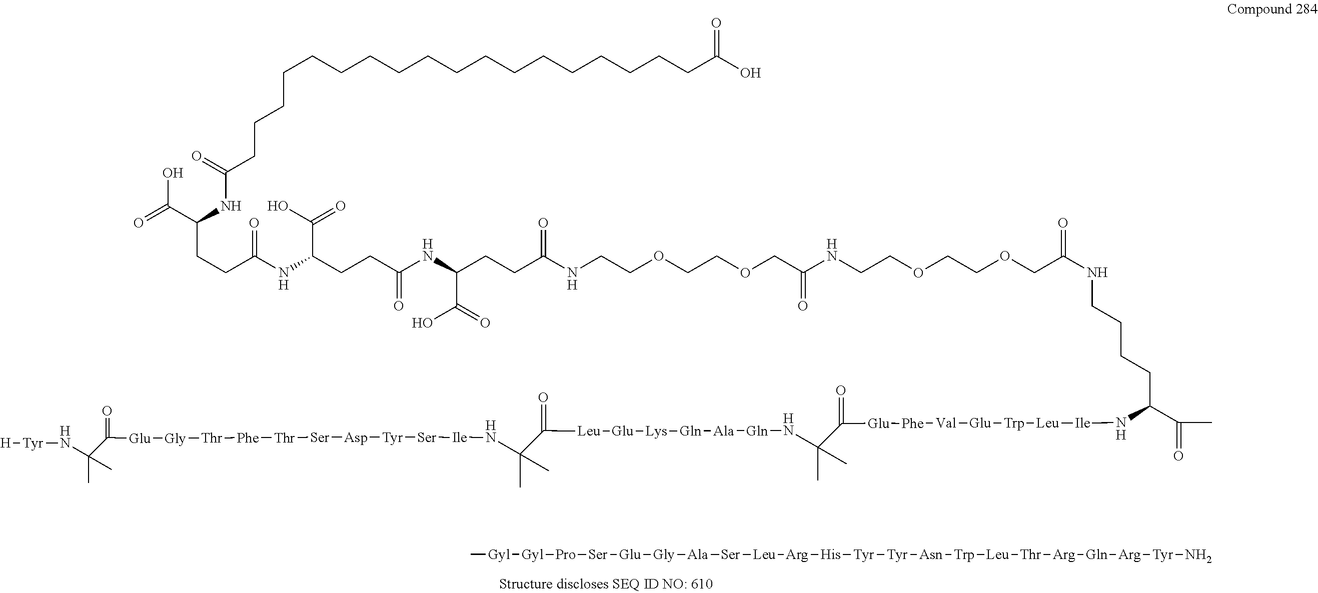

(SEQ ID NO: 610)
Y-Aib-E-G-T-F-T-S-D-Y-S-I-Aib-L-E-K-Q-A-Q-Aib-E-F-V-E-W-L-I-K(1,3)-G-G-P-S-E-G-A-S-L-R-
H-Y-Y-N-W-L-T-R-Q-R-Y-NH2.C18DA-gGlu-gGlu-gGlu-OEG-OEG(1,2)

MW (calculated): 6747.5 Da
Synthesis and purification methods: S02; P03
LCMS: A02-A; Rt: 7.1 min; m/3: 2249.9 m/4: 1687.7 m/5:

Compound 285
(SEQ ID NO: 611)
Y-Aib-E-G-T-F-T-S-D-Y-S-I-Aib-L-E-K-Q-A-Q-Aib-A-F-V-E-W-L-I-K(1,3)-G-G-P-E-E-G-A-S-L-R-H-
Y-Y-N-W-L-T-R-Q-R-Y-NH2.C20DA-gGlu-gGlu-gGlu-gGlu-Glu-gGlu-eLys(1,2)

MW (calculated): 6984.8 Da
Synthesis and purification methods: S02; P03
LCMS: A02-B; Rt: 10.7 min; m/3: 2329.3 m/4: 1747.2 m/5:

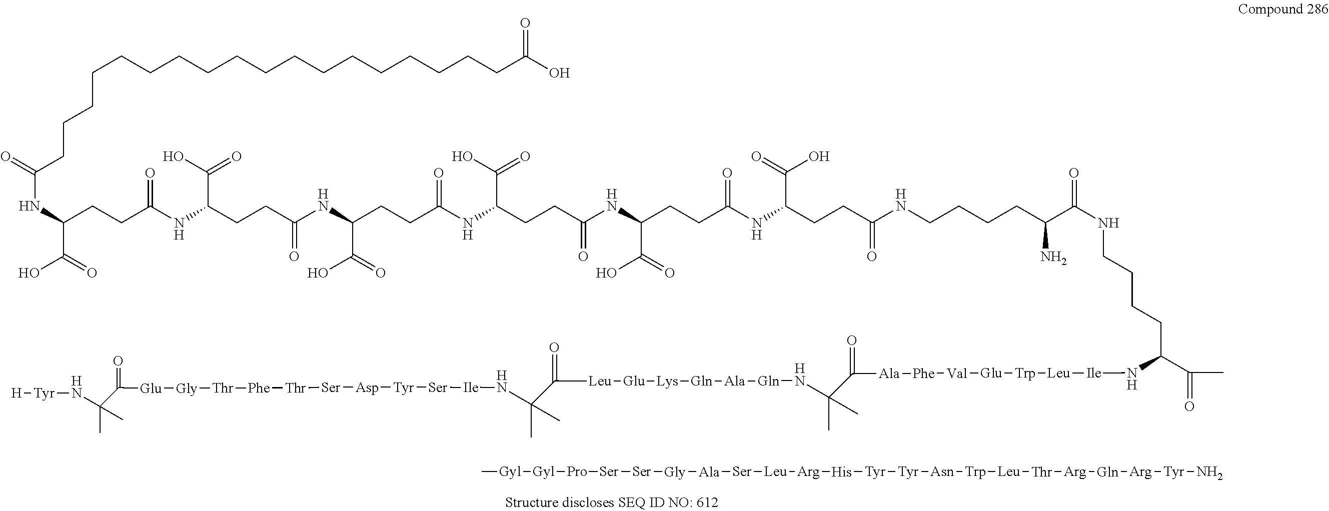

(SEQ ID NO: 612)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-R-H-
Y-Y-N-W-L-T-R-Q-R-Y-NH2.C20DA-gGlu-gGlu-gGlu-gGlu-gGlu-Glu-eLys(1,2)

MW (calculated): 6942.8 Da
Synthesis and purification methods: 501; P02
LCMS: A01-A; Rt: 12.7 min; m/3: 2315.4 m/4: 1736.7 m/5:

Compound 287
(SEQ ID NO: 613)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-R-
H-Y-Y-N-W-L-T-R-Q-R-Y-NH2.C20DA-gGlu-gGlu-gGlu-gGlu-OEG-OEG(1,2)

MW (calculated): 6846.7 Da
Synthesis and purification methods: S02; P03
LCMS: A02-A; Rt: 7.9 min; m/3: 2283.2 m/4: 1712.5 m/5:

Compound 288
(SEQ ID NO: 614)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-R-H-
Y-Y-N-W-L-T-R-Q-R-Y-NH2.C20DA-gGlu-gGlu-gGlu-gGlu-gGlu-gGlu-gGlu-eLys(1,2)

MW (calculated): 7071.9 Da
Synthesis and purification methods: 501; P02
LCMS: A01-A; Rt: 12.9 min; m/3: m/4: 1768.6 m/5:

Compound 289
(SEQ ID NO: 615)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-R-H-
Y-Y-N-W-L-T-R-Q-R-Y-NH2.C20DA-gGlu-gGlu-gGlu-gGlu-gGlu(1,2)

MW (calculated): 6685.5 Da
Synthesis and purification methods: 501; P02
LCMS: A01-A; Rt: 13.6 min; m/3: m/4: 1672.2 m/5:

Compound 290
(SEQ ID NO: 616)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-R-H-Y-
Y-N-W-L-T-R-Q-R-Y-NH2.C20DA-gGlu-gGlu-gGlu-Glu-gGlu-eLys(1,2)

MW (calculated): 6813.7 Da
Synthesis and purification methods: 501; P02
LCMS: A01-A; Rt: 13.0 min; m/3: m/4: m/5: 1363.6

Compound 291
(SEQ ID NOS 617
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-R-H-
Y-Y-N-W-L-T-R-Q-R-Y-NH2.C18DA-E-E-E-E(1,2)

MW (calculated): 6657.4 Da
Synthesis and purification methods: S02; P03
LCMS: A02-B; Rt: 10.6 min; m/3: 2117.4 m/4: 1633.3 m/5:

Compound 292
(SEQ ID NO: 618)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-R-
H-Y-Y-N-W-L-T-R-Q-R-Y-NH2.C18DA-gGlu-gGlu-gGlu-gGlu-gGlu(1,2)

MW (calculated): 6657.4 Da
Synthesis and purification methods: S02; P03
LCMS: A02-B; Rt: 10.6 min; m/3: 2220.6 m/4: 1665.5 m/5:

Compound 293
(SEQ ID NO: 619)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-
R-H-Y-Y-N-W-L-T-R-Q-R-Y-NH2.C18DA-gGlu-Glu-gGlu-gGlu-gGlu-gGlu-eLys(1,2)

MW (calculated): 6914.7 Da
Synthesis and purification methods: S02; P03
LCMS: A02-A; Rt: 6.8 min; m/3: 2306.3 m/4: 1729.9 m/5:

Compound 294
(SEQ ID NO: 620)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-R-
H-Y-Y-N-W-L-T-R-Q-R-Y-NH2.C18DA-gGlu-gGlu-gGlu-gGlu(1,2)

MW (calculated): 6528.3 Da
Synthesis and purification methods: S02; P03
LCMS: A02-B; Rt: 10.5 min; m/3: 2177.2 m/4: 1633.1 m/5:

Compound 295
(SEQ ID NO: 621)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-
R-H-Y-Y-N-W-L-T-R-Q-R-Y-NH2.C18DA-gGlu-gGlu-glu-gGlu-gGlu-eLys(1,2)

MW (calculated): 6785.6 Da
Synthesis and purification methods: S02; P03
LCMS: A02-B; Rt: 9.4 min; m/3: 2262.9 m/4: 1697.4 m/5:

Compound 296
(SEQ ID NO: 622)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-R-H-
Y-Y-N-W-L-T-R-Q-R-Y-NH2.C18DA-gGlu-gGlu-gGlu-gGlu-gGlu-Glu(1,2)

MW (calculated): 6786.5 Da
Synthesis and purification methods: S02; P03
LCMS: A02-B; Rt: 10.1 min; m/3: 2263.2 m/4: 1697.7 m/5:

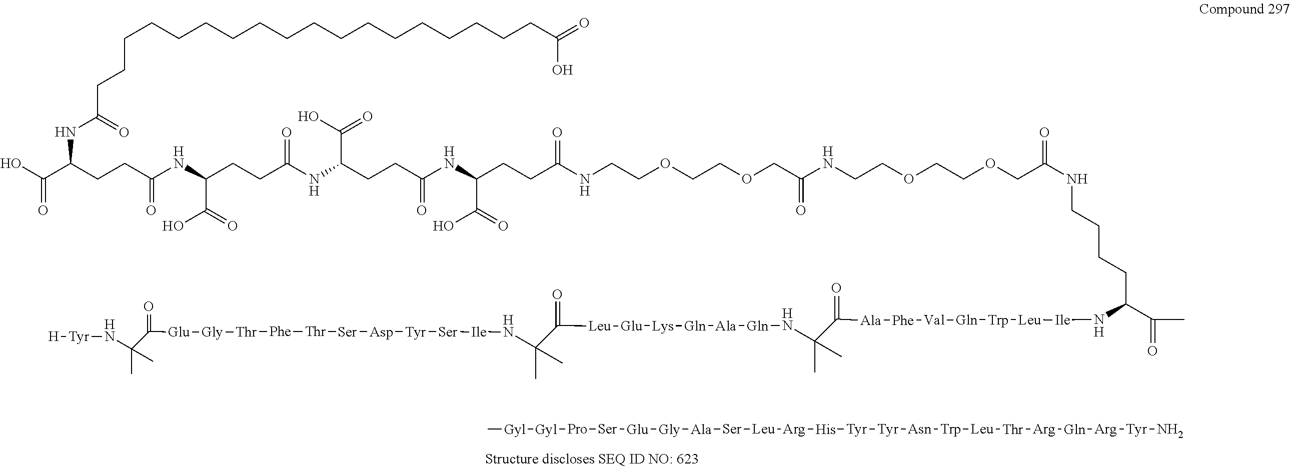

(SEQ ID NO: 623)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-
R-H-Y-Y-N-W-L-T-R-Q-R-Y-NH2.C18DA-gGlu-gGlu-gGlu-gGlu-OEG-OEG(1,2)

MW (calculated): 6818.6 Da
Synthesis and purification methods: S02; P03
LCMS: A02-B; Rt: 10.2 min; m/3: 2273.9 m/4: 1705.6 m/5:

Compound 298
(SEQ ID NO: 624)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-
R-H-Y-Y-N-W-L-T-R-Q-R-Y-NH2.C18DA-E-E-E-OEG-OEG(1,2)

MW (calculated): 6689.5 Da
Synthesis and purification methods: S02; P03
LCMS: A02-B; Rt: 10.6 min; m/3: 2230.7 m/4: 1673.3 m/5:

Compound 299
(SEQ ID NO: 625)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-R-
H-Y-Y-N-W-L-T-R-Q-R-Y-NH2.C18DA-gGlu-gGlu-gGlu-OEG-OEG(1,2)

MW (calculated): 6689.5 Da
Synthesis and purification methods: S02; P03
LCMS: A02-B; Rt: 10.4 min; m/3: 2230.9 m/4: 1673.4 m/5:

Compound 300
(SEQ ID NO: 626)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-
R-H-Y-Y-N-W-L-T-R-Q-R-Y-NH2.C18DA-gGlu-gGlu-gGlu-OEG(1,2)

MW (calculated): 6544.4 Da
Synthesis and purification methods: S02; P03
LCMS: A02-B; Rt: 9.3 min; m/3: 2182.3 m/4: 1637.3 m/5:

Compound 301
(SEQ ID NO: 627)
Y-Aib-E-G-T-F-T-S-D-Y-S-I-Aib-L-E-K-Q-A-Q-Aib-A-F-V-E-W-L-I-K(1,3)-G-G-P-S-E-G-A-S-
L-R-H-Y-Y-N-W-L-T-R-Q-R-Y-NH2.C20DA-gGlu-gGlu-gGlu-gGlu-gGlu-AHX(1,2)

MW (calculated): 6798.6 Da
Synthesis and purification methods: S02; P03
LCMS: A02-B; Rt: 10.4 min; m/3: 2267.1 m/4: 1700.9 m/5:

Compound 302
(SEQ ID NOS 628
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-
R-H-Y-Y-N-W-L-T-R-Q-R-Y-NH2.C18DA-E-E-E-E-E-E(1,2)

MW (calculated): 6786.5 Da
Synthesis and purification methods: S02; P03
LCMS: A02-B; Rt: 10.5 min; m/3: 2263.1 m/4: 1697.6 m/5:

Compound 303
(SEQ ID NO: 629)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-R-
H-Y-Y-N-W-L-T-R-Q-R-Y-NH2.C18DA-gGlu-gGlu-gGlu-gGlu-gGlu-gGlu-AHX(1,2)

MW (calculated): 6899.7 Da
Synthesis and purification methods: S02; P03
LCMS: A02-A; Rt: 7.2 min; m/3: 2301.3 m/4: 1726.1 m/5:

Compound 304
(SEQ ID NO: 630)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-R-
H-Y-Y-N-W-L-T-R-Q-R-Y-NH2.C18DA-gGlu-gGlu-gGlu-gGlu-gGlu-AHX(1,2)

MW (calculated): 6770.6 Da
Synthesis and purification methods: S02; P03
LCMS: A02-A; Rt: 7.2 min; m/3: 2258.0 m/4: 1694.0 m/5:

Compound 305
(SEQ ID NO: 631)
Y-Aib-E-G-T-F-T-S-D-Y-S-I-Aib-L-E-K-Q-A-Q-Aib-A-F-V-E-W-L-I-K(1,3)-G-G-P-S-E-G-A-S-L-
R-H-Y-Y-N-W-L-T-R-Q-R-Y-NH2.C20DA-gGlu-gGlu-gGlu-gGlu-gGlu-gGlu(1,2)

MW (calculated): 6814.6 Da
Synthesis and purification methods: 501; P02
LCMS: A01-B; Rt: 35.5 min; m/3: m/4: m/5: 1363.7

Ref. 1
(SEQ ID NOS 632
NMeY-Aib-E-G-T-aMeF-T-S-D-K(1,3)-S-I-Aib-L-E-K-Q-R-Q-Iva-E-F-V-R-H-L-L-N-K-Aib-T-R-
Q-R-Y-NH2.C16A-G-G-G-G(1,2)

and 641, respectively)
Example 41 of EP3467106
Solubility: <1 mg/ml (pH 7)

Ref. 2
(SEQ ID NOS 633
NMeY-Aib-E-G-T-aMeF-T-S-D-K(1,3)-S-I-Aib-L-E-K-Q-R-Q-Iva-E-F-V-R-H-L-L-N-K-Aib-T-R-
Q-R-Y-NH2.C18DA-G-G-G-G(1,2)

and 642, respectively)
Example 42 of EP3467106
Solubility: <1 mg/ml (pH 7)

### Examples

The following examples demonstrate certain specific embodiments of the present invention. The following examples were carried out using standard techniques that are well known and routine to those of skill in the art, except where otherwise described in detail.

### Example 1: hGLPR/hGIPR/hNPY2R CreLuc Assay in 0.5% and 100% Plasma

CHO and HEK-293 recombinant cell lines express a luciferase reporter gene under control of the cAMP responsive element (CRE). As a second recombinant protein the GLP-1 and NPY2 receptors were expressed in the reporter gene HEK cells, and the GIP receptor was expressed in the reporter gene CHO cells, respectively. Stimulation of these recombinant cells with an agonist leads to an increase of intracellular cAMP levels. In the presence of cAMP, the transcription factor CRE binding protein (CREB) binds to the CRE and to the CREB binding protein (CBP), which leads to transcription of the luciferase reporter gene. Peptides upon serial-dilution in 100% DMSO were transferred into 384-well assay plates with 5 μl pre-dispensed assay buffer (1×HBSS, 20 mM HEPES, pH7.4 supplemented with 0.5% human plasma) using acoustic dispensing on a Labcyte ECHO. Cryo-preserved transgenic reporter gene cells were thawed in assay buffer. 20 μl (10.000 cells/well) were added to the plate with the peptides and incubated for 4 hours at 37° C. in a humidified incubator. Following incubation, assay plates are equilibrated to room temperature, followed by addition of 25 μl of Bright-GIo™ Luciferase reagent, incubation at room temperature for 10 minutes and analysis of luminescence (Envision Reader). Concentration response evaluation of compounds was performed with 8 concentrations of peptides (covering four decades). EC50 values were calculated by non-linear regression using sigmoid concentration-response with variable slope. The same assays were run in the presence of 100% plasma with no additional buffer constituents.

The results are summarized in Table 3, below.

TABLE 3

| | hGLP1R | hGLP1R | hGIPR | hGIPR | hNPY2R | hNPY2R |
|---|---|---|---|---|---|---|
| | 0.5% hP | 100% hP | 0.5% hP | 100% hP | 0.5% hP | 100% hP |
| Compound | (nM) | (nM) | (nM) | (nM) | (nM) | (nM) |
| 2 | 0.1 | 1.39 | 0.35 | 1.56 | >99.95 | 563.5 |
| 3 | 0.45 | 14.42 | 0.53 | 6.56 | 4.45 | 22.99 |
| 4 | 0.09 | 1.74 | 0.25 | 1.66 | 66.41 | 182.42 |
| 5 | 0.11 | 1.43 | 0.17 | 1.07 | >99.92 | >999.16 |
| 6 | 0.9 | 42.8 | 2.99 | 31.07 | 16.85 | 86.7 |
| 7 | 0.13 | 1.58 | 0.23 | 0.63 | 16.88 | 36.7 |
| 8 | 0.04 | 1.05 | 0.12 | 1.72 | 11.56 | 43.04 |
| 9 | 0.46 | 6.64 | 0.21 | 2.03 | 15.18 | 18.94 |
| 10 | 0.11 | 2.59 | 0.07 | 1.25 | 18.19 | 42.16 |
| 11 | 0.11 | 6.69 | 0.17 | 2.54 | 10.31 | 51.52 |
| 12 | 0.11 | 4.76 | 0.14 | 1.8 | 11.91 | 45.76 |
| 13 | 0.05 | 0.92 | 0.09 | 0.77 | 9.54 | 31.52 |
| 14 | 0.4 | 7.13 | 0.71 | 2.22 | 6.85 | 28.5 |
| 15 | 0.16 | 4.11 | 0.32 | 2.43 | 6.01 | 15.43 |
| 16 | 1.48 | 30.32 | 0.66 | 4.53 | 28.83 | 197.24 |
| 17 | 0.14 | 2.28 | 0.43 | 2.23 | >100.05 | 449.79 |
| 18 | 0.12 | 1.94 | 0.36 | 1.98 | 85.67 | 223.56 |
| 19 | 0.5 | 10.16 | 0.61 | 6.5 | 9.41 | 19.34 |
| 20 | 0.87 | 24.33 | 0.93 | 7.75 | 19.08 | 62.04 |
| 21 | 0.11 | 1.66 | 0.14 | 0.87 | >99.92 | >999.16 |
| 22 | 0.45 | 15.88 | 0.54 | 6.62 | 8.06 | 21.34 |
| 23 | 0.47 | 37.69 | 0.56 | 11.44 | 10.14 | 72.44 |
| 24 | 1.77 | 15.33 | 4.1 | 17.68 | 26.79 | 94.47 |
| 25 | 0.67 | 14.24 | 0.98 | 6.97 | 12.97 | 38.26 |
| 26 | 0.07 | 1.18 | 0.09 | 0.7 | >100.10 | 670.27 |
| 27 | 0.1 | 1.97 | 0.17 | 1.11 | 7.16 | 21.01 |
| 28 | 0.22 | 6.51 | 0.38 | 3.08 | 10.35 | 46.01 |
| 29 | 0.13 | 6.74 | 0.23 | 4.08 | 8.79 | 74.5 |
| 30 | 0.08 | 1.08 | 0.13 | 1.06 | 13.29 | 56.25 |
| 31 | 0.06 | 2.94 | 0.15 | 2.63 | 13.4 | 43.46 |
| 32 | 0.07 | 4.74 | 0.16 | 1.15 | 12.23 | 49.28 |
| 33 | 0.14 | 3.17 | 0.25 | 1.51 | 9.93 | 26.68 |
| 34 | 0.09 | 1.39 | 0.13 | 1.61 | 14.56 | 66.74 |
| 35 | 0.07 | 1.3 | 0.06 | 0.96 | 11.57 | 29.32 |
| 36 | 0.28 | 7.78 | 0.26 | 1.57 | 8.03 | 26.82 |
| 37 | 0.11 | 5.13 | 0.24 | 1.44 | 9.53 | 35.54 |
| 38 | 0.06 | 1.28 | 0.04 | 0.7 | 6.9 | 17.77 |
| 39 | 0.09 | 0.82 | 0.06 | 0.4 | 26.93 | 61.47 |
| 40 | 0.46 | 9.04 | 0.48 | 4.36 | 11.9 | 26.39 |
| 41 | 0.06 | 1.47 | 0.07 | 0.62 | 10.2 | 34.65 |
| 42 | 0.36 | 7.15 | 0.15 | 1.34 | 8.82 | 19.05 |
| 43 | 0.41 | 5.91 | 0.64 | 1.97 | 13.06 | 21.09 |
| 44 | 0.07 | 2.12 | 0.05 | 1.14 | 9.35 | 24.5 |
| 45 | 0.11 | 1.6 | 0.14 | 1.15 | 17.22 | 46.52 |
| 46 | 0.21 | 5.94 | 0.2 | 1.21 | 10.17 | 35.98 |
| 47 | 0.07 | 1.14 | 0.25 | 0.73 | 7.9 | 30.51 |
| 48 | 0.07 | 3.1 | 0.07 | 0.68 | 9.77 | 35.17 |
| 49 | 0.12 | 2.08 | 0.07 | 1.25 | 12.48 | 36.76 |
| 50 | 0.13 | 3.75 | 0.12 | 1.47 | 11.76 | 52.46 |
| 51 | 0.15 | 4.66 | 0.23 | 1.37 | 13.75 | 42.17 |
| 52 | 0.09 | 3.17 | 0.12 | 0.65 | 18.67 | 93.48 |
| 53 | 0.09 | 3.46 | 0.13 | 1.38 | 13.22 | 50.59 |
| 54 | 0.22 | 7.71 | 0.24 | 1.4 | 14.04 | 60.15 |
| 55 | 0.09 | 4.17 | 0.12 | 0.82 | 16.02 | 53.38 |
| 56 | 0.25 | 8.97 | 0.27 | 1.02 | 27.25 | 48.76 |
| 57 | 0.14 | 6.45 | 0.18 | 1.17 | 9.74 | 33.63 |
| 58 | 0.05 | 2 | 0.24 | 0.31 | 10.18 | 22.77 |
| 59 | 0.39 | 8.81 | 0.49 | 5.75 | 10.8 | 29.93 |
| 60 | 0.08 | 1.69 | 0.33 | 0.35 | 7.43 | 23.08 |
| 61 | 0.11 | 3.95 | 0.36 | 0.56 | 6.25 | 37.56 |
| 62 | 0.78 | 9.23 | 0.48 | 5.35 | 13.59 | 40.54 |
| 63 | 0.08 | 3 | 0.13 | 0.89 | 15.19 | 74.32 |
| 64 | 0.29 | 11.18 | 0.18 | 2.37 | 16.98 | 95.34 |
| 65 | 2.65 | 49.32 | 1.03 | 15.73 | 10.95 | 24.23 |
| 66 | 0.24 | 11.6 | 0.64 | 3.06 | 18.75 | 85.56 |
| 67 | 0.38 | 27.37 | 0.29 | 6.18 | 13.7 | 78.86 |
| 68 | 0.12 | 2.19 | 0.32 | 2.05 | 53.9 | 139.48 |
| 69 | 1.48 | 27.82 | 0.8 | 2.95 | 8.41 | 26.57 |
| 70 | 0.3 | 22.18 | 0.28 | 2.62 | 16.25 | 67.23 |
| 71 | 0.14 | 1.13 | 0.13 | 0.64 | >99.96 | >999.60 |
| 72 | 0.15 | 2.87 | 0.29 | 2.12 | 29.08 | 148.95 |
| 73 | 0.19 | 4.76 | 0.11 | 1.61 | 32.24 | 107.04 |
| 74 | 0.08 | 1.13 | 0.1 | 0.62 | 53.38 | 110.3 |
| 75 | 0.35 | 23.86 | 0.54 | 12.44 | 7.12 | 33.47 |
| 76 | 0.1 | 1.91 | 0.2 | 1.39 | 31.47 | 157.58 |
| 77 | 0.24 | 16.18 | 0.32 | 2.16 | 13.74 | 46.31 |
| 78 | 0.64 | 12.22 | 0.53 | 6.64 | 12.86 | 24.61 |
| 79 | 0.11 | 2.09 | 0.29 | 1.86 | 87.62 | 265.3 |
| 80 | 1.6 | 16.33 | 0.79 | 6.76 | 12.83 | 54.79 |
| 81 | 0.6 | 15.36 | 0.27 | 1.79 | 11.07 | 36.36 |
| 82 | — | — | — | — | — | — |
| 83 | 0.17 | 2.87 | 0.13 | 1.06 | 28.8 | 80.57 |
| 84 | 0.13 | 2.63 | 0.23 | 1.43 | 8.31 | 35.22 |
| 85 | 0.15 | 2.75 | 0.22 | 1.11 | 10.29 | 34.38 |
| 86 | 0.25 | 2.47 | 0.48 | 1.61 | 23.73 | 53.15 |
| 87 | 0.08 | 2 | 0.58 | 5.65 | 20.73 | 73.38 |
| 88 | 1.12 | 7.18 | 0.53 | 1.58 | 20.42 | 45.44 |
| 89 | 0.25 | 8.9 | 1.01 | 1.7 | 14.82 | 42.38 |
| 90 | 0.3 | 5.39 | 1.03 | 6.75 | 18.37 | 71.89 |
| 91 | 0.17 | 4.16 | 0.21 | 1.72 | 16.19 | 53.12 |
| 92 | 0.08 | 3.16 | 0.33 | 1.64 | 7.48 | 27.94 |
| 93 | 0.22 | 8.97 | 0.14 | 2.35 | 15.3 | 85.73 |
| 94 | 0.09 | 1.98 | 0.22 | 1.04 | 12.27 | 40.19 |
| 95 | 0.13 | 2.76 | 0.13 | 0.75 | 20.62 | 56.94 |
| 96 | 0.14 | 2.12 | 0.19 | 1.07 | 15.5 | 44.84 |
| 97 | 0.15 | 3.08 | 0.14 | 0.95 | 14.91 | 70.4 |
| 98 | 0.31 | 8.22 | 0.44 | 3.41 | 15.05 | 64.19 |
| 99 | 0.41 | 6.72 | 0.91 | 8.52 | 16.79 | 85.9 |
| 100 | 0.12 | 2.21 | 0.1 | 0.87 | 11.18 | 47.96 |
| 101 | 0.1 | 2.5 | 0.15 | 1.43 | 12.06 | 26.53 |
| 102 | 0.33 | 3.48 | 0.98 | 4.01 | 24.85 | 80.66 |
| 103 | 0.13 | 3.19 | 0.26 | 1.73 | 11.97 | 50.66 |
| 104 | 0.09 | 2.06 | 0.06 | 0.6 | 13.36 | 50.74 |
| 105 | 0.14 | 1.66 | 0.2 | 0.87 | 10.41 | 68.98 |
| 106 | 0.14 | 3.56 | 0.26 | 2.17 | 20.51 | 74.23 |
| 107 | 0.13 | 1.88 | 0.27 | 0.99 | 16.66 | 58.46 |
| 108 | 0.4 | 6.16 | 0.3 | 2.14 | 14.75 | 57.36 |
| 109 | 0.28 | 3.32 | 0.09 | 0.64 | 10.85 | 52.24 |
| 110 | 0.15 | 1.53 | 0.15 | 1.14 | 17.48 | 42.54 |
| 111 | 0.12 | 5.32 | 0.55 | 1.96 | 13.4 | 36.82 |
| 112 | 0.06 | 1.7 | 0.19 | 0.83 | 8.05 | 27.57 |
| 113 | 0.19 | 3.72 | 0.56 | 3.84 | 19.53 | 49.94 |
| 114 | 0.15 | 5.56 | 0.27 | 2.34 | 7.73 | 44.83 |
| 115 | 0.12 | 3.45 | 0.51 | 1.89 | 7.8 | 30.65 |
| 116 | 0.14 | 2.48 | 0.43 | 3.01 | 13.49 | 85.72 |
| 117 | 0.13 | 1.8 | 0.14 | 1.18 | 6.81 | 24.6 |
| 118 | 0.05 | 0.84 | 0.06 | 0.55 | 6.2 | 26.19 |
| 119 | 0.26 | 4.46 | 0.63 | 4.31 | 23.13 | 80.06 |
| 120 | 0.11 | 4.65 | 0.34 | 1.44 | 10.74 | 39.2 |
| 121 | 0.18 | 7.09 | 0.65 | 7.29 | 23.78 | 88.92 |
| 122 | 0.22 | 4.97 | 0.08 | 1.53 | 26.33 | 78.92 |
| 123 | 0.24 | 7.88 | 0.42 | 4.11 | 6.12 | 31.33 |
| 124 | 0.11 | 1.95 | 0.26 | 0.89 | 16.19 | 39.9 |
| 125 | 0.23 | 2.85 | 0.28 | 1.72 | 30.94 | 99.26 |
| 126 | 0.18 | 3.36 | 0.1 | 1.27 | 15.09 | 41.75 |
| 127 | 0.04 | 1.6 | 0.05 | 0.84 | 13.19 | 66.26 |
| 128 | 0.4 | 3.7 | 0.11 | 0.9 | 24.79 | 51.9 |
| 129 | 0.04 | 0.85 | 0.06 | 0.5 | 30.89 | 87.62 |
| 130 | 0.13 | 1.18 | 0.11 | 0.98 | 17.99 | 54.78 |
| 131 | 0.11 | 2.84 | 0.35 | 2.94 | 18.22 | 71.51 |
| 132 | 0.14 | 3.76 | 0.22 | 2.07 | 11.28 | 45.43 |
| 133 | 0.16 | 3.01 | 0.48 | 2.28 | 14.13 | 41.44 |
| 134 | 0.1 | 3.94 | 0.09 | 0.83 | 10.11 | 39.95 |
| 135 | 0.24 | 2.3 | 0.11 | 1.07 | 8.4 | 21.47 |
| 136 | 0.32 | 4.19 | 0.31 | 2.03 | 15.13 | 64.7 |
| 137 | 0.12 | 2.22 | 0.28 | 1.5 | 60.25 | 92.99 |
| 138 | 0.19 | 5.09 | 0.13 | 0.81 | 18.32 | 28.36 |
| 139 | 0.07 | 1.62 | 0.22 | 1.04 | 18.3 | 43.21 |
| 140 | 0.12 | 3.11 | 0.22 | 1.2 | 12.43 | 45.88 |
| 141 | 0.25 | 7.93 | 0.4 | 1.32 | 11.81 | 35.25 |
| 142 | 0.12 | 3.34 | 0.28 | 1.15 | 12.95 | 28.17 |
| 143 | 0.13 | 4.24 | 0.22 | 0.67 | 13.05 | 42.25 |
| 144 | 0.16 | 5.59 | 1.15 | 0.79 | 15.84 | 48.71 |
| 145 | 0.31 | 7.29 | 0.28 | 1.23 | 11.95 | 31.84 |
| 146 | 0.14 | 4.27 | 0.77 | 0.73 | 14.02 | 38.75 |
| 147 | 0.08 | 1.54 | 0.19 | 1.6 | 23.31 | 65.56 |
| 148 | 0.08 | 1.46 | 0.07 | 0.47 | 11.01 | 21.05 |
| 149 | 0.15 | 4.76 | 0.58 | 0.79 | 18.63 | 40.26 |
| 150 | 0.31 | 8.35 | 0.27 | 1.65 | 16.73 | 62.74 |
| 151 | 0.26 | 4.39 | 0.91 | 1.28 | 18.9 | 42.31 |
| 152 | 0.13 | 4.24 | 0.4 | 0.96 | 12.56 | 39.06 |
| 153 | 0.25 | 6.12 | 0.22 | 1.21 | 18.95 | 58.54 |
| 154 | 0.26 | 6.65 | 0.4 | 1.07 | 10.71 | 21.61 |
| 155 | 0.15 | 7.18 | 0.14 | 1.43 | 17.03 | 94.96 |
| 156 | 0.18 | 6.5 | 0.16 | 0.72 | 13.53 | 26.53 |
| 157 | 0.35 | 9.05 | 0.38 | 1.58 | 23.58 | 86.93 |
| 158 | 0.09 | 3.25 | 0.11 | 0.84 | 15.77 | 39.92 |
| 159 | 0.19 | 5.17 | 0.35 | 0.91 | 13.76 | 34.6 |
| 160 | 0.3 | 6.31 | 0.33 | 0.91 | 8.23 | 21.93 |
| 161 | 0.21 | 8.06 | 0.14 | 1.23 | 11.52 | 41.67 |
| 162 | 0.2 | 8.2 | 0.25 | 1.61 | 25.79 | 92.57 |
| 163 | 0.1 | 4.72 | 0.26 | 0.87 | 21.29 | 55.16 |
| 164 | 0.28 | 6.48 | 0.25 | 1.26 | 24.03 | 50.07 |
| 165 | 0.13 | 4.81 | 0.33 | 1.88 | 8.91 | 35.96 |
| 166 | 0.07 | 4.24 | 0.08 | 0.64 | 12.52 | 57.85 |
| 167 | 0.04 | 0.82 | 0.14 | 0.49 | 10.47 | 26.52 |
| 168 | 0.11 | 3.59 | 0.2 | 1.5 | 6.05 | 36.11 |
| 169 | 0.13 | 7.3 | 0.26 | 3.15 | 10.28 | 50.31 |
| 170 | 0.09 | 0.98 | 0.24 | 1.15 | 19.17 | 33.16 |
| 171 | 0.27 | 4.17 | 0.2 | 0.83 | 7.51 | 30.4 |
| 172 | 0.07 | 2 | 0.21 | 1.83 | 20.24 | 86.06 |
| 173 | 0.11 | 3.07 | 0.27 | 0.94 | 12.36 | 24.88 |
| 174 | 0.22 | 7.71 | 0.17 | 1.26 | 11.47 | 39.23 |
| 175 | 0.1 | 5.3 | 0.29 | 1.53 | 10.54 | 48.74 |
| 176 | 0.29 | 9.56 | 0.68 | 3.37 | 30.38 | 197.38 |
| 177 | 0.16 | 12.51 | 0.28 | 2.83 | 18.71 | 114.77 |
| 178 | 0.08 | 5.51 | 0.12 | 5.86 | 13.33 | 115.3 |
| 179 | 0.22 | 10.49 | 0.38 | 3.34 | 18.47 | 111.4 |
| 180 | 0.1 | 3.61 | 0.2 | 3.88 | 19.62 | 106.65 |
| 181 | 0.14 | 3.23 | 0.16 | 1.23 | 16.51 | 101.9 |
| 182 | 0.13 | 2.73 | 0.21 | 1.39 | 70.43 | 256.14 |
| 183 | 0.22 | 12.82 | 0.28 | 6.83 | 9.34 | 56.56 |
| 184 | 0.18 | 1.87 | 0.28 | 1.37 | 45.96 | 125.45 |
| 185 | 0.4 | 10.98 | 0.33 | 5.59 | 13.28 | 66.76 |
| 186 | 0.11 | 1.25 | 0.11 | 0.77 | >99.93 | 904.16 |
| 187 | — | — | — | — | — | — |
| 188 | 0.15 | 5.93 | 0.12 | 1.16 | 16.85 | 104.64 |
| 189 | 0.21 | 10.22 | 0.31 | 5.06 | 8.03 | 45.81 |
| 190 | 0.1 | 1.09 | 0.13 | 1.18 | >100.04 | >1,000.41 |
| 191 | 0.17 | 2.92 | 0.23 | 2 | >99.91 | >999.10 |
| 192 | 0.13 | 2.01 | 0.28 | 1.95 | 20.47 | 127.66 |
| 193 | 0.36 | 20.78 | 0.5 | 3.4 | 13.6 | 73.26 |
| 194 | 0.29 | 14.21 | 0.2 | 1.83 | 13.79 | 93.71 |
| 195 | 0.13 | 1.9 | 0.25 | 1.42 | 26.11 | 137.48 |
| 196 | 0.2 | 13.62 | 0.25 | 3.68 | 25.19 | 151.51 |
| 197 | 0.31 | 6.06 | 0.43 | 3.61 | 42.6 | 157.5 |
| 198 | 0.47 | 15.75 | 0.26 | 5.01 | 13.07 | 69.87 |
| 199 | 1.26 | 13.82 | 0.94 | 5.5 | 29.98 | 106.21 |
| 200 | 0.48 | 25.84 | 0.37 | 3.56 | 12.07 | 64.33 |
| 201 | 0.28 | 10.41 | 0.49 | 1.03 | 16.27 | 30.96 |
| 202 | 0.21 | 10.39 | 0.26 | 1.81 | 12.93 | 64.54 |
| 203 | 0.33 | 27.01 | 0.5 | 12.45 | 9.07 | 54.09 |
| 204 | 0.12 | 3.14 | 0.35 | 2.88 | >99.89 | >998.91 |
| 205 | 0.44 | 12.32 | 0.33 | 1.87 | 10.58 | 37.68 |
| 206 | 0.43 | 17.23 | 0.25 | 2.61 | 13.4 | 61.69 |
| 207 | 0.36 | 12.68 | 0.63 | 1.66 | 11.01 | 50.3 |
| 208 | 0.11 | 1.51 | 0.07 | 0.81 | >99.96 | >999.60 |
| 209 | 0.48 | 16.69 | 0.81 | 8.57 | 35.16 | 171.39 |
| 210 | 0.19 | 3.39 | 0.28 | 2.3 | 54.14 | 430.21 |
| 211 | 0.74 | 25.89 | 0.85 | 9.79 | 11.19 | 56.26 |
| 212 | 0.11 | 3.69 | 0.24 | 1.83 | 13.47 | 121.57 |
| 213 | 0.16 | 3.03 | 0.34 | 1.53 | >100.08 | >1,000.80 |
| 214 | 0.19 | 2.65 | 0.23 | 1.81 | >99.92 | 283.12 |
| 215 | 0.66 | 14.03 | 0.5 | 2.94 | 12.37 | 22.49 |
| 216 | 0.31 | 10.56 | 9.45 | 64.16 | 2.81 | 16.77 |
| 217 | 1.34 | 17.21 | 0.74 | 8.85 | 18.6 | 35.37 |
| 218 | 0.16 | 3.3 | 0.36 | 2.35 | 87.39 | 623.01 |
| 219 | 0.61 | 16.1 | 0.46 | 2.43 | 12.82 | 43.4 |
| 220 | 0.17 | 5.28 | 0.29 | 1.83 | 10.24 | 42.63 |
| 221 | 0.11 | 2.23 | 0.14 | 0.92 | 12.36 | 49.33 |
| 222 | 0.24 | 4.43 | 0.19 | 1.85 | 13.32 | 41.58 |
| 223 | 0.18 | 4.39 | 0.39 | 3.16 | 13.2 | 57.55 |
| 224 | 0.13 | 3.24 | 0.43 | 1.35 | 10.56 | 31.58 |
| 225 | 0.12 | 1.93 | 0.28 | 1.23 | 14.87 | 50.16 |
| 226 | 0.24 | 7.6 | 0.43 | 2.6 | 11.36 | 46.25 |
| 227 | 0.13 | 3.07 | 0.06 | 1.23 | 10.24 | 44.24 |
| 228 | 0.12 | 4.18 | 0.23 | 1.52 | 9.6 | 30.12 |
| 229 | 0.1 | 3.31 | 0.22 | 1.42 | 10.83 | 27.24 |
| 230 | 0.29 | 8.23 | 0.28 | 1.43 | 15.41 | 59.13 |
| 231 | 0.14 | 3.28 | 0.33 | 1.43 | 19.18 | 52.98 |
| 232 | 0.21 | 4.88 | 0.19 | 1.64 | 15.26 | 36.8 |
| 233 | 0.17 | 1.7 | 0.08 | 0.75 | 12.56 | 27.14 |
| 234 | 0.35 | 2.66 | 0.74 | 3.27 | 25.13 | 48.11 |
| 235 | 0.12 | 4.64 | 0.34 | 2.81 | 13.86 | 77.74 |
| 236 | 0.26 | 3.42 | 0.3 | 1.41 | 30.97 | 86.57 |
| 237 | 0.26 | 3.11 | 0.1 | 1.17 | 17.98 | 45.55 |
| 238 | 0.1 | 2.55 | 0.52 | 1.59 | 11.61 | 49.44 |
| 239 | 0.11 | 4.76 | 0.35 | 1.55 | 13.59 | 56.98 |
| 240 | 0.14 | 6.74 | 0.33 | 1.97 | 10.68 | 77.5 |
| 241 | 0.77 | 9.65 | 0.69 | 4.36 | 16.65 | 34.14 |
| 242 | 0.13 | 4.69 | 0.32 | 1.72 | 7.54 | 28.81 |
| 243 | 0.12 | 5.96 | 0.54 | 2.97 | 9.16 | 50.65 |
| 244 | 0.19 | 5.98 | 0.32 | 2.32 | 13.49 | 58.26 |
| 245 | 0.09 | 3.78 | 0.37 | 1.74 | 24.49 | 66.88 |
| 246 | 0.11 | 2.7 | 0.16 | 0.95 | 12.78 | 28.23 |
| 247 | 0.11 | 2.2 | 0.26 | 1.57 | 11.94 | 64.65 |
| 248 | 0.25 | 8 | 0.12 | 0.79 | 18.66 | 48.4 |
| 249 | 0.13 | 1.78 | 0.19 | 1.25 | 17.84 | 50 |
| 250 | 0.08 | 2.03 | 0.2 | 1.25 | 12.16 | 37.36 |
| 251 | 0.49 | 7.16 | 0.16 | 0.98 | 20.55 | 48.73 |
| 252 | 0.1 | 2.59 | 0.29 | 1.25 | 14.46 | 33.51 |
| 253 | 0.08 | 0.74 | 0.24 | 0.64 | 13.44 | 39.32 |
| 254 | 0.07 | 1.69 | 0.22 | 0.63 | 17.77 | 30.86 |
| 255 | 0.34 | 7.31 | 0.46 | 1.23 | 17.31 | 40.89 |
| 256 | 0.18 | 4.59 | 0.23 | 1.56 | 10.88 | 26.97 |
| 257 | 0.08 | 2.48 | 0.14 | 1.48 | 16.95 | 96.29 |
| 258 | 0.17 | 2.52 | 0.16 | 1.23 | 22.28 | 72 |
| 259 | 0.09 | 2.55 | 0.22 | 0.91 | 10.71 | 29.32 |
| 260 | 0.13 | 2.78 | 0.31 | 2.53 | 26.42 | 69.62 |
| 261 | 0.15 | 3.91 | 0.18 | 1.15 | 24.1 | 64.01 |
| 262 | 0.13 | 4.28 | 0.08 | 0.53 | 13.21 | 58.24 |
| 263 | 0.32 | 5.21 | 0.12 | 1.5 | 15.75 | 83.23 |
| 264 | 0.11 | 3.95 | 0.28 | 1.82 | 16.75 | 81.23 |
| 265 | 0.1 | 2.13 | 0.39 | 0.79 | 8.83 | 22.96 |
| 266 | 0.1 | 2.21 | 0.13 | 0.65 | 7.2 | 22.85 |
| 267 | 0.07 | 1.44 | 0.12 | 1.18 | 9.25 | 27.63 |
| 268 | 0.08 | 1.55 | 0.17 | 1.01 | 9.86 | 47.29 |
| 269 | 0.14 | 3.86 | 0.46 | 1.23 | 9.56 | 32.48 |
| 270 | 0.07 | 1.69 | 0.12 | 0.71 | 9.52 | 21.12 |
| 271 | 0.53 | 21.42 | 0.62 | 2.62 | 16.12 | 79.98 |
| 272 | 0.31 | 15.57 | 0.24 | 3.01 | 23.8 | 112.77 |
| 273 | 0.75 | 19.21 | 0.38 | 1.52 | 19.27 | 76.26 |
| 274 | 0.41 | 17.64 | 0.23 | 1.78 | 18.07 | 74.64 |
| 275 | 0.5 | 21.72 | 0.27 | 1.75 | 19.95 | 60.04 |
| 276 | 0.35 | 16.69 | 0.37 | 1.82 | 20.69 | 73.68 |
| 277 | 0.19 | 1.29 | 0.11 | 0.68 | >99.94 | 1,122.87 |
| 278 | 0.56 | 12.96 | 0.14 | 1.38 | 17.5 | 88.62 |
| 279 | 0.72 | 13.37 | 0.71 | 1.76 | 32.35 | 103.04 |
| 280 | 0.44 | 14.65 | 0.38 | 2.44 | 15.64 | 91.36 |
| 281 | 0.07 | 1.78 | 0.28 | 0.69 | 22.96 | 168.79 |
| 282 | 0.51 | 24.57 | 0.17 | 1.03 | 17.28 | 71.37 |
| 283 | 0.13 | 3.86 | 0.23 | 1.86 | 14.67 | 104.36 |
| 284 | 0.51 | 12.56 | 0.33 | 0.8 | 18.81 | 44.66 |
| 285 | 0.34 | 11.63 | 1.57 | 1.54 | 15.75 | 48.8 |
| 286 | 0.27 | 5.97 | 0.42 | 2.03 | 15.74 | 43.43 |
| 287 | 0.27 | 6.34 | 0.34 | 2.42 | 13.27 | 45.94 |
| 288 | 0.17 | 3.21 | 0.51 | 2.43 | 14.28 | 36.8 |
| 289 | 0.14 | 3.51 | 0.7 | 1.75 | 11.05 | 56.32 |
| 290 | 0.13 | 2.87 | 0.89 | 1.18 | 9.68 | 46.34 |
| 291 | 0.1 | 2.35 | 0.18 | 0.7 | 14.3 | 55.31 |
| 292 | 0.13 | 5.01 | 0.16 | 2.46 | 19.95 | 69.97 |
| 293 | 0.18 | 4.21 | 0.26 | 1.33 | 13.58 | 37.53 |
| 294 | 0.08 | 3.2 | 0.21 | 0.89 | 14.24 | 55.17 |
| 295 | 0.14 | 3.53 | 0.3 | 0.98 | 10.9 | 30.19 |
| 296 | 0.11 | 3.5 | 1.39 | 1.11 | 14.08 | 56.52 |
| 297 | 0.12 | 3.74 | 0.25 | 0.93 | 14.36 | 40.55 |
| 298 | 0.11 | 5.22 | 0.13 | 1.27 | 12.12 | 66.54 |
| 299 | 0.08 | 2.97 | 0.4 | 0.71 | 11 | 31.62 |
| 300 | 0.11 | 6.33 | 0.16 | 1.81 | 20.02 | 89.26 |
| 301 | 0.23 | 11.28 | 0.58 | 3.27 | 25.38 | 99.29 |
| 302 | 0.15 | 6.05 | 0.33 | 3.87 | 31.86 | 173.13 |
| 303 | 0.2 | 4.42 | 0.36 | 2.36 | 33.31 | 121.65 |
| 304 | 0.35 | 10.27 | 0.23 | 2.35 | 35.71 | 114.13 |
| 305 | 0.29 | 7.08 | 0.65 | 2.81 | 18.25 | 101.08 |

### Example 2: Solubility

Peptides (as TFA-salts) were weighed out in a filter unit (Mini-UniPrep Syringieless Filter 0.45 prn. Whatman PVDF), and 0.1 M phosphate buffer at pH 7 was added to achieve 10 mg/mL final peptide concentration. The peptides were dissolved by shaking the filter units horizontally at 600 rpm for 2 hours at room temperature. Samples were then filtered to remove any insoluble particles. Controls were prepared by weighing out the corresponding peptide solid material and dissolving it in an appropriate vehicle (e.g. ACN:H₂O) to a final concentration of 1 mg/mL. Both, the control and sample were analyzed by reversed phase chromatography. The area under the peak of the sample was compared to the control and the solubility was calculated based on that ratio. The pH was measured and recorded for each sample.

### Example 3: Mouse PK

Pharmacokinetic parameters of the peptides were determined after intravenous application to NMRI mice. Male NMRI mice were obtained from Janvier Laboratories (France) weighing 30 to 40 g. Mice were housed in standard cages with a 12:12 hour light:dark cycle and standard food and water access ad libitum. Each test peptide was dissolved in 50 mM phosphate buffer (pH 7.4)/3.5% mannitol. Intravenous application of 30-60 nmol/kg was performed via the tail vein. Serial blood samples from conscious mice were collected from the saphenous vein in EDTA-containing vials at different time points up to 56 hours post-dosing. Subsequently, plasma was prepared by centrifugation for 5 min at approximately 5000 rpm and stored at −20° C. until quantification of the peptide plasma levels by liquid chromatography mass spectrometry (LC-MS). Individual plasma concentration—time profiles were analysed by a non-compartmental approach and the resulting pharmacokinetic parameters were calculated. Mouse mean residence times (MRT) of GGY peptides according to the invention have been measured and summarized in table 4 (below).

TABLE 4

| Compound No | Mouse MRT (h) |
|---|---|
| 27 | 6.7 |
| 83 | 10.3 |
| 85 | 12.6 |
| 139 | 10 |
| 145 | 8 |
| 214 | 10.1 |
| 220 | 12.1 |
| 221 | 11 |
| 222 | 11.8 |
| 223 | 8.8 |
| 224 | 10.2 |
| 225 | 13 |
| 255 | 11 |
| 259 | 8.4 |
| 265 | 8.5 |
| 284 | 6.8 |
| 286 | 11 |
| 297 | 7.5 |
| Ref. 1 | <1 |
| Ref. 2 | 4.3 |

### Example 4: Effect on Acute Food Intake in Normal NMRI Mice

Male NMRI mice were obtained from Janvier (Janvier Labs, France) or from Charles River (Charles River Research Models & Services Germany GmbH) at the age of 3 weeks. The animals get a microchip (Datamars, Slim Microchip T-SL) for individual identification after delivery. They were housed with 4 mice per cage under a 12/12 dark-light cycle, light off at 3 p.m. Room temperature was controlled to 21° C. +/−1° C. and humidity 60%+/−20%. Mice had ad libitum access to regular rodent chow (KLIBA Nafag 3040 or Altromin 1324, Brogaarden, Denmark) and tap water.

Latest transfer into the HM2-System of MBRose, Denmark (real-time monitoring system of food- and water intake) of the animals 5 days before study start, to allow acclimatization to experimental conditions. As the animals were uniquely identified with the microchips, each individual animal was identified by its own microchip upon entry and exit from the food channel via antennae. Randomization of the mice for each study group (n=8; age earliest 6 weeks old) was based on food intake (middle of last three days 24 h-Intervall) and bodyweight directly before start of the study. A vehicle treated (50 mM phosphate buffer pH7.4 with 3.5% Mannitol) group was included in each experiment. To have the same nutrition standard for each animal the food access was locked eight hours before dark phase. One hour before night, the animals were once treated subcutaneously with the test peptide. Food intake was reported hourly for a period of 24 h. The food intake was normalized [%] to the average food intake of the vehicle group and the value are summarized in table 5 (below).

TABLE 5

| | Acute Food Intake 24 h |
|---|---|
| Compound No | 30 nmol/kg [% CTL] |
| 27 | 41 |
| 28 | 67 |
| 83 | 64 |
| 145 | 43 |
| 220 | 45 |
| 221 | 51 |
| 222 | 72 |
| 259 | 36 |
| 265 | 50 |
| 276 | 60 |
| 286 | 58 |

### Items A

1. A polypeptide according to the general structure of Formula (I) or a pharmaceutically acceptable salt thereof,

(I),
(SEQ ID NO: 647)
Z1-Z2-Z3

wherein,
Z1 is a hybrid polypeptide comprising the amino acid sequence, Y-Aib-X₃-G-T-F-T-S-D-X₁₀-S-I-X₁₃-L-X₁₅-X₁₆-X₁₇-A-X₁₉-X₂₀-X₂₁-F-X₂₃-X₂₄-X₂₅-L-X₂₇-K (SEQ ID NO: 646),
wherein X₃ is selected as E or D; X₁₀ is selected as Y or L; X₁₃ is selected as Aib or L; X₁₅ is selected as E, D, or A; X₁₆ is selected as K, E, D, Aib, G, LysAc, A, L, S, Q, or R; X₁₇ is selected as Q, E, K, or A; X₁₉ is selected as Q or E; X₂₀ is selected as Aib, D-Asp, or D-Arg; X₂₁ is selected as A, E or K; X₂₃ is selected as V or I; X₂₄ is selected as E or Q; X₂₅ is selected as W or Y; X₂₇ is I or L; or Z1 is a derivative thereof having 1 amino acid substitution;
Z2 is a linker comprising of the amino acid sequence G-G-X₃₁-X₃₂-X₃₃-X₃₄, wherein X₃₁ is selected as P, G, or Q; X₃₂ is selected as S, E, or R; X₃₃ is selected as S, E, Y, or T; X₃₄ is selected as G, P, Y, or A; or Z2 is a derivative thereof having 1 amino acid substitution;
Z3 is a polypeptide comprising the amino acid sequence,
X₃₅-X₃₆-X₃₇-X₃₈-X₃₉-Y-X₄₁-X₄₂-X₄₃-X₄₄-T-X₄₆-X₄₇-X₄₈-X₄₉,
wherein X₃₅ is selected as A, Q, I, V, E, or L; X₃₆ is selected as S, E, T, D, or L; X₃₇ is selected as L, Aib, V, D-Leu, I or Tle; X₃₈ is selected as R, L, or Aib; X₃₉ is selected as H, Aib, D-His, or Tle; X₄₁ is selected as Y, L, Aib, I, or Tle; X₄₂ is selected as N or Aib; X₄₃ is selected as W, H, L, R, or Aib; X₄₄ is selected as L, Aib, D-Arg, D-Asp, or Tle; X₄₆ is selected as R, hArg, or D-Arg; X₄₇ is selected as Q, bh-Gln, or NMeQ; X₄₈ is selected as R or NMeR; X₄₉ is selected as Y, Tle, Chg, D-Tyr, Phg; or Z3 is a derivative thereof having 1 amino acid substitution.

2. A polypeptide according to item 1, wherein X₃ in Z1 is selected as E.

3. A polypeptide according to any of the preceding items, wherein X₁₀ in Z1 is selected as Y.

4. A polypeptide according to any of the preceding items, wherein X₁₃ in Z1 is selected as Aib.

5. A polypeptide according to any of the preceding items, wherein X₁₅ in Z1 is selected as E.

6. A polypeptide according to any of the preceding items, wherein X₁₆ in Z1 is selected as K.

7. A polypeptide according to any of the preceding items, wherein X₁₇ in Z1 is selected as Q.

8. A polypeptide according to any of the preceding items, wherein X₁₉ in Z1 is selected as Q.

9. A polypeptide according to any of the preceding items, wherein X₂₀ in Z1 is selected as Aib.

10. A polypeptide according to any of the preceding items, wherein X₂₁ in Z1 is selected as A or E, preferably A.

11. A polypeptide according to any of the preceding items, wherein X₂₃ in Z1 is selected as V.

12. A polypeptide according to any of the preceding items, wherein X₂₄ in Z1 is selected as E or Q, preferably E.

13. A polypeptide according to any of the preceding items, wherein X₂₅ in Z1 is selected as W.

14. A polypeptide according to any of the preceding items, wherein X₂₇ in Z1 is selected as I.

15. A polypeptide according to any of the preceding items, wherein X₃₁ in Z2 is selected as P.

16. A polypeptide according to any of the preceding items, wherein X₃₂ in Z2 is selected as S.

17. A polypeptide according to any of the preceding items, wherein X₃₃ in Z2 is selected as E or S, preferably S.

18. A polypeptide according to any of the preceding items, wherein X₃₄ in Z2 is selected as G.

19. A polypeptide according to any of the preceding items, wherein X₃₅ in Z3 is selected as A, Q or V, preferably A.

20. A polypeptide according to any of the preceding items, wherein X₃₆ in Z3 is selected as S.

21. A polypeptide according to any of the preceding items, wherein X₃₇ in Z3 is selected as I or L, preferably L.

22. A polypeptide according to any of the preceding items, wherein X₃₈ in Z3 is selected as R.

23. A polypeptide according to any of the preceding items, wherein X₃₉ in Z3 is selected as H.

24. A polypeptide according to any of the preceding items, wherein X₄₁ in Z3 is selected as Y.

25. A polypeptide according to any of the preceding items, wherein X₄₂ in Z3 is selected as N.

26. A polypeptide according to any of the preceding items, wherein X₄₃ in Z3 is selected as W.

27. A polypeptide according to any of the preceding items, wherein X₄₄ in Z3 is selected as L.

28. A polypeptide according to any of the preceding items, wherein X₄₆ in Z3 is selected as R.

29. A polypeptide according to any of the preceding items, wherein X₄₇ in Z3 is selected as Q or NMeQ, preferably Q.

30. A polypeptide according to any of the preceding items, wherein X₄₈ in Z3 is selected as R or NMeR, preferably R.

31. A polypeptide according to any of the preceding items, wherein X₄₉ in Z3 is selected as Y.

32. A polypeptide according to any of the preceding items, wherein X₃₅X₃₆X₃₇X₃₈X₃₉ are selected as ASLRH (SEQ ID NO: 645).

33. A polypeptide according to any of the preceding items, wherein X₄₁X₄₂X₄₃X44 are selected as YNWL (SEQ ID NO: 326).

34. A polypeptide according to any of the preceding items, wherein X₄₆X₄₇X₄₈X₄₉ are selected as RQRY (SEQ ID NO: 327).

35. A polypeptide according to any of the preceding items, wherein the polypeptide is able to bind and/or activate the GLP-1, GIP and hY2 (hNPY2) receptors.

### Items B

1. A polypeptide according to the general structure of Formula (I) or a pharmaceutically acceptable salt thereof,

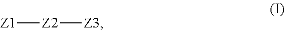

wherein,
Z1 is a hybrid polypeptide comprising the amino acid sequence,
Y-Aib-E-G-T-F-T-S-D-Y-S-I-Aib-L-E-K-Q-A-Q-Aib-A-F-V-E-W-L-I-K (SEQ ID NO: 306), or a derivative thereof having 1, 2, or 3 substitution(s);
Z2 is a linker;
Z3 is a polypeptide comprising the amino acid sequence,
A-S-L-R-H-Y-Y-N-W-L-T-R-Q-R-Y (SEQ ID NO: 307)
or a derivative thereof having 1, 2, 3, 4, or 5 substitution(s).

2. A polypeptide according to claim 1, wherein the linker consists of 1-10 amino acid residues.

3. A polypeptide according to any of the preceding claims, wherein Z2 has the amino acid sequence GGPSEG (SEQ ID NO: 311) or is a derivative thereof having 1, 2, 3, or 4 amino acid substitution(s).

4. A polypeptide according to claim 3, wherein the 1, 2, 3, or 4 amino acid substitution(s) in Z2 is/are present in any of the positions X₃₁, X₃₂, X₃₃, or X₃₄.

5. A polypeptide according to any of the preceding claims, wherein Z2 has the amino acid sequence GGX₃₁X₃₂X₃₃X₃₄, wherein X₃₁ is selected as P, G, or Q; X₃₂ is selected as S, E, or R; X₃₃ is selected as S, E, or Y; X₃₄ is selected as G, P, Y, or A; or is a derivative thereof having 1 amino acid substitution.

6. A polypeptide according to any of the preceding claims, wherein the 1, 2 or 3 substitution(s) in the derivate of Z1 is present in any of the amino acid positions X₃, X₁₀, X₁₃, X₁₅, X₁₆, X₁₇, X₁₉, X₂₀, X₂₁, X₂₃, X₂₄, X₂₅, or X₂₇.

7. A polypeptide according to any of the preceding claims, wherein the 1, 2, 3, 4, or 5 substitution(s) in the derivate of Z3 is/are present in any of the amino acid positions X₃₅, X₃₆, X₃₇, X₃₈, X₃₉, X₄₁, X₄₂, X₄₃, X₄₄, X₄₆, X₄₇, X₄₈, or X₄₉.

8. A polypeptide according to any of the preceding claims, wherein the derivate of Z3 has 1, 2, or 3 substitution(s), preferably 1 or 2 substitution(s), most preferably 1 substitution.

9. A polypeptide according to any of the preceding claims, wherein the derivate of Z1 has 1, or 2 substitution(s), preferably 1 substitution.

10. A polypeptide according to any of the preceding claims, wherein the substitution(s), in the derivate of Z1, is selected as follows:
X₃ is selected as D; X₁₀ is selected as L; X₁₃ is selected as L; X₁₅ is selected as D or A; X₁₆ is selected as E, D, Aib, G, LysAc, A, L, S, Q, or R; X₁₇ is selected as E, K, or A; X₁₉ is selected as E; X₂₀ is selected as D-Asp, or D-Arg; X₂₁ is selected as E; X₂₃ is selected as I; X₂₄ is selected as Q; X₂₅ is selected as Y; X₂₇ is selected as L.

11. A polypeptide according to any of the preceding claims, wherein the substitution(s), in the derivate of Z3, is selected as follows: X₃₅ is selected as Q, I, V, E, or L; X₃₆ is selected as E, T, D, or L; X₃₇ is selected as Aib, V, D-Leu, or I; X₃₈ is selected as L or Aib; X₃₉ is selected as Aib, D-His, or Tle; X₄₁ is selected as L, Aib, I, or Tle; X₄₂ is selected as Aib; X₄₃ is selected as H, L, R, or Aib; X₄₄ is selected as Aib, D-Arg, or D-Asp; X₄₆ is selected as hArg or D-Arg; X₄₇ is selected as bh-Gln or NMeQ; X₄₈ is selected as NMeR; X₄₉ is selected as Tle, Chg, D-Tyr, Phg.

12. A polypeptide according to any of the preceding claims, wherein the polypeptide is lipidated at a lysine (K) residue, preferably at the lysine (K) residue in amino acid position X₂₈.

13. A polypeptide according to any of the preceding claims, wherein the polypeptide is lipidated with a structure of general formula L-Y¹—Y²—Y³—Y⁴—Y⁵—Y⁶—Y⁷—Y⁸, wherein L is a lipid selected from C18DA or C20DA and further wherein each of Y¹—Y⁸ are independently selected from: not present, [γE], [OEG], [eLys], or [AHX].

14. A polypeptide according to any of the preceding claims, wherein the lipid is selected from a list consisting of C18DA, C18DA[γE]-, C18DA[γE][γE]-, C18DA[γE][γE][γE]-, C18DA[γE][γE][γE][γE]-, C18DA[γE][γE][γE][γE][γE]-, C18DA[γE][γE][γE][γE][γE][γE]-, C18DA[γE][γE][γE][γE][γE][γE][γE]-, C20DA, C20DA[γE]-, C20DA[γE][γE]-, C20DA[γE][γE][γE]-, C20DA[γE][γE][γE][γE]-, C20DA[γE][γE][γE][γE][γE]-, C20DA[γE][γE][γE][γE][γE][γE]-, C20DA[γE][γE][γE][γE][γE][γE][γE]-, C18DA[eLys], C18DA[γE][eLys]-, C18DA[γE][γE][eLys]-, C18DA[γE][γE][γE][eLys]-, C18DA[γE][γE][γE][γE][eLys]-, C18DA[γE][γE][γE][γE][γE][eLys]-, C18DA[γE][γE][γE][γE][γE][γE][eLys]-, C20DA[eLys], C20DA[γE][eLys]-, C20DA[γE][γE][eLys]-, C20DA[γE][γE][γE][eLys]-, C20DA[γE][γE][γE][γE][eLys]-, C20DA[γE][γE][γE][γE][γE][eLys]-, C20DA[γE][γE][γE][γE][γE][γE][eLys]-, C18DA[OEG][OEG]-, C18DA[γE][OEG][OEG]-, C18DA[γE][γE][OEG][OEG]-, C18DA[γE][γE][γE][OEG][OEG]-, C18DA[γE][γE][γE][γE][OEG][OEG]-, C18DA[γE][γE][γE][γE][γE][OEG][OEG]- , C20DA[OEG][OEG]-, C20DA[γE][OEG][OEG]-, C20DA[γE][γE][OEG][OEG]-, C20DA[γE][γE][γE][OEG][OEG]-, C20DA[γE][γE][γE][γE][OEG][OEG]-, C20DA[γE][γE][γE][γE][γE][OEG][OEG]-, C18DA[OEG]-, C18DA[γE][OEG]-, C18DA[γE][γE][OEG]-, C18DA[γE][γE][γE][OEG]-, C18DA[γE][γE][γE][γE][OEG]-, C18DA[γE][γE][γE][γE][γE][OEG]-, C18DA[γE][γE][γE][γE][γE][γE][OEG]-, C20DA[OEG]-, C20DA[γE][OEG]-, C20DA[γE][γE][OEG]-, C20DA[γE][γE][γE][OEG]-, C20DA[γE][γE][γE][γE][OEG]-, C20DA[γE][γE][γE][γE][γE][OEG]-, C20DA[γE][γE][γE][γE][γE][γE][OEG]-, C18DA[OEG][eLys]-, C18DA[γE][OEG][eLys]-, C18DA[γE][γE][OEG][eLys]-, C18DA[γE][γE][γE][OEG][eLys]-, C18DA[γE][γE][γE][γE][OEG][eLys]-, C18DA[γE][γE][γE][γE][γE][OEG][eLys]-, C20DA[OEG][eLys]-, C20DA[γE][OEG][eLys]-, C20DA[γE][γE][OEG][eLys]-, C20DA[γE][γE][γE][OEG][eLys]-, C20DA[γE][γE][γE][γE][OEG][eLys]-, C20DA[γE][γE][γE][γE][γE][OEG][eLys]-, C18DA[OEG][OEG][eLys]-, C18DA[γE][OEG][OEG][eLys]-, C18DA[γE][γE][OEG][OEG][eLys]-, C18DA[γE][γE][γE][OEG][OEG][eLys]-, C18DA[γE][γE][γE][γE][OEG][OEG][eLys]-, C20DA[OEG][OEG][eLys]-, C20DA[γE][OEG][OEG][eLys]-, C20DA[γE][γE][OEG][OEG][eLys]-, C20DA[γE][γE][γE][OEG][OEG][eLys]-, C20DA[γE][γE][γE][γE][OEG][OEG][eLys]-, C18DA[AHX], C18DA[γE][AHX]-, C18DA[γE][γE][AHX]-, C18DA[γE][γE][γE][AHX]-, C18DA[γE][γE][γE][γE][AHX]-, C18DA[γE][γE][γE][γE][γE][AHX]-, C18DA[γE][γE][γE][γE][γE][γE][AHX]-, C20DA[AHX], C20DA[γE][AHX]-, C20DA[γE][γE][AHX]-, C20DA[γE][γE][γE][AHX]-, C20DA[γE][γE][γE][γE][AHX]-, C20DA[γE][γE][γE][γE][γE][AHX]-, C20DA[γE][γE][γE][γE][γE][γE][AHX]-.

15. A polypeptide according to any of the preceding claims, wherein the polypeptide is selected from group consisting of Compound 1 to Compound 305.

### Item C

1. A polypeptide according to the general structure of Formula (I) or a salt or a pharmaceutically acceptable salt thereof,

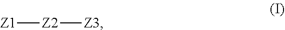

wherein,
Z1 is a hybrid polypeptide X₁-X₂₈ consisting of or comprising the amino acid sequence, Y-Aib-E-G-T-F-T-S-D-Y-S-I-Aib-L-E-K-Q-A-Q-Aib-A-F-V-E-W-L-I-K (SEQ ID NO: 306), or a derivative thereof having 1, 2, or 3 amino acid substitution(s);
Z2 is a peptide X₂₉-X₃₄ consisting of or comprising the amino acid sequence GGPSEG (SEQ ID NO: 311) or a derivative thereof having 1, 2, 3 or 4 amino acid substitution(s);
Z3 is a polypeptide X₃₅-X₄₉ consisting of or comprising the amino acid sequence, A-S-L-R-H-Y-Y-N-W-L-T-R-Q-R-Y (SEQ ID NO: 307)
or a derivative thereof having 1, 2, 3, 4, or 5 amino acid substitution(s);
preferably, wherein Z1-Z2-Z3 has 0, 1, 2, 3, 4, 5 or 6 amino acid substitutions.

2. A polypeptide according to item 1,
wherein Z1 consists of or comprises the amino acid sequence Y-Aib-E-G-T-F-T-S-D-Y-S-I-Aib-L-E-K-Q-A-Q-Aib-A-F-V-E-W-L-I-K (SEQ ID NO: 306) or is a derivative thereof having 1, 2 or 3 amino acid substitution(s) at any of the amino acid positions X₃, X₁₀, X₁₃, X₁₅, X₁₆, X₁₇, X₁₉, X₂₀, X₂₁, X₂₃, X₂₄, X₂₅, or X₂₇, and the substitution(s) is/are selected as follows: X₃ is selected as D; X₁₀ is selected as L; X₁₃ is selected as L; X₁₅ is selected as D or A; X₁₆ is selected as E, D, Aib, G, LysAc, A, L, S, Q, or R; X₁₇ is selected as E, K, or A; X₁₉ is selected as E; X₂₀ is selected as D-Asp, or D-Arg; X₂₁ is selected as E or K; X₂₃ is selected as I; X₂₄ is selected as Q; X₂₅ is selected as Y; X₂₇ is selected as L;
wherein Z2 consists of or comprises the amino acid sequence GGX₃₁X₃₂X₃₃X₃₄, wherein X₃₁ is selected as P, G, or Q; X₃₂ is selected as S, E, or R; X₃₃ is selected as S, E, Y, or T; X₃₄ is selected as G, P, Y, or A;
wherein Z3 consists of or comprises the amino acid sequence A-S-L-R-H-Y-Y-N-W-L-T-R-Q-R-Y (SEQ ID NO: 307) or is a derivative thereof having 1, 2, 3, 4, or 5 substitution(s) at any of the amino acid positions X₃₅, X₃₆, X₃₇, X₃₈, X₃₉, X₄₁, X₄₂, X₄₃, X₄₄, X₄₆, X₄₇, X₄₈, or X₄₉, and the substitution(s) is/are selected as follows: X₃₅ is selected as Q, I, V, E, or L; X₃₆ is selected as E, T, D, or L; X₃₇ is selected as Aib, V, D-Leu, I, or TIe; X₃₈ is selected as L or Aib; X₃₉ is selected as Aib, D-His, or TIe; X₄₁ is selected as L, Aib, I, or TIe; X₄₂ is selected as Aib; X₄₃ is selected as H, L, R, or Aib; X44 is selected as Aib, D-Arg, D-Asp, or Tle; X₄₆ is selected as hArg or D-Arg; X₄₇ is selected as bh-Gln or NMeQ; X₄₈ is selected as NMeR; X₄₉ is selected as TIe, Chg, D-Tyr, Phg.

3. A polypeptide according to any one of the preceding items,
wherein Z1 consists of or comprises the amino acid sequence Y-Aib-E-G-T-F-T-S-D-Y-S-I-Aib-L-E-K-Q-A-Q-Aib-A-F-V-E-W-L-I-K (SEQ ID NO: 306) or is a derivative thereof having 1, 2 or 3 amino acid substitution(s) at any of the amino acid positions X₃, X₁₀, X₁₃, X₁₅, X₁₆, X₁₇, X₁₉, X₂₀, X₂₁, X₂₃, X₂₄, X₂₅, or X₂₇, and the substitution(s) is/are selected as follows: X₃ is selected as D; X₁₀ is selected as L; X₁₃ is selected as L; X₁₅ is selected as D; X₁₆ is selected as E, D, G, A, S, Q, or R; X₁₇ is selected as E, or A; X₁₉ is selected as E; X₂₁ is selected as E or K; X₂₃ is selected as I; X₂₄ is selected as Q; X₂₅ is selected as Y; X₂₇ is selected as L;
wherein Z2 consists of or comprises the amino acid sequence G-G-X₃₁-X₃₂-X₃₃-X₃₄ wherein X₃₁ is selected as P or Q; X₃₂ is selected as S or E; X₃₃ is selected as S, E, Y or T; X₃₄ is selected as G, P, Y, or A;
wherein Z3 consists of or comprises the amino acid sequence A-S-L-R-H-Y-Y-N-W-L-T-R-Q-R-Y (SEQ ID NO: 307) or is a derivative thereof having 1, 2, or 3 substitution(s) at any of the amino acid positions X₃₅, X₃₆, X₃₇, X₃₈, X₃₉, X₄₁, X₄₂, X₄₃, X₄₄, X₄₆, X₄₇, X₄₈, or X₄₉, and the substitution(s) is/are selected as follows: X₃₅ is selected as Q, I, V, E, or L; X₃₆ is selected as T; X₃₇ is selected as Aib, V, I or TIe; X₃₈ is selected as Aib; X₃₉ is selected as Tle; X₄₁ is selected as I, L, or Tle; X44 is selected as Tle; X₄₇ is selected as N MeQ; X₄₈ is selected as NMeR.

4. A polypeptide according to any one of the preceding items,
wherein Z1 consists of or comprises the amino acid sequence Y-Aib-E-G-T-F-T-S-D-Y-S-I-Aib-L-E-K-Q-A-Q-Aib-A-F-V-E-W-L-I-K (SEQ ID NO: 306) or is a derivative thereof having 1, 2 or 3 amino acid substitution(s) at any of the amino acid positions X₃, X₁₀, X₁₃, X₁₅, X₁₆, X₁₇, X₂₁, X₂₃, X₂₄, or X₂₅, and the substitution(s) is/are selected as follows: X₃ is selected as D; X₁₀ is selected as L; X₁₃ is selected as L; X₁₅ is selected as D; X₁₆ is selected as E, D, G, A, S, Q, or R; X₁₇ is selected as E, or A; X₂₁ is selected as E; X₂₃ is selected as I; X₂₄ is selected as Q; X₂₅ is selected as Y;
wherein Z2 consists of or comprises the amino acid sequence G-G-X₃₁-X₃₂-X₃₃-X₃₄ wherein X₃₁ is selected as P or Q; X₃₂ is selected as S or E; X₃₃ is selected as S, E, Y or T; X₃₄ is selected as G, P, Y, or A;
wherein Z3 consists of or comprises the amino acid sequence A-S-L-R-H-Y-Y-N-W-L-T-R-Q-R-Y (SEQ ID NO: 307) or is a derivative thereof having 1, or 2 amino acid substitution(s) at any of the amino acid positions X₃₅, X₃₆, X₃₇, X₃₈, X₃₉, X₄₁, X44, X₄₇, X₄₈, or X₄₉, and the substitution(s) is/are selected as follows: X₃₅ is selected as Q, I, V, E, or L; X₃₆ is selected as T; X₃₇ is selected as Aib, V, I or TIe; X₃₈ is selected as Aib; X₃₉ is selected as Tle; X₄₁ is selected as I, L, or Tle; X44 is selected as Tle; X₄₇ is selected as N MeQ; X₄₈ is selected as NMeR.

5. A polypeptide according to any one of the preceding items,
wherein Z1 consists of or comprises the amino acid sequence Y-Aib-E-G-T-F-T-S-D-Y-S-I-Aib-L-E-K-Q-A-Q-Aib-A-F-V-E-W-L-I-K (SEQ ID NO: 306) or is a derivative thereof having 1 or 2 amino acid substitution(s), preferably 1, at any of the amino acid positions X₂₁, or X₂₄, and the substitution(s) is/are selected as follows: X₂₁ is selected as A or E; X₂₄ is selected as E or Q;
wherein Z2 consists of or comprises the amino acid sequence GGPSEG (SEQ ID NO: 311) or GGPSSG (SEQ ID NO: 320);
wherein Z3 consists of or comprises the amino acid sequence A-S-L-R-H-Y-Y-N-W-L-T-R-Q-R-Y (SEQ ID NO: 307) or is a derivative thereof having 1, or 2 amino acid substitution(s), preferably 1, at any of the amino acid positions X₃₅, X₃₇, X₄₇, or X₄₈, and the substitution(s) is/are selected as follows: X₃₅ is selected as Q, V; X₃₇ is selected as I; X₄₇ is selected as NMeQ; X₄₈ is selected as NMeR.

1. A polypeptide according to the general structure of Formula (I),
Z1-Z2-Z3 (I),
wherein,
Z1 is a hybrid polypeptide consisting of the amino acid sequence X1-X₂₈ Y-Aib-E-G-T-F-T-S-D-Y-S-I-Aib-L-E-K-Q-A-Q-Aib-A-F-V-E-W-L-I-K (SEQ ID NO: 306),
or a derivative thereof having 1, 2, or 3 amino acid substitution(s);
Z2 is a peptide consisting of the amino acid sequence X₂₉-X₃₄ GGPSEG (SEQ ID NO: 311),
or a derivative thereof having 1, 2, 3 or 4 amino acid substitution(s);
Z3 is a polypeptide consisting of the amino acid sequence X₃₅-X₄₉ A-S-L-R-H-Y-Y-N-W-L-T-R-Q-R-Y (SEQ ID NO: 307),
or a derivative thereof having 1, 2, 3, 4, or 5 amino acid substitution(s).

2. The polypeptide according to claim 1,
wherein in Z1 the 1, 2 or 3 amino acid substitution(s) is/are present at any of the amino acid positions X₃, X₁₀, X₁₃, X₁₅, X₁₆, X₁₇, X₁₉, X20, X₂₁, X₂₃, X₂₄, X₂₅, or X₂₇, and the substitution(s) is/are selected as follows: X₃ is selected as D; X10 is selected as L; X₁₃ is selected as L; X₁₅ is selected as D or A; X₁₆ is selected as E, D, Aib, G, LysAc, A, L, S, Q, or R; X₁₇ is selected as E, K, or A; X₁₉ is selected as E; X₂₀ is selected as D-Asp, or D-Arg; X₂₁ is selected as E or K; X₂₃ is selected as I; X₂₄ is selected as Q; X₂₅ is selected as Y; X₂₇ is selected as L;
wherein Z2 consist of the amino acid GGX₃₁X₃₂X₃₃X₃₄, wherein X₃₁ is selected as P, G, or Q;
X₃₂ is selected as S, E, or R; X₃₃ is selected as S, E, Y, or T; X₃₄ is selected as G, P, Y, or A;
wherein in Z3 the 1, 2, 3, 4, or 5 substitution(s) is/are present at any of the amino acid positions X₃₅, X₃₆, X₃₇, X₃₈, X₃₉, X₄₁, X₄₂, X₄₃, X₄₄, X₄₆, X₄₇, X₄₈, or X₄₉, and the substitution(s) is/are selected as follows: X₃₅ is selected as Q, I, V, E, or L; X₃₆ is selected as E, T, D, or L; X₃₇ is selected as Aib, V, D-Leu, I, or Tle; X₃₈ is selected as L or Aib; X₃₉ is selected as Aib, D-His, or Tle; X₄₁ is selected as L, Aib, I, or Tle; X₄₂ is selected as Aib; X₄₃ is selected as H, L, R, or Aib;
X₄₄ is selected as Aib, D-Arg, D-Asp, or Tle; X₄₆ is selected as hArg or D-Arg; X₄₇ is selected as bh-Gln or NMeQ; X₄₈ is selected as NMeR; X₄₉ is selected as Tle, Chg, D-Tyr, Phg.

3. The polypeptide according to claim 1, wherein in Z1 the 1, 2 or 3 amino acid substitution(s) is/are present in any of the amino acid positions X₃, X₁₀, X₁₃, X₁₅, X₁₆, X₁₇, X₁₉, X20, X₂₁, X₂₃, X₂₄, X₂₅, or X₂₇, and the substitution(s) is/are selected as follows: X₃ is selected as D; X10 is selected as L; X₁₃ is selected as L; X₁₅ is selected as D; X₁₆ is selected as E, D, G, A, S, Q, or R; X₁₇ is selected as E, or A; X₁₉ is selected as E; X₂₁ is selected as E or K; X₂₃ is selected as I; X₂₄ is selected as Q; X₂₅ is selected as Y; X₂₇ is selected as L;
wherein Z2 consists of the amino acid sequence G-G-X₃₁-X₃₂-X₃₃-X₃₄ wherein X31 is selected as P or Q; X₃₂ is selected as S or E; X₃₃ is selected as S, E, Y or T; X₃₄ is selected as G, P, Y, or A;
wherein in Z3 the 1, 2, or 3 substitution(s) is/are present in any of the amino acid positions X₃₅, X₃₆, X₃₇, X₃₈, X₃₉, X₄₁, X₄₂, X₄₃, X₄₄, X₄₆, X₄₇, X₄₈, or X₄₉, and the substitution(s) is/are selected as follows: X₃₅ is selected as Q, I, V, E, or L; X₃₆ is selected as T; X₃₇ is selected as Aib, V, I or Tle; X₃₈ is selected as Aib; X₃₉ is selected as Tle; X₄₁ is selected as I, L, or Tle; X₄₄ is selected as Tle; X₄₇ is selected as NMeQ; X₄₈ is selected as NMeR.

4. A polypeptide according to the general structure of Formula (I),
Z1-Z2-Z3 (I) (SEQ ID NO: 647),
wherein,
Z1 is a hybrid polypeptide consisting of the amino acid sequence,
(SEQ ID NO: 646)
Y-Aib-X₃-G-T-F-T-S-D-X₁₀-S-I-X₁₃-L-X₁₅-X₁₆-X₁₇-A-X₁₉-
X₂₀-X₂₁-F-X₂₃-X₂₄-X₂₅-L-X₂₇-K,
wherein X₃ is selected as E or D; X10 is selected as Y or L; X₁₃ is selected as Aib or L; X₁₅ is selected as E, D, or A; X₁₆ is selected as K, E, D, Aib, G, LysAc, A, L, S, Q, or R; X₁₇ is selected as Q, E, K, or A; X₁₉ is selected as Q or E; X₂₀ is selected as Aib, D-Asp, or D-Arg; X₂₁ is selected as A, E or K; X₂₃ is selected as V or I; X₂₄ is selected as E or Q; X₂₅ is selected as W or Y; X₂₇ is I or L;
Z2 is a linker consisting of the amino acid sequence G-G-X₃₁-X₃₂-X₃₃-X₃₄, wherein X₃₁ is selected as P, G, or Q; X₃₂ is selected as S, E, or R; X₃₃ is selected as S, E, Y, or T; X₃₄ is selected as G, P, Y, or A;
Z3 is a polypeptide consisting of the amino acid sequence,
X₃₅-X₃₆-X₃₇-X₃₈-X₃₉-Y-X₄₁-X₄₂-X₄₃-X44-T-X₄₆-X₄₇-X₄8-X₄₉,
wherein X₃₅ is selected as A, Q, I, V, E, or L; X₃₆ is selected as S, E, T, D, or L; X₃₇ is selected as L, Aib, V, D-Leu, I or Tle; X₃₈ is selected as R, L, or Aib; X₃₉ is selected as H, Aib, D-His, or Tle; X₄₁ is selected as Y, L, Aib, I, or Tle; X₄₂ is selected as N or Aib; X₄₃ is selected as W, H, L, R, or Aib; X₄₄ is selected as L, Aib, D-Arg, D-Asp, or Tle; X₄₆ is selected as R, hArg, or D-Arg; X₄₇ is selected as Q, bh-Gln, or NMeQ; X₄₈ is selected as R or NMeR; X₄₉ is selected as Y, Tle, Chg, D-Tyr, Phg.

5. The polypeptide according to claim 4, wherein in Z3
X₃₅ is selected as A, E, I, L, Q, and V; X₃₆ is selected as S or T; X₃₇ is selected as Aib, I, L, Tle and V; X₃₈ is selected as Aib or R; X₃₉ is selected as H or Tle; X₄₁ is selected as I, L, Tle or Y;
X₄₂ is selected as N; X₄₃ is selected as W; X₄₄ is selected as L or Tle; X₄₆ is selected as R; X₄₇ is selected as NMeQ or Q; X₄₈ is selected as NMeR or R; X₄₉ is selected as Y.

6. The polypeptide according to claim 4, wherein in Z1
X₃ is selected as E or D; X10 is selected as Y or L; X₁₃ is selected as Aib or L; X₁₅ is selected as E or D; X₁₆ is selected as K, E, D, G, A, S, Q, or R; X₁₇ is selected as Q, E, or A; X₁₉ is selected as E or Q; X₂₀ is selected as Aib; X₂₁ is selected as A, E or K; X₂₃ is selected as V or I; X₂₄ is selected as E or Q; X₂₅ is selected as W or Y; X₂₇ is selected as I or L.

7. The polypeptide according to claim 4, wherein Z2 consists of the amino acid sequence G-G-X₃₁-X₃₂-X₃₃-X₃₄ wherein X31 is selected as P or Q; X₃₂ is selected as S or E; X₃₃ is selected as S, E, Y or T; X₃₄ is selected as G, P, Y, or A; preferably, wherein Z2 is selected from the group consisting of: GGPEEG (SEQ ID NO: 313), GGPEEP (SEQ ID NO: 314), GGPESG (SEQ ID NO: 315), GGPESP (SEQ ID NO: 316), GGPSEG (SEQ ID NO: 311), GGPSEP (SEQ ID NO: 317), GGPSEY (SEQ ID NO: 318), GGPSSA (SEQ ID NO: 319), GGPSSG (SEQ ID NO: 320), GGPSSP (SEQ ID NO: 321), GGPSSY (SEQ ID NO: 322), GGPSTG (SEQ ID NO: 323), GGPSYG (SEQ ID NO: 324), GGQSSG (SEQ ID NO: 325), GGPRSG (SEQ ID NO: 650), GGPRYY (SEQ ID NO: 651), GGPSYY (SEQ ID NO: 652).

8. The polypeptide according to claim 4, wherein in Z1 X₃ is selected as E; X₁₀ is selected as Y; X₁₃ is selected as Aib; X₁₅ is selected as E; X₁₆ is selected as K; X₁₇ is selected as Q; X₁₉ is selected as Q; X₂₀ is selected as Aib; X₂₁ is selected as A or E; X₂₃ is selected as V; X₂₄ is selected as E or Q; X₂₅ is selected as W; X₂₇ is I;
wherein Z2 consists of the amino acid sequence GGPSEG (SEQ ID NO: 311) or GGPSSG (SEQ ID NO: 320);
wherein in Z3 X₃₅ is selected as A, Q or V; X₃₆ is selected as S; X₃₇ is selected as I or L; X₃₈ is selected as R; X₃₉ is selected as H; X₄₁ is selected as Y; X₄₂ is selected as N; X₄₃ is selected as W; X₄₄ is selected as L; X₄₆ is selected as R; X₄₇ is selected as Q or NMeQ; X₄₈ is selected as R or NMeR; X₄₉ is selected as Y.

9. The polypeptide according to claim 4, wherein the polypeptide is lipidated at a lysine (K) residue, preferably at the lysine (K) residue in amino acid position X₂₈.

10. The polypeptide according to claim 4, wherein the polypeptide is lipidated with a structure of general formula L-Y¹—Y²—Y³—Y⁴—Y⁵—Y⁶-Y⁷—Y⁸, wherein L is a lipid selected from 17-carboxy-heptadecanoyl (C18DA) and 19-carboxy-nonadecanoyl (C20DA), and wherein each of Y¹—Y⁸ are independently selected from: not present, [γE], [E], [OEG], [eLys], or [AHX].

11. The polypeptide according to claim 4, wherein the polypeptide is lipidated with a
a structure of general formula L-Y¹—Y²—Y³—Y⁴—Y⁵—Y⁶—Y⁷—Y⁸, wherein L is a lipid selected from 17-carboxy-heptadecanoyl (C18DA) and 19-carboxy-nonadecanoyl (C20DA), wherein each of Y¹—Y⁸ are independently selected from: not present, [γE], [E], [OEG], [eLys], or [AHX], wherein each of Y¹—Y³ are [γE] or each of Y¹—Y³ are [E], wherein Y⁴ is selected from: [γE], [E], or [OEG], and wherein each of Y⁵—Y⁸ are independently selected from: not present, [γE], [E], [OEG], [eLys], or [AHX].

12. The polypeptide according to claim 4, wherein the polypeptide is selected from group consisting of
SEQ ID NO: 328, SEQ ID NO: 329, SEQ ID NO: 330, SEQ ID NO: 331, SEQ
ID SEQ ID NO: 337, NO: 333, SEQ ID NO: 334, SEQ ID NO: 335, SEQ ID NO: 336, SEQ ID
SEQ ID NO: 338, SEQ ID NO: 339, SEQ ID NO: 340, SEQ ID NO: 341, SEQ ID
NO: 342, SEQ ID NO: 343, SEQ ID NO: 344, SEQ ID NO: 345, SEQ ID NO: 346, SEQ ID
NO: 347, SEQ ID NO: 348, SEQ ID NO: 349, SEQ ID NO: 350, SEQ ID NO: 351, SEQ ID
NO: 352, SEQ ID NO: 353, SEQ ID NO: 354, SEQ ID NO: 355, SEQ ID NO: 356, SEQ ID
NO: 357, SEQ ID NO: 358, SEQ ID NO: 359, SEQ ID NO: 360, SEQ ID NO: 361, SEQ ID
NO: 362, SEQ ID NO: 363, SEQ ID NO: 364, SEQ ID NO: 365, SEQ ID NO: 366, SEQ ID
NO: 367, SEQ ID NO: 368, SEQ ID NO: 369, SEQ ID NO: 370, SEQ ID NO: 371, SEQ ID
NO: 372, SEQ ID NO: 373, SEQ ID NO: 374, SEQ ID NO: 375, SEQ ID NO: 376, SEQ ID
NO: 377, SEQ ID NO: 378, SEQ ID NO: 379, SEQ ID NO: 380, SEQ ID NO: 381, SEQ ID
NO: 382, SEQ ID NO: 383, SEQ ID NO: 384, SEQ ID NO: 385, SEQ ID NO: 386, SEQ ID
NO: 387, SEQ ID NO: 388, SEQ ID NO: 389 and SEQ ID NO: 635, respectively (Y-Aib-E-G-T-F-
T-S-D-Y-S-I-Aib-L-E-K-Q-A-Q-Aib-A-F-V-Q-W-L-I-K(1,3)-G-G-P-E-S-G-A-S-L-R-H-Y-Y-
N-W-L-T-R-Q-R-Y-NH2.C18DA-E-E-E-E-E-eLys(1,2)), SEQ ID NO: 390, SEQ ID NO: 391,
SEQ ID NO: 392, SEQ ID NO: 393, SEQ ID NO: 394, SEQ ID NO: 395, SEQ ID NO: 396, SEQ
ID NO: 397, SEQ ID NO: 398, SEQ ID NO: 399, SEQ ID NO: 400, SEQ ID NO: 401, SEQ ID
NO: 402, SEQ ID NO: 403, SEQ ID NO: 404, SEQ ID NO: 405, SEQ ID NO: 406, SEQ ID
NO: 407, SEQ ID NO: 408, SEQ ID NO: 409, SEQ ID NO: 410, SEQ ID NO: 411, SEQ ID NO:
412, SEQ ID NO: 413, SEQ ID NO: 414, SEQ ID NO: 415, SEQ ID NO: 416, SEQ ID NO: 417,
SEQ ID NO: 418, SEQ ID NO: 419, SEQ ID NO: 420, SEQ ID NO: 421, SEQ ID NO: 422.
SEQ ID NO: 423, SEQ ID NO: 424, SEQ ID NO: 425, SEQ ID NO: 426, SEQ ID NO: 427,
SEQ ID NO: 428, SEQ ID NO: 429, SEQ ID NO: 430, SEQ ID NO: 431, SEQ ID NO: 432,
SEQ ID NO: 433, SEQ ID NO: 434, SEQ ID NO: 435, SEQ ID NO: 436, SEQ ID NO: 437,
SEQ ID NO: 438, SEQ ID NO: 439, SEQ ID NO: 450, SEQ ID NO: 451, SEQ ID NO: 452, SEQ
ID NO: 453, SEQ ID NO: 454, SEQ ID NO: 455, SEQ ID NO: 456, SEQ ID NO: 457, SEQ ID
NO: 458, SEQ ID NO: 459, SEQ ID NO: 460, SEQ ID NO: 461, SEQ ID NO: 462, SEQ ID NO:
463, SEQ ID NO: 464, SEQ ID NO: 465, SEQ ID NO: 466, SEQ ID NO: 467, SEQ ID NO: 468,
SEQ ID NO: 469, SEQ ID NO: 470, SEQ ID NO: 471, SEQ ID NO: 472, SEQ ID NO: 473, SEQ
ID NO: 474, SEQ ID NO: 475, SEQ ID NO: 476, SEQ ID NO: 477, SEQ ID NO: 478, SEQ ID
NO: 479, SEQ ID NO: 480, SEQ ID NO: 481 and SEQ ID NO: 635, respectively (Y-Aib-E-G-T-
F-T-S-D-Y-S-I-Aib-L-E-K-Q-A-Q-Aib-A-F-V-Q-W-L-I-K(1,3)-G-G-P-E-E-G-A-S-L-R-H-Y-
Y-N-W-L-T-R-Q-R-Y-NH2.C18DA-E-E-E-E-E-eLys(1,2)), SEQ ID NO: 482, SEQ ID NO:
483, SEQ ID NO: 484, SEQ ID NO: 485, SEQ ID NO: 486, SEQ ID NO: 487, SEQ ID NO: 488,
SEQ ID NO: 489, SEQ ID NO: 490, SEQ ID NO: 491, SEQ ID NO: 492, SEQ ID NO: 493, SEQ
ID NO: 494, SEQ ID NO: 495, SEQ ID NO: 496, SEQ ID NO: 497, SEQ ID NO: 498, SEQ ID
NO: 499, SEQ ID NO: 500, SEQ ID NO: 501, SEQ ID NO: 502 and SEQ ID NO: 636,
respectively (Y-Aib-E-G-T-F-T-S-D-Y-S-I-Aib-L-E-K-Q-A-Q-Aib-E-F-V-Q-W-L-I-K(1,3)-G-
G-P-S-E-G-A-S-L-R-H-Y-Y-N-W-L-T-R-Q-R-Y-NH2.C18DA-E-E-E-E-E(1,2)), SEQ ID NO:
503 and SEQ ID NO: 634, respectively (Y-Aib-E-G-T-F-T-S-D-Y-S-I-Aib-L-E-K-Q-A-Q-Aib-
A-F-V-Q-W-L-I-K(1,3)-G-G-P-E-E-G-A-S-L-R-H-Y-Y-N-W-L-T-R-Q-R-Y-NH2.C18DA-E-E-
E-E-OEG-OEG(1,2)), SEQ ID NO: 504, SEQ ID NO: 505 and SEQ ID NO: 637, respectively,
(Y-Aib-E-G-T-F-T-S-D-Y-S-I-Aib-L-E-K-Q-A-Q-Aib-E-F-V-Q-W-L-I-K(1,3)-G-G-P-S-E-G-
A-S-L-R-H-Y-Y-N-W-L-T-R-Q-R-Y-NH2.C18DA-E-E-E-E-E-E(1,2)), SEQ ID NO: 506, SEQ
ID NO: 507, SEQ ID NO: 508, SEQ ID NO: 509, SEQ ID NO: 510, SEQ ID NO: 511, SEQ ID
NO: 512, SEQ ID NO: 513, SEQ ID NO: 514, SEQ ID NO: 515, SEQ ID NO: 516, SEQ ID NO:
517, SEQ ID NO: 518, SEQ ID NO: 519, SEQ ID NO: 520, SEQ ID NO: 521, SEQ ID NO: 522
and SEQ ID NO: 643, respectively (Y-Aib-E-G-T-F-T-S-D-Y-S-I-Aib-L-E-K-Q-A-Q-Aib-A-F-
V-Q-W-L-I-K(1,3)-G-G-P-E-E-G-A-S-L-R-H-Y-Y-N-W-L-T-R-Q-R-Y-NH2.C18DA-E-E-E-E-
E-E-eLys(1,2)), SEQ ID NO: 523, SEQ ID NO: 524, SEQ ID NO: 525, SEQ ID NO: 526, SEQ
ID NO: 527, SEQ ID NO: 528, SEQ ID NO: 529, SEQ ID NO: 530, SEQ ID NO: 531, SEQ ID
NO: 532, SEQ ID NO: 533, SEQ ID NO: 534, SEQ ID NO: 535, SEQ ID NO: 536, SEQ ID NO:
537, SEQ ID NO: 538, SEQ ID NO: 539, SEQ ID NO: 540, SEQ ID NO: 541, SEQ ID NO: 542,
SEQ ID NO: 543, SEQ ID NO: 544, SEQ ID NO: 545, SEQ ID NO: 546, SEQ ID NO: 547,
SEQ ID NO: 548, SEQ ID NO: 549, SEQ ID NO: 550, SEQ ID NO: 551, SEQ ID NO: 552, SEQ
ID NO: 553, SEQ ID NO: 554, SEQ ID NO: 555, SEQ ID NO: 556, SEQ ID NO: 557, SEQ ID
NO: 558, SEQ ID NO: 559, SEQ ID NO: 560, SEQ ID NO: 561, SEQ ID NO: 562, SEQ ID NO:
563, SEQ ID NO: 564, SEQ ID NO: 565 and SEQ ID NO: 638, respectively (Y-Aib-E-G-T-F-T-
S-D-Y-S-I-Aib-L-E-K-Q-A-Q-Aib-A-F-V-Q-W-L-I-K(1,3)-G-G-P-S-E-G-A-S-L-R-H-Y-Y-N-
W-L-T-R-Q-R-Y-NH2.C20DA-E-E-E-E-OEG-OEG(1,2)), SEQ ID NO: 566 and SEQ ID
NO: 639, respectively, Y-Aib-E-G-T-F-T-S-D-Y-S-I-Aib-L-E-K-Q-A-Q-Aib-A-F-V-Q-W-L-I-
K(1,3)-G-G-P-S-E-G-A-S-L-R-H-Y-Y-N-W-L-T-R-Q-R-Y-NH2.C20DA-E-E-E-E-E-
eLys(1,2)), SEQ ID NO: 567, SEQ ID NO: 568, SEQ ID NO: 569, SEQ ID NO: 570, SEQ ID
NO: 571, SEQ ID NO: 572, SEQ ID NO: 573, SEQ ID NO: 574, SEQ ID NO: 575 and SEQ ID
NO: 643, respectively (Y-Aib-E-G-T-F-T-S-D-Y-S-I-Aib-L-E-K-Q-A-Q-Aib-A-F-V-Q-W-L-I-
K(1,3)-G-G-P-S-E-G-A-S-L-R-H-Y-Y-N-W-L-T-R-Q-R-Y-NH2.C18DA-E-E-E-E-E-E-
eLys(1,2)), SEQ ID NO: 576, SEQ ID NO: 577, SEQ ID NO: 578, SEQ ID NO: 579, SEQ ID
NO: 580, SEQ ID NO: 581, SEQ ID NO: 582, SEQ ID NO: 583 and SEQ ID NO: 636,
respectively (Y-Aib-E-G-T-F-T-S-D-Y-S-I-Aib-L-E-K-Q-A-Q-Aib-A-F-V-Q-W-L-I-K(1,3)-G-
G-P-S-E-G-A-S-L-R-H-Y-Y-N-W-L-T-R-Q-R-Y-NH2.C18DA-E-E-E-E-E(1,2)), SEQ ID NO:
584, SEQ ID NO: 585, SEQ ID NO: 586, SEQ ID NO: 587, SEQ ID NO: 588, SEQ ID NO: 589.
SEQ ID NO: 590 and SEQ ID NO: 637, respectively (Y-Aib-E-G-T-F-T-S-D-Y-S-I-Aib-L-E-K-
Q-A-Q-Aib-A-F-V-Q-W-L-I-K(1,3)-G-G-P-S-E-G-A-S-L-R-H-Y-Y-N-W-L-T-R-Q-R-Y-
NH2.C18DA-E-E-E-E-E-E(1,2)), SEQ ID NO: 591, SEQ ID NO: 592, SEQ ID NO: 593, SEQ
ID NO: 594, SEQ ID NO: 595, SEQ ID NO: 596, SEQ ID NO: 597, SEQ ID NO: 598, SEQ ID
NO: 599, SEQ ID NO: 600, SEQ ID NO: 601, SEQ ID NO: 602, SEQ ID NO: 603, SEQ ID NO:
604, SEQ ID NO: 605, SEQ ID NO: 606, SEQ ID NO: 607 and SEQ ID NO: 640, respectively
(Y-Aib-E-G-T-F-T-S-D-Y-S-I-Aib-L-E-K-Q-A-Q-Aib-A-F-V-Q-W-L-I-K(1,3)-G-G-P-S-E-G-
A-S-L-R-H-Y-Y-N-W-L-T-R-Q-R-Y-NH2.C18DA-E-E-E-E(1,2)), SEQ ID NO: 608, SEQ ID
NO: 609, SEQ ID NO: 610, SEQ ID NO: 611, SEQ ID NO: 612, SEQ ID NO: 613, SEQ ID NO:
614, SEQ ID NO: 615, SEQ ID NO: 616, SEQ ID NO: 617 and SEQ ID NO: 640, respectively
(Y-Aib-E-G-T-F-T-S-D-Y-S-I-Aib-L-E-K-Q-A-Q-Aib-A-F-V-E-W-L-I-K(1,3)-G-G-P-S-E-G-
A-S-L-R-H-Y-Y-N-W-L-T-R-Q-R-Y-NH2.C18DA-E-E-E-E(1,2)), SEQ ID NO: 618, SEQ ID
NO: 619, SEQ ID NO: 620, SEQ ID NO: 621, SEQ ID NO: 622, SEQ ID NO: 623, SEQ ID NO:
624, SEQ ID NO: 625, SEQ ID NO: 626, SEQ ID NO: 627, SEQ ID NO: 628 and SEQ ID
NO: 637, respectively (Y-Aib-E-G-T-F-T-S-D-Y-S-I-Aib-L-E-K-Q-A-Q-Aib-A-F-V-E-W-L-I-
K(1,3)-G-G-P-S-E-G-A-S-L-R-H-Y-Y-N-W-L-T-R-Q-R-Y-NH2.C18DA-E-E-E-E-E-E(1,2)),
SEQ ID NO: 629, SEQ ID NO: 630, and SEQ ID NO: 631.

13. The polypeptide according to claim 4, wherein the polypeptide is in the form of a salt or in the form of a pharmaceutically acceptable salt.

14. The polypeptide according to claim 13, wherein the polypeptide or a pharmaceutically acceptable salt thereof is comprised in a pharmaceutical composition together with one or more pharmaceutically acceptable carrier and/or excipients.

15. A method of binding to and/or activating the GLP-1, GIP and/or hNPY2 receptors in a mammal, wherein the method comprises administering to a patient in need thereof a polypeptide according to claim 4.

16. A method to treat a disease, disorder, or condition, wherein the method comprises administering to a patent in need thereof one or more polypeptides according to claim 4, wherein the disease, disorder, or condition is one or more of:
overweight, chronic weight management, obesity, symptomatic obesity, eating disorders;
insulin resistance, diabetes, type 1 diabetes, type 2 diabetes, prediabetes;
endocrine obesity: Cushing syndrome, hypothyroidism, insulinoma, obese type 2 diabetes, pseudohypoparathyroidism, hypogonadism;
obesity associated endocrine disorders: polycystic ovary syndrome (PCOS);
central obesity: hypothalamic obesity, frontal lobe syndrome, Kleine-Levin syndrome;
hereditary obesity: Prader-Willi syndrome, Laurence-Moon-Biedl syndrome;
drug-induced obesity: steroid-induced obesity, phenothiazine-induced obesity, insulin-induced obesity, sulfonylurea agent-induced obesity, β-blocker-induced obesity;
co-morbidities of obesity and/or overweight: associated type 2 diabetes, associated high blood pressure, associated NAFLD, associated NASH, associated DKD, associated CKD, associated osteoporosis, associated sleep apnoe, associated cancers, associated asthma;
hyperlipidemia: hypertriglyceridemia, hypercholesterolemia, high LDL-cholesterolemia, low HDL-cholesterolemia, postprandial hyperlipemia;
metabolic syndrome: abdominal obesity, high blood pressure, high blood sugar, high serum triglycerides, low serum high-density lipoprotein (HDL);
liver diseases: metabolic associated fatty liver disease (MAFLD), non-alcoholic fatty liver disease (NAFLD), non-alcoholic steato-hepatitis (NASH), portal hypertension;
cardiovascular diseases: hypertension, atherosclerosis, stroke, cardiac failure;
kidney diseases: diabetic kidney disease (DKD), chronic kidney disease (CKD);
neurodegenerative diseases: Alzheimer's disease, or Parkinson's disease.
